(19) United States
(12) Patent Application Publication
(10) Pub. No.: US 2025/0163088 A1
(43) Pub. Date: May 22, 2025

(54) HALF-SANDWICH TRANSITION METAL COMPLEXES AND USES THEREOF

(71) Applicant: DEBRECENI EGYETEM, Debrecen (HU)

(72) Inventors: Péter BAY, Debrecen (HU); Éva BOKOR, Debrecen (HU); László SOMSÁK, Debrecen (HU); István KACSIR, Miskolc (HU); Péter BUGLYÓ, Debrecen (HU); Adrienn SIPOS, Debrecen (HU); Gábor KARDOS, Debrecen (HU); Zoltán TÓTH, Debrecen (HU); György Attila KIS, Debrecen (HU)

(21) Appl. No.: 18/693,144

(22) PCT Filed: Sep. 19, 2022
(86) PCT No.: PCT/HU2022/050067

§ 371 Date: Mar. 18, 2024

(30) Foreign Application Priority Data
Sep. 17, 2021 (HU) P2100326

(51) Int. Cl. C07F 17/02; A61K 31/555; A61P 31/04

(52) U.S. Cl. CPC C07F 17/02; A61K 31/555; A61P 31/04

(57) ABSTRACT

The invention relates to half-sandwich transition metal complexes and uses thereof, in particular in neo-plastic disease or as antifungal agent

This is the national stage of International Application PCT/HU2022/050067, filed Sep. 19, 2022.

### FIELD OF THE INVENTION

The invention relates to Half-sandwich transition metal complexes and uses thereof.

### BACKGROUND ART

Metal based drugs used in cancer therapy are square planar platinum(II) complexes, e. g. cisplatin (compound 1), oxaliplatin (2) and carboplatin (3) being registered worldwide. Although Pt complexes are versatile tools of the trade, their applicability has shortcomings, such as the development of platinum resistance in tumors (Holditch et al., 2019; Manohar and Leung, 2018; McMullen et al., 2020; Mukherjea et al., 2020; Yu et al., 2020), the latter two being very characteristic for cisplatin.

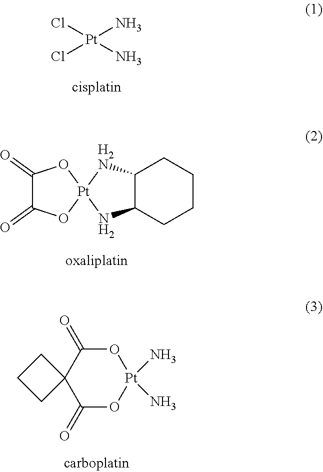

According to Ndagi Umar et al. (Ndagi et al., 2017), in anti-cancer Pt complexes, conjugation with sugars can result in a number of improved properties, e.g. improved solubility, reduction of adverse side effects, and improved uptake by cells.

Ruthenium complexes can be promising alternatives to platinum complexes due to their similar chemical characteristics in terms of ligand exchange (Kenny and Marmion, 2019). Importantly, multiple studies have pointed out the reduced toxicity for Ru complexes as compared to Pt complexes both in cellular and animal models (Gano et al., 2019; Liu et al., 2019; Mello-Andrade et al., 2018; Mihajlovic et al., 2020).

Among the Ru(III)- and Ru(II)-based complexes a great number of derivatives were tested as anticancer metallodrugs and two of them, the imidazolium salt of tetrachlorido(dimethylsulfoxide)(imidazole)ruthenium(III) (NAMI-A) (4) and sodium tetrachloridobis(indazole)ruthenium(III) (NKP1339) (5) reached clinical trials (Burris et al., 2016; Fernandes, 2019; Kenny and Marmion, 2019; Meier-Menches et al., 2018; Zeng et al., 2017). In good agreement with that NAMI-A (Leijen et al., 2015), further compounds KP1019/1339 (IT-139, BOLD100) (Burris et al., 2016) or TLD-1433 (Kulkarni et al., 2022) are also already in different phases of clinical trials against neoplastic diseases as bladder or lung cancer. Of note, NAMI-A and KP-1019 are monodentate (similar to cisplatin), while TLD-1433 is a bidentate complex.

The half-sandwich type Ru(II)-arene organometallics (Melchart and Sadler, 2006), due to a high structural variability, represent one of the most widely investigated compound classes in the development of new candidates of multi-targeted metallodrugs (Bononi et al., 2020; Kenny and Marmion, 2019). The presence of a η⁶-arene or η⁵-arenyl residue in the coordination sphere contributes to the stabilization of the +2 oxidation state of the metal ion and to the maintenance of the hydrophilic/lipophilic balance of the whole molecule. The remaining three coordination sites of the Ru(II) ion are usually occupied by at least one leaving group and mono- or bidentate ligands, as it can be seen in RAPTA-T and —C(6) and RM175 (7), respectively, which among others, have become leads for Ru(II)-based complexes (Fernandes, 2019; Habtemariam et al., 2006; Melchart and Sadler, 2006).

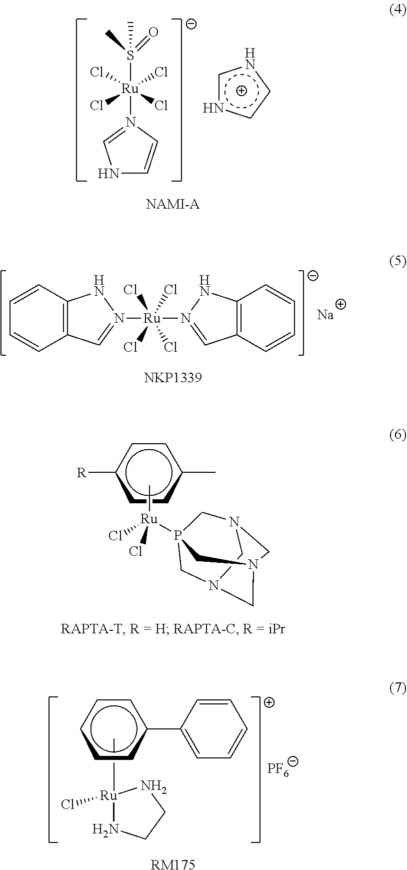

Due to the biological relevance of sugars, the incorporation of a carbohydrate-containing ligand into platinum group metal complexes in general (Hartinger et al., 2008; Kenny and Marmion, 2019), and into Ru(II)-arene/arenyl complexes in particular (Fernandes, 2019), seems a rational choice for further drug design. Thus, several functional features of carbohydrates, such as their contribution to different cellular phenomena (e.g. to cell-cell recognition and adhesion), their crucial role in cellular energy supply, their binding capacities to carbohydrate specific proteins (e.g. lectins, glucose transporters and glycoenzymes) can be exploited to obtain new platinum group metal complexes with anticancer potential (Fernandes, 2019; Hartinger et al., 2008).

For several sugar containing half-sandwich Ru(II) complexes, such as RAPTA analogues with glycofuranose-based phosphite ligands (Fernandes, 2019) (compound 8) and Ru(II) complexes having 2,3-diamino-2,3-dideoxy-hexopyranoside (Böge et al., 2015) (compound 9) or 1,4-bis(β-D-glycopyranosyl)tetrazene-type N,N-chelating ligands (Hamala et al., 2020) (compound 10), the antiproliferative activity has already been justified. Hamala and colleagues (Hamala et al., 2020) showed that the inhibitory activity of carbohydrate-based ruthenium complexes was enhanced when the hydroxyl groups of the sugar units were protected by esters and an increasing length of the acyl chain improved the inhibitory efficacy (acetyl <propionyl <butyryl). However, their compounds were non-selective and induced apoptotic ccll death at low micromolar concentrations.

In addition, certain cyclopentadienyl-ruthenium(II) complexes with sugar-based heterocyclic mono-(compound 11, compound 12) or bidentate ligands (compound 13) have also been shown to display low micromolar cytotoxic activity in human cervical carcinoma (HeLa) and colon cancer HCT116 cell lines (Florindo et al., 2014; Florindo et al., 2016; Florindo et al., 2015).

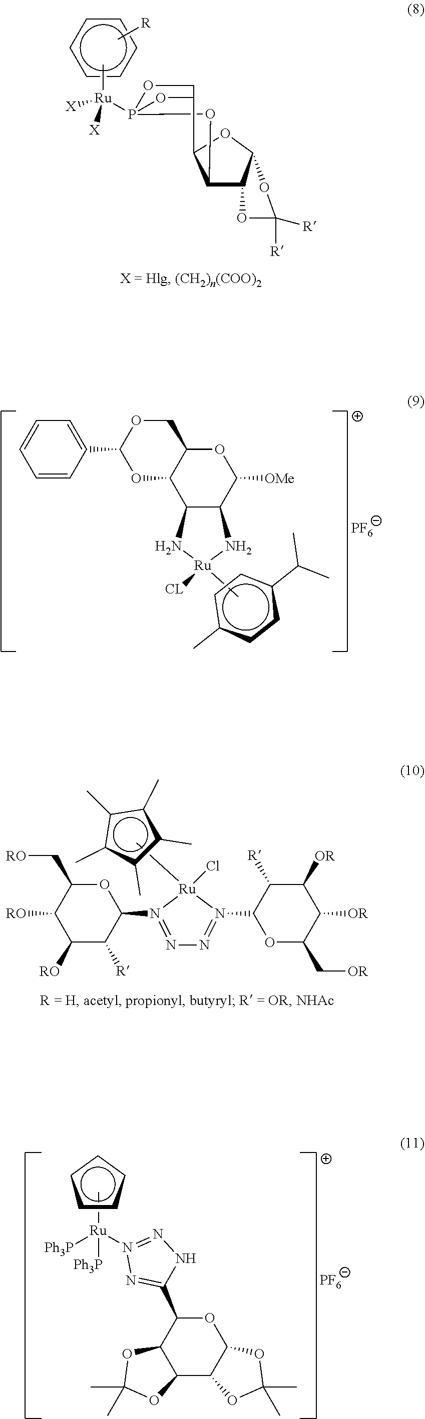

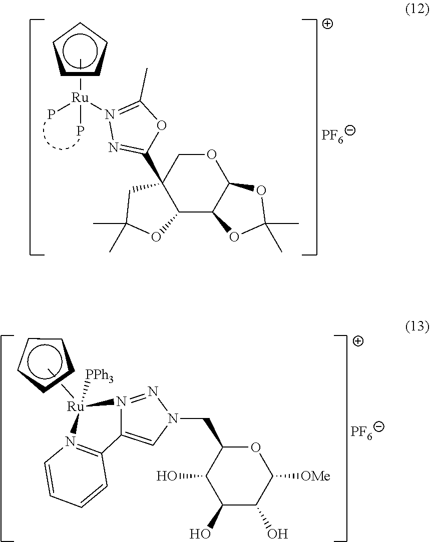

More closely, the compounds disclosed in (25) Florindo et al. (2016) comprise methyl-α-D-mannopyranoside and methyl-α-D-glucopyranoside as a monosaccharide unit without a protecting group (compound 13).

Xiao, N. et al. (Xiao et al., 2017) have synthesized homoleptic and heteroleptic complexes of Ru(TAGP-tapy)₃Cl₂ (Xiao et al., 2017-tetra-O-acetyl-β-D-glucopyranosyl)-1H-1,2,3-triazol-4-yl]-pyridine) bearing a clustered glucose-derived ligand and Ru(bpy)₂(TAGP-tapy)Cl₂ (bpy is 2,2′-bipyridine). One of the compounds [Ru(bpy)₂(TAGP-tapy)Cl₂, compound 14] comprises a triazole ring and a sugar unit with acetyl protecting groups. The authors do not mention that they would expect an antitumor effect from the synthesized compounds.

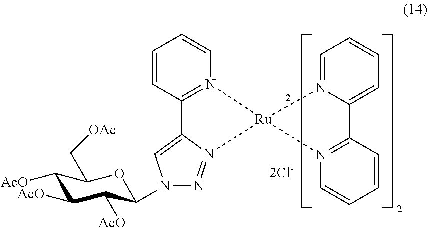

Similar ligands have been prepared and studied in the art.

Bokor E. et al. (Bokor et al., 2010) have searched for effective glycogen phosphorylase inhibitors, as glycogen phosphorylase is a validated drug target for type 2 diabetes mellitus and thus has the potential to provide new therapies for the disease. The authors used a copper(I)-catalyzed azide-alkyne cycloaddition reaction to produce O-acetyl- or O-benzoyl-protected 1-D-glucopyranosyl-1,2,3-triazoles. In the introduction, they describe that the use of the 1,2,3-triazole ring as an amide substitute is becoming more common because it is stable under many chemical conditions. It is mentioned that N—(β-D-glucopyranosyl)-4-phenyl-1,2,3-triazol-1-yl-acetamide is also a glycogen phosphorylase inhibitor. It is also noted in the introduction that 1-glycosyl-1,2,3-triazole compounds have also been investigated as anti-cancer agents. By removing the protecting groups, the authors obtained test compounds that were tested for their glycogen phosphorylase-b inhibitory activity. 1-(β-D-glucopyranosyl)-1,2,3-triazoles proved to be the best inhibitors from several series of compounds, their inhibition equilibrium constants fell in the lower regions of the micromolar range. The 1-(β-D-glucopyranosyl)-1,2,3-triazoles and N-acyl-β-D-glucopyranosylamines were similar in their inhibitory properties (Chrysina et al., 2009). The authors note that the higher activity of β-anomers than α-anomers is consistent with the literature. However, the authors did not use a nitrogen-containing heterocycle as the triazole-linked R group.

Mohammed, A. I. et al. (Mohamed, 2017) have prepared peracetylated D-glucose-based bis-1,2,3-triazoles and found that they have a mild antibacterial effect only. The strength of the antibacterial effect primarily did not depend on the presence or removal of the protecting group, but on the other chemical properties of the compound.

Samsulová, V. (Šamšulová et al., 2019) has prepared compounds comprising substituted pyridine and a triazole ring and the author has observed that these have an antibacterial activity. However, neither Mohammed A. I. et al., nor Samsulová, V. used metal complexes.

Unlike organic drug molecules the biological effects of metal complexes can be modulated by a wider array of parameters including size and charge of the species, hard-soft character of the metal ion, stability, inertness and geometry of the complex, just to mention a few. In this regard the present inventors have carried out a comprehensive study of Ru-sugar-conjugate based complexes, in which A) the role of the metal chelating part of the ligand, B) the basicity and binding strength of the coordinating donor atoms and C) the effect of the lipophilic/hydrophilic character of the complex, tuned by the presence/absence of various protecting groups at the sugar moiety were explored, may provide with a more detailed outlook to the structure-activity relationship (SAR) of these types of complexes.

### BRIEF DESCRIPTION OF THE INVENTION

To the best of the inventors' knowledge, no real C- and N-glycopyranosyl heterocyclic ligands as potential bidentate chelators have so far been used to obtain VIIIB group transition metal arene/arenyl complexes. In the present invention the inventors disclose a set of C- and N-glycopyranosyl azoles and their half-sandwich metal-complexes and synthesis thereof. Anticancer potential of the above ligands and their complexes have been studied. The compounds of the invention have also been tested for their antimicrobial effect.

The invention relates to a half-sandwich type compound of general formula (I)

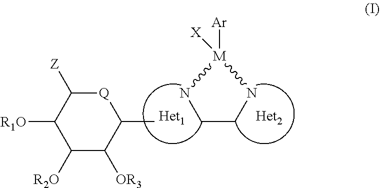

wherein in said formula
M is a transition metal ion selected from the group consisting of Ru, Os, Rh, Ir, Pd and Pt, preferably selected from Ru, Os, Rh, Ir,
X is a halogen selected from the group consisting of Cl, Br and I, preferably Cl,
Ar is a penta- or hexahapto coordinating capping (cap) molecule having at least one 5 or 6 membered aromatic ring, preferably being substituted with one or more, preferably or one to five or one to six, respectively, straight or branched chain C₁-C₆ alkyl, preferably C₁-C₃ alkyl, wherein the aromatic ring atoms are bound to the transition metal ion, preferably
   Ar is a capping molecule having a C₆ or C₅ aromatic ring substituted with one to six or one to five straight or branched chain C₁-C₃ alkyl, respectively,
Q is O, S or CH₂, preferably O or CH₂, more preferably O,
Het₁ is a 5 or 6, preferably 5 membered N-heterocycle, preferably aromatic N-heterocycle, comprising at least one N and optionally further heteoatom(s), preferably 1 or 2 further heteroatom(s) selected from N, S and O,
Het₂ is a 5 to 12 membered heterocycle comprising a 5 or 6 membered, preferably 6 membered aromatic N-heterocycle, wherein
   in an embodiment Het₂ is a 6 membered aromatic N-heterocycle, or
   in an embodiment, preferably, Het₂ is a 10 membered aromatic N-heterocycle,
wherein the nitrogens of Het₁ and Het₂ as shown in the formula are forming (N,N) chelate with the transition metal ion,
R₁, R₂, and R₃ are selected from H, C₁-C₂₀ alkyl, C₂-C₂₀ alkenyl, C₂-C₂₀ alkynyl, C₂-C₂₀ alkyl-carbonyl, C₃-C₂₀ alkenyl-carbonyl, C₃-C₂₀ alkynyl-carbonyl, C₆-C₁₀ aryl, C₇-C₂₀ aralkyl, C₈-C₂₀ aralkyl-carbonyl, C₇-C₁₁ aryl-carbonyl (aroyl), wherein at least one, preferably at least two of R₁, R₂, and R₃ is different from H, in particular at least two of R₁, R₂, and R₃ is C₇-C₁₁ aryl-carbonyl (aroyl), preferably C₇ aryl-carbonyl,
wherein the alkyl, alkenyl and aralkyl groups may be of branched or straight chain, unless specifically defined differently and
Z is selected from H, C₁-C₁₀ alkyl, C₂-C₁₀ alkenyl, C₂-C₁₀ alkynyl, —CH₂OR₄, wherein R₄ is selected from any group as defined for R₁, R₂, and R₃, in particular R₄ is C₇-C₁₁ aryl-carbonyl (aroyl), preferably C₇ aryl-carbonyl,
wherein any aryl group may be unsubstituted or substituted with one or more C₁-C₁₀ alkyl, C₂-C₁₀ alkenyl, C₂-C₁₀ alkynyl, wherein the number of carbon atoms in R₁, R₂ or R₃ is not more than 20,
or any salt or solvate thereof.

Thus, the meaning of M is a transition metal ion selected from the group consisting of group VIIIA (in more recent nomenclature groups 8-10) of 4d and 5d series of the periodic table, i.e. Ru, Os, Rh, Ir, Pd and Pt. Preferably the transition metal ion has the following oxidation states: Ru(II), Os(II), Rh(III), Ir(III), Pd(II) and Pt(II),
preferably the transition metal ions being selected from Ru, Os, Rh, Ir, in particular having the following oxidation states: Ru(II), Os(II), Rh(III), Ir(III), in particular selected from.
Ar is a penta- or hexahapto coordinating cap molecule having at least one 5 or 6 membered aromatic ring, being substituted with one to fiver or one to six C₁-C₆ alkyl, preferably C₁-C₃ alkyl, wherein the aromatic ring atoms are bound to the transition metal ion,
X is preferably Cl or Br, more preferably Cl,
Q is preferably O or CH₂, more preferably O,
Het₁ is a 5 or 6 membered, preferably 5 membered aromatic N-heterocycle (N-heteroaryl), wherein the N-heterocycle comprises at least one further heteroatom selected from N, O and S, preferably N and O; more preferably comprises at least one further N,
Het₂ is a 5 to 12 membered heterocycle comprising a 5 or 6 membered, preferably 6 membered N-heterocycle, preferably an aromatic N-heterocycle (N-heteroaryl);
wherein the nitrogens (N) of Het₁ and Het₂ as shown in the formula are forming (N,N) chelate with the transition metal ion,
   wherein preferably
   in an embodiment Het₂ is a 6 membered aromatic N-heterocycle, or
   in an embodiment, preferably, Het₂ is a 10 membered aromatic N-heterocycle
R₁, R₂, and R₃ are selected from H, C₁-C₂₀ alkyl, C₂-C₂₀ alkenyl, C₂-C₂₀ alkynyl, C₂-C₂₀ alkyl-carbonyl, C₃-C₂₀ alkenyl-carbonyl, C₃-C₂₀ alkynyl-carbonyl, C₆-C₁₀ aryl, C₇-C₂₀ aralkyl (including alkylaryl), C₈-C₂₀ aralkyl-carbonyl, C₇-C₁₁ aryl-carbonyl (aroyl), in particular C₇-C₁₁ aryl-carbonyl (aroyl), wherein at least one, preferably at least two of R₁, R₂, and R₃ is different from H,
   wherein the alkyl, alkenyl and aralkyl groups may be of branched or straight chain, unless specifically defined differently and
   wherein any aryl group may be unsubstituted or substituted with one or more C₁-C₁₀ alkyl, C₂-C₁₀ alkenyl, C₂-C₁₀ alkynyl, wherein the number of carbon atoms in R₁, R₂ or R₃ is not more than 20, preferably the aryl group is unsubstituted,
Z is selected from H, C₁-C₁₀ alkyl, C₂-C₁₀ alkenyl, C₂-C₁₀ alkynyl, —CH₂OR₄, preferably Z is H or —CH₂OR₄, wherein R₄ is selected from any group as defined for R₁, R₂, and R₃, in particular R₄ is C₇-C₁₁ aryl-carbonyl (aroyl),
or any salt or solvate thereof.

Preferably the invention relates to a half-sandwich type compound of general formula (I) for use in a neoplastic disease or in a bacterial disease.

Preferably one or more, preferably at least two of R₁, R₂, R₃ and R₄ is selected from C₆-C₁₀ aryl, C₄-C₂₀ alkyl, C₄-C₂₀ alkenyl, C₄-C₂₀ alkynyl, C₅-C₂₀ alkyl-carbonyl, C₅-C₂₀ alkenyl-carbonyl, C₅-C₂₀ alkynyl-carbonyl, C₇-C₂₀ aralkyl (including alkylaryl), C₈-C₂₀ aralkyl-carbonyl and C₇-C₁₁ aryl-carbonyl (aroyl), in particular C₇-C₁₁ aryl-carbonyl (aroyl).

In an embodiment Het₂ comprises a heterocycle selected from the group consisting of the benzologues of the 6 membered aromatic N-heterocycle, preferably selected from the group consisting of quinoline, izoquinolin and 2-izoquinoline.

Preferably
in an embodiment Het₂ is a 6 membered aromatic N-heterocycle, or
in an embodiment, preferably, Het₂ is a 10 membered aromatic N-heterocycle

Preferably at least one or more preferably at least two of R₁, R₂, and R₃ is/are selected from C₆-C₁₀ aryl, C₄-C₂₀ alkyl, C₄-C₂₀ alkenyl, C₄-C₂₀ alkynyl, C₅-C₂₀ alkyl-carbonyl, C₅-C₂₀ alkenyl-carbonyl, C₅-C₂₀ alkynyl-carbonyl, C₇-C₂₀ aralkyl, C₈-C₂₀ aralkyl-carbonyl, C₇-C₁₁ aryl-carbonyl (aroyl),
preferably at least two, in particular at least three of R₁, R₂, R₃ and R₄ is selected from C₆-C₁₀ aryl, C₄-C₂₀ alkyl, C₄-C₂₀ alkenyl, C₄-C₂₀ alkynyl, C₅-C₂₀ alkyl-carbonyl, C₅-C₂₀ alkenyl-carbonyl, C₅-C₂₀ alkynyl-carbonyl, C₇-C₂₀ aralkyl (including alkylaryl), C₈-C₂₀ aralkyl-carbonyl and C₇-C₁₁ aryl-carbonyl (aroyl), in particular C₇-C₁₁ aryl-carbonyl (aroyl),
   preferably C₆-C₁₀ aryl, C₄-C₁₀ alkyl, C₄-C₁₀ alkenyl, C₄-C₁₀ alkynyl, C₅-C₁₀ alkyl-carbonyl, C₅-C₁₀ alkenyl-carbonyl, C₅-C₁₀ alkynyl-carbonyl, C₇-C₁₅ aralkyl (including alkylaryl), C₈-C₁₅ aralkyl-carbonyl, C₇-C₁₁ aryl-carbonyl,
   preferably C₆-C₁₀ aryl, C₄-C₁₀ alkyl, C₅-C₁₀ alkyl-carbonyl, C₇-C₁₀ aralkyl (including alkylaryl), C₈-C₁₀ aralkyl-carbonyl, C₇-C₁₁ aryl-carbonyl (aroyl), in particular C₇-C₁₁ aryl-carbonyl (aroyl),
   preferably C₄-C₁₀ alkyl, C₅-C₁₀ alkyl-carbonyl, C₈-C₁₀ aralkyl-carbonyl, C₇-C₁₁ aryl-carbonyl.

Preferably at least one or more preferably at least two of R₁, R₂, and R₃, preferably at least two, in particular at least three of R₁, R₂, R₃ and R₄ is/are selected from C₅-C₂₀ (preferably C₁₀) alkyl-carbonyl, C₅-C₂₀ (preferably C₁₀) alkenyl-carbonyl, C₅-C₂₀ (preferably C₁₀) alkynyl-carbonyl, C₈-C₂₀ (preferably C₁₅) aralkyl-carbonyl, C₇-C₁₁ aryl-carbonyl (aroyl), in particular C₇-C₁₁ aryl-carbonyl (aroyl), preferably C₇ aryl-carbonyl,
preferably C₅-C₂₀ alkyl-carbonyl, C₈-C₂₀ aralkyl-carbonyl, C₇-C₁₁ aryl-carbonyl (aroyl), C₈-C₂₀ (preferably C₁₅) aralkyl-carbonyl, C₇-C₁₁ (preferably C₇) aryl-carbonyl (aroyl), in particular C₇-C₁₁ aryl-carbonyl (aroyl), preferably C₇ aryl-carbonyl,
preferably C₅-C₂₀ alkyl-carbonyl, C₅-C₂₀ alkenyl-carbonyl, C₅-C₂₀ alkynyl-carbonyl, C₈-C₂₀ aralkyl-carbonyl, C₇-C₁₁ aryl-carbonyl (aroyl), in particular C₇-C₁₁ aryl-carbonyl (aroyl).

In a particular embodiment said compound has a distribution coefficient (log D) of at least 1.4, preferably at least 1.6, more preferably at least 1.8, highly preferably at least 2.0. In a particular embodiment the distribution coefficient (log D) is not higher than 4.0, in particular 3.5 or 3.0. In a particular embodiment the log D is 1.4 to 4, preferably 1.6 or 1.8 to 4, in particular 1.6 or 1.8 to 3.5, in particular 2 to 4 or 2 to 3.5. In particular, log D is measured between n-octanol saturated with aqueous PBS solution (pH=7.40), and aqueous PBS solution, preferably as disclosed herein.

In a particular embodiment in formula (I),
M is a metal ion selected from the group consisting of Ru, Os, Rh, Ir, preferably selected from Ru and Os and Ir, or preferably selected from Ru and Os or Rh and Ir,
Ar is a capping molecule having a C₆ or C₅ aromatic ring substituted with one or more, preferably one to six or one to five C₁-C₃ alkyl, respectively,
(preferably Ar is a C₆ aromatic ring substituted with one or more, preferably one to six, preferably 1 to 3, preferably 2, C₁-C₃ alkyl, and M is Ru or Os),
X is a halogen selected from the group consisting of Cl and Br,
Q is O, S or CH₂, preferably O,
Het₁ is a 5 membered aromatic N-heterocycle preferably comprising at least one further N,
optionally comprising at least one, preferably one further heteroatom selected from O, S, N,
Het₂ is a 6 membered aromatic N-heterocycle
R₁, R₂, and R₃ are selected from H, C₁-C₁₀ alkyl, C₂-C₁₀ alkenyl, C₂-C₁₀ alkynyl, C₂-C₁₀ alkyl-carbonyl, C₃-C₁₀ alkenyl-carbonyl, C₃-C₁₀ alkynyl-carbonyl, C₆-C₁₀ aryl, C₇-C₁₅ aralkyl (including alkylaryl), C₈-C₁₅ aralkyl-carbonyl, C₇-C₁₁ aryl-carbonyl, in particular C₇-C₁₁ aryl-carbonyl, wherein at least one, preferably at least two of R₁, R₂, and R₃ is different from H,
wherein at least one or preferably at least two of R₁, R₂, and R₃ is selected from
C₄-C₁₀ alkyl, C₄-C₁₀ alkenyl, C₄-C₁₀ alkynyl, C₅-C₁₀ alkyl-carbonyl, C₅-C₁₀ alkenyl-carbonyl, C₅-C₁₀ alkynyl-carbonyl, C₆-C₁₀ aryl, C₇-C₁₅ aralkyl (including alkylaryl), C₈-C₁₅ aralkyl-carbonyl, C₇-C₁₁ aryl-carbonyl,
Z is selected from H, C₁-C₁₀ alkyl, C₂-C₁₀ alkenyl, C₂-C₁₀ alkynyl, —CH₂OR₄, or OR₄, wherein R₄ is selected from C₁-C₁₀ alkyl, C₂-C₁₀ alkenyl, C₂-C₁₀ alkynyl, C₂-C₁₀ alkyl-carbonyl, C₃-C₁₀ alkenyl-carbonyl, C₃-C₁₀ alkynyl-carbonyl, C₆-C₁₀ aryl, C₇-C₁₅ aralkyl, C₈-C₁₅ aralkyl-carbonyl, C₇-C₁₁ aryl-carbonyl, in particular C₇-C₁₁ aryl-carbonyl,
preferably R₄ is selected from C₄-C₁₀ alkyl, C₄-C₁₀ alkenyl, C₄-C₁₀ alkynyl, C₅-C₁₀ alkyl-carbonyl, C₅-C₁₀ alkenyl-carbonyl, C₅-C₁₀ alkynyl-carbonyl, C₆-C₁₀ aryl, C₇-C₁₅ aralkyl (including alkylaryl), C₈-C₁₅ aralkyl-carbonyl, C₇-C₁₁ aryl-carbonyl, in particular C₇-C₁₁ aryl-carbonyl.

In a particular embodiment the compound of the invention has general formula (II)

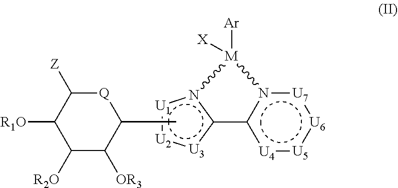

wherein
M is a metal ion selected from the group consisting of Ru, Os, Rh and Ir, preferably Ru and Os,
Ar is a capping molecule having a C₆ or C₅ aromatic ring substituted with one or more, preferably one to six or one to five C₁-C₃ alkyl,
   preferably
   Ar is a capping molecule having a C₆ aromatic ring substituted with one or more, preferably one to six or one to five C₁-C₃ alkyl, provided that M is Ru(II) or Os(II)
   Ar is a capping molecule having a C₅ aromatic ring substituted with one or more, preferably one to five C₁-C₃ alkyl, provided that M is Rh(III) or Ir(III)
X is a halogen selected from the group consisting of Cl and Br,
Q, is as defined in any of claim 1 or 2, preferably Q is O or CH₂, more preferably O,
U₁, U₂, and U₃ are selected from N, NH, O, S, C and CH, wherein they, together with the rest of the Het₁ ring moiety form an aromatic five-membered N-heterocycle,
U₄, U₅, U₆ and U₇ are selected from N, C and CH, or any of the pairs selected from the group consisting of U₄ and U₅; U₅ and U₆ as well as U₆ and U₇ form a fused (anellated) benzene ring or any of the pairs is part of a fused benzene ring, preferably quinoline,
R₁, R₂ and R₃ are as defined in any of claim 1 or 2, preferably at least two of R₁, R₂, and R₃ is selected from H, C₄-C₁₀ alkyl, C₄-C₁₀ alkenyl, C₄-C₁₀ alkynyl, C₅-C₁₀ alkyl-carbonyl, C₅-C₁₀ alkenyl-carbonyl, C₅-C₁₀ alkynyl-carbonyl, C₆-C₁₀ aryl, C₇-C₁₅ aralkyl, C₈-C₁₅ aralkyl-carbonyl, C₇-C₁₁ aryl-carbonyl, in particular C₇-C₁₁ aryl-carbonyl, and the aryl groups are substituted or unsubstituted as defined hereinabove, preferably R₁, R₂, and R₃ are different from H,
   more preferably each of R₁, R₂ and R₃ is, independently, selected from
      C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl (including alkylaryl), C₈-C₁₁ aralkyl-carbonyl, C₇-C₁₁ aryl-carbonyl, in particular C₇-C₁₁ aryl-carbonyl, wherein any aryl group may be unsubstituted or substituted with one or more C₁-C₄ alkyl, C₂-C₄ alkenyl, C₂-C₄ alkynyl, wherein the number of carbon atoms in R₁, R₂ or R₃ is not more than 12,
Z is as defined in any of claim 1 or 2, preferably Z is selected from H, C₁-C₁₀ alkyl, C₂-C₁₀ alkenyl, C₂-C₁₀ alkynyl, —CH₂OR₄, or OR₄ wherein R₄ is a group as defined herein for any of R₁, R₂ and R₃,
   wherein more preferably Z is selected from H, C₁-C₈ alkyl, C₂-C₈ alkenyl, C₂-C₈ alkynyl, —CH₂OR₄, wherein R₄ is selected from C₁-C₈ alkyl, C₂-C₈ alkenyl, C₂-C₈ alkynyl, C₂-C₈ alkyl-carbonyl, C₃-C₈ alkenyl-carbonyl, C₃-C₈ alkynyl-carbonyl, C₆-C₁₀ aryl, C₇-C₁₅ aralkyl, C₈-C₁₅ aralkyl-carbonyl, C₇-C₁₁ aryl-carbonyl, in particular C₇-C₁₁ aryl-carbonyl, preferably R₄ is different from H,
      more preferably R₄ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl (including alkylaryl), C₈-C₁₁ aralkyl-carbonyl, C₇-C₁₁ aryl-carbonyl, in particular C₇-C₁₁ aryl-carbonyl, wherein any aryl group may be unsubstituted or substituted with one or more C₁-C₄ alkyl, C₂-C₄ alkenyl, C₂-C₄ alkynyl, wherein the number of carbon atoms in R₄ is not more than 12.

In a preferred embodiment the invention relates to the complex or any complex for use in a disease as disclosed herein in a particular a bacterial disease or a neoplastic disease as detailed below.
In a preferred embodiment the compound has general formula (II.1)

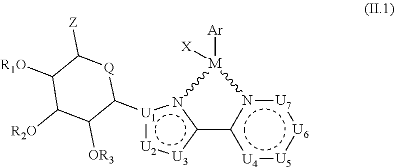

wherein M, Ar, X, Q, U₁, U₂, U₃, U₄, U₅, U₆ and U₇, R₁, R₂ and R₃ and Z are as defined above or in claim 3, preferably
M is a metal ion selected from the group consisting of Ru, Os, Ir and Rh, in particular Ru, Os, and Ir, preferably Ru and Os, in particular Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl,
Q, is O or CH₂, preferably O,
U₁ is C or N and U₂ and U₃ are selected from N, NH, O, C and CH, wherein they, together with the rest of the Het₁ ring moiety form an aromatic five-membered N-heterocycle, more preferably U₁ is C, U₂ is CH or N and U₃ is O or NH, respectively,
   U₄, U₅, U₆ and U₇ are selected from N, NH, O, C and CH, wherein U₄, U₅, U₆ and U₇ together with the rest of the Het₂ ring moiety form an aromatic six-membered N-heterocycle, more preferably each of U₄, U₅, U₆ and U₇ are CH, OR
   U₄, U₅, U₆ and U₇ are selected from N, C and CH, or any of the pairs selected from the group consisting of U₄ and U₅; U₅ and U₆ as well as U₆ and U₇ form a fused (anellated) benzene ring or any of the pairs is part of a fused benzene ring, preferably quinoline,
R₁, R₂ and R₃ are as defined in any of claim 1 or 2, wherein at least two of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl (aroyl), wherein any aryl group may be unsubstituted or substituted with one or more C₁-C₄ alkyl, C₂-C₄ alkenyl, C₂-C₄ alkynyl, wherein the number of carbon atoms in R₁, R₂ or R₃ is not more than 12.
Z is selected from H, C₁-C₈ alkyl, C₂-C₈ alkenyl, C₂-C₈ alkynyl, —CH₂OR₄, or OR₄, wherein R₄ is selected from C₁-C₈ alkyl, C₂-C₈ alkenyl, C₂-C₈ alkynyl, C₂-C₈ alkyl-carbonyl, C₃-C₈ alkenyl-carbonyl, C₃-C₈ alkynyl-carbonyl, C₆-C₁₀ aryl, C₇-C₁₅ aralkyl, C₈-C₁₅ aralkyl-carbonyl, C₇-C₁₁ aryl-carbonyl,
   preferably R₄ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
wherein any aryl group may be unsubstituted or substituted with one or more C₁-C₄ alkyl, C₂-C₄ alkenyl, C₂-C₄ alkynyl, wherein the number of carbon atoms in R₄ is not more than 12.
In a particular embodiment the aryl groups are unsubstituted, wherein preferably each aryl group is unsubstituted.

In a preferred embodiment the invention relates to the complex or any complex for use in a disease as disclosed herein in a particular a bacterial disease or a neoplastic disease as detailed below.

In a further particularly preferred embodiment said compound has general formula (II.2)

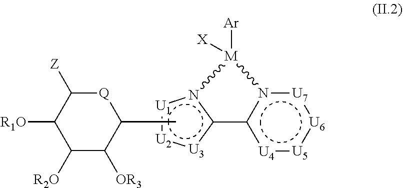

wherein M, Ar, X, Q, U₁, U₂, U₃, U₄, U₅, U₆ and U₇, R₁, R₂ and R₃ and Z are as defined in claim 3,
   preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
U₂ is C or N and U₁, and U₃ are selected from N, NH, O, C and CH, wherein they, together with the rest of the
Het₁ ring moiety form an aromatic five-membered N-heterocycle, more preferably U₁ is N and U₃ is O or CH,
   U₄, U₅, U₆ and U₇ are selected from N, NH, O, C and CH, wherein U₄, U₅, U₆ and U₇ together with the rest of the Het₂ ring moiety form an aromatic six-membered N-heterocycle, more preferably each of U₄, U₅, U₆ and U₇ are CH, OR
   U₄, U₅, U₆ and U₇ are selected from N, C and CH, or any of the pairs selected from the group consisting of U₄ and U₅; U₅ and U₆ as well as U₆ and U₇ form a fused (anellated) benzene ring or any of the pairs is part of a fused benzene ring, preferably quinoline,
R₁, R₂ and R₃ are as defined for formula (I), wherein at least two of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl, wherein preferably each aryl group is unsubstituted,
Z is selected from H, C₁-C₈ alkyl, C₂-C₈ alkenyl, C₂-C₈ alkynyl, —CH₂OR₄, or OR₄, wherein R₄ is selected from C₁-C₈ alkyl, C₂-C₈ alkenyl, C₂-C₈ alkynyl, C₂-C₈ alkyl-carbonyl, C₃-C₈ alkenyl-carbonyl, C₃-C₈ alkynyl-carbonyl, C₆-C₁₀ aryl, C₇-C₁₅ aralkyl, C₈-C₁₅ aralkyl-carbonyl, C₇-C₁₁ aryl-carbonyl, in particular C₇-C₁₁ aryl-carbonyl, wherein preferably each aryl group is unsubstituted,
   preferably R₄ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl.
Preferably the invention relates to a half-sandwich type compound of general formula (II), (II.1) and/or (II.2) for use in a neoplastic disease or in a bacterial disease.

In a preferred variant of this embodiment said compound has formula (III)

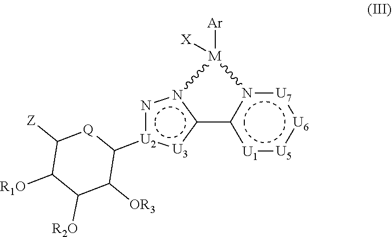

wherein M, Ar, X, Q, U₂, U₃, R₁, R₂ and R₃ and Z are as defined for any of formulae (II) or, in particular, (II.2), preferably M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl,
Q, is O or CH₂, preferably O,
U₂ is C or N and U₃ is O or NH, provided that U₂ is C, and U₃ is CH or N provided that U₂ is N,
U₄ to U₇ are as defined above for any of formulae (II) or, in particular, any of formulae (II.2)
R₁, R₂ and R₃ are as defined for any of formulae (II) or, in particular, any of formulae (II.2), wherein preferably each of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
   more preferably each of R₁, R₂ and R₃ are selected from C₄-C₈ alkyl, C₅-C₈ alkyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
Z is selected from H, C₁-C₈ alkyl, C₂-C₈ alkenyl, C₂-C₈ alkynyl, or —CH₂OR₄, wherein R₄ is as defined in this claims for any of R₁, R₂, and R₃
wherein preferably each aryl group is unsubstituted.

In a particularly preferred embodiment the compound of the invention has formula (III.1)

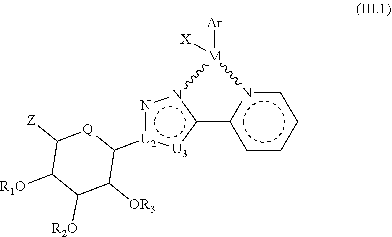

wherein M, Ar, X, Q, U₂, U₃, R₁, R₂ and R₃ and Z are as defined for any of formulae (II) or (II.2), in particular for formula (III), preferably
M is a metal ion selected from the group consisting of Ru, Os, Ir and Rh, in particular Ru, Os and Ir preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
U₂ is C or N and U₃ is O or NH, provided that U₂ is C, and U₃ is CH or N provided that U₂ is N,
R₁, R₂ and R₃ are as defined for any of formulae II, II.2 or III, preferably as defined in claim 5, wherein preferably each of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
   more preferably each of R₁, R₂ and R₃ are selected from C₄-C₈ alkyl, C₅-C₈ alkyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
Z is selected from H, C₁-C₈ alkyl, C₂-C₈ alkenyl, C₂-C₈ alkynyl, or —CH₂OR₄, wherein R₄ is as defined in these claims for any of R₁, R₂, and R₃,
wherein preferably each aryl group is unsubstituted.

In a particularly preferred embodiment the compound of the invention has formula (III.1.b1), (III.1.b2) or (III.1.b3), in particular (II.1.b1)

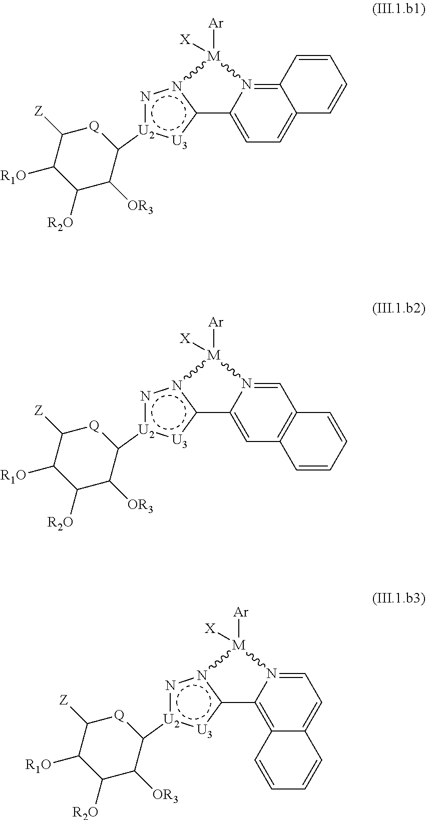

wherein M, Ar, X, Q, U₂, U₃, R₁, R₂ and R₃ and Z are as defined for any of formulae (II) or (II.2), in particular for formula (III), preferably
M is a metal ion selected from the group consisting of Ru, Os, Ir and Rh, in particular Ru, Os and Ir preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
U₂ is C or N and U₃ is O or NH, provided that U₂ is C, and U₃ is CH or N provided that U₂ is N,
R₁, R₂ and R₃ are as defined for any of formulae II, II.2 or III, preferably as defined in claim 5, wherein preferably each of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
   more preferably each of R₁, R₂ and R₃ are selected from C₄-C₈ alkyl, C₅-C₈ alkyl-carbonyl, C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
   highly preferably C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
Z is selected from H, C₁-C₈ alkyl, C₂-C₈ alkenyl, C₂-C₈ alkynyl, or —CH₂OR₄, wherein R₄ is as defined in this embodiment for any of R₁, R₂, and R₃,
wherein preferably each aryl group is unsubstituted.

In a further particular embodiment the invention relates to a compound having formula (IV)

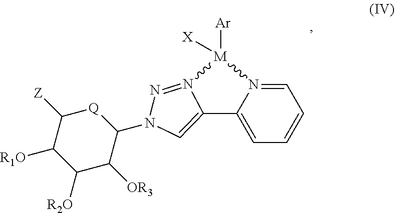

wherein M, Ar, X, Q, R₁, R₂ and R₃ and Z are as defined for formula (II), preferably as defined for formula (II.2) or (III) or (III.1),
   preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
R₁, R₂ and R₃ are as defined for formula (III.1), wherein preferably each of R₁, R₂, and R₃ is selected from C₄-C₈ alkyl, C₅-C₈ alkyl-carbonyl, C₅-C₈ alkenyl-carbonyl, C₅-C₈ alkynyl-carbonyl, C₈-C₁₁ aralkyl-carbonyl, C₇-C₁₁ aryl-carbonyl, in particular C₇-C₁₁ aryl-carbonyl,
   more preferably each of R₁, R₂ and R₃ are selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl,
Z is selected from H, C₁-C₈ alkyl, C₂-C₈ alkenyl, C₂-C₈ alkynyl, —CH₂OR₄, or OR₄, wherein R₄ is as defined in this claims for any of R₁, R₂, and R₃
Said compound has a formula preferably selected from the group of formulae (IV.1) and (IV.2)

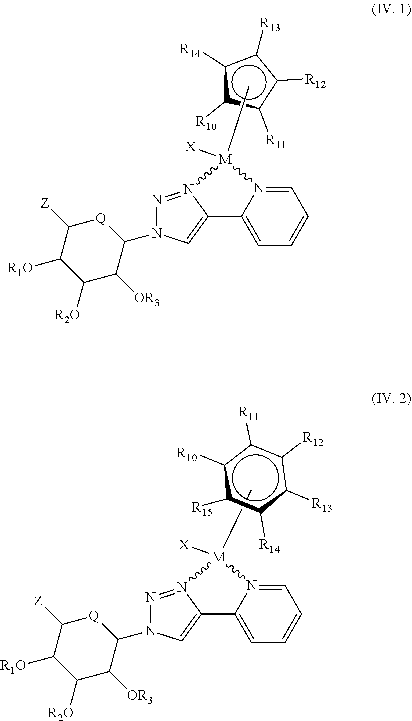

wherein in formula (IV.1)
X, Q, R₁, R₂ and R₃ and Z are as defined in claim 7,
M is Ir or Rh, and
R₁₀, R₁₁, R₁₂, R₁₃, and R₁₄ are selected from, independently, H and C₁-C₃ alkyl groups, preferably C₁-C₂ alkyl groups, preferably each of R₁₀, R₁₁, R₁₂, R₁₃, and R₁₄ being identical, preferably methyl,
preferably having the formula of (IV.1.1)

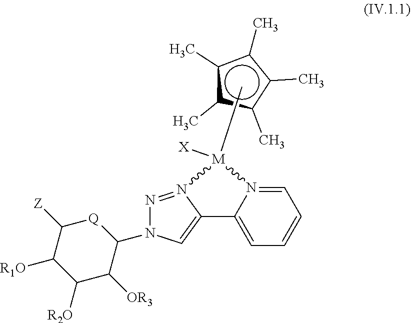

wherein in formula (IV.1.1)
X, Q, R₁, R₂ and R₃ and Z are as defined in claim 7,
   preferably
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in this claims for any of R₁, R₂, and R₃
M is Ir or Rh,
OR
wherein in formula (IV.2)
X, Q, R₁, R₂ and R₃ and Z are as defined in claim 7,
M is Ru or Os, and
R₁₀, R₁₁, R₁₂, R₁₃, R₁₄ and R₁₅ are selected from, independently, H and C₁-C₃ alkyl groups, preferably at least two of R₁₀, R₁₁, R₁₂, R₁₃, R₁₄ and R₁₅, preferably two, being selected from C₁-C₃ alkyl groups,
preferably from methyl and isopropyl, highly preferably in a para position,
preferably having the formula of (IV.2.1)

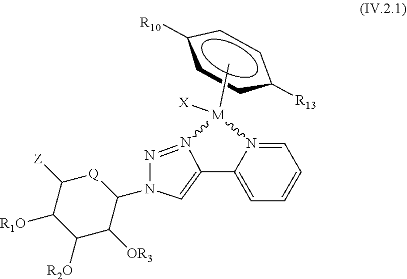

wherein in formula (IV.2.1)
X, Q, R₁, R₂ and R₃ and Z are as defined in claim 7,
   preferably
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in this claims for any of R₁, R₂, and R₃
M is Ru or Os, and
R₁₀, and R₁₃ are selected from C₁-C₃ alkyl, preferably methyl and isopropyl
wherein each aryl group is unsubstituted.

In a further particular embodiment the invention relates to a compound having formula (IV.b1), (IV.b2) or (IV.b3), in particular formula (IV.b1),

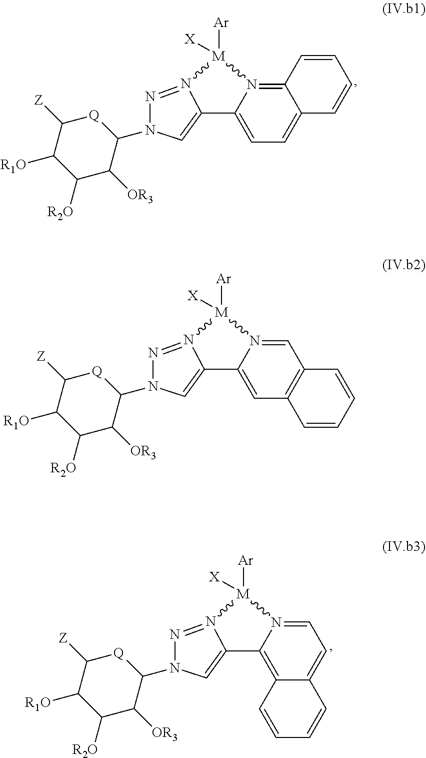

wherein M, Ar, X, Q, R₁, R₂ and R₃ and Z are as defined for formula (II), preferably as defined for formula (II.2) or (III) or (III.1),
   preferably
M is a metal ion selected from the group consisting of Ru, Os, Ir and Rh, in particular Ru, Os, and Ir, preferably Ru and Os, in particular Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
R₁, R₂ and R₃ are as defined for formula (III.1), wherein preferably each of R₁, R₂, and R₃ is selected from C₄-C₈ alkyl, C₅-C₈ alkyl-carbonyl, C₅-C₈ alkenyl-carbonyl, C₅-C₈ alkynyl-carbonyl, C₈-C₁₁ aralkyl-carbonyl, C₇-C₁₁ aryl-carbonyl, in particular C₇-C₁₁ aryl-carbonyl,
   more preferably each of R₁, R₂ and R₃ is selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl,
   highly preferably each of R₁, R₂ and R₃ is selected from C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
Z is selected from H, C₁-C₈ alkyl, C₂-C₈ alkenyl, C₂-C₈ alkynyl, —CH₂OR₄, or OR₄, wherein R₄ is as defined in this embodiment for any of R₁, R₂, and R₃
Said compound preferably has a formula preferably selected from the group of formulae (IV.1.b1), (IV.1.b2) or (IV.1.b3), in particular (IV.1.b1),

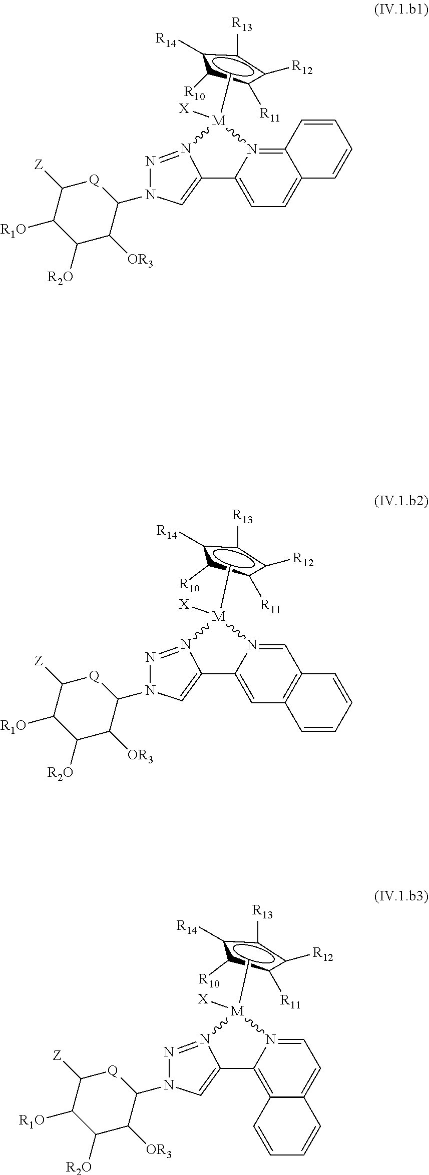

wherein in formula (IV.1b1), (IV.1.b2) and (IV.1.b3), in particular (IV.1b1)
X, Q, R₁, R₂ and R₃ and Z are as defined herein, preferably for formulae IV,
M is Ir or Rh, and
R₁₀, R₁₁, R₁₂, R₁₃, and R₁₄ are selected from, independently, H and C₁-C₃ alkyl groups, preferably C₁-C₂ alkyl groups, preferably each of R₁₀, R₁₁, R₁₂, R₁₃, and R₁₄ being identical, preferably methyl,
preferably pentamethyl,
wherein in formulae (IV.1b1), (IV.1.b2) and (IV.1.b3), in particular (IV.1b1)
X, Q, R₁, R₂ and R₃ and Z are as defined in herein, preferably for preferably for formulae IV,
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl, preferably C₇ aryl-carbonyl,
   highly preferably each of R₁, R₂, and R₃ is selected from C₈-C₁₁ aralkyl-carbonyl, C₇-C₁₁ aryl-carbonyl, in particular C₇-C₁₁ aryl-carbonyl, preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in this claims for any of R₁, R₂, and R₃ highly preferably R₄ is selected from C₈-C₁₁ aralkyl-carbonyl, C₇-C₁₁ aryl-carbonyl, in particular C₇-C₁₁ aryl-carbonyl, preferably C₇ aryl-carbonyl, and M is Ir or Rh, preferably Ir.

Said compound preferably has a formula preferably selected from the group of formulae (IV.2.b1), (IV.2.b2) and (IV.2.b3), in particular (IV.2.b1),

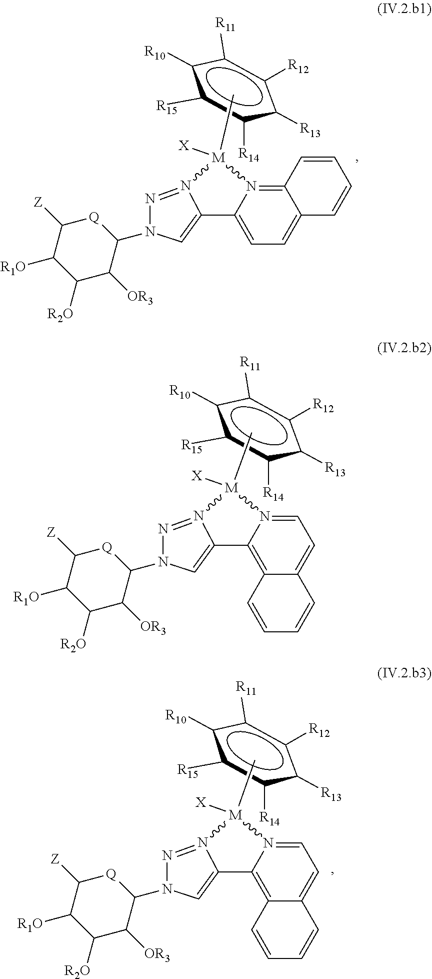

wherein in formula (IV.2.b1), (IV.2.b2) and (IV.2.b3), in particular (IV.2.b1)
X, Q, R₁, R₂ and R₃ and Z are as defined herein, preferably for formulae IV, preferably for formulae (IV.b1), (IV.b2) or (IV.b3), in particular formula (IV.b1),
M is Ru or Os, preferably Os, and
R₁₀, R₁₁, R₁₂, R₁₃, R₁₄ and R₁₅ are selected from, independently, H and C₁-C₃ alkyl groups, preferably at least two of R₁₀, R₁₁, R₁₂, R₁₃, R₁₄ and R₁₅, preferably two, being selected from C₁-C₃ alkyl groups,
preferably from methyl and isopropyl, highly preferably in a para position,
preferably having the formula of (IV.2.1)
wherein in formulae (IV.1.b1), (IV.1.b2) and (IV.1.b3), in particular (IV.1.b1)
X, Q, R₁, R₂ and R₃ and Z are as defined in herein, preferably for formulae (IV.2.b1), (IV.2.b2) and (IV.2.b3), in particular (IV.2.b1)
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl, preferably C₇ aryl-carbonyl,
      highly preferably each of R₁, R₂, and R₃ is selected from C₈-C₁₁ aralkyl-carbonyl, C₇-C₁₁ aryl-carbonyl, in particular C₇-C₁₁ aryl-carbonyl, preferably C₇ aryl-carbonyl,
         Z is selected from H, C₁-C₆ alkyl, C₂-C₆ alkenyl, C₂-C₆ alkynyl, —CH₂OR₄, or OR₄, wherein R₄ is as defined in this claims for any of R₁, R₂, and R₃ highly preferably R₄ is selected from C₈-C₁₁ aralkyl-carbonyl, C₇-C₁₁ aryl-carbonyl, in particular C₇-C₁₁ aryl-carbonyl, preferably C₇ aryl-carbonyl, and
         and M is Ru or Os preferably Os.

In a further particular embodiment the invention relates to a compound having formula (V)

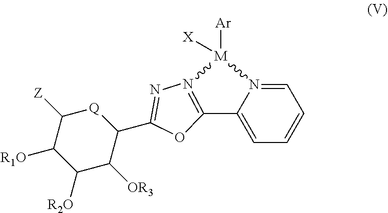

wherein M, Ar, X, Q, R₁, R₂ and R₃ and Z are as defined for formula (II), preferably as defined for formula (II.2) or (III) or (111.1),
   preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
R₁, R₂ and R₃ are as defined in claim 6, wherein preferably each of R₁, R₂, and R₃ is selected from C₄-C₈ alkyl, alkyl-carbonyl, C₅-C₈ alkenyl-carbonyl, C₅-C₈ alkynyl-carbonyl, C₈-C₁₁ aralkyl-carbonyl, C₇-C₁₁ aryl-carbonyl,
   more preferably each of R₁, R₂ and R₃ are selected from C₄-C₈ alkyl, C₅-C₈ alkyl-carbonyl, C₈-C₁₁ aralkyl-carbonyl, C₇-C₁₁ aryl-carbonyl,
   Z is selected from H, C₁-C₈ alkyl, C₂-C₈ alkenyl, C₂-C₈ alkynyl, —CH₂OR₄, or OR₄, wherein R₄ is as defined in this claim for any of R₁, R₂, and R₃.

Said compound has a formula preferably selected from the group of formulae (V.1) and (V.2)

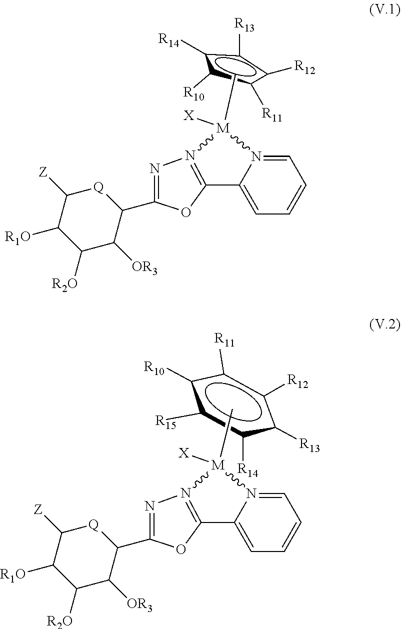

wherein in formula (V.1)
X, Q, R₁, R₂ and R₃ and Z are as defined for formulae (V) or in claim 9,
M is Ir or Rh, and R₁₀, R₁₁, R₁₂, R₁₃, and R₁₄ are selected from, independently, H and C₁-C₃ alkyl groups, preferably C₁-C₂ alkyl groups, preferably each of R₁₀, R₁₁, R₁₂, R₁₃, and R₁₄ being identical, preferably methyl, preferably having the formula of (V.1.1)

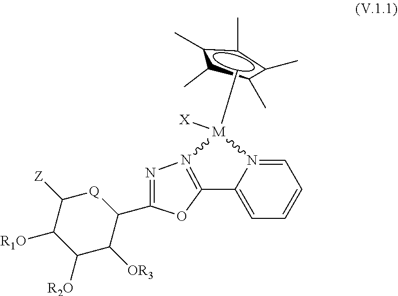

wherein in formula (V.1.1)
X, Q, R₁, R₂ and R₃ and Z are as defined for formulae (V) or in claim 9,
   preferably
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in these claims for any of R₁, R₂, and R₃
M is Ir or Rh,
OR
wherein in formula (V.2)
X, Q, R₁, R₂ and R₃ and Z are as defined in claim 9,
M is Ru or Os, and
R₁₀, R₁₁, R₁₂, R₁₃, R₁₄ and R₁₅ are selected from, independently, H and C₁-C₃ alkyl groups, preferably at least two of R₁₀, R₁₁, R₁₂, R₁₃, R₁₄ and R₁₅, preferably two, being selected from C₁-C₃ alkyl groups,
preferably from methyl and isopropyl, highly preferably in a para position,
preferably having the formula of (V.2.1)

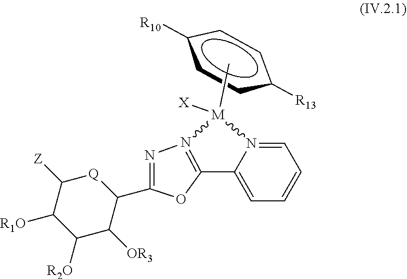

wherein in formula (V.2.1)
X, Q, R₁, R₂ and R₃ and Z are as defined in claim 7,
   preferably
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in these claims for any of R₁, R₂, and R₃
M is Ru or Os, and
R₁₀, and R₁₃ are selected from C₁-C₃ alkyl, preferably methyl and isopropyl,
wherein each aryl group is unsubstituted.

In a further particular embodiment the invention relates to a compound having formula (V.b1), (V.b2) or (V.b3), in particular formula (V.b1),

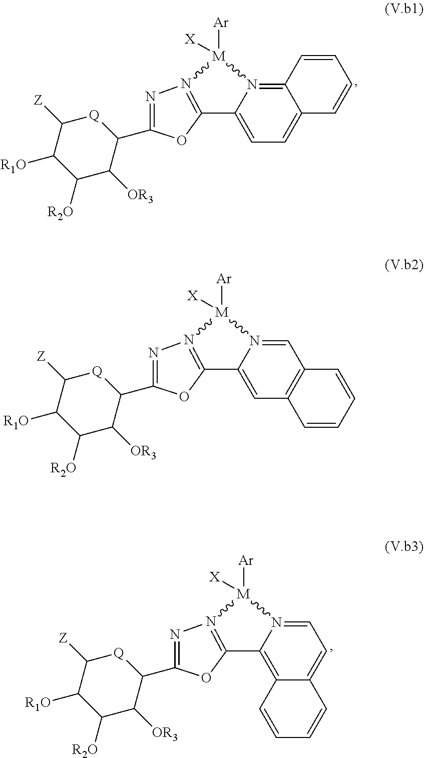

wherein M, Ar, X, Q, R₁, R₂ and R₃ and Z are as defined for formula (II), preferably as defined for formula (II.2) or (III) or (III.1),
   preferably
M is a metal ion selected from the group consisting of Ru, Os, Ir and Rh, in particular Ru, Os, and Ir, preferably
Ru and Os, in particular Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
R₁, R₂ and R₃ are as defined for formula (III.1), wherein preferably each of R₁, R₂, and R₃ is selected from C₄-C₈ alkyl, C₅-C₈ alkyl-carbonyl, C₅-C₈ alkenyl-carbonyl, C₅-C₈ alkynyl-carbonyl, C₈-C₁₁ aralkyl-carbonyl, C₇-C₁₁ aryl-carbonyl, in particular C₇-C₁₁ aryl-carbonyl,
   more preferably each of R₁, R₂ and R₃ is selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl,
   highly preferably each of R₁, R₂ and R₃ is selected from C₆ aryl, C₇-C₁₀ aralkyl, C₈-C₁₁ aralkyl-carbonyl, C₇-C₁₁ aryl-carbonyl, in particular C₇-C₁₁ aryl-carbonyl,
Z is selected from H, C₁-C₈ alkyl, C₂-C₈ alkenyl, C₂-C₈ alkynyl, —CH₂OR₄, or OR₄, wherein R₄ is as defined in this embodiment for any of R₁, R₂, and R₃

Said compound preferably has a formula preferably selected from the group of formulae (IV.1.b1), (IV.1.b2) or (IV.1.b3), in particular (IV.1.b1),

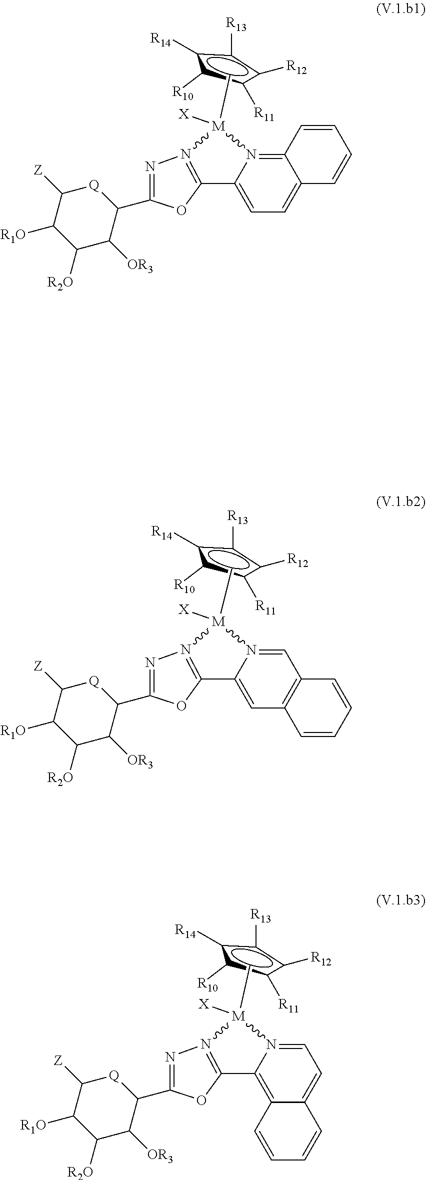

wherein in formula (IV.1.b1), (IV.1.b2) and (IV.1.b3), in particular (IV.1.b1)
X, Q, R₁, R₂ and R₃ and Z are as defined herein, preferably for formulae IV,
M is Ir or Rh, and
R₁₀, R₁₁, R₁₂, R₁₃, and R₁₄ are selected from, independently, H and C₁-C₃ alkyl groups, preferably C₁-C₂ alkyl groups, preferably each of R₁₀, R₁₁, R₁₂, R₁₃, and R₁₄ being identical, preferably methyl, preferably pentamethyl,
wherein in formulae (IV.1.b1), (IV.1.b2) and (IV.1.b3), in particular (IV.1.b1)
X, Q, R₁, R₂ and R₃ and Z are as defined in herein, preferably for preferably for formulae IV,
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl, preferably C₇ aryl-carbonyl,
   highly preferably each of R₁, R₂, and R₃ is selected from C₈-C₁₁ aralkyl-carbonyl, C₇-C₁₁ aryl-carbonyl, in particular C₇-C₁₁ aryl-carbonyl, preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in this claims for any of R₁, R₂, and R₃ highly preferably R₄ is selected from C₈-C₁₁ aralkyl-carbonyl, C₇-C₁₁ aryl-carbonyl, in particular C₇-C₁₁ aryl-carbonyl, preferably C₇ aryl-carbonyl, and M is Ir or Rh, preferably Ir.

Said compound preferably has a formula preferably selected from the group of formulae (IV.2.b1), (IV.2.b2) and (IV.2.b3), in particular (IV.2.b1),

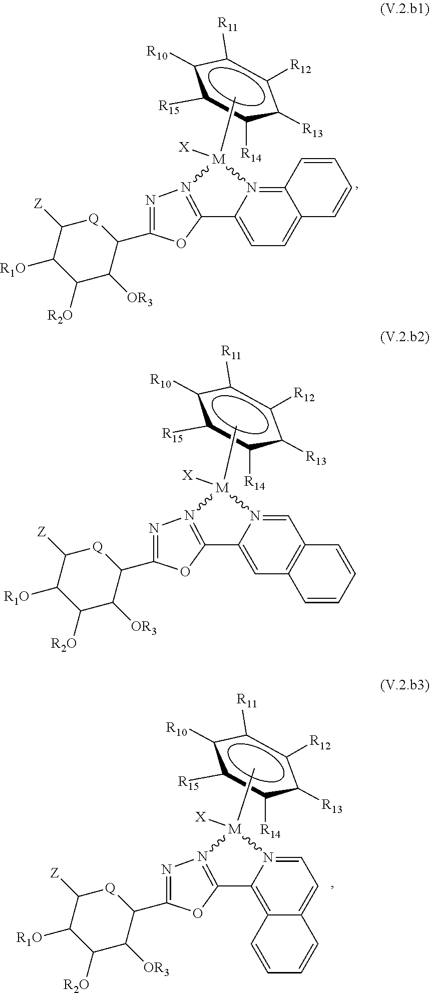

wherein in formula (V.2.b1), (V.2.b2) and (V.2.b3), in particular (V.2.b1)
X, Q, R₁, R₂ and R₃ and Z are as defined herein, preferably for formulae IV, preferably for formulae (IV.b1), (IV.b2) or (IV.b3), in particular formula (V.b1),
M is Ru or Os, preferably Os, and
R₁₀, R₁₁, R₁₂, R₁₃, R₁₄ and R₁₅ are selected from, independently, H and C₁-C₃ alkyl groups, preferably at least two of R₁₀, R₁₁, R₁₂, R₁₃, R₁₄ and R₁₅, preferably two, being selected from C₁-C₃ alkyl groups,
preferably from methyl and isopropyl, highly preferably in a para position,
preferably having the formula of (V.2.1)
wherein in formulae (V.1b1), (V.1.b2) and (V.1.b3), in particular IV1b1)
X, Q, R₁, R₂ and R₃ and Z are as defined in herein, preferably for formulae (V.2.b1), (V.2.b2) and (V.2.b3), in particular (V.2.b1)
   X is Cl,
   Q is O,
   each of R₁, R₂, and R₃ is selected from C₄-C₈ alkyl, C₅-C₈ alkyl-carbonyl, C₈-C₁₁ aralkyl-carbonyl, C₇-C₁₁ aryl-carbonyl, in particular C₇-C₁₁ aryl-carbonyl, preferably C₇ aryl-carbonyl,
   highly preferably each of R₁, R₂, and R₃ is selected from C₈-C₁₁ aralkyl-carbonyl, C₇-C₁₁ aryl-carbonyl, in particular C₇-C₁ aryl-carbonyl, preferably C₇ aryl-carbonyl,
   Z is selected from H, C₁-C₆ alkyl, C₂-C₆ alkenyl, C₂-C₆ alkynyl, —CH₂OR₄, or OR₄, wherein R₄ is as defined in these claims for any of R₁, R₂, and R₃ highly preferably R₄ is selected from C₈-C₁₁ aralkyl-carbonyl, C₇-C₁₁ aryl-carbonyl, in particular C₇-C₁₁ aryl-carbonyl, preferably C₇ aryl-carbonyl, and
      and M is Ru or Os preferably Os.

Preferably, in any of formulae (I) to (V), including preferred and particular options

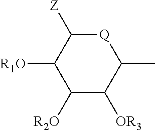

moiety is a glycopyranosyl ring O-substituted with R₁, R₂, R₃ and R₄ as defined for any of formulae (I) to (V), respectively,
in particular an L-glycopyranosyl ring,
in particular an glycopyranosyl ring (preferably of L-configuration) linked via an alpha linkage to ring Het₁.
L-glycopyranosyl sugar analogues are preferred. D-glycopyranosyl ring sugar analogues are also included.
Both alpha linkage and beta linkage compounds are included.
in a particularly preferred embodiment each of R₁, R₂, and R₃ is selected from C₈-C₁₁ aralkyl-carbonyl, C₇-C₁₁ aryl-carbonyl, in particular C₇-C₁₁ aryl-carbonyl, preferably C₇ aryl-carbonyl,
Z is selected from H, C₁-C₆ alkyl, C₂-C₆ alkenyl, C₂-C₆ alkynyl, —CH₂OR₄, or OR₄, preferably —CH₂OR₄, or OR₄, in particular —CH₂OR₄, wherein R₄ is as defined in this claims for any of R₁, R₂, and R₃, in particular R₄ is C₇-C₁₁ aryl-carbonyl, preferably C₇ aryl-carbonyl,
In a particularly highly preferred embodiment each of R₁, R₂, R₃ and R₄ is benzoyl.

Particularly preferably said compound has a distribution coefficient (log D) of at least 1.4, preferably at least 1.6, more preferably at least 1.8, highly preferably at least 2.0. In a particular embodiment the distribution coefficient (log D) is not higher than 4.0, in particular 3.5 or 3.0. In a particular embodiment the log D is 1.4 to 4, preferably 1.6 or 1.8 to 4, in particular 1.6 or 1.8 to 3.5, in particular 2 to 4 or 2 to 3.5. In particular, log D is measured between n-octanol saturated with aqueous PBS solution (pH=7.4), and aqueous PBS solution, preferably as disclosed herein.

Highly preferably log D is measured with the method described by Kozsup et al. (Kozsup et al., 2020).

Highly preferably, the compound is selected from the group consisting of
a complex of a transition metal ion selected from the group consisting of group VIIIA (groups 8-10) of 4d and 5d series, i.e. Ru, Os, Rh, Ir, Pd and Pt, preferably selected from Ru, Os, Rh, Ir, highly preferably from Ru and Os, in particular Os with any of the following ligands:
1-(2′,3′,4′,6′-Tetra-O-benzoyl-β-D-glucopyranosyl)-4-(pyridin-2-yl)-1,2,3-triazole,
2-(2′,3′,4′,6′-Tetra-O-benzoyl-β-D-glucopyranosyl)-5-(pyridin-2-yl)-1,3,4-oxadiazole,
2-(2′,3′,4′-Tri-O-benzoyl-β-D-xylopyranosyl)-5-(pyridin-2-yl)-1,3,4-oxadiazole,
2-(2′,3′,4′,6′-Tetra-O-benzoyl-β-D-galactopyranosyl)-5-(pyridin-2-yl)-1,3,4-oxadiazole,
   with a halogen selected from the group consisting of Cl, Br and I, preferably Cl and Br, and
   with a substituted or unsubstituted penta- or hexahapto coordinating cap molecule having at least one 5 or 6 membered aromatic ring.

Highly preferably, the compound is selected from the group consisting of
a complex of a transition metal ion selected from the group consisting of group VIIIA (groups 8-10) of 4d and 5d series, i.e. Ru, Os, Rh, Ir, Pd and Pt, preferably selected from Ru, Os, Rh, Ir, highly preferably from Ru and Os, in particular Os, with any of the following ligands:
1-(2′,3′,4′,6′-Tetra-O-benzoyl-β-D-glucopyranosyl)-4-(quinolin-2-yl)-1,2,3-triazole
2-(2′,3′,4′,6′-Tetra-O-benzoyl-β-D-glucopyranosyl)-4-(quinolin-2-yl)-1,3,4-oxadiazole
2-(2′,3′,4′-Tri-O-benzoyl-β-D-xylopyranosyl)-5-(quinolin-2-yl)-1,3,4-oxadiazole
2-(2′,3′,4′,6′-Tetra-O-benzoyl-β-D-galactopyranosyl)-5-(quinolin-2-yl)-1,3,4-oxadiazole
   with a halogen selected from the group consisting of Cl, Br and I, preferably Cl and Br, and
   with a substituted or unsubstituted penta- or hexahapto coordinating cap molecule having at least one 5 or 6 membered aromatic ring.

In a particularly preferred embodiment the C7¬ C11 aryl ¬ carbonyl (aroyl) is benzoyl.

In a further aspect of the present invention the compound is for use as a medicament, preferably in human medicine or in veterinary medicine.

Preferably the complex, any indication is as defined herein is any of the complexes listed above in any of formulae above, eg. formulae (I) to (V) and variants as given above.

In a particularly preferred embodiment of the present invention the compound(s) in the medicament or used for treatment is/are selected from compounds defined by formula (II), in particular for formula (III), in particularly preferred embodiment by formulae (IV) and (V) including preferred formulae. Preferred formulae include, unless specifically defined differently, compounds identified both by roman and arab numbers, like (II.1, II.2, III.1, IV.1, V.2) etc.

In particular, the compound is used in human medicine or for use in human medicine.

In particular, the compound for use as a veterinary agent or is used as a veterinary agent.

In an embodiment the compound is used in a combination therapy with an other antineoplastic or antibacterial agent. In a particular embodiment the combination therapy is with eg. PARP inhibitors.

In a particular embodiment the complexes are administered together with
inductors of primary or secondary reactive oxygen or reactive nitrogen species production (e.g. doxorubicine
any form or radiotherapy (Kim et al., 2019)).
inhibitors of the DNA damage response (e.g. PARP inhibitors (Curtin and Szabo, 2013; Curtin and Szabo, 2020)),
compound that can potentiate the DNA damage by any means or block pathways that interact with DNA damage response (e.g. PI3K inhibitors)
an antimetabolite cytostatic drugs, or drugs that modulate directly or indirectly mitochondrial metabolism or modulate the intermediary metabolism.

It is contemplated that the compounds may potentiate the effects of the elements of chemoradiotherapy.

The compound of the invention preferably has a non toxic character in particular wherein the R1, R2, R3 and/or R4 or each of them are selected from C8¬ C11 aralkyl¬ carbonyl, C7¬ C11 aryl¬ carbonyl, in particular C7¬ C11 aryl¬ carbonyl, preferably C7 aryl¬ carbonyl, in particular benzoyl.

In particular, the compound is used in a vertebrate. Preferably, the compound is used in a mammalian subject. In a preferred method the mammalian subject is selected from livestock animals and/or from companion animal species. In particular the mammalian subject is bovine, pig, a rodent e.g. murine, rabbit, cavy, guinea pig, etc., sheep, horse, a camelid, a felid, like cat, a canine, like dog etc. and also includes wild animals.

The compound may also be used in fishes, amphibians, reptiles and birds.

In particular, birds include poultry like chicken, turkey, duck, goose etc. and also include wild birds.

In a particularly preferred embodiment of the present invention the compound is for use as a medicament is selected from compounds defined by formula (II), in particular for formula (III), in particularly preferred embodiment by formulae (IV) and (V) including preferred formulae. Preferred formulae include, unless specifically defined differently, compounds identified both by roman and arab numbers, like (II.1, II.2, III.1, IV.1, V.2) etc.

In a further aspect of the present invention the compound is for use as an anti-neoplastic agent in a subject having a neoplasm or a patient endangered by development of a neoplasm or recurrence of a neoplasm wherein preferably the subject is a mammalian, highly preferably a human subject.

Preferably the compound is used as an anti-cancer agent in a patient having cancer or a patient endangered by development of cancer (e.g. recurrence of cancer).

In particular, the compound for use is for use in the treatment of a neoplasm (or cancer) selected from a carcinoma and a sarcoma. In particular the carcinoma is adenocarcinoma or squamous cell carcinoma. In particular, the sarcoma is selected from osteosarcoma or osteogenic sarcoma, chondrosarcoma, leiomyosarcoma, rhabdomyosarcoma, mesothelial sarcoma or mesothelioma, fibrosarcoma, angiosarcoma or hemangioendothelioma, liposarcoma, glioma or astrocytoma, myxosarcoma, and mesenchymous or mixed mesodermal tumors.

The neoplasm, without limitation may be eg. an ovarian cancer, a breast cancer, a skin cancer, a pancraetic cancer, a glioblastoma, etc.

In an embodiment the neoplasm may be a hematologic neoplasm.

In an embodiment the neoplasm is any neoplasm listed herein or listed in (Kumar et al., 2017).

In a further aspect of the present invention the compound is for use against pathogenic bacterial infection in a subject, wherein preferably the subject is a mammalian subject, highly preferably a human subject. In particular, the pathogenic bacteria are Gram-positive bacteria. In a particular embodiment the Gram-positive bacteria are selected from cocci, in particular the group consisting of Streptococcus, Staphylococcus, Enterococcus, in particular Staphylococcus and Enterococcus. In a particular embodiment the Gram-positive bacteria are selected from bacilli, in particular Clostridium, Corynebacterium, Listeria, Bacillus.

In a particular embodiment the Gram-positive bacteria are antibiotic resistant, preferably methicillin-resistant or vancomycin resistant, more preferably a methicillin-resistant Staphylococcus or a vancomycin-resistant Enterococcus.

In a particular embodiment the Gram-positive bacteria, are clinical antibiotic resistant bacteria, e.g. of a clinical resistant strain. In particular embodiment the clinical resistant bacterium is a methicillin-resistant strain or a vancomycin resistant strain.

The invention also relates to a method for treatment wherein a medicament comprising the compound of the invention or comprising the complex of the invention is administered to a patient in need of said medicament. Said patient in need may need prevention of a condition as defined herein or may have a condition or disease as defined herein e.g. a neoplastic disease or a fungal disease.

The compound of the invention is any of the complexes as given above e.g. for any of the formulae (I) to (V) and variants as given above.

In a particularly preferred embodiment of the present invention the compound(s) in the medicament or used for treatment is/are selected from compounds defined by formula (II), in particular for formula (III), in particularly preferred embodiment by formulae (IV) and (V) including preferred formulae. Preferred formulae include, unless specifically defined differently, compounds identified both by roman and arab numbers, like (II.1, II.2, III.1, IV.1, V.2) etc.

The invention also relates to the use of the compound of the invention in a non-therapeutic method. For example the compound may be used in experimental e.g. laboratory animals e.g. for research or drug testing.

The invention also relates to the use of the compound of the invention as an antibacterial agent in a non-therapeutic method.

In a particularly preferred embodiment of the present invention non-therapeutic antibacterial compounds are selected from compounds defined by formula (II), in particular for formula (III), in particularly preferred embodiment by formulae (IV) and (V) including preferred formulae. Preferred formulae include, unless specifically defined differently, compounds identified both by roman and arab numbers, like (II.1, II.2, III.1, IV.1, V.2) etc.

The invention also relates to the use of the compound of the invention as an antibacterial agent in vitro.

The compound of the invention is any of the complexes as given above e.g. for any of the formulae (I) to (V) and variants as given above.

In particular embodiment the compound of the invention is used as antibacterial cleaning agent.

In particular embodiment the compound of the invention is used as aseptic or disinfecting agents e.g. in aseptic or disinfecting compositions, e.g. in sanitizers for artificial and/or biological surfaces or e.g. in skin sanitizers including sanitizer of hand.

In particular embodiment the compound of the invention is used as preservative for preservation of biological material in the laboratory or in the medical field including organs or tissues or cultures.

In particular embodiment the compound of the invention is used as anti-bacterial agent e.g. as preservative, e.g. in food or animal feed. Preferably, the food industry is dairy industry or dry production industry.

In particular embodiment the compound of the invention is used as anti-bacterial agent in agriculture, e.g. as bacterial agent against bacterial pests, e.g. as antibacterial wash, e.g. for antibacterial protection of plants or crops, etc.

In a particular embodiment the invention relates to a composition comprising the compound of the invention as an active agent and at least one further substance. Preferably if the use so requires, the further substance is a biologically acceptable carrier, formulation agent or excipients.

In a particular embodiment the invention relates to a pharmaceutical composition comprising the compound of the invention as an active agent and at least one pharmaceutically acceptable carrier, formulation agent or excipient.

In preferred embodiments the pharmaceutical composition is formulated as disclosed hereinbelow in the chapter pharmaceutical preparations.

In a further aspect the invention relates to a method for synthesis of a heterocyclic N,N-chelating ligand wherein the compound of formula (XI)

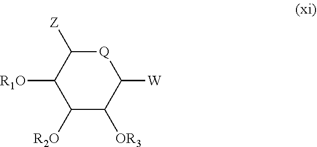

is treated with the compound of formula (xii)

Y-Het₂ (xii)

wherein
R₁, R₂, R₃, Z and Q have the meaning as defined for any of formulae (I) to (V) including preferred variants, Het₂ have the meaning as defined for any of formulae (I) to (V) including preferred variants,
   W is a polyN moiety
   Y is a functional group,
   wherein W and Y once reacted form Het₁, thereby obtaining compound (xiii)

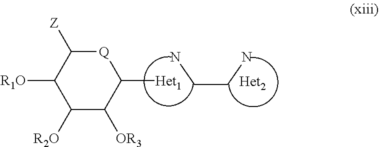

wherein the
R₁, R₂, R₃, Z and Q have the meaning as defined for any of formulae (I) to (V) including preferred variants,
Het₂ have the meaning as defined for any of formulae (I) to (V) including preferred variants.
In a preferred embodiment
W is azide (—N₃),
Y is ethynyl, and

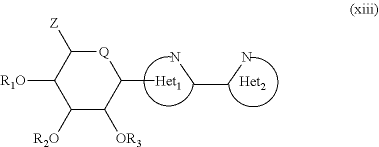

and (xi) and (xii) are reacted by an azide-alkyne cycloaddition, preferably by copper(I)-catalyzed azide-alkyne cycloaddition, to form a ligand compound having the formula (xiii), wherein Het₁ is a triazole linked through a N to group (x), and
wherein the
R₁, R₂, R₃, Z and Q have the meaning as defined hereinabove,
Het₂ have the meaning as defined hereinabove.
Preferably
W is a (x) substituted tetrazole,
Y is carboxyl, and

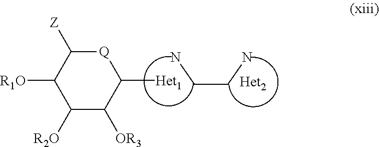

(xi) is reacted with (xii), preferably in the presence of 2-picolinic acid in the presence of DCC,
to form a ligand compound having the formula (xiii), wherein Het₁ is an oxadiazole linked through a carbon atom to group (x), and
wherein the
R₁, R₂, R₃, Z and Q have the meaning as defined hereinabove,
Het₂ have the meaning as defined hereinabove.

In a particular embodiment the invention relates to a method for synthesis of a cationic half sandwich transition metal complexes as defined herein wherein the
a dimer of the transition metal ion with two cap molecules as defined in claim 1 of formula (xiv),
[(Ar)MX₂]₂ (xiv)
is reacted with the ligand compound (xiii) to form the complex of formula (I).

In particular,
the reaction is carried out in the presence of TI[ION] TIPF₆, and
the reaction between (xiii) and (xii) results in a precipitate of TiX with the halogenide which is removed and the complex of any of claims 1 to 10 is formed in the form of a salt with [ION]
wherein [ION] is any suitable anion useful as an inert counter ion, preferably PF₆⁻ ion, wherein optionally the obtained crude salt is purified, washed and/or dried.

### Definitions

A “subject” as used herein is an individual of an animal species, preferably a vertebrate, more preferably a mammalian or avian species, in particular a mammalian species, highly preferably the individual is a primate, a hominid or a human.

A “patient” is a subject who is or intended to be under medical or veterinarian observation, supervision, diagnosis or treatment.

A “treatment” of a subject refers to any process, action, application, therapy, or the like, wherein the subject or patient is under aid, in particular medical or veterinarian aid with the object of improving the subject's or patient's condition, either directly or indirectly. Improving the subject's condition may include improving an aesthetic condition (cosmetic treatment) and/or may include, in particular, restoring or maintaining normal function of an organ or tissue, preferably at least partly restoring or maintaining health (medical or veterinarian treatment).

Treatment typically refers to the administration of an effective amount of a compound or composition described herein. Unless specified differently, a therapeutic treatment includes both medical or veterinarian treatment and prevention (or prophylaxis) i.e. prevention of the onset of a disease as well.

A “composition” of the invention is a composition of matter which comprises at least one compound of the invention as an active agent and at least one further substance. Preferably the compound of the invention is present in an effective amount. Compositions may also comprise further biologically active substances useful e.g. in a combination therapy. Furthermore, the compositions may comprise biologically acceptable carriers, formulation agents, excipients etc. which may be known in the art.

The term “effective amount” qualifies the amount of a compound required to exert the effect of the active agent in a composition. A “therapeutically effective amount” is sufficient to relieve or prevent (or prevent worsening of) one or more of the symptoms or characteristic parameters of a condition, e.g. a disorder or disease.

A “group” (or “moiety”) is used herein as a part of a molecule which can be derived in principle by removing another part, like a hydrogen atom.

As used herein, the term “alkyl” alone or in combinations means a straight or branched-chain (if appropriate) hydrocarbon group containing preferably from 1 to 20, preferably 1 to 15, 1 to 10 or 1 to 8 carbon atom(s) or 1 to 6 or 1 to 4, 1 to 3 or 1 to 2 carbon atom(s) [i.e. “C₁₋₂₀”, “C₁₋₁₅”, “C₁₋₁₀” “C₁₋₈”, “C₁₋₆” or “C₁₋₄”, “C₁₋₃” or “C₁₋₂” alkyl groups, respectively], such as methyl, ethyl, propyl, isopropyl, butyl, sec-butyl and t-butyl. As an example “C₁₋₆alkyl” includes methyl group, ethyl group, isopropyl group, butyl group, n-butyl group, isobutyl group, sec-butyl group, t-butyl group, pentyl group, isopentyl group, 2,3-dimethylpropyl group, hexyl group etc.

As used herein, the term “alkoxy” means an alkyl-O— group in which the alkyl group is as previously described. The bond to the rest of the molecule or complex, i.e. the parent moiety is through the oxygen (if to a carbon atom, ether oxygen).

The term “alkoxy alkyl” means an alkyl group which is substituted by an alkoxy group, i.e. an alkyl-O— group as previously described. The bond to the alkyl moiety is through the oxygen, i.e. it is an ether oxygen.

As used herein, the terms “alkyl-carbonyl”, “alkenyl-carbonyl” and “alkynyl-carbonyl” mean a moiety having carbonyl group substituted with an alkyl group, alkenyl group and alkynyl group, respectively, wherein bond to the parent moiety is through the carbon of the carbonyl group. An “alkyl-carbonyl”, “alkenyl-carbonyl” and “alkynyl-carbonyl” are also called alkanoyl, alkenoyl and alkynoyl, respectively.

An “alkenyl” as used herein, alone or in combinations, means a straight or branched-chain unsaturated hydrocarbon group containing at least one carbon-carbon double bond, said hydrocarbon group containing preferably from 2 to 20, preferably 2 to 15, 2 to 10 or 2 to 8 carbon atoms or 2 to 6, 2 to 4, 2 to 3 or 2 carbon atoms [i.e. “C₂₋₂₀”, “C₂₋₁₅”, “C₂₋₁₀”, “C₂₋₈”, “C₂₋₆” or “C₂₋₄”, “C₂₋₃” or “C₂” alkyl groups, respectively].

An “alkynyl” as used herein, alone or in combinations, means a straight or branched-chain unsaturated hydrocarbon group containing at least one carbon-carbon triple bond, said hydrocarbon group containing preferably from 2 to 20, preferably 2 to 15, 2 to 10 or 2 to 8 carbon atoms or 2 to 6, 2 to 4, 2 to 3 or 2 carbon atoms [i.e. “C₂₋₂₀”, “C₂₋₁₅”, “C₂₋₁₀”, “C₂₋₈”, “C₂₋₆” or “C₂₋₄”, “C₂₋₃” or “C₂” alkyl groups, respectively].

The term “cycloalkyl” as used herein is a non-aromatic carbon-based alkyl ring composed of at least three carbon atoms.

The meaning of “straight chain alkyl” is an alkyl in which each non-terminal and non-first carbon atom of the group binds covalently to two other carbon atoms. In other words a “straight chain alkyl” forms a single central carbon chain and does not comprise any carbon atom binding to the central carbon chain by replacing a H of said chain.

The meaning of “branched chain alkyl” is an alkyl in which there is at least one non-terminal and non-first carbon atom of the group which binds covalently to three or four other carbon atoms. In other words a “branched chain alkyl” has a central carbon chain which is the longest chain of consecutive carbon atoms in the group and comprises at least one carbon atom of an alkyl substituent binding to the central carbon chain.

A “heterocyclic” ring as used herein is a cyclic moiety that has, besides carbon atom(s), atoms of at least one non-carbon element as member(s) of its ring(s). A heterocycle may comprise multiple rings, e.g. it may comprise an aromatic heterocycle and, fused to the aromatic heterocycle another ring which may or may not be aromatic; i.e. if it is not aromatic it may form a cyclic substituent of the aromatic heterocycle. In a preferred embodiment, if the heteroaryl comprises multiple, in particular two fused rings, both rings are aromatic. Preferably the ring(s) of the heterocyclic moiety is/are 5 to 6 membered ring(s).

An “N-heterocyclic” ring as used herein is a heterocyclic ring comprising at least one N as a ring-forming heteroatom. The “N-heterocyclic” ring may comprise other heteroatoms, typically N, S and O atoms as well.

The term “heterocycloalkyl” refers to a “heterocyclic” ring which is derivable from cycloalkyl group as defined above, wherein at least one of the carbon atoms of the ring is replaced with a heteroatom such as, but not limited to, nitrogen or oxygen.

An “aromatic” moiety as used herein can be described as a planar cyclic moiety (a ring) wherein the single bonds (called σ-bonds) between the ring-forming atoms are formed from overlap of hybridized atomic sp2-orbitals in line between the carbon nuclei, wherein a system of delocalized π-bonds are formed from overlap of atomic p-orbitals of each of the ring forming atoms above and below the plane of the ring and wherein the number of π electrons, which is provided by the ring-forming atoms, participates in according to molecular orbital theory, must be equal to 4n+2 (Hückel's rule), in which n=1, 2, 3, etc., preferably 1 or 2, for a single ring with six π electrons, n =1. The ring-forming atoms typically provide one or two π electrons to the delocalized π electron system.

The term “heteroaryl” is defined herein as a group or molecule that contains an aromatic heterocycle, preferably a moiety that has at least one heteroatom, as “member”, incorporated within an aromatic ring. Examples of heteroatoms include nitrogen, oxygen and sulfur, preferably nitrogen and oxygen. In an embodiment a heteroaryl may comprise an aromatic heterocycle and, fused to the aromatic heterocycle another ring which may or may not be aromatic; i.e. if it is not aromatic it may form a cyclic substituent of the aromatic heterocycle. In a preferred embodiment, if the heteroaryl comprises multiple, in particular two fused rings, both rings are aromatic. Members of a heteroaryl relate to the ring-forming atoms, either carbon atom(s) or heteroatom(s).

The term “aryl” as used herein is a group that contains any carbon-based aromatic ring which is preferably a mono- or bicyclic group, wherein the bicyclic group is preferably comprises two fused rings. In a preferred embodiment the aryl group consists of carbon as ring atoms, i.e. “members” only. In a broader meaning the term aryl also includes optionally “heteroaryl”. Optionally, the term “aryl” is limited to non-heteroaryl which is also included into the term aryl and defines a group that contains an aromatic group that does not contain a heteroatom.

An aryl group may be substituted or unsubstituted (i.e. optionally substituted). If the aryl group is substituted it may be substituted with any substituent, and examples of the substituent include C₁₋₄ alkyl, C₂₋₄ alkenyl, C₁₋₃ alkyloxy, C₁₋₃ alkanoyl, C₁₋₃ alkylamine, C₁₋₃ alkylamide, halogen, etc.

“N-heteroaryl” as used herein is an aromatic N-heterocycle, i.e. an aromatic heterocycle having at least one ring N. Optionally, an N-heteroaryl comprises further heteroatoms e.g. nitrogen, oxygen and sulfur, preferably nitrogen and oxygen.

The term “aralkyl” as used herein refers to an aryl alkyl group which is linked to the parent molecule through the alkyl group, which may be further optionally substituted with one or more, preferably one to three or one to two alkyl substituents. Thus, the aryl group may be substituted with an alkyl substituent, preferably each substituent being not larger than a C₁₋₄ alkyl.

“Aryl” or “heteroaryl” may comprise a monocyclic ring, a condensed ring, or a polycyclic ring in which a single ring is bounded by a single bond, preferably a monocyclic or bicyclic ring.

An “aryl-carbonyl” is a moiety having carbonyl group substituted with an aryl group, wherein bond to the parent moiety is through the carbon of the carbonyl group. An “aryl-carbonyl” may be also called aroyl.

An “aralkyl-carbonyl” is a moiety having carbonyl group substituted with an aralkyl group, wherein binding to the parent moiety is through the carbon of the carbonyl group. An “aryl-carbonyl” may be also called aralkanoyl.

A “cap” or “capping” is used herein as a moiety of half-sandwich type metal comprising organometallic compounds which have an aromatic group in the coordination sphere which contributes to the stabilization of the metal ion and to the maintenance of the hydrophilic/lipophilic balance of the whole molecule. Cap molecules having an aromatic ring are preferably a η⁶-arene or η⁵-arenyl residues. In a preferred embodiment the half-sandwich type metal comprising organometallic compounds have a bidentate chelating ligand coordinated to the metal ion.

A “chelate” comprises at least two “coordinate bond” or “dative bond”, i.e. a two-center, two-electron bond in which the two electrons derive from the same atom, typically a non-binding electron pair is coordinated to a metal ion. Such a bond is marked in an undulating line in the formulae.

As used herein, the term “fused ring” means that the ring is fused with at least one other ring to form a group of a compound which comprises two or more rings wherein a single bond between two member atoms of the rings is, together with said two members, common in, i.e. shared by the two rings. An example of fused rings is a polycyclic aryl. A polycyclic aryl is understood herein as a group that contains multiple rings of a carbon-based group among which at least one ring is an aryl and which optionally may also comprise a cycloalkyl and/or a heterocycloalkyl.

A “substituted” moiety comprises a substituent selected from the groups and moieties as defined herein; however a substituent is preferably smaller, i.e. shorter, i.e. consists of not more, preferably less atoms than the moiety which is/are substituted thereby. In the present invention, “optionally substituted”, i.e. “unsubstituted or substituted” means that it may be substituted with any substituent.

The singular forms “a”, “an” and “the”, or at least “a”, “an”, include plural reference unless the context clearly dictates otherwise.

The term “comprises” or “comprising” or “including” are to be construed here as having a non-exhaustive meaning and allow the addition or involvement of further features or method steps or components to anything which comprises the listed features or method steps or components. “Comprising” can be substituted by “including” if the practice of a given language variant so requires or can be limited to “consisting essentially of” if other members or components are not essential to reduce the invention to practice.

### BRIEF DESCRIPTION OF THE DRAWINGS

FIG. 1. Superposition of the ¹H NMR (A) and ¹³C NMR (B) spectra of Ru-7 (black), ligand L-7 (green) and Ru-dimer (blue) in CDCl₃.

FIG. 2. A representative example for the stability of the complexes in aqueous medium—Time dependence of the 1H NMR spectra of Ru-3a in D2O over a 2 day period. By the addition of KCl (100 eq.) the original spectrum could be recovered indicating only Cl-/D20 exchange during the period of time of the study.

FIG. 3. Screening of ruthenium complexes for cytostatic properties. A-B 1×10⁴ (on panel A, MTT assays) or 3×10³ (on panel B, SRB assays) A2780 cells were plated to 96-well plates then (A) cells were treated with the compounds indicated for 4 hours then MTT assay was performed. (B) Another set of cells were treated with the compounds indicated for 48 hours then SRB assay was performed. Those molecules that were used in the subsequent studies are highlighted in color. All assays were performed in duplicates, all data represent three biological replicates. Values were normalized for vehicle treated cells: absorbance for vehicle treated cells equal to 1.

FIG. 4. Assessment of the bioactive ruthenium complexes for acute toxicity. 1×10⁴ A2780 cells, 8×10³ ID8 cells and 6×10³ primary fibroblasts were plated to 96-well plates. Cells were treated with the compounds in the concentrations indicated for 4 hours, then MTT assay was performed. Data is represented as average ±SEM, from three biological replicates, individual assays were performed in duplicates. Normality was tested, Ru-4/L4 fibroblast, dataset had normal distribution, in Ru-4/L4 ID8, dataset normality was achieved by logarithmic transformation, finally in Ru-4/L-4 A2780, Ru-6/L-6 A2780, Ru-8/L-8 A2780, Ru-2a/L-2a A2780, Ru-6/L-6 ID8, Ru-8/L-8 ID8, Ru-2a/L-2a ID8, Ru-4/L-4 fibroblast, Ru-8/L-8 fibroblast, Ru-2a/L-2a fibroblast datasets were normalized using the Box-Cox normalization method. Statistical significance was determined using a two-way ANOVAtest, all measurement points were compared with each other. *, ** and *** indicate statistically significant differences between vehicle-treated (control) and free ligand/ruthenium complex-treated cells at p<0.05, p<0.01, and p<0.001, respectively. #, ##, and ### indicate statistically significant differences between free ligand-treated and ruthenium complex-treated cells at p<0.05, p<0.01, and p<0.001, respectively. Values were normalized for vehicle treated cells. Absorbance for vehicle treated cells equal to 1.

FIG. 5. Assessment of the bioactive ruthenium complexes for cytostatic activity. 3×10³ A2780 cells, 2×10³ ID8 cells and 4×10³ primary fibroblasts were plated to 96-well plates. Cells were treated with the compounds in the concentrations indicated for 48 hours, then SRB assay was performed. Data is represented as average ±SEM, from three biological replicates, individual assays were performed in duplicates. Normality was tested, Ru-4/L-4 A2780, Ru-6/L-6 A2780, Ru-8/L-8 A2780, Ru-4/L-4 ID8, Ru-6/L-6 ID8, Ru-8/L-8 ID8, Ru-2a/L-2a ID8, datasets had normal distribution, in Ru-2a/L-2a A2780, Ru-6/L-6 fibroblast datasets normality was achieved by logarithmic transformation, finally in Ru-4/L-4 fibroblast, Ru-8/L-8 fibroblast, Ru-2a/L-2a fibroblast datasets were normalized using the Box-Cox normalization method. Statistical significance was determined using a two-way ANOVA test, all measurement points were compared with each other. *, ** and *** indicate statistically significant differences between vehicle-treated (control) and free ligand/ruthenium complex-treated cells at p<0.05, p<0.01, and p<0.001, respectively. #, ##, and ### indicate statistically significant differences between free ligand-treated and ruthenium complex-treated cells at p<0.05, p<0.01, and p<0.001, respectively. Nonlinear regression was performed on the data. Values were normalized for vehicle treated cells. Absorbance for vehicle treated cells equal to 1.

FIG. 6. Ruthenium complexes do not induce cell death. 1×10⁶ A2780 cells were plated in 6-well plates and were treated with the ruthenium complexes (Ru-2a at 1.3 μM, Ru-4 at 6.2 μM, Ru-6 at 8.5 μM and Ru-8 at 3.7 μM) and 300 μM hydrogen peroxide for 48 hours. Cells were then stained with Annexin V and propidium iodide (PI) and cells were subjected to flow cytometry as described in Materials and Methods. The percentage of cells in the quadrants are plotted. Data is represented as average ±SEM, from three biological replicates, individual assays were performed in duplicates. Normality was tested, on all datasets Box-Cox normalization was performed to achieve normal distribution. Statistical significance was determined using a two-way ANOVA test, all measurement points were compared with each other. ** and *** indicate statistically significant differences between vehicle-treated (control) and treated cells (ruthenium complexes or 300 μM H₂O₂) corresponding to the same quadrant (e.g. vehicle-treated double negative cells vs. Ru-2a-treated double negative cells) at p<0.01, and p<0.001, respectively.

FIG. 7. Determination of the cytotoxic and cytostatic activity of platinum compounds. A 1×10⁴ A2780 cells, 8×10³ ID8 cells and 6×10³ primary fibroblasts were plated to 96-well plates. Cells were treated with the compounds in the concentrations indicated for 4 hours then MTT assay was performed. B 3×10³ A2780 cells, 2×10³ ID8 cells and 4×10³ primary fibroblasts were plated to 96-well plates. Cells were treated with the compounds in the concentrations indicated for 48 hours then SRB assay was performed. Nonlinear regression was performed on the data. Values were normalized for vehicle treated cells. Absorbance for vehicle treated cells equal to 1.

FIG. 8. Ruthenium complexes induce reactive oxygen species production. A 1×10⁶ A2780 cells were plated to 6-well plates. Cells were treated with Ru-2a at 1.3 μM, Ru-4 at 6.2 μM, Ru-6 at 8.5 μM and Ru-8 at 3.7 μM for 48 hours. Then cells were harvested, protein lysate was prepared. Subsequently, 20 μg protein was separated by SDS-PAGE on an 8% gel, followed by Western blotting and the blot was stained with 4HNE and actin antibodies. Densitometry values were normalized for the control. Data is represented as average ±SEM, from three biological replicates. Since in the control group there was no variance, we did not perform statistical analysis. Values were normalized for vehicle treated cells. Absorbance for vehicle treated cells equal to 1.

FIG. 9. Reduced thiols and vitamin E can block the cytostatic effects of ruthenium complexes. A 3×10³ A2780 cells were plated to 96-well plates. Cells were treated with the reduced thiol compounds in the concentrations indicated for 48 hours then SRB assay was performed. Data is represented as average ±SEM, from three biological replicates. The individual SRB assays were performed in duplicates. Normality was tested, Ru-4/L-4, datasets had normal distribution, in Ru-2a/L-2a, Ru-6/L-6 and Ru-8/L-8 dataset normality was achieved by logarithmic transformation. Statistical significance was determined using a two-way ANOVA test, all measurement points were compared with each other. B 3×10³ A2780 cells were plated to 96-well plates. Cells were treated with the compounds in the concentrations indicated for 48 hours then SRB assay was performed. Data is represented as average ±SEM, from three biological replicates. The individual SRB assays were performed in duplicates. Normality was tested, Ru-4/L-4, Ru-8/L-8 datasets had normal distribution, in Ru-2a/L-2a and Ru-6/L-6 dataset normality was achieved by logarithmic transformation. C 3×10³ A2780 cells were plated to 96-well plates. Cells were treated with Trolox in the concentration indicated for 48 hours then SRB assay was performed. Data is represented as average ±SEM, from three biological replicates. The individual SRB assays were performed in duplicates. Normality was tested, Ru-2a/L-2a, Ru-6/L-6 and Ru-8/L-8 datasets had normal distribution, in Ru-4/L-4 dataset normality was achieved by logarithmic transformation. Statistical significance was determined using a two-way ANOVA test, all measurement points were compared with each other. *, ** and *** indicate statistically significant differences between vehicle-treated (control) and antioxidant (GSH, NAC, vitamin E or Trolox)-treated cells at p<0.05, p<0.01, and p<0.001, respectively. #, ##, and ### indicate statistically significant differences between vehicle-treated (control) and ruthenium-treated cells at p<0.05, p<0.01, and p<0.001, respectively. Values were normalized for vehicle treated cells. Absorbance for vehicle treated cells equal to 1.

FIG. 10. Rucaparib potentiates the cytostatic effects of ruthenium complexes. 3×10³ A2780 cells were plated to 96-well plates. Cells were treated with Rucaparib at 3 μM concentration for 48 hours, then SRB assay was performed. Data is represented as average ±SEM, from four biological replicates. Nonlinear regression was performed to obtain the IC50 values. Values were normalized for vehicle treated cells. Absorbance for vehicle treated cells equal to 1.

FIG. 11. Assessment of the bioactive ruthenium complexes on models for glioblastoma, breast cancer and pancreatic adenocarcinoma for cytostatic activity. 2×10³ U251 cells (glioblastoma model), 3×10³ MCF7 cells (breast cancer) and 2×10³ Capan2 (pancreatic adenocarcinoma) were plated to 96-well plates. Cells were treated with the compounds in the concentrations indicated for 48 hours, then SRB assay was performed. Data is represented as average ±SEM, from three biological replicates, individual assays were performed in duplicates. Normality was tested, Ru-4/L-4 U251, Ru-8/L8 U251, Ru-2a/L-2a U251, Ru-4/L-4 MCF7, Ru-8/L8 MCF7, Ru-4/L-4 Capan2, Ru-8/L8 Capan2, Ru-2a/L-2a Capan2 datasets had normal distribution, in Ru-6/L-6 U251, Ru-2a/L-2a MCF7, Ru-6/L-6 Capan2 datasets normality was achieved by logarithmic transformation, finally in Ru-6/L-6 MCF7 datasets were normalized using the Box-Cox normalization method. Statistical significance was determined using a two-way ANOVA test, all measurement points were compared with each other. *, ** and *** indicate statistically significant differences between vehicle-treated (control) and free ligand/ruthenium complex-treated cells at p<0.05, p<0.01, and p<0.001, respectively. #, ##, and ### indicate statistically significant differences between free ligand-treated and ruthenium complex-treated cells at p<0.05, p<0.01, and p<0.001, respectively. Nonlinear regression was performed on the data. Values were normalized for vehicle treated cells. Absorbance for vehicle treated cells equal to 1.

FIG. 12. Assessment of non-sugar ruthenium complexes on A2780 cells for cytostatic activity. 3×10³ A2780 cells were plated to 96-well plates. Cells were treated with the compounds in the concentrations indicated for 48 hours, then SRB assay was performed. Data is represented as average ±SEM, from three biological replicates, individual assays were performed in duplicates. Normality was tested, both datasets were normalized using the Box-Cox normalization method. Statistical significance was determined using a two-way ANOVA test, all measurement points were compared with each other. *** indicates statistically significant differences between vehicle-treated (control) and free ligand/ruthenium complex-treated cells at p<0.001. Nonlinear regression was performed on the data. Values were normalized for vehicle treated cells. Absorbance for vehicle treated cells equal to 1.

FIG. 13. Structure-function relationship assessment of the active Ru compounds.

FIG. 14. Screening of Ru, Rh, Os and Ir complexes with L-2a ligand and dimers for cytostatic properties. A-B 1×10⁴ (on panel A, MTT assays) or 3×10³ (on panel B, SRB assays) A2780 cells were plated to 96-well plates then (A) cells were treated with the compounds indicated for 4 hours then MTT assay was performed. (B) Another set of cells were treated with the compounds indicated for 48 hours then SRB assay was performed. All assays were performed in duplicates, all data represent three biological replicates. Values were normalized for vehicle treated cells. Absorbance for vehicle treated cells equal to 1. C The compounds that were used in the assay are indicated on FIG. 14C.

FIG. 15. Screening of Ru, Rh, Os and Ir complexes with L-4 ligand and dimers for cytostatic properties. Figures A-B show experiments with the same parameters as described for FIG. 14. C The compounds that were used in the assay are indicated on FIG. 15C.

FIG. 16. Screening of Ru, Rh, Os and Ir complexes with L-6 ligand and dimers for cytostatic properties. Figures A-B show experiments with the same parameters as described for FIG. 14. C The compounds that were used in the assay are indicated on FIG. 16C.

FIG. 17. Screening of Ru, Rh, Os and Ir complexes with L-8 ligand and dimers for cytostatic properties. Figures A-B show experiments with the same parameters as described for FIG. 14. C The compounds that were used in the assay are indicated on FIG. 17C.

FIG. 18. Determination of the cytotoxic activity of Ru, Rh, Os and Ir complexes with L-2a ligand and dimers. A 6×10³ primary fibroblasts were plated to 96-well plates. Cells were treated with the compounds in the concentrations indicated for 4 hours then MTT assay was performed. B 4×10³ primary fibroblasts were plated to 96-well plates. Cells were treated with the compounds in the concentrations indicated for 48 hours then SRB assay was performed. Nonlinear regression was performed on the data. Values were normalized for vehicle treated cells. Absorbance for vehicle treated cells equal to 1.

The compounds that were used in the assay are indicated on FIG. 14C.

FIG. 19. Determination of the cytotoxic activity of Ru, Rh, Os and Ir complexes with L-4 ligand and dimers. Figures A-B show experiments with the same parameters as described for FIG. 18.

The compounds that were used in the assay are indicated on FIG. 15C.

FIG. 20. Determination of the cytotoxic activity of Ru, Rh, Os and Ir complexes with L-6 ligand and dimers. Figures A-B show experiments with the same parameters as described for FIG. 18.

The compounds that were used in the assay are indicated on FIG. 16C.

FIG. 21. Determination of the cytotoxic activity of Ru, Rh, Os and Ir complexes with L-8 ligand and dimers. Figures A-B show experiments with the same parameters as described for FIG. 18.

The compounds that were used in the assay are indicated on FIG. 17C.

FIG. 22. Osmium complexes induce reactive oxygen species production. A 1×10⁶ A2780 cells were plated to 6-well plates. Cells were treated with Os-2a at 0.73 μM for the indicated time. Then cells were harvested, protein lysate was prepared. Subsequently, 20 μg protein was separated by SDS-PAGE on an 8% gel, followed by Western blotting and the blot was stained with 4HNE and actin antibodies.

FIG. 23. The interrelation between Nrf2 and ruthenium/osmium complexes in A2780 ovarian cancer cells. A2780 cells were treated with Nrf2 inhibitor ML385 at 1.2 μM concentration in a combination with a concentration series of Os-2a for 48 hours, then SRB assay was performed. Data is represented as average ±SEM, from two biological replicates. Nonlinear regression was performed to obtain the IC50 values. Values were normalized for vehicle treated cells.

FIG. 24. A2780 were treated with the indicated complexes at the corresponding IC50 values. Changes to cellular metabolism was detected using the Seahorse Flux analyzer. The ECAR and OCR/ECAR values were expressed as fold change compared to the untreated controls.

FIG. 25. A2780 cells were treated with 2-deoxy-glucose at 0.8 mM concentration in a combination with a concentration series of Os-2a for 48 hours, then SRB assay was performed. Data is represented as average ±SEM, from two biological replicates. Nonlinear regression was performed to obtain the IC50 values. Values were normalized for vehicle treated cells.

FIG. 26. Cisplatin resistant A2780 cells were treated with the complexes indicated. After 48 hours cell proliferation was assessed by SRB assay. Nonlinear regression was performed to obtain the IC50 values. Values were normalized for vehicle treated cells. The IC50 values were compared to the previously determined values on control A2780 cells.

FIG. 27, Determination of acute cytotoxicity and long-term cytostatic activity of cisplatin on cisplatin-resistant A2780 cells.

FIG. 28. Determination of the cytotoxic activity of Ru, Rh, Os and Ir complexes with L-2b ligand and the free ligand. Figure shows experiments with the same parameters as described for FIG. 18.

FIG. 29. Assessment of cell death on A2780 cell using Annexin-propidium iodide double staining.

FIG. 30. The indicated complexes were applied on the indicated cell lines. After 48 hours cell proliferation was assessed by SRB assay.

FIG. 31. Cisplatin resistant A2780 cells were treated with the complexes indicated. After 48 hours cell proliferation was assessed by SRB assay. Nonlinear regression was performed to obtain the IC50 values. Values were normalized for vehicle treated cells. The IC50 values were compared to the previously determined values on control A2780 cells.

FIG. 32. A2780 cells were treated with the indicated complexes with or without vitamin E. After 48 hours cell proliferation was assessed by SRB assay. Nonlinear regression was performed to obtain the IC50 values.

FIG. 33. The indicated complexes were tested on a set of VRE and MRSA clinical isolates and on reference strains. MIC values were determined.

FIG. 34-36. Determination of the cytotoxic activity of Ru, Rh, Os and Ir complexes with L-C₃-C₇ ligand and the corresponding free ligand. FIGS. 33-35 shows experiments with the same parameters as described for FIG. 18.

FIG. 37. Correlation between the log D and the IC50 values in alkyl-protected complexes.

FIG. 38. Minimum Inhibitory Concentration (MIC) measurements of bacterium strains. (A) Staphylococcus aureus ref. strain ATCC43300, (B) methicillin-sensitive Staphylococcus aureus (MSSA) clinical isolates, (C) methicillin-resistant Staphylococcus aureus (MRSA) clinical isolates, (D) Enterococcus faecalis ref. strain ATCC29112, (E) vancomycin-sensitive Enterococcus faecium (VSE) clinical isolates and (F) vancomycin-resistant Enterococcus faecium (VRE) clinical isolates were tested against the complexes as listed.

FIG. 39. Minimum Inhibitory Concentration (MIC) measurements of bacterium strains. (A) Staphylococcus aureus ref. strain ATCC43300, (B) methicillin-resistant Staphylococcus aureus (MRSA) clinical isolates, (C) Enterococcus faecalis ref. strain ATCC29112, and (D) vancomycin-resistant Enterococcus faecium (VRE) clinical isolates were tested against the complexes as listed.

### DETAILED DESCRIPTION OF THE INVENTION

Half-sandwich type organometallic complexes having a transition metal ion and bidentate ligands comprising a carbohydrate-based group linked to an azole ring and another nitrogen-comprising aromatic heterocycle were synthesized and found to have a cytostatic and cytotoxic effects in several in vitro tumor models, while lacking or having low level cytotoxicity towards non-transformed fibroblasts. The complexes also showed an antibiotic effect towards Gram-positive bacteria.

The experimental data suggest that the complexes may act by generating oxidative stress in a membrane dependent mechanism.

The present inventors have prepared a set of half-sandwich type organometallic complexes having a transition metal ion of the VIIIb group of 4d and 5d series, preferably Ru, Os, Rh, Ir. The bidentate ligands in these complexes comprise a carbohydrate-based group (preferably a monosaccharide-based group characterized by a pyranose type sugar ring) and a group with two nitrogen-comprising aromatic heterocycles the one linked to the carbohydrate-based group being an azole ring.

The compounds of the invention were identified to display long-term cytostatic effects, but little rapid toxicity on two different ovarian cancer cell lines. These compounds were not toxic towards primary human fibroblasts. The practical absence of toxicity on non-transformed fibroblasts is surprising in view of the high cytostatic effect even if certain other ruthenium complexes were found to be non-toxic (Gano et al., 2019; Liu et al., 2019; Mello-Andrade et al., 2018; Mihajlovic et al., 2020). The ligands themselves had no biological activity and the presence of the transition metal ion (ruthenium(II), osmium(II), iridium(III) or rhodium(III)) was necessary. The IC50 values of the active compounds were comparable or superior to the currently applied platinum compounds (cisplatin, oxaliplatin and carboplatin) and other sugar-containing ruthenium complexes (Berger et al., 2008; Florindo et al., 2014; Hamala et al., 2020; Hanif et al., 2013) of the art. The compounds were found to be active upon long term application, in SRB assays, similar to platinum compounds.

Initially, a set of exemplary Ru(II) compounds were analysed as shown in Table 1 and on FIG. 13.

To this end, 4-(pyridin-2-yl)- and 4-(quinolin-2-yl)-1-(β-D-glucopyranosyl)-1,2,3-triazoles (N-glycosyl derivatives) and 2-(β-D-glycosyl)-5-(pyridin-2-yl)-1,3,4-oxadiazoles (C-glycosyl derivatives) were prepared by 1,3-dipolar cycloaddition reactions. Treatment of these N,N-chelators with dichloro(η⁶-p-cymene)ruthenium(II) dimer ([(η⁶-p-cym)RuCl₂]₂) in the presence of TIPF₆ yielded the expected Ru(II)-centered complexes with the general formula [(η⁶-p-cym)Ru^{II}(N—N)Cl] PF₆ as mixtures of diastereomers.

The compounds were tested in methylthiazolyldiphenyl-tetrazolium bromide (MTT) reduction assay, to assess short term toxicity and a sulphorhodamine B (SRB) proliferation assay for measuring long term cytostasis or cytotoxicity on A2780 human ovarian carcinoma cells.

The MTT assay is a colorimetric assay for measuring cellular metabolic activity, wherein the mitochondrial metabolic activity is assessed by the activity of NAD(P)H-dependent cellular oxidoreductase enzymes, which can reduce the tetrazolium dye (MTT) to its insoluble formazan while its color is changed. The assay is suitable for measuring the number of viable cells, thereby short term cytotoxicity or cytostatic activity (shift from proliferation to quiescence).

The sulforhodamine B (SRB) assay was developed to measure drug-induced cytotoxicity and cytostasis or proliferation for drug-screening applications during a longer time-span e.g. two days. SRB is a protein dye that can form an electrostatic complex with the basic amino acid residues of proteins under moderately acidic conditions, which provides a sensitive linear response with cell number and/or cellular protein measured. The SRB assay possesses a colorimetric end point and the color development is rapid and stable. In addition, this assay does not depend on the metabolic activity of the cells and therefore shows less interference with the testing compounds.

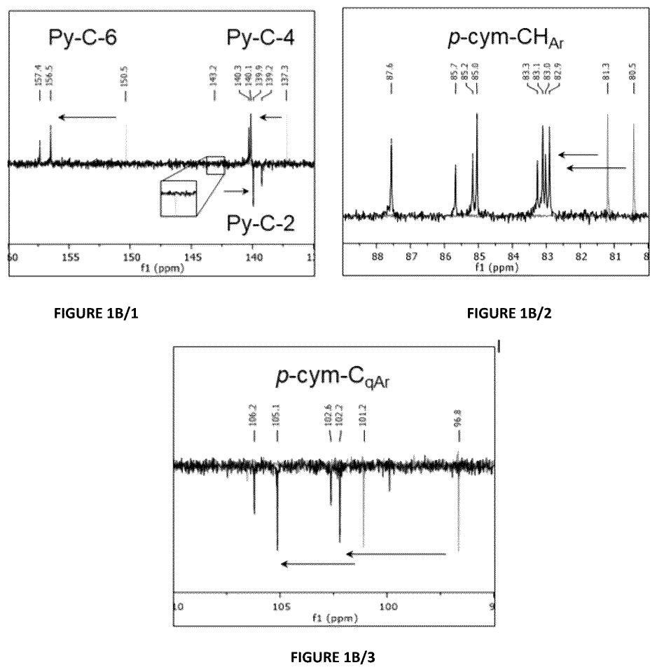

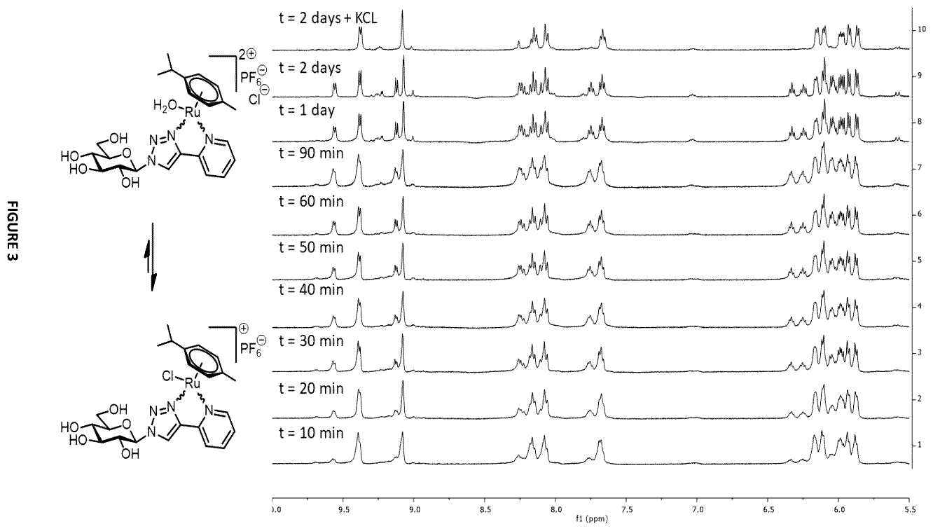

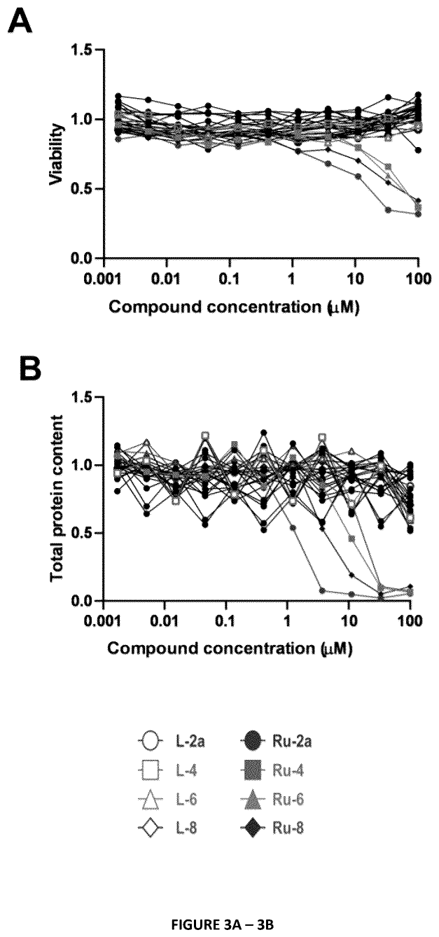

In the first set of experiments four Ru complexes, Ru-2a, Ru-4, Ru-6, Ru-8 were found to have cytostatic properties both in long-term SRB and short-term MTT assays on A2780 cells (FIG. 3A, B). The uncomplexed ligands L-2a, L-4, L-6, L-8 had no biological activity (FIG. 3A, B).

In a more detailed study these four active complexes and the corresponding ligands were tested on two ovarian cancer cell lines (A2780 and ID8) and on human primary skin fibroblasts to assess anti-neoplastic effect and toxicity on non-neoplastic cells. As a brief summary, the four active complexes, Ru-2a, Ru-4, Ru-6, Ru-8 proved to have different effect in MMT assays Ru-2a and Ru-8 being more effective on neoplastic (A2780 and ID8 cells) cells than Ru-4, Ru-6 whereas none of the complexes had any effect on non-neoplastic primary fibroblasts.

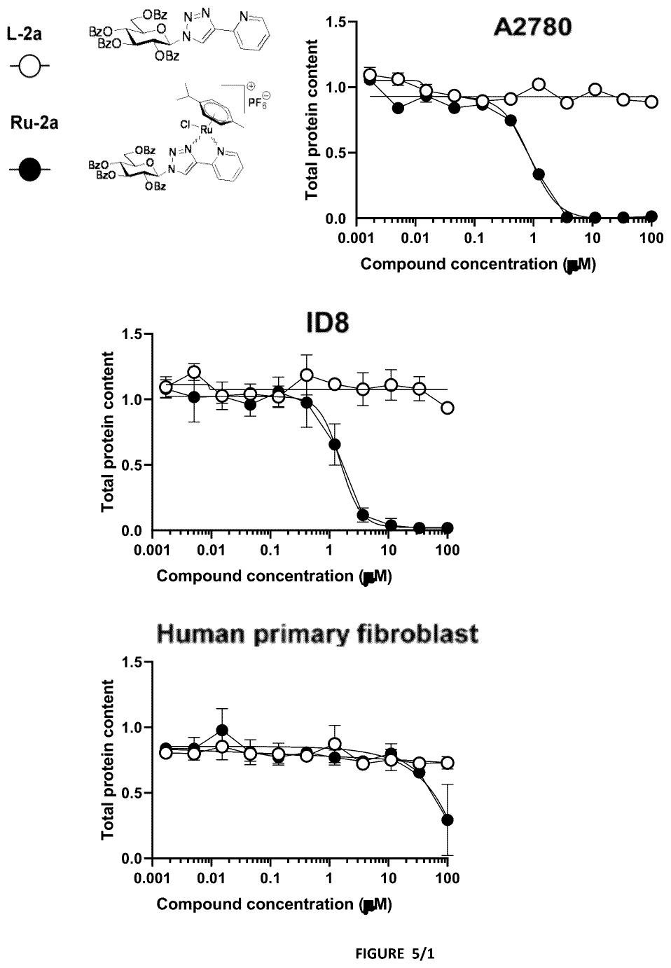

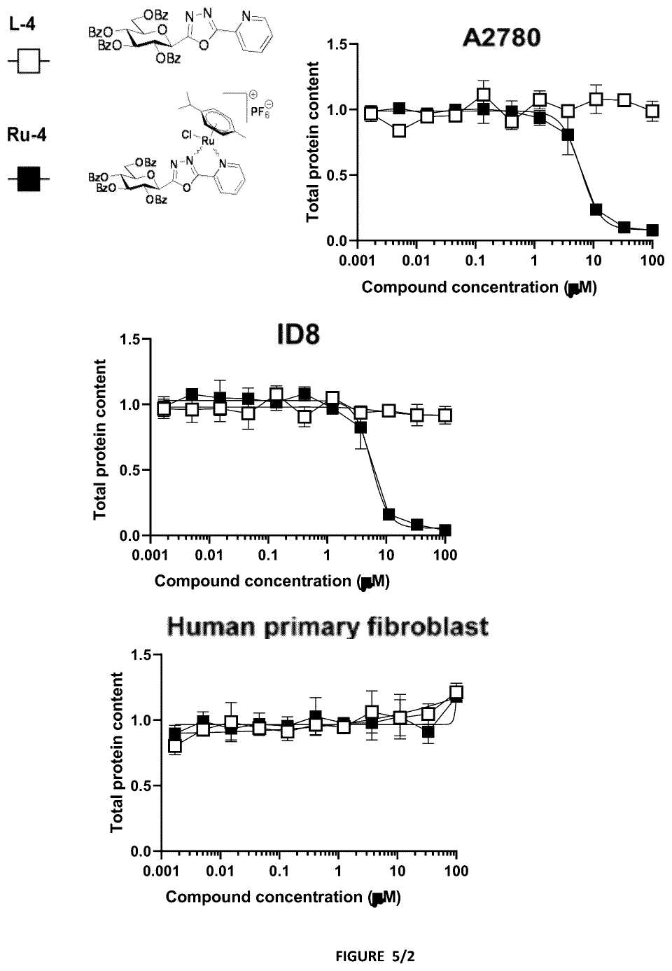

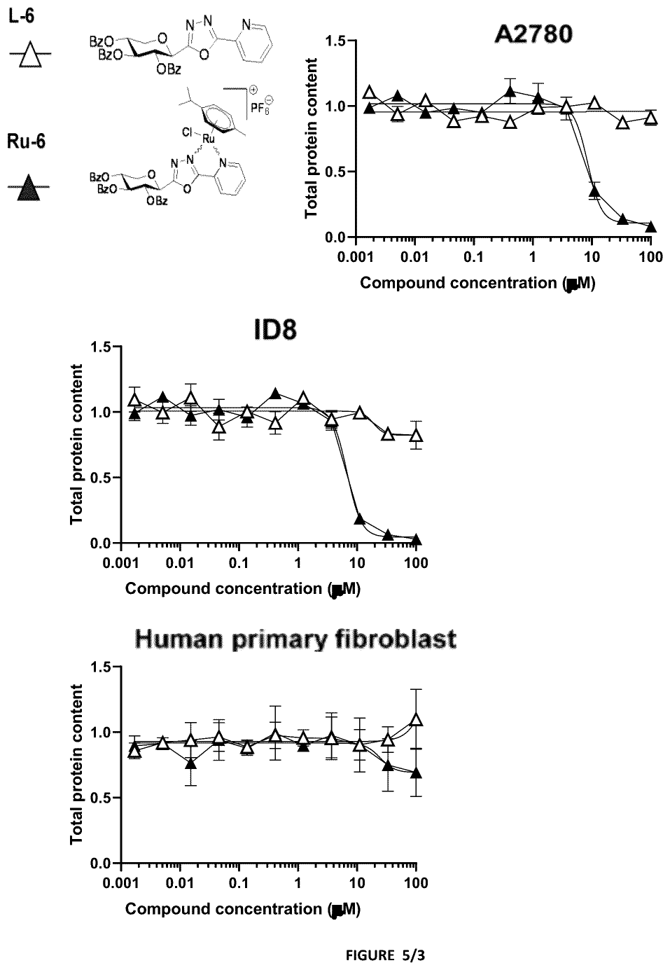

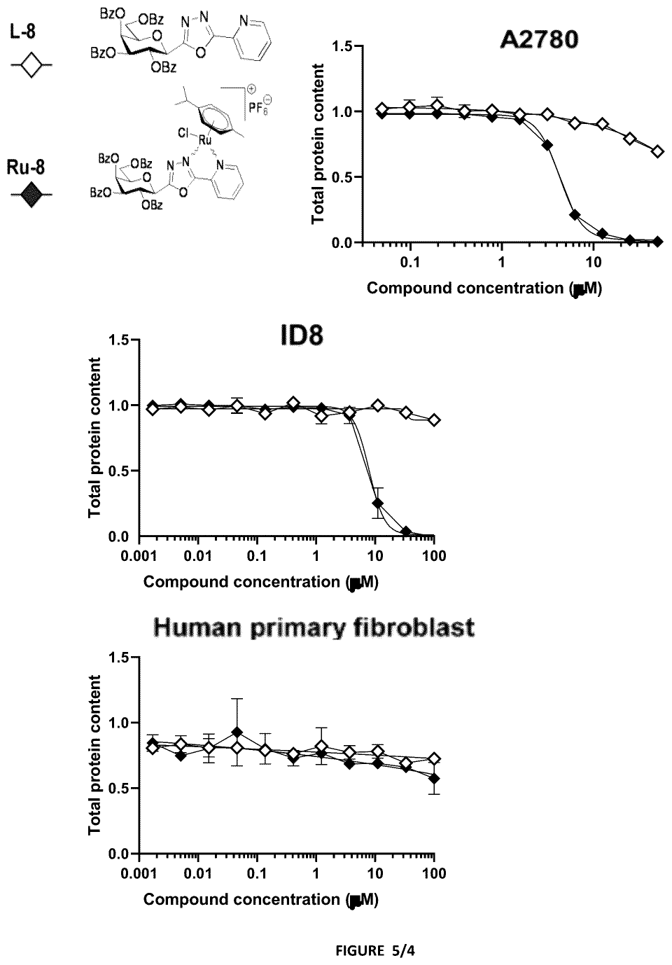

As to long-term SRB assays all complexes were effective on A2780 and ID8 cells (FIG. 5), whereas no compound but the Ru-2a and Ru-6 reduced the SRB signal on fibroblasts, however, at 100 μM only.

The possibility that long-term effect was due to enhanced cell-death has been excluded by Annexin V-propidium iodide (PI) double staining cell death measurements, a method for detecting viable, apoptotic or necrotic cells. Specifically, the Annexin V corresponding signal provides a very sensitive method for detecting cellular apoptosis, while the propidium iodide (PI) is used to detect necrotic or late apoptotic cells. Viable cells remain unstained.

The above results plausibly indicate an antineoplastic effect of the compound with a non-toxic profile on non-transformed/primary cells.

With the above measurements a relationship between the biological activity and the structure of the active molecules has been assessed. For sake of illustration all complexes in this exemplary series have been compared to Ru-4 (see FIG. 13) that had considerable cytostatic potential in SRB assays on ovarian cancer cells, while had no activity on primary fibroblasts (FIG. 13).

It could be concluded that an important structural feature, that plays a key role in the biological activity of the molecules, is the presence and size of the protecting groups on the hydroxyl groups of the carbohydrate moieties. All bioactive molecules (i.e. Ru-2a, Ru-4, Ru-6, Ru-8 in the first set of experiments) have large apolar group or groups, exemplified by O-benzoyl groups on the sugar moiety (FIG. 13). Surprisingly, changing the O-benzoyl groups to smaller O-acetyl groups (Ru-4 vs. Ru-5) or complete deprotection (Ru-4 vs. Ru-10) abrogated the inhibitory activity.

In good agreement with that, the replacement of the protected monosaccharide in the molecule with a single phenyl group lowered the cytostatic capacity of the ruthenium complex (Ru-13) or fully abrogated it (Ru-14). This finding also corroborates the inevitable role of the sugar or sugar analogue moiety in determining the biological activity, while also supported the view that apolar or aromatic groups other than O-benzoyl groups also work. The lipophilic character has been assessed by measuring distribution between n-octanol and PBS buffer wherein n-octanol was saturated with aqueous PBS solution and vice versa wherein the distribution coefficient (D) was calculated. Apparently, increasing the lipophilic character of the compounds improves the cytostatic properties of sugar-based ruthenium complexes. This is underlined by the log D values in Table 1 and also in Table 4 (see below in Example 2.5 Inhibitory properties and cooperative effect of Ru complexes and known platinum compounds) to

TABLE 1.1
Distribution coefficient of the synthesized Ru complexes (logD)

| Complex | | LogD |
|---|---|---|
| Ru-1a | 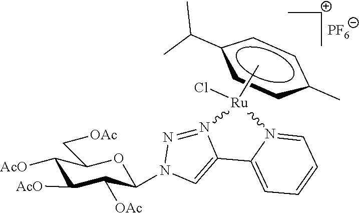 | −1.09 |
| Ru-1b | 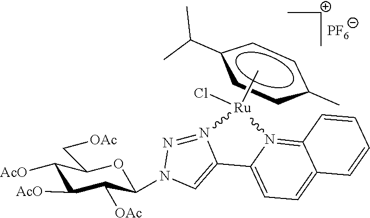 | −0.97 |
| Ru-2a | 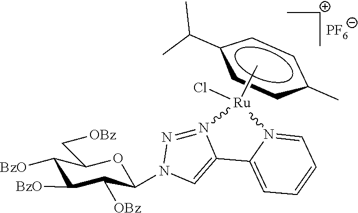 | 2.85 |
| Ru-3a | 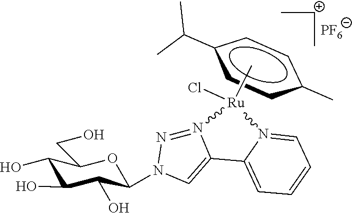 | −1.85 |
| Ru-3b | 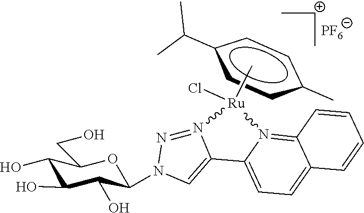 | −1.30 |
| Ru-4 | 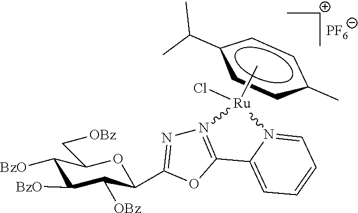 | 2.41 |
| Ru-5 | 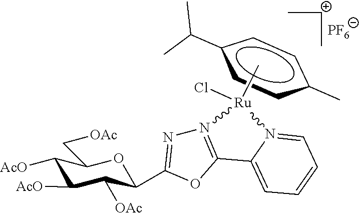 | −1.80 |
| Ru-6 | 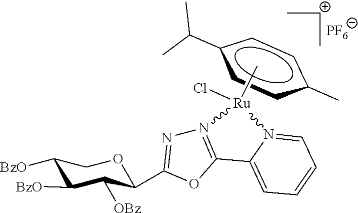 | 2.04 |
| Ru-7 | 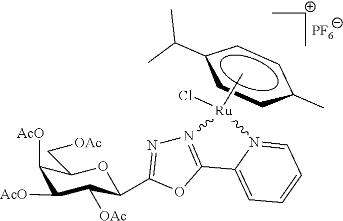 | −1.29 |
| Ru-8 | 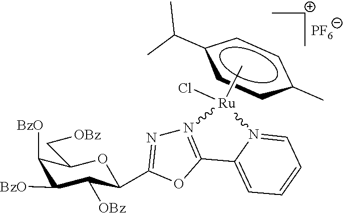 | 2.44 |
| Ru-9 | 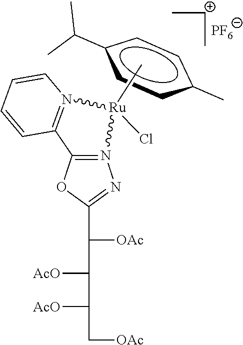 | −1.35 |
| Ru-10 | 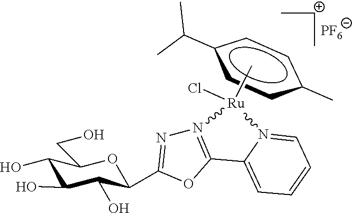 | −1.93 |
| Ru-11 | 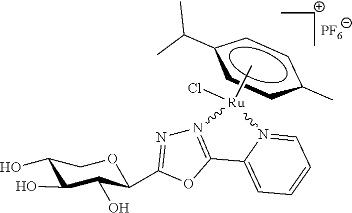 | −1.73 |
| Ru-12 | 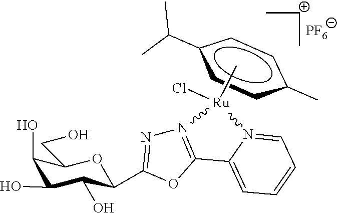 | −1.73 |
| Ru-13 | 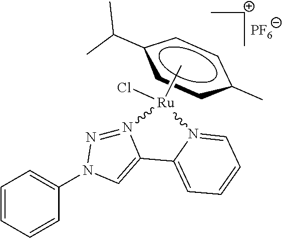 | 0.44 |
| Ru-14 | 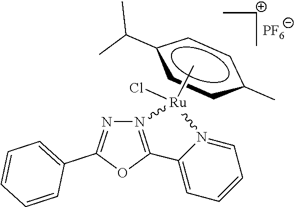 | 1.63 |

TABLE 1.2
Distribution coefficient of the synthesized complexes (logD)

| Compl. | | LogD |
|---|---|---|
| Os-2a | 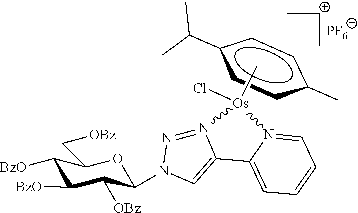 | 2.33 |
| Os-4 | 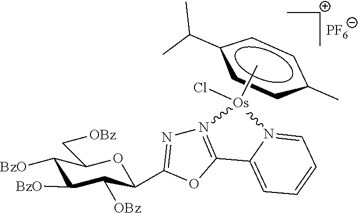 | 3.32 |
| Os-6 | 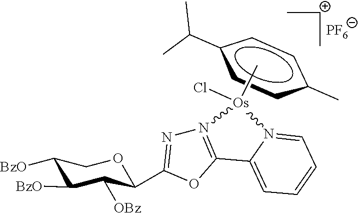 | 2.36 |
| Os-8 | 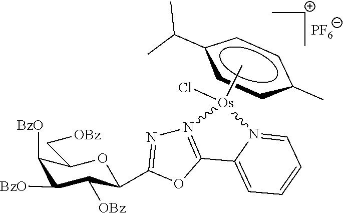 | 2.87 |
| Ir-2a | 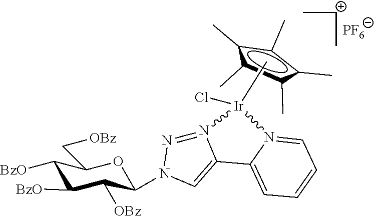 | 2.36 |
| Ir-4 | 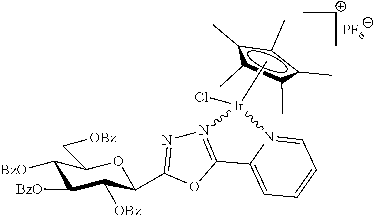 | 1.46 |
| Ir-6 | 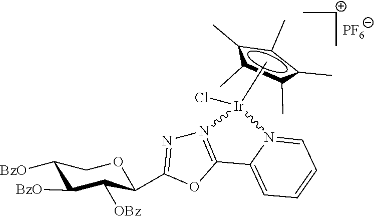 | 1.80 |
| Ir-8 | 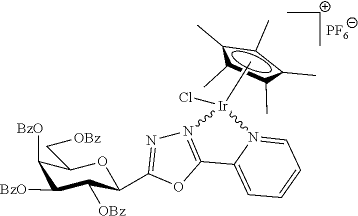 | 1.46 |
| Rh-2a | 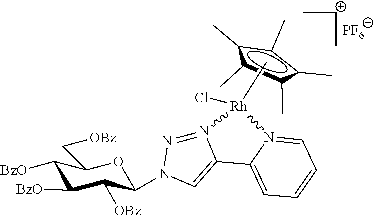 | 1.85 |
| Rh-4 | 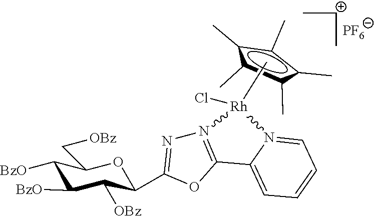 | 1.60 |
| Rh-6 | 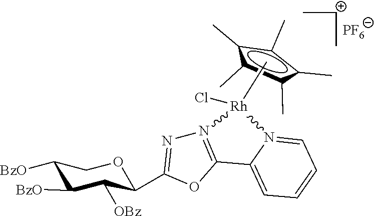 | 1.80 |
| Rh-8 | 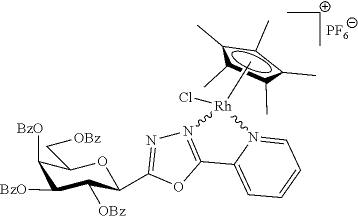 | 1.64 |

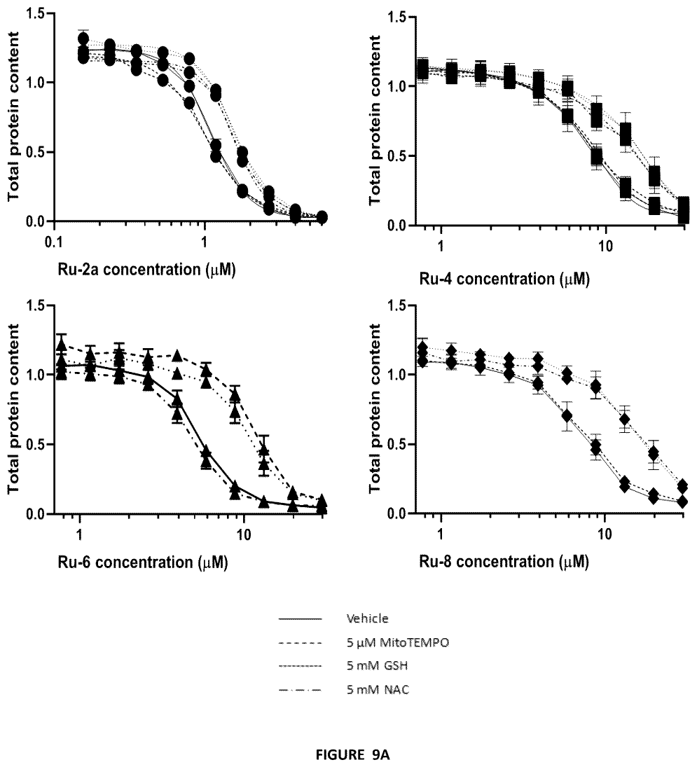

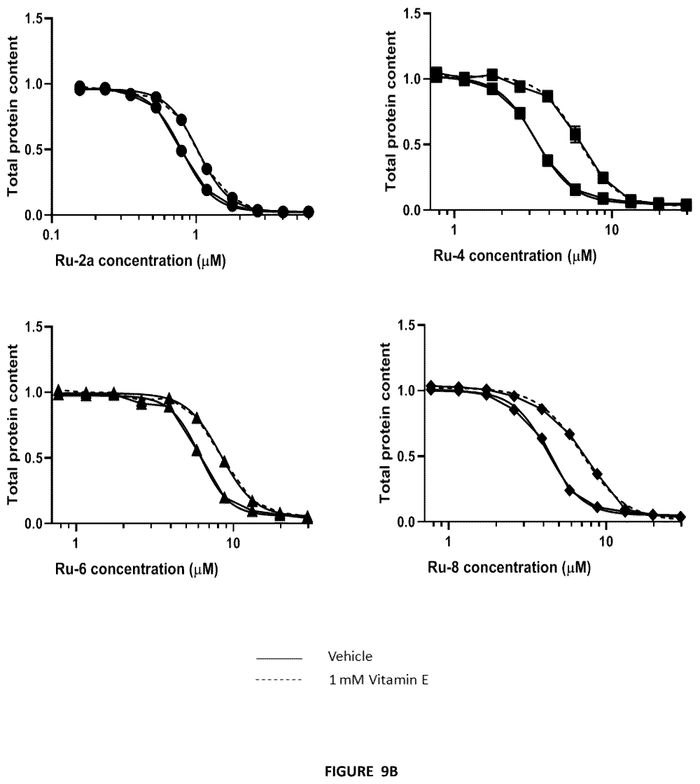

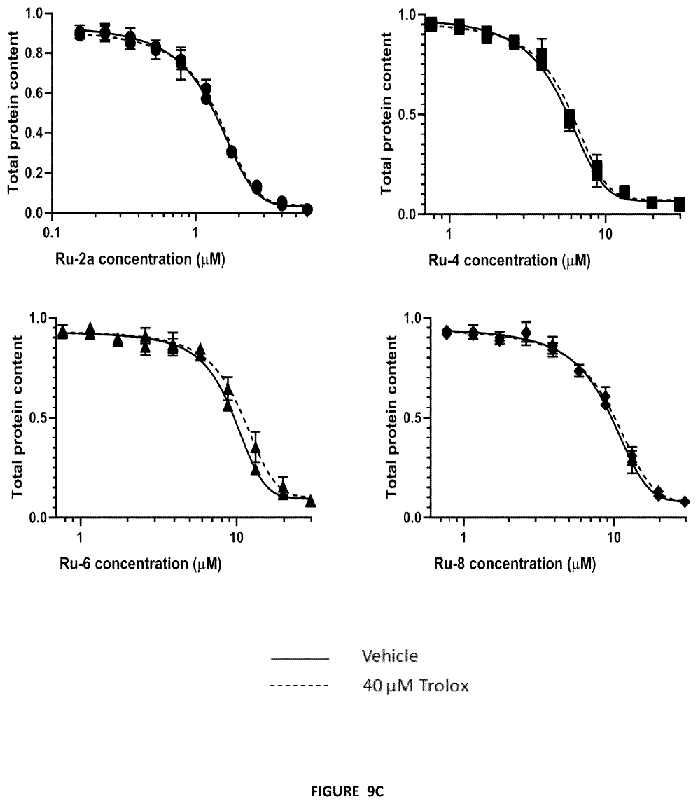

As to the mechanism of action, it has been assessed whether oxidative stress had a role in the cytostatic effects of the compounds of the invention (see Example 3). Apparently, mitochondria are not the source of the reactive oxygen species, as tested by using MitoTEMPO, a mitochondrially targeted antioxidant that can efficiently detoxify mitochondria-derived reactive oxygen species (Hegedűs et al., 2021; Sári et al., 2020). MitoTEMPO did not modulate the effects of Ru-2a, Ru-4, Ru-6 and Ru-8 (FIG. 9A).

Cytostatic effects of exemplary ruthenium complexes Ru-2a, Ru-4, Ru-6 and Ru-8 have been reverted by strong reductants. However, excess amounts of GSH or NAC, soft Lewis bases with thiol groups, can lead to the disassembly of the inventive complexes (which are soft Lewis acids).

Therefore, to bypass that possibility, vitamin E and a derivative of vitamin E lacking the apolar phytyl chain, Trolox, were applied as antioxidants without thiol group. Interestingly, application of vitamin E, similarly to GSH and NAC, attenuated the cytostatic effects (FIG. 9B), whereas Trolox did not provide protection for the cells against ruthenium compounds (FIG. 9C).

Thus it can be plausibly assumed that large, apolar protective groups could cause changes in the biophysical properties of the membranes. Without being bound by theory this can facilitate the membrane permeability or membrane entry of these compounds. The fact that Trolox, a derivative of vitamin E that lacks the long apolar phytyl chain, was ineffective in protecting cells against cytostasis induced by ruthenium compounds supports that apolar groups of the sugar or sugar-analogue and in turn membrane binding plays a role in the effect of the compounds. Taken together these findings point to the causative role of reactive oxygen species production in cytostasis and suggest that oxidative stress in apolar, lipid-containing compartments contribute to the activity of the compounds of the invention as also discussed below.

These facts provide support for claiming that compounds having apolar groups linked to the sugar or sugar analogue moiety and having a high hydrophobicity are effective in the present invention.

Furthermore, it has been found that two other structural features have impact on bioactivity: A) modification of the carbohydrate moiety from glucose to xylose by a formal removal of the hydroxymethyl group at position 5 (Ru-6) or changing the configuration of the C-4 centre (glucose to galactose as in Ru-8) and B) replacement of the 1,3,4-oxadiazole ring by 1,2,3-triazole (Ru-2a). While all these compounds were proved to be active, these changes increased the rapid toxicity of the molecules or even rendered the molecules toxic on primary cells (FIG. 13) to some extent. In addition, a triazole ring in the molecule ensured better cytostatic properties than an oxadiazole ring (Ru-13 vs. Ru-14 and Ru-2a vs. Ru-4).

It was also a surprising observation that the binding of the active compounds showed high level of cooperativity as suggested by the value of the Hill slope, being 2-3 for Ru-2a, Ru-4, Ru-6, Ru-8.

Cooperativity is a feature which normally cannot be predicted, in particular without detailed information on the structures and binding. A study of Hanif et al. (Hanif et al., 2013) assessing RAPTA-analogs (general formula 8) illustrates this by showing that a single compound was identified with a steep inhibitory curve suggesting cooperative binding whereas cooperative binding was not characteristic for other sugar-containing ruthenium complexes. The Hill equation reflects the binding of ligands to macromolecules as a function of the ligand concentration, whereby the Hill slope (or coefficient; i.e. the slope of the response curve) is a measure of ultrasensitivity and correlates with the cooperativity of the binding between the molecules under examination.

This suggests that the binding of the complexes of the invention facilitates the binding of the subsequent molecules. The Hill slope was also determined for the reference platinum-based drugs, however, these molecules did not show signs of cooperative binding as the Hill slope was ˜1. Apparently, the binding and, probably, the mode of action of the ruthenium complexes identified in this study differs from those of the reference platinum compounds.

This cooperativity is an unforeseen feature and may well contribute the activity of the compounds of the invention.

To test whether the observations in accordance with the invention apply to the group of transition metals analogous to Ru, specifically the 4d and 5d transition metals of the VIIIb group in further preferred embodiments a series of half-sandwich complexes of with real glycosyl azole type bidentate ligands and with Ru(II), Os(II), Rh(III) and Ir(III) were synthesized.

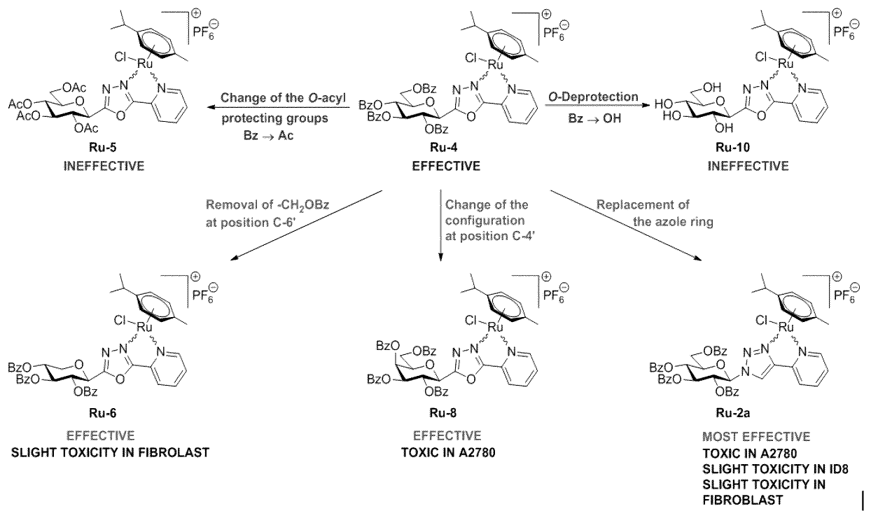

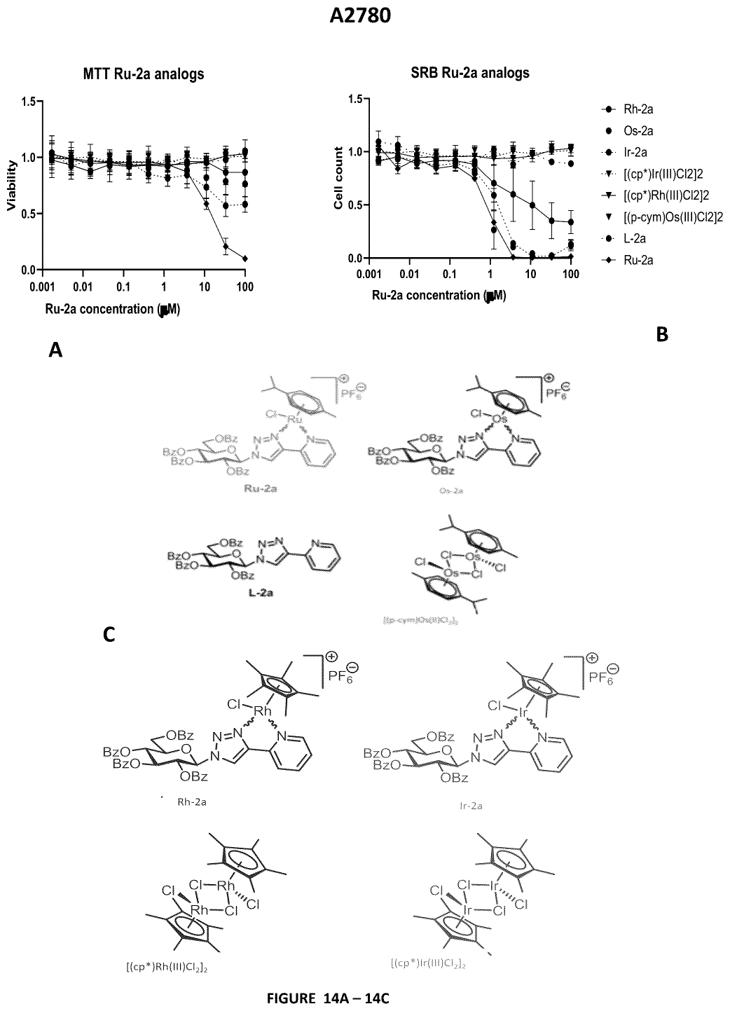

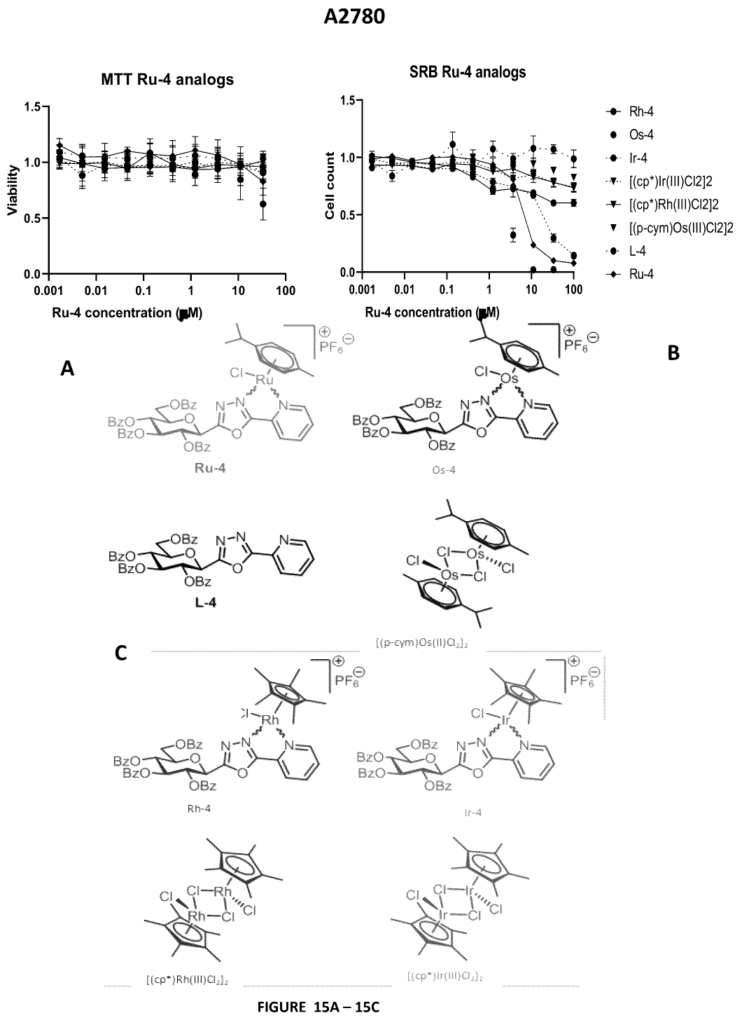

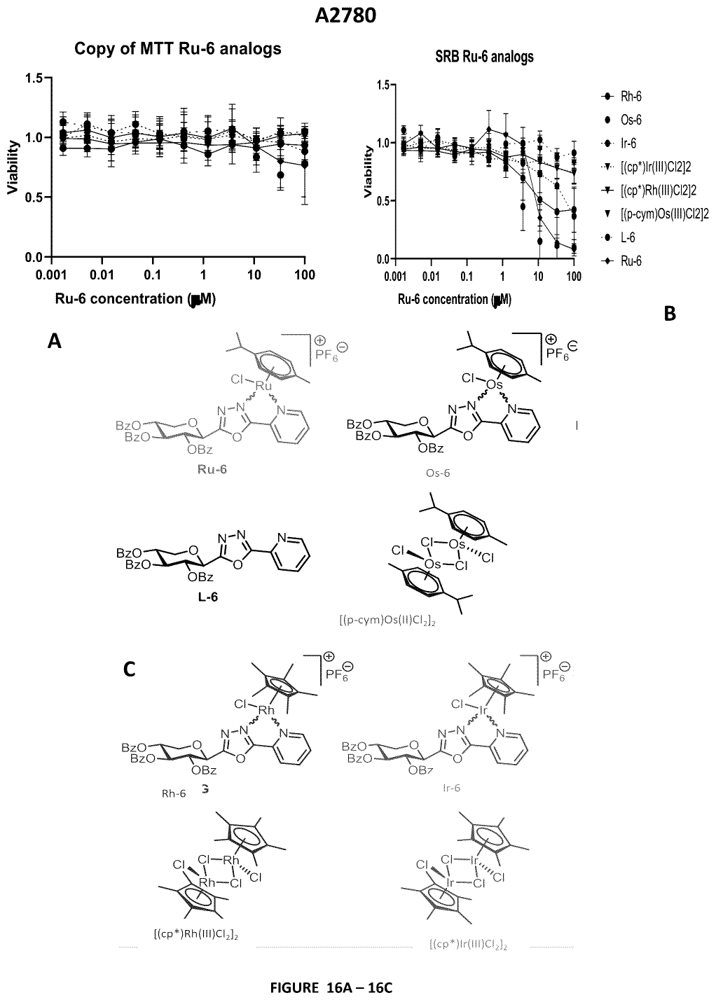

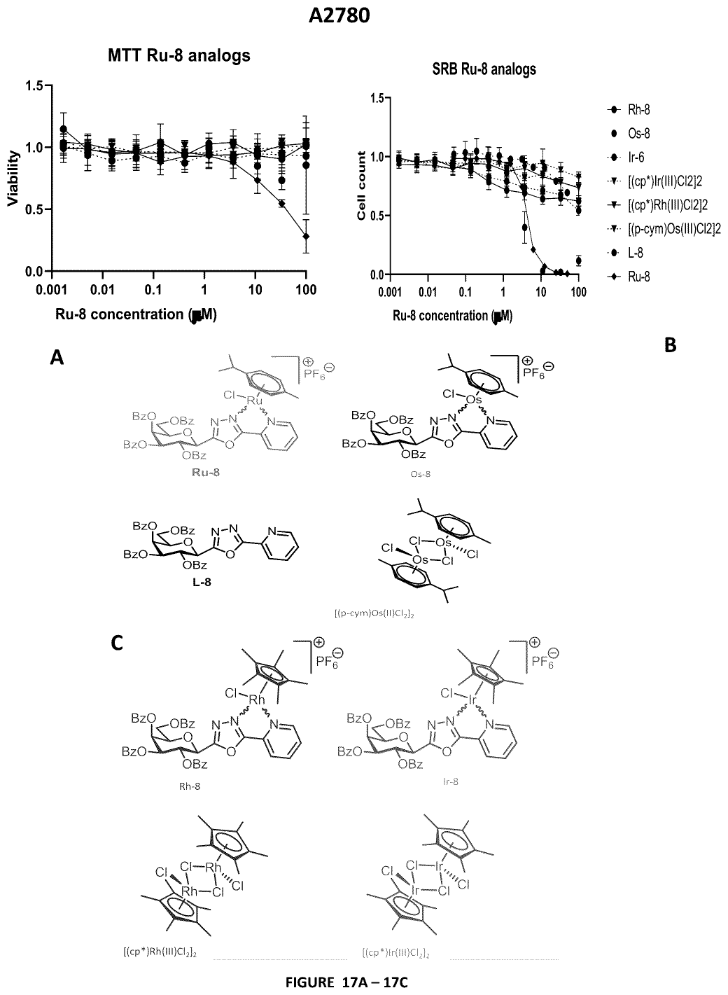

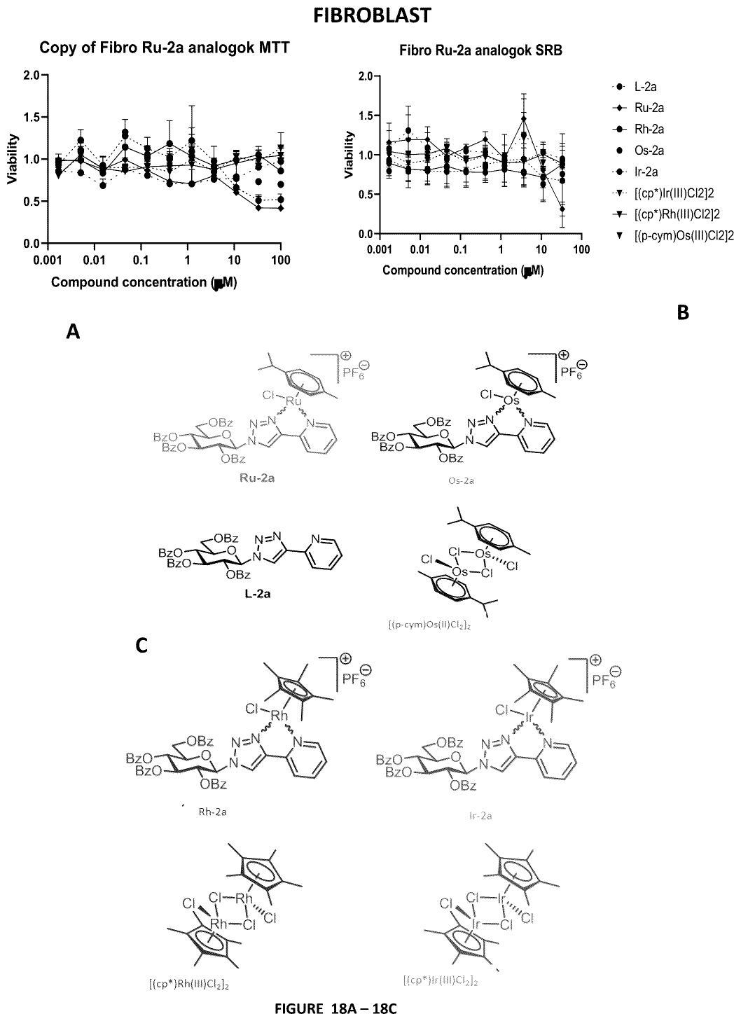

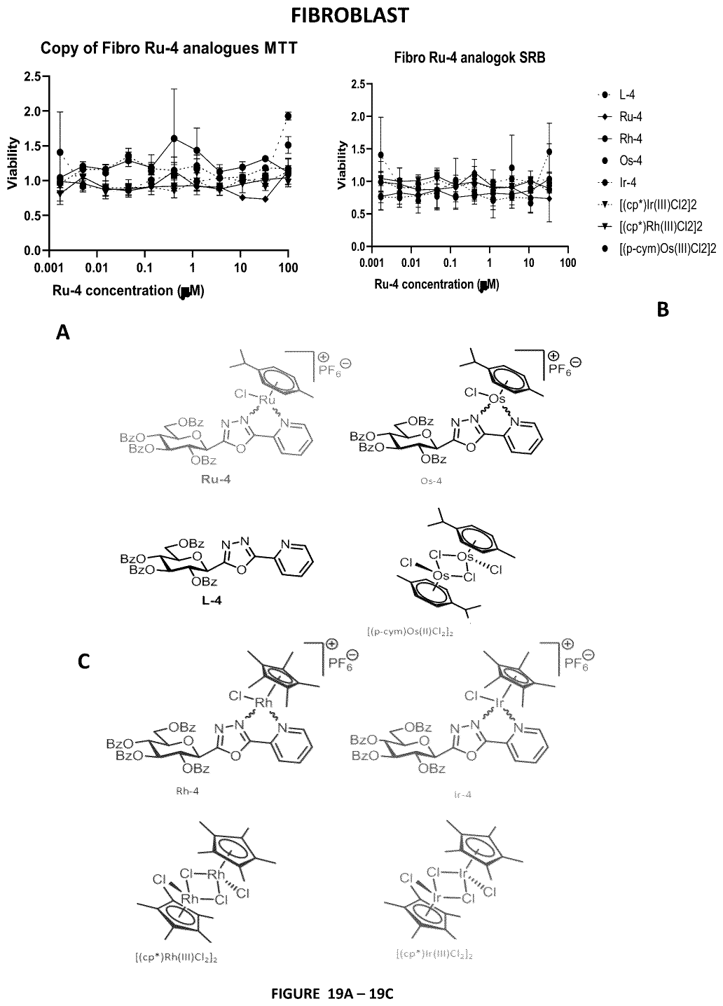

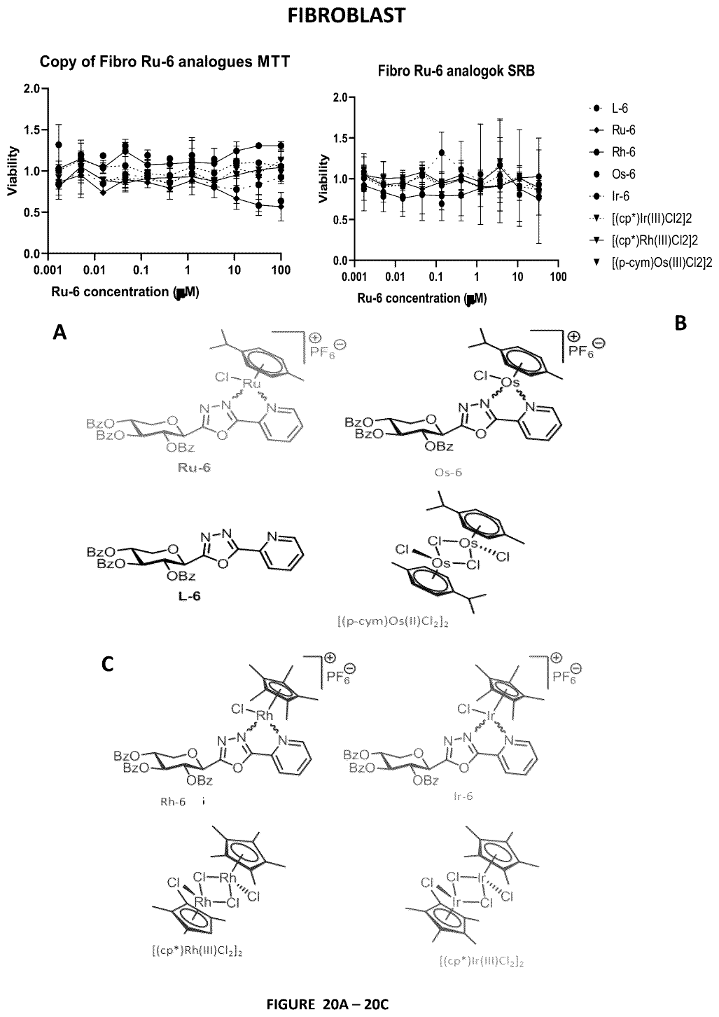

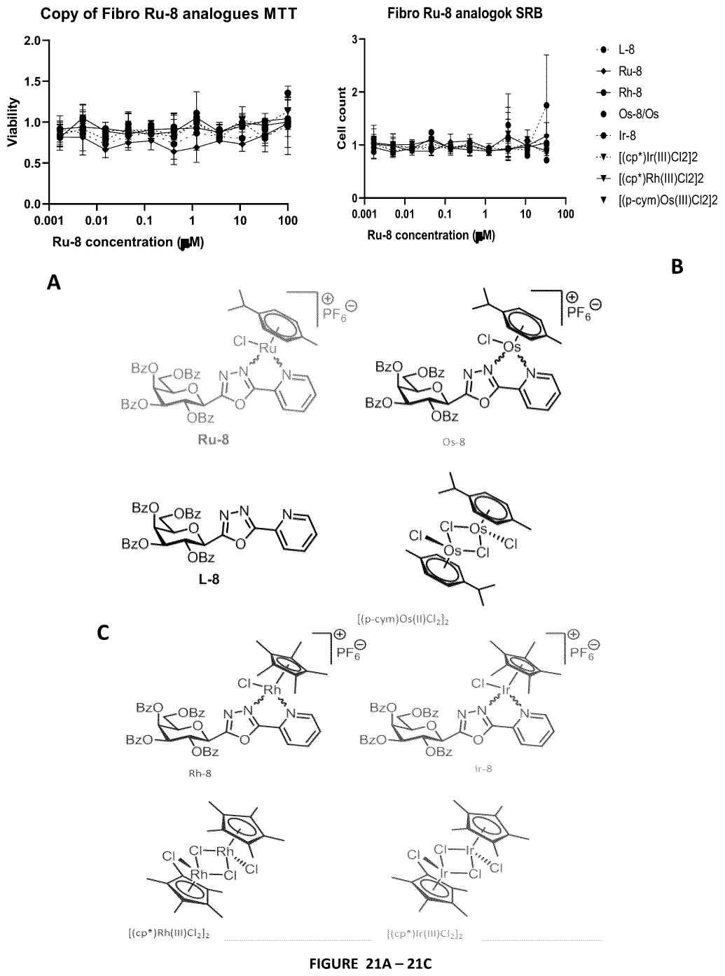

In this further series of experiments each of the ligands L-2a, L-4, L-6 and L-8 were used with each of Os, Rh and Ir, plus Ru, and the obtained complexes were compared in MTT and SRB assays on ovarian adenocarcinoma cell line A2780 as well as on primary fibroblasts with the above described Ru complexes (see FIGS. 14 to 21). Also in this set of experiments two different arene caps were tested, specifically the 1,2,3,4,5-pentamethylcyclopentadienyl ion ligand, i.e. Cp*=η5-C5Methyl5 which has a single negative charge and was used for Rh(III) and Ir(III) complexes wherein the oxidation state of the transition metal is (III) and the η6-p-cymene (para-Cymene or 4-Isopropyltoluene) for Ru(II) and Os(II) wherein the oxidation state of the transition metal is (II). Moreover, the dimers of the transition metal ions with the respective cap ligands (denoted as [(cp*)Ir(II)Cl2]2, [(cp*)Rh(III) Cl2]2, and [(p-cym)Os(III) Cl2]2) have also been tested for cytostatic effect and toxicity.

In SRB assays each of the complexes with L-2a ligands proved to be active, with L-4 ligands Rh-4 was found to be slightly active only, with L-6 ligands Rh-6 and Ir-6 were less active than Ru-6 and Os-6, whereas the same two metals with L-8 (Rh-6 and Ir-6) was found to be essentially inactive under measurement conditions while the activity of Ru-8 and Os-8 has been maintained.

As to healthy cells essentially none of the metal ion complexes were proved to be active on primary fibroblasts (except perhaps Ru-2a in SRB-assay and Ru-2a and Os-2a in MTT assay at high concentrations, in 1-2-fold higher concentrations above the IC50 values). Each of them was much more active in SRB on neoplastic cells than on primary fibroblasts.

As to the results of the MTT assay, it can be said that the most of the compounds were found to be inactive in this short-term measurement. Ru-2a was clearly active and perhaps Ir-2a and Ru-8 showed some activity on A2780 cells, whereas no compound has shown activity on primary fibroblasts, except maybe some uncertain and weak activity by Ru-2a in both MTT and SRB assay and Ir-2a in MTT assay between 10 μM and 100 μM. Neither the dimer complexes without ligands nor the ligands themselves provided any significant activity in any assays, which fact reflexes the unique and specific nature of the compounds of the invention.

The general message here again is that the half-sandwich complexes with a bulky apolar group on the monosaccharide or monosaccharide analog unit have cytostatic activity in neoplastic model whereas they are essentially non-toxic in short term model or on healthy cells.

There is an apparent difference in the activity of the complexes with various transition metals. In the experiment Ru and Os have proven to be superior in most cases, which is an unforeseen result. In certain examples Os was observed to be somewhat less MTT-positive, which, unexpectedly, suggests a lower toxicity. Both Ru and Os had a high cytostatic activity. Complexes with Ir and Rh are typically less active Ir being generally more cytostatic than Rh. However, it is apparent that with each transition metal we find complexes of the structure of the invention which have cytostatic effect against neoplastic cells and there are promisingly active compounds in each group which makes it plausible that valuable compounds can be found throughout the scope of the invention.

Complexes with Quinoline Het2 Heterocycle

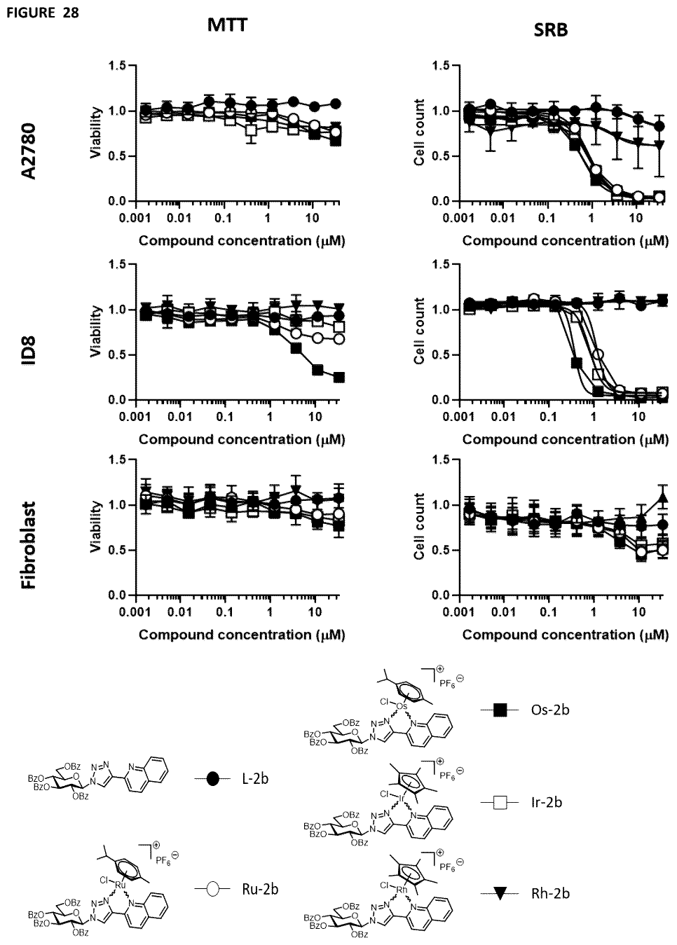

In a still further set of experiments, the quinoline complexes (Ru-2b, Os-2b, Ir-2b, Rh-2b) were tested on A2780 and ID8 ovarian cancer cells, and on non-transformed primary human fibroblasts, used as controls, in a concentration range up to 33.3 μM (FIG. 28). In concentrations exceeding 33.3 μM the complexes frequently precipitated. All of the complexes and the free ligand exerted little rapid cytotoxicity in MTT assays, not more than 35% in any of the cell lines assessed in the peak concentration, except for the Os-2b complex with 75% toxicity on ID8 cells (FIG. 28, Table 6). In SRB assays that detects cytostasis, the Ru-2b, Os-2b and Ir-2b complexes completely blocked cell proliferation with submicromolar or low micromolar IC₅₀ values both in A2780 and ID8 cells (FIG. 28, Table 6). The complexes were more efficient in ID8 cells as compared to A2780 cells (FIG. 28, Table 6). The free ligand L-2b and the Rh-2b complex had only little cytostatic activity both in A2780 and ID8 cells (FIG. 28, Table 6). On primary non-transformed fibroblasts, used as controls, the complexes exerted negligible toxicity and low cytostatic activity (FIG. 28, Table 2). The cytostatic activity was not more than 50% in the peak concentration. Similar to the pyridine complexes, the quinoline complexes had Hill coefficients >1 suggesting cooperative binding to cellular target molecule(s).

In somewhat more detail, in the experiments shown on FIG. 28 3×10³ A2780 cells, 2×10³ ID8 cells and 4×10³ primary fibroblasts were plated to 96 well plates. Cells were treated with the compounds in the concentrations indicated for either 4 hours for an MTT assay or for 48 hours for an SRB assay. Data is represented as average ±SD, from three biological replicates; individual assays were performed in duplicates. Values were normalized for vehicle treated cells, absorbance for vehicle treated cells equals to 1. Statistical significance was assessed using two-way ANOVA test comparing the free ligand and each complex as a complex-ligand pair. Before the test normality was assessed using the D'Agostino-Pearson test. The ID8 SRB Ir-2b+L-2b dataset had lognormal distribution. The A2780 MTT Ru-2b+L-2b, A2780 SRB Os-2b+L-17, Fibroblast SRB Ru-2b+L-2b were transformed using the Box-Cox method to achieve normal distribution. All other datasets had normal distribution. For better visibility the p values are presented in an excel sheet at https://figshare.com/s/2c942a812caea8869a67. Nonlinear regression was performed on the A2780 SRB and the ID8 SRB datasets.

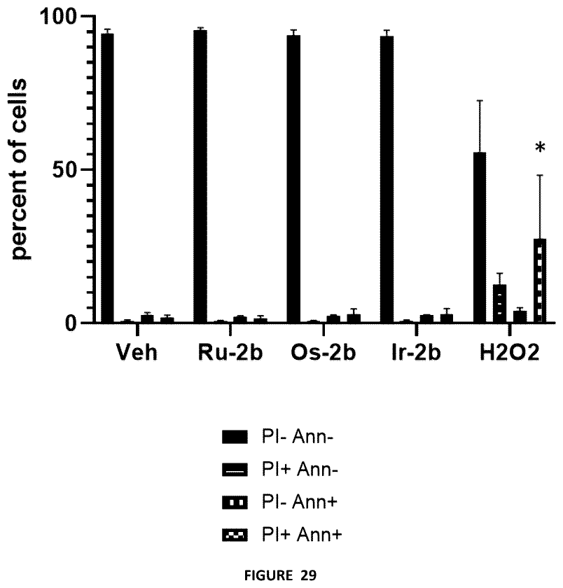

Also the toxic effects of the active Ru, Os and Ir complexes were verified by Annexin V-FITC propidium iodide (PI) double staining. The complexes were applied in concentrations corresponding to their respective IC₅₀ values and we have not detected increases in the apoptotic (Annexin V positive population) or the necrotic (PI positive and Annexin V—PI double positive populations) as opposed to the hydrogen peroxide-treated cells used as positive control (FIG. 29). Essentially, it can be concluded that quinoline complexes do not induce cell death.

In more detail, in the experiments shown on FIG. 29 2×10⁶ A2780 cells were plated in 12 well plates and were treated with the complexes (Ru-2b at 0.846 μM, Os-2b at 0.578 μM and Ir-2b at 0.891 μM) and 300 μM hydrogen peroxide for 2 hours. Cells were then stained with Annexin V and propidium iodide (PI) and cells were subjected to flow cytometry as described in Materials and Methods. The percent of cells in the quadrants are plotted. Data is represented as average ±SD, from three biological replicates, individual assays were performed in duplicates. Normal distribution was achieved by Box-Cox normalization of the data. Statistical significance was determined using a two-way ANOVA test, all measurement points were compared with each other. * indicate statistically significant differences between vehicle-treated (control) and treated cells (complexes or 300 μM H₂O₂) corresponding to the same quadrant (e.g. vehicle-treated double negative cells vs. H₂O₂-treated double negative cells) at p<0.05.

Based on these results, Rh-2b complex was omitted from further experiments.

Thus, in summary, quinoline-containing complexes are active in cisplatin-resistant cells.

### Quinoline-Containing Complexes are Active in Cisplatin-Resistant Cells

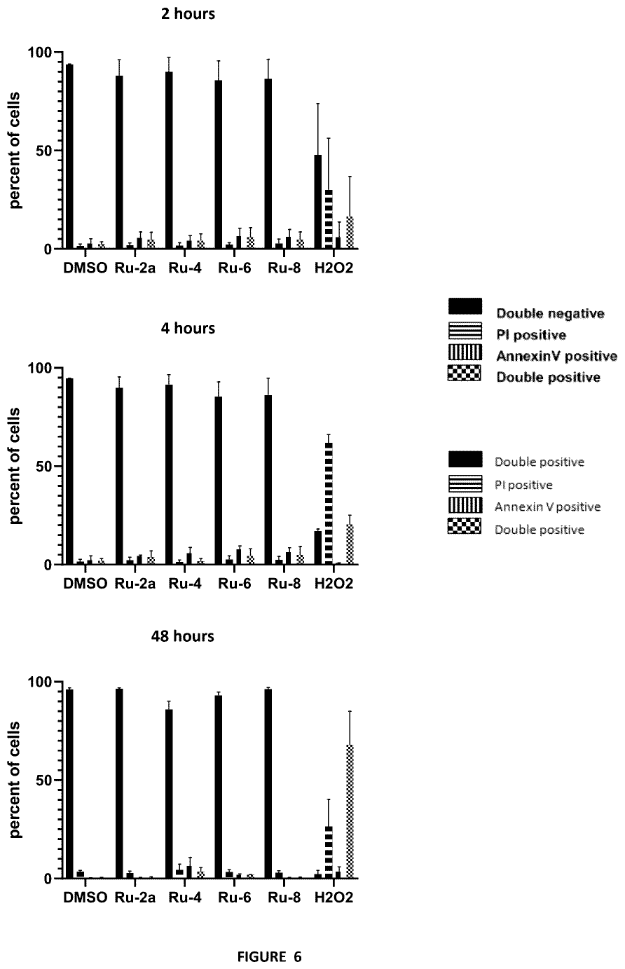

As noted above, one of the major drawbacks of platinum-based drugs is due to cisplatin resistance. We tested the three complexes with efficient cytostatic properties (Ru-2b, Os-2b and Ir-2b) on a cisplatin-resistant A2780 cell line. The complexes did not exert direct toxicity in MTT assays on the cisplatin-resistant cells (FIG. 6, Table 2).

Nevertheless, we have observed important differences in cell proliferation. The IC₅₀ value of cisplatin was 1.21 μM in our previous study on cisplatin-sensitive A2780 cells [16]. The IC₅₀ value increased to 16.47 μM in the cisplatin resistant cell line (13.6 fold increase) in SRB assays. In contrast to that, the IC₅₀ value of Ru-2b (0.8466 μM vs. 1.183 μM, 1.40 fold change) and Ir-2b (0.891 μM vs. 1.535 μM, 1.72 fold change) increased, although not to the same extent as for cisplatin. Furthermore, the IC₅₀ value of Os-2b (0.5777 μM vs. 0.476 μM) was left technically unchanged when comparing the cisplatin sensitive and cisplatin resistant cell lines (FIG. 6, Table 2). Apparently, these complexes are not detoxified strongly in cisplatin resistant cells suggesting that these compounds can be used to overcome cisplatin resistance.

Complexes with Alkanoyl Substituted Carbohydrate Moiety

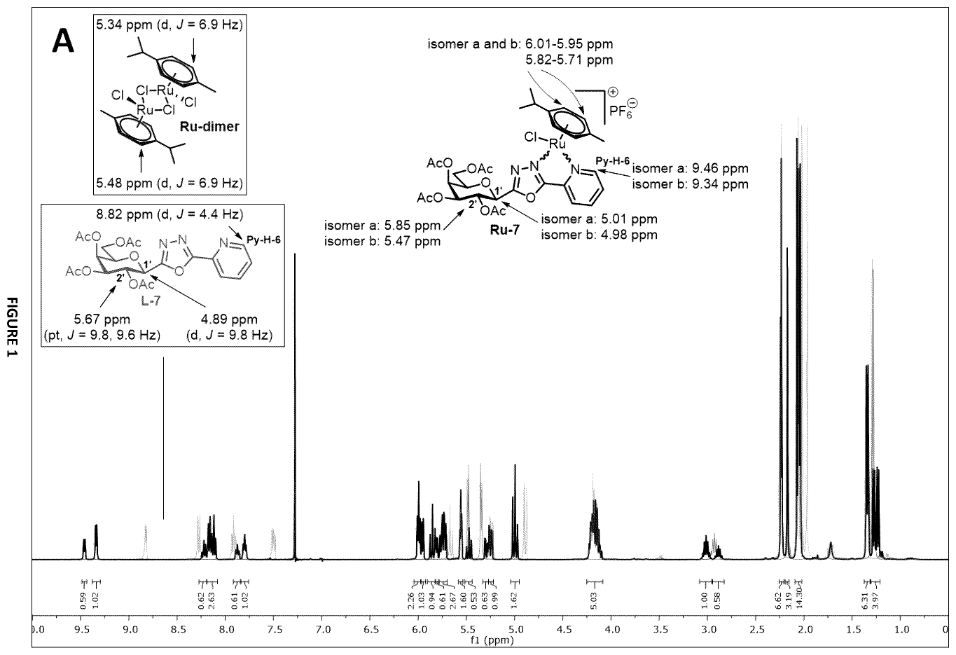

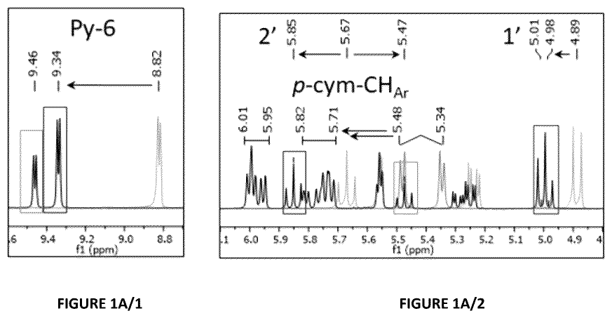

In a FOURTH set of experiments a set of osmium complexes (similar to Ia [17] in FIG. 1) and their sugar-derived ligands, in which the benzoyl protective groups on the carbohydrate moiety were replaced by aliphatic acyl groups of different chain length (straight chain C₃-C₇—CO). Osmium was selected as a central ion, since osmium complexes had the best biological activity in the previous studies. These compounds were tested on A2780 ovarian cancer cells for toxicity and cytostasis. Toxicity was assessed using the MTT assay that measures the activity of mitochondrial complex I. The inventors chose 4 hours long treatment as in an MTT assay this time point is suitable to detect rapid, direct toxicity. Cytostasis was assessed after 48 hours of treatment using the SRB assay that assesses protein content that corresponds to cell count. The characteristics of the compounds are detailed in Table 2cikk.

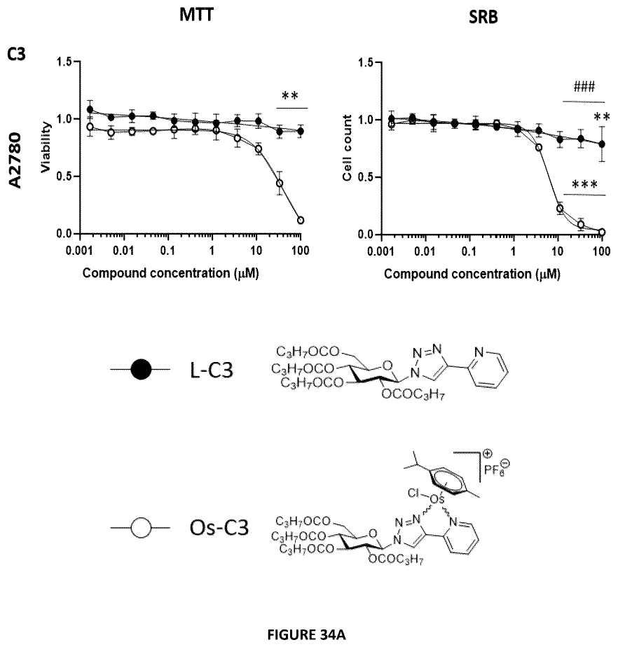

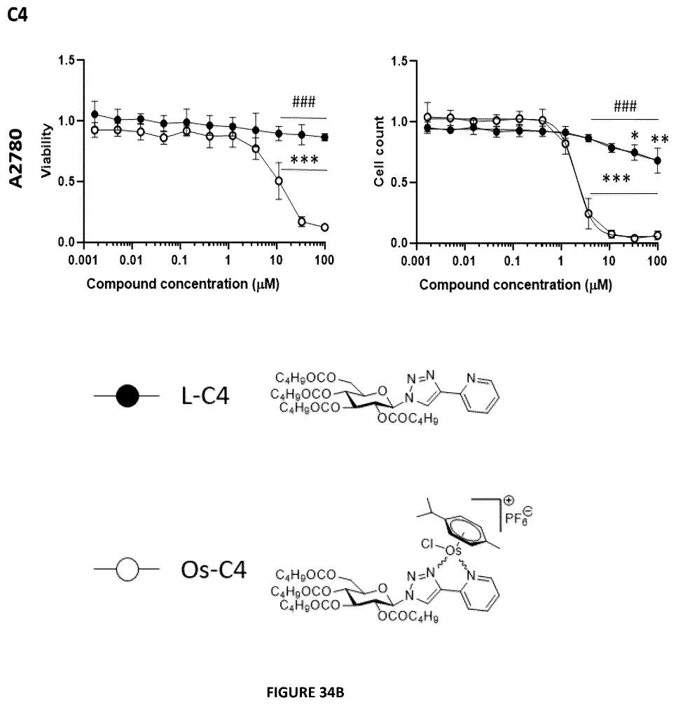

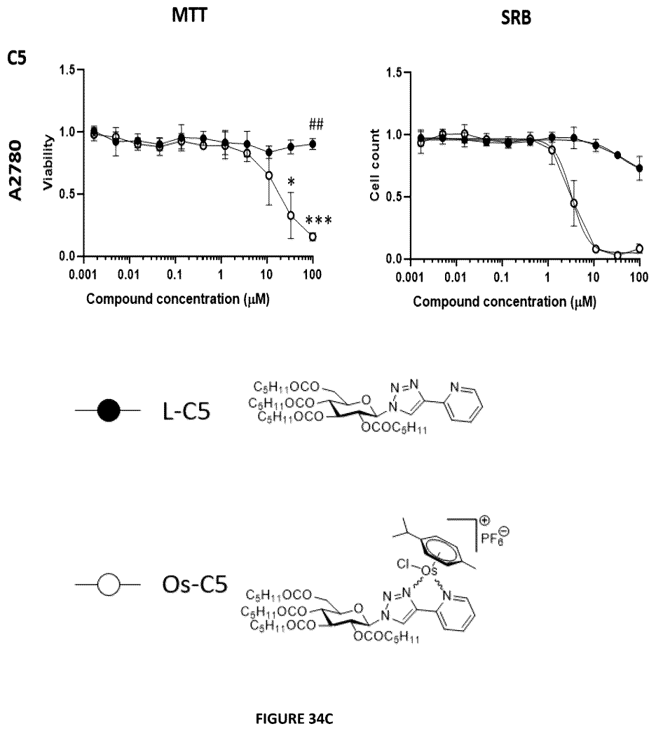

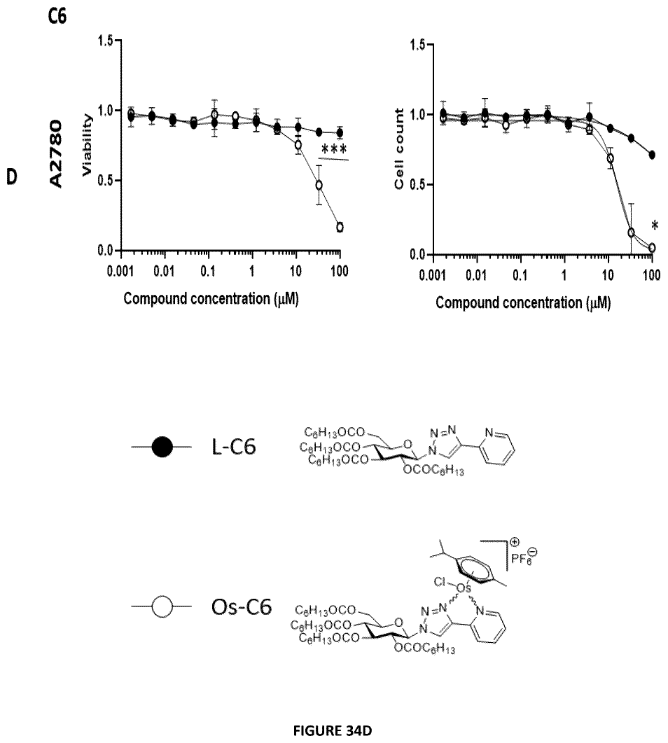

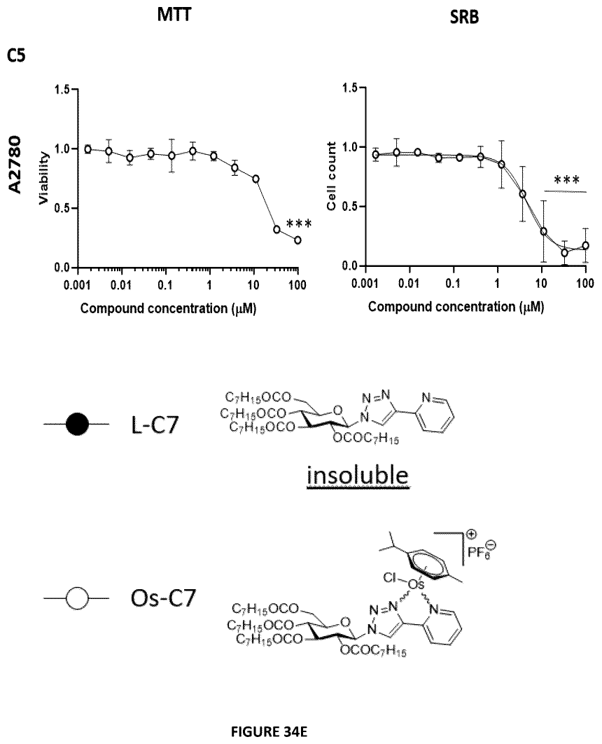

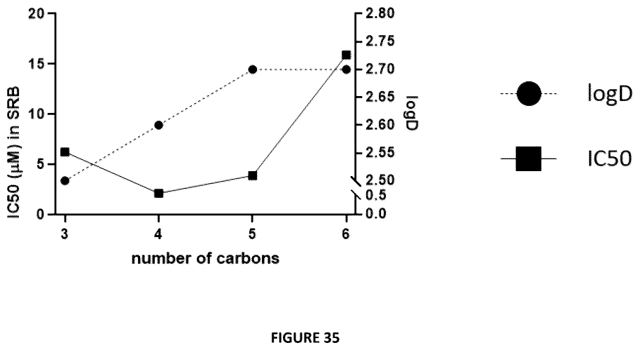

The compounds had high log D value indicating a strong apolar character up to the point that the free ligand L-16 with the longest C₇H₁₅—CO alkanoyl protective groups proved to be insoluble, hence, it was not suitable for testing. All complexes Os-C3-Os-C7, but not the free ligands L-C3-L-C6, exerted rapid toxicity on A2780 cells in MTT assays (FIGS. 34 to 36, Table 6) with cytotoxicity over 80%. Long term treatment of A2780 cells with the O-alkanoylated compounds resulted in lower cell proliferation with IC₅₀ values in the micromolar range (FIGS. 34 to 36, Table 6). The Hill coefficients, highlighting binding mode of the compounds (Gesztelyi et al., 2012), were above 2 suggesting cooperative binding of the test substances (Table 6). The free ligands had no more than 30% inhibition at the peak concentration (FIGS. 34 to 36, Table 6). When the IC₅₀ value of the complexes were plotted against the log D values we found that the optimum alkyl chain length to provide the best IC₅₀ value was 4 carbons long (FIG. 3).

Using alkanoyl protective groups instead of benzoyl groups we published earlier (showed that the aliphatic acyl protection significantly deteriorated the biological properties of the complexes, namely, the compounds exerted rapid toxicity and their IC₅₀ values were higher than even Os-2a (0.73 μM for Os-2a vs. 2.104 μM for Os-C4. Therefore, these complexes, while showing somewhat lower activity, were omitted from further testing.

Taken together, the present inventors have carried out structure-activity relationship investigations to assess whether the structure of the protective groups or modification of the heterocycle distal to the carbohydrate moiety affected the biological activity of the complexes. Namely, the replacement of the O-benzoyl protecting groups of the complexes with pyridine, in particular the most effective complex Os-2a, identified above, by aliphatic acyl protecting groups with homologously increasing chain-length has been accomplished (FIG. 1). In addition, an increase of the size of the sugar aglycon part in and its Ru(II), Ir(III) and Rh(III) analogs by changing the pyridine ring to quinoline has also been performed as illustrated below. Previous work relates to the first stage of the study.

Previous work

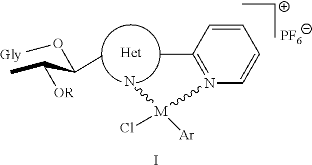

Gly = D-gluco, D-galacto, D-xylo
R = Bz, Ac, H
Het = 1,2,3-triazole, 1,3,4- and 1,2,4-oxadiazole
Ar = p-cymene with M = Ru(II) or Os(II)
pentamethylcyclopentadienyl with M = Ir(III) or Rh(III)

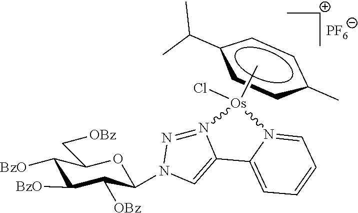

Ia Most efficient complex of the series:
IC₅₀ = 0.73 μM against A2780 ovarian cancer
MIC = 0.3-5.0 μM on multiresistant bacteria
(Staphylococcus aureus, Enterococcus species)
Target compounds of this study

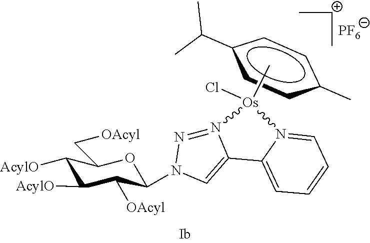

Acyl = COCₙH₂ₙ₊₁, n = 3-7

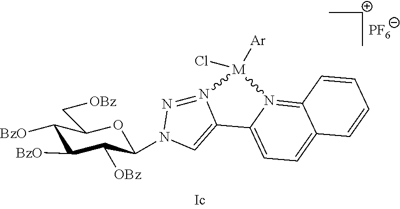

Ar = p-cymene with M = Ru(II) or Os(II)
pentamethylcyclopentadienyl with M = Ir(III) or Rh(III)

In the first stage of this study the present inventors have identified a set of carbohydrate-based half sandwich-type complexes with platinum-group metal ions, such as ruthenium(II), osmium(II), iridium(III) and rhodium(III). A main feature driving the biological activity of these complexes was their apolar nature (log D >+2). The O-benzoyl protective groups on the carbohydrate moiety proved to be particularly preferred in bringing about the apolar character and biological activity of the complexes, whereas the removal of the O-Bz protective groups or their replacement with 0-acetyl groups abolished the biological activity of the complexes.

In a further stage of the study the present inventors have assessed a further set of compounds where the hydroxyl groups of the carbohydrate moiety were protected by aliphatic acyl groups with increasing chain length (C₃→C₇—CO). Increasing the length of the acyl chain up to 4 carbons (C₃—CO) improved the biological activity of the complexes (butyryl<pentanoyl). Ru(II) complexes, with similar structure to those evaluated in this manuscript, with acetyl protective groups were biologically inactive in the same model system. However, when the acyl chain contained 5 or more carbons (C≥5-CO) the IC₅₀ values increased above that of the C₄—CO-protected complex (pentanoyl<hexanoyl<heptanoyl), despite the continuous increase in the log D value. Likely, increasing length of the acyl chain increased the apolar character of the complexes and thus decreased their solubility in cell medium impairing the cytostatic activity.

Furthermore, in contrast to the complexes with O-Bz protective groups, the O-alkanoyl-protected complexes exerted toxicity to the cells and their IC₅₀ values were inferior to those of the complexes with O-Bz-protective groups. Apparently, the nature of the protective groups strongly influences the biological activity of the complexes. Thus, surprisingly aroyl residues provide superior performance over the alkanoyl groups.

In a still further stage the present inventors have prepared another ligand, where the pyridine moiety was replaced with a larger quinoline moiety. They have synthesized Ru(II), Os(II), Ir(III) and Rh(III) complexes of that free ligand. The IC₅₀ values of the quinoline complexes were, surprisingly, superior to the pyridine containing complexes. The pyridine-containing complexes had higher IC₅₀ values on ID8 ovarian cancer cells than on A2780 ovarian cancer cells, however, the quinoline-containing complexes had similar submicromolar IC₅₀ values on both ovarian cancer cell lines further supporting their superior biological activity.

Unexpectedly, none of the complexes was toxic or cytostatic on primary, non-transformed human dermal fibroblasts pointing out the selectivity of these compounds to cancer cells. Selectivity is not a common feature of Ru(II) or Os(II) complexes.

In addition to the selectivity of the quinoline-containing complexes towards transformed cancer cells apparently the complexes have widespread cytostatic activity among other carcinomas (as Capan2 cells), sarcomas (as SAOS cells) or hematological malignancies (as L428 cells). These observations are similar in case of both pyridine-containing osmium, ruthenium and iridium complexes and the quinoline-containing complexes. While other Ru complexes have also been found active in the art, a selective cytostatic and not cytotoxic character seems to be rather unique.

Cisplatin resistance is a major drawback for the use of the currently registered platinum-based drugs (Mukherjea et al., 2020; Yu et al., 2020, Sipos et al., 2021). We provided evidence that the quinoline-containing compounds, we describe hereby, have IC₅₀ values in cisplatin resistant A2780 cells similar to the control, cisplatin-sensitive A2780 cells. Importantly, complex Os-7 had the same IC₅₀ value on both cell lines. Structurally unrelated Ru(II) and Os(II) complexes [40,41] can overcome cisplatin resistance in A2780 and SKOV3 cell models, similar to our compounds.

In addition to the selective cytostatic activity, the complexes of the invention in particular the pyridine and quinoline complexes, the latter being particularly preferred, exerted bacteriostatic activity. Interestingly, the Ir-2b complex had no bacteriostatic activity, although, iridium complexes comprising pyridine proved to be active. The MIC values of the Ru-2b and Os-2b complexes were in the low micromolar range similar to their pyridine-containing counterparts both the VRE and the MRSA isolates. VRE isolates were more susceptible to Ru-7 and Os-7 than MRSA isolates that aligns well with our previous observation. Importantly, the complexes are active on multiresistant clinical isolates suggesting that these compounds represent a novel class of antibiotics.

As to the mechanism of action, without being bound by theory, the cytostatic activity of the complexes were dependent on the generation of reactive oxygen species (ROS). In fact, ROS production was evidenced among other platinum group metal complexes (Fernandes, 2019; Mihajlovic et al., 2020; Xu et al., 2018). These and other studies report that ROS production in tumor cells is limited and even minute increases in oxidative stress leads to cytostasis. The pyridine-containing complexes of similar structure have been found to induce oxidative stress and oxidative stress has central role in their cytostatic activity. In the case of the quinoline-based complexes, vitamin E protected cells against the cytostatic effects of the osmium, ruthenium and iridium complexes that underlines the pivotal role of oxidative stress elicited by the complexes. Of note, vitamin E has a long, apolar phytyl chain; if this phytyl chain is removed the protective capacity was lost in the case of ruthenium complexes of similar structure as the ones we report here. This observation together with the apolar nature of the complexes suggest that the complexes likely target apolar compartments in cells.

Based on the inhibitory effect of vitamin E and the fact that we showed increases in 4HNE levels in A2780 cells upon treatment by the complexes we assessed the timecourse of 4HNE production. We used two osmium complexes with different chemical structure Os-2a, a triazole and Os-4, an oxadiazole complex. In both cases 4HNE production was induced early, already 30 minutes post-treatment. These data suggest that ROS production in probably a primary event upon the use of the complexes and not secondary (e.g. due to mitochondrial damage, etc.).

NRF2 is a key transcriptional regulator of the cellular antioxidant defense system (Smolkova et al., 2020). Treatment of cells with an NRF2 inhibitor (ML385) potentiated the effects of the complexes that is in good correlation with the ROS production mechanism of cytostasis.

Oxidative stress frequently impacts on cellular metabolism (Bai et al., 2015; Hill et al., 2018; Modis et al., 2012) and cellular metabolism is linked to cancer cell behavior (Hanahan and Weinberg, 2000, 2011), therefore, we assessed the interaction between treatment with the complexes and core cellular metabolic pathways. Treatment with the complexes induced the extracellular acidification rate that is a proxy readout for glycolysis. Furthermore, treatment with the complexes decreased the OCR/ECAR values that correspond to the ratio between the mitochondrial oxidation and glycolysis. As next step, we blocked glycolysis with 2-deoxyglucose (2DG) that supported the inhibitory effect of Os-2a on cell proliferation. This suggests that the complexes rearrange cellular intermediary metabolism.

The above results may be rationalized by taking into consideration the most labile character of the organorhodium complexes among the studied metal compounds. This may lead to dissociation of the Rh(III) complex before reaching the target organ in the cells while for the rest of the studied metal complexes the intact species can act due to the significantly slower ligand exchange reactions. Another point to be considered is that the appropriate organoosmium and -iridium complexes exhibit in general lower redox stability than the half-sandwich type analogous Ru or Rh complexes. The more facile oxidation might provide support to their higher biological activity in the biological assays and to the assumption that their effect is connected to ROS generation. Furthermore, when the Ru/Os and Rh/Ir pairs are compared the pentahapto bound Cp* ligand forms in general more stable M-C bonds than the hexahapto p-cymene making the half-sandwich type organometallic complexes more resistant to redox reactions for the latter pair of metals.

As a brief conclusion, in the biological study of the complexes in ovarian adenocarcinoma cells antineoplastic properties characterized by little acute toxicity but rather long term cytostasis were identified. The bioactive ruthenium, osmium and iridium complexes had micromolar IC50 values on A2780 cells (and in case of ruthenium on ID8 cells), whereas some rhodium complexes showed weaker activity, while the compounds had little or no activity on primary, non-transformed human fibroblasts highlighting the low toxicity and selectivity of these compounds towards transformed cancer cells.

The presence of the sugar moiety equipped with large hydrophobic protective groups on the hydroxyl groups was found to be important for the biological activity. Furthermore, replacing the O-benzoyl protective groups of the carbohydrate moiety to straight chain O-acyl groups worsened the cytostatic ability of the complexes and rendered them toxic. The replacement of the pyridine substituent with a quinoline moiety improved the IC₅₀ value of the complexes. The complexes were active in a wide variety of carcinoma, sarcoma and lymphoma cell lines. Furthermore, it has been shown that ROS production plays a central role in the biological activity of the complexes. Importantly, the bioactive derivatives were active on platinum resistant cells suggesting that these complexes may be able to overcome cisplatin resistance in vivo.

The bioactive transition metal complexes, identified hereby, show a cooperative binding to yet unidentified cellular target(s) and induce oxidative stress, as supported by our findings, in a membrane dependent manner.

The present inventors provided evidence that complexes of the invention proved to be active in cell models of ovarian cancer, glioblastoma, breast cancer and pancreatic adenocarcinoma, although some preference towards ovarian cancer cells could be observed, suggesting the anti-neoplastic effect is general.

The measured effect and facts about mechanism make it plausible that the complexes of the invention provide a general anti-neoplastic effect.

The invention relates to the compounds disclosed according to the present invention for use in the treatment of neoplasms. Neoplasm can be a carcinoma or a sarcoma.

A “neoplasm” is a type of abnormal and excessive growth of tissue. (The process that occurs to form or produce a neoplasm is called “neoplasia”.) The growth of a neoplasm is uncoordinated with that of the normal surrounding tissue, and persists in growing abnormally, even if the original trigger is removed. This abnormal growth usually forms a mass, when it may be called a tumor.

A number of neoplasms of each type are listed in (Kumar et al., 2017).

Below we provide a few examples without limitation.

A “carcinoma” is a neoplasm which arises from transformed cells of epithelial origin, including epithelial tissue of the skin, or the tissue that lines internal organs, such as the liver or kidneys. Carcinomas may be confined to the primary location of origin; however, they may also spread to other parts of the body.

Carcinomas are divided into two major subtypes: adenocarcinomas, which develop in an organ or gland, and squamous cell carcinomas, which originate in the squamous epithelium. Treatments against carcinomas and in particular adenocarcinomas are particularized herein and can form preferred embodiments.

A “sarcoma” is a neoplasm which arises from transformed cells of mesenchymal, in particular connective tissue origin. Connective tissue includes bone, cartilage, tendon, muscle, fat, vascular or hematopoietic tissues, and sarcomas can arise, among others, in any of these types of tissues.

Sarcomas include osteosarcoma or osteogenic sarcoma, chondrosarcoma, leiomyosarcoma, rhabdomyosarcoma, mesothelial sarcoma or mesothelioma, fibrosarcoma, angiosarcoma or hemangioendothelioma, liposarcoma, glioma or astrocytoma, myxosarcoma, and mesenchymous or mixed mesodermal tumors.

Neoplasms include neoplasms of brain or central nervous system; neoplasms of haematopoietic or lymphoid tissues; malignant neoplasms; in situ neoplasms; benign neoplasms; and neoplasms of uncertain behavior.

The neoplasms of the central nervous system, preferably the brain, include primary neoplasms of brain (including gliomas of the brain, astroblastoma of the brain); primary neoplasms of meninges (including meningiomas); primary neoplasm of spinal cord, cranial nerves or remaining parts of central nervous system (including gliomas). Anti-neoplastic effect against neoplasms of the CNS, in particular CNS-cancers is particular or even preferred.

In situ neoplasms comprise in situ carcinomas of the oral cavity, esophagus, stomach, digestive organs, respiratory system, breast, genital organs, bladder, urinary organs, eye or endocrine glands; skin, and melanomas.

Anti-neoplastic effect in these organs are particular or even preferred embodiment of the invention whereas treatments against neoplasms or cancers of the respiratory system or the breast, urogenital organs, particularly the female reproductive organs are more preferred and anti ovarian cancer effect is particularly preferred.

Neoplastic diseases of the digestive system and those of the endocrine system also provide a serious health problem. Treatments against such neoplasms, in particular if malignant, are also particular embodiments.

“Hematologic neoplasms” (or neoplasms of the hematopoietic and/or lymphoid tissues), in particular malignancies are neoplasms that affect the blood, bone marrow and the lymphatic system or which are of hematopoietic and/or lymphoid origin. Hematologic neoplasms fall into three categories: leukemia, lymphoma, and myeloma.

The neoplasms of haematopoietic or lymphoid tissues include myeloproliferative neoplasms (including mastocytosis); myelodysplastic syndromes (including refractory anemia, refractory thrombocytopenia, refractory neutropenia); myelodysplastic and myeloproliferative neoplasms (including chronic myelomonocytic leukemia); myeloid and lymphoid neoplasms; acute myeloid leukemias; acute leukemias; precursor lymphoid neoplasms (including precursor B-lymphoblastic neoplasms, precursor T-lymphoblastic neoplasms); mature B-cell neoplasms (including follicular lymphoma, diffuse large B-cell lymphoma, plasma cell neoplasms, B-cell lymphomas); mature T-cell or NK-cell neoplasms; Hodgkin lymphoma; histiocytic or dendritic cell neoplasms (including histiocytic sarcoma, follicular dendritic cell sarcoma); and immunodeficiency-associated lymphoproliferative disorders.

Leukemias are cancers of the bone marrow. Examples of leukemia include acute myeloid leukemia, chronic myeloid leukemia, acute or chronic lymphoid leukemia, lymphocytic leukemia, and lymphoblastic leukemia.

Lymphomas develop in the glands or nodes of the lymphatic system. Lymphomas may also occur in specific organs such as the stomach, breast or brain. The lymphomas are subclassified into two categories: Hodgkin lymphoma and non-Hodgkin lymphoma.

The experiments and data on the mechanism provided herein a broad anti-neoplastic effect of the compounds of the invention.

To assess the biological activity of the compounds from a different point of view, antimicrobial effect of the compounds has been tested in multiple models.

The bacterial species used for testing are briefly reviewed hereinbelow.

Such exemplary models were Staphylococcus aureus (ref. strain ATCC43300) and Enterococcus faecalis (ref. strain ATCC29122), clinical isolates (6 methicillin-sensitive Staphylococcus aureus MSSA isolates and 6 methicillin-resistant Staphylococcus aureus (MRSA) isolates as well as 8 clinical isolates of Enterococcus faecium. In a further experiment clinical isolates of vancomycin-sensitive Enterococcus (VSE) and vancomycin-resistant Enterococcus (VRE) were tested against the complexes tested above.

Gram-negative bacteria subjected to examination with the compositions of the invention include Escherichia coli, Acinetobacter baumannii and Pseudomonas aeruginosa (data not shown).

Enterococcus is a large genus of lactic acid bacteria. Two species are common commensal organisms in the intestines of humans: E. faecalis and E. faecium. Enterococcus species can also cause infections such as urinary tract infections, bacteremia, bacterial endocarditis, diverticulitis, meningitis, and bacterial peritonitis. From a medical standpoint, an important feature of this genus is the high level of intrinsic antibiotic resistance. Some enterococci are intrinsically resistant to β-lactam-based antibiotics (penicillins, cephalosporins, carbapenems), as well as many aminoglycosides. In the last two decades, particularly virulent strains of Enterococcus that are resistant to vancomycin (vancomycin-resistant Enterococcus, or VRE) have emerged in nosocomial infections of hospitalized patients.

Enterococcus faecium is a Gram-positive, non-hemolytic bacterium in the genus Enterococcus. It can be commensal (innocuous, coexisting organism) in the gastrointestinal tract of humans and animals, but it may also be pathogenic, causing diseases such as wound infection, sepsis or urinary tract infection. There are strains of this bacterium that have developed multi-drug antibiotic resistance. Enterococcus faecium has been a leading cause of multi-drug resistant enterococcal infections worldwide. Approximately 40% of medical intensive care units reportedly found that the majority of device-associated infections (namely, infections due to central lines and urinary drainage catheters) were due to vancomycin- and ampicillin-resistant E. faecium. It is difficult to fight infections caused by E. faecium since not many antimicrobial solutions are available.

Enterococcus faecalis is a Gram-positive, commensal bacterium inhabiting the human gastrointestinal tract. Like other species in the genus Enterococcus, E. faecalis is found in healthy humans. However, as an opportunistic pathogen, E. faecalis can cause life-threatening infections (such as endocarditis, sepsis, urinary tract infections), especially in the nosocomial environment, where the naturally high levels of antibiotic resistance found in E. faecalis contribute to the burden caused.

The genus Staphylococcus includes at least 40 species. Many of these species are non-pathogenic and reside normally on the skin of humans and other animals. Human pathogenic Staphylococcus species include Staphylococcus aureus (including methicillin-resistant Staphylococcus aureus, or MRSA, and vancomycin-intermediate/vancomycin-resistant S. aureus, or VISA/VRSA), Staphylococcus epidermidis, Staphylococcus saprophyticus, Staphylococcus lugdunensis; animal pathogenic Staphylococcus species include Staphylococcus schleiferi, Staphylococcus pseudintermedius and Staphylococcus caprae.

Staphylococcus aureus is a Gram-positive bacterium, and is a usual member of the microbiota of the human body, frequently found in the upper respiratory tract and on the skin. S. aureus can also become a pathogen, being a common cause of skin infections including abscesses, respiratory infections such as pneumonia, and food poisoning. Pathogenic strains often promote infections by producing virulence factors such as potent protein toxins, and the expression of a cell-surface protein that binds and inactivates antibodies. The emergence of antibiotic-resistant strains of S. aureus such as methicillin-resistant S. aureus (MRSA) is a worldwide problem in clinical medicine. S. aureus can cause a range of illnesses, from minor skin infections, such as pimples, impetigo, boils, cellulitis, folliculitis, carbuncles, scalded skin syndrome, and abscesses, to life-threatening diseases such as pneumonia, meningitis, osteomyelitis, endocarditis, toxic shock syndrome, bacteremia, and sepsis. It is still one of the five most common causes of hospital-acquired infections and is often the cause of wound infections following surgery.

Bacteria constitute a large domain of prokaryotic microorganisms. Broadly speaking, there are two different types of cell wall in bacteria that classify bacteria into Gram-positive and Gram-negative bacteria. Gram-positive bacteria possess a thick cell wall that contains many layers of peptidoglycan and lipotheichoic acids. In contrast, Gram-negative bacteria have a relatively thin cell wall consisting of a few layers of peptidoglycan surrounded by a second lipid membrane containing lipopolysaccharides and lipoproteins. These differences in structure can produce differences in antibiotic susceptibility.

Gram-positive bacteria have a thick but porous peptidoglycan layer in the cell wall (thicker than the Gram-negative bacteria), but they do not have an outer membrane whereas Gram-negative bacteria have. This is why Gram-positive bacteria usually allow a more easy access to their cell membrane than Gram-negative bacteria.

The results of the experiments with species and isolates mentioned above are shown on FIG. 38.

The measurements were carried out by broth microdilution method.

Broth microdilution is the reference method for antimicrobial susceptibility testing of rapidly growing aerobic bacteria, except for mecillinam and fosfomycin, where agar dilution is the reference method.

EUCAST (EUROPEAN COMMITTEE ON ANTIMICROBIAL SUSCEPTIBILITY TESTING, European Society of Clinical Microbiology and Infectious Diseases; see EUCAST reading guide for broth microdilution, Version 3.0 January 2021, also available at https://eucast.org/ast_of_bacteria/mic_determination/) recommends testing according to the International Standard ISO 20776-1.

Results are recorded as the lowest concentration of antimicrobial agent that inhibits visible growth of a microorganism, the Minimum Inhibitory Concentration (MIC), expressed in mg/L or μg/mL.

The bioactive complexes were also surprisingly bacteriostatic on MRSA and VRE clinical isolates with low micromolar MIC values.

While most complexes tested have turned out to show some activity, below the inevitably anti-bacterial complexes are showed by underlining and the most active ones in bold for each strain/isolates type. Complexes marked as (−) have not been tested in the experiment.

Staphylococcus aureus Reference Strains
(Ru-2a, Os-2a, Ir-2a, Rh-2a (−), Ru-4, Os-4, Ir-4 (−), Ru-6, Os-6, Ir-6(−), Ru-8, Os-8)

### MSSA Isolates

(Ru-2a, Os-2a, Ir-2a, Rh-2a (−), Ru-4, Os-4, Ir-4, Ru-6, Os-6, Ir-6, Ru-8, Os-8, Ir-8)

### MRSA Isolates

(Ru-2a, Os-2a, Ir-2a, Rh-2a (−), Ru-4, Os-4, Ir-4, Ru-6, Os-6, Ir-6, Ru-8, Os-8, Ir-8)
   Enterococcus faecalis Reference Strain
(Ru-2a, Os-2a, Ir-2a, Rh-2a (−), Ru-4, Os-4, Ir-4 (−), Ru-6, Os-6, Ir-6(−), Ru-8, Os-8, all very active)

### VSE Isolates

(Ru-2a, Os-2a, Ir-2a, Rh-2a, Ru-4, Os-4, Ir-4 (−), Ru-6, Os-6, Ir-6(−), Ru-8 (−), Os-8)

### VRE Isolates

(Ru-2a, Os-2a, Ir-2a, Rh-2a, Ru-4, Os-4, Ir-4, Ru-6, Os-6, Ir-6(−), Ru-8 (−), Os-8)

We may conclude that Ru, Os and Ir complexes are preferred, wherein complexes with ligand 2a have shown a superior activity (see FIG. 38). We find highly active examples among each ligands, against one or more strains, wherein preferably Ru and Os complexes are often more active but for example Ir-6 proved to be highly active against MSSA isolates and Ir-8 proved to be highly active against MRSA isolates. Against resistant clinical isolates the L-2a complexes of Ru, Os and Ir proved to be the most reliably active.

Our results show that each of the tested complexes has shown some activity on both the reference strains and on clinical isolates.

Taken together, here again, rhodium complexes were less active than others. Complexes with ligands L-6 and L8 also provided generally a high antibacterial activity whereas those with L-4 might be sometimes less active but this difference may be incidental or sporadic.

As explained above the compounds of the invention were ineffective against a plurality of Gram-negative bacteria.

Moreover the compounds have been tested and found inactive on clinical Candida isolates (anti-fungal effect; data not shown). Exemplary Candida strains used in the experiment were Candida albicans (SC5314) and Candida auris (ATCC21092).

As a general observation, the exemplary complexes of the invention have turned out to be active against the Gram-positive bacteria used in the experiment. We have duly noted the fact that the complexes of the invention proved to be active against Gram-positive bacteria only. Against tested Gram-negative bacteria experiments provided negative results, i.e. the complexes of the invention had no bacteriocidal or bacteriostatic effect in these bacteria having different cell wall structure.

It is to be mentioned here that the complexes of the invention also proved to be ineffective against Candida, which have a different cell wall composition (mostly contain chitin and β-D-glucan). It may well be that these differences in cell wall structures, in case of Gram-negative bacteria and yeasts, can protect the cells from the complexes of the invention which cannot reach the cytoplasma membrane.

The skilled person will understand that the above results support that the complexes of the invention are plausibly effective in a broad spectrum of Gram-positive bacteria a few further example of which are listed below for sake of illustration.

Gram-positive bacteria that are pathogenic to humans include bacteria from the Streptococcus, Staphylococcus, Corynebacterium, Listeria, Bacillus, Clostridium, and Enterococcus genera, and other bacteria from the phyla Firmicutes and Actinobacteria.

Streptococcus species are responsible for many cases of streptococcal pharyngitis, pink eye, meningitis, bacterial pneumonia, endocarditis, erysipelas, and necrotizing fasciitis. Human pathogenic Streptococcus species include S. pyogenes, S. agalactiae, S. dysgalactiae, S. gallolyticus, S. anginosus, S. sanguinis, S. mitis, S. mutans, and S. pneumoniae.

Bacteria from the genus Corynebacterium can also cause human disease, for example Corynebacterium diphtheriae can cause diphtheria.

From the Bacillus genus, two species are medically significant from a human point of view: Bacillus anthracis (which causes anthrax) and Bacillus cereus (which causes food poisoning).

The Clostridium genus includes several significant human pathogens, such as Clostridium difficile (which was later reclassified into the Clostridioides genus as Clostridioides difficile), Clostridium botulinum, Clostridium perfringens, Clostridium tetani, Clostridium sordellii, Clostridium novyi and Clostridium welchii.

Gram-positive bacterial infections include, without limitation, infections by Gram-positive cocci, like Toxic shock syndrome and infections caused by Staphylococcus and Streptococcus, as well as Gram-positive bacilli, like Diphtheria, Anthrax, Erysipelothricosis, Listeriosis and various enterococcal infections.

Thus, the compounds of the invention and compositions comprising them are provided for use against the bacterial infections and in the treatment of the related diseases, as far as regulations allow.

Gram-positive bacteria are usual contaminants in the food industry. Such bacteria are common in dairies and in dry production environments. Cross-contamination may affect food quality. Pathogenic bacteria present in food processing environments may cause food spoilage and also health risk for consumers both humans and non-human animals. The invention also relates to use of the inventive compounds in food industry including animal feed industry.

Gram-positive bacteria are often cause infections in patients with cancer (Shelburne and Musher, 2011). Presently an increased number of finding of Gram-positive bacteria in cancer patients have lead therapeutic suggestions. Thus, in a particular embodiment the compounds of the present invention are used against Gram-positive bacterial infections in cancer patients.

It will readily be understood by a person skilled in the art that the antibacterial effect of the inventive complexes gives rise to both medical uses, like antibiotics or medical disinfection agents, and non-medical antibacterial uses as well.

In a therapeutic treatment the compounds of the invention can be used as an antibiotic against bacteria, preferably against Gram-positive bacteria.

Antibiotic resistance is a major health problem today and this is a particular danger in nosocomial infections. As shown herein, the present inventive compounds have proved to be active against a number of antibiotic resistant strains and hospital isolates.

Antibiotics can be administered in various routes.

Oral administration has been known as the safest and most convenient route. In the present invention due to hydrophobicity limited aqueous solubility may be associated with poor bioavailability when administered orally.

There are several ways to improve solubility in oral preparations including e.g. drug-loaded microbial biopolymeric nanocarrier or inclusion complexes like those with cyclodextrines etc.

Such preparations may also be administered via the parenteral route which is quite often the route of choice due to manageable dosage, bioavailability and absence of first-pass metabolism.

Advanced technology formulation methods may be used here as well. Such methods include nanoparticle formulations like nanostructured lipid carriers, inclusion complexes may be used here as well to increase solubility in the bloodstream etc.

Transdermal delivery systems have also been developed to deliver antibiotics to the systemic circulation. This way may also help to overcome the antimicrobial resistance. Microemulsions may be an appropriate choice of getting the molecules through the epithelial barrier. Moreover, microneedles may also be utilized for a similar purpose.

The inhalation route may be the preferred option in case of respiratory tract and organ infections. The compounds of the invention are not volatile in themselves and would require formulation into solution or suspension which can be administered by nebulizers or metered-dose inhalers. Alternatively, preparing dry particles is also an option which may require dry powder inhalers to administer into the respiratory system. Excipients in aerosols may include usual additives like L-leucine, lactose, magnesium stearate or poly(lactic-acid) or poly(lactic-co-glycolid acid) microspheres.

In bacterial diseases of the skin and skin-structures the topical route may be preferred. Topical formulations may include various copolymers of poly(lactic-acid), poly(ethylene-glycol), cationic polycarbonate etc. Well known formulations of liposomes, polymers, micelles, etc. have been developed by way of nanotechnology in recent times.

The complexes of the invention may also be used as antibacterial agent in cavities of the body like in the oral cavity, in the nose, wherein liquids, e.g. rinses or washes or inhalation as described above is a preferred route. Moreover, the cavities of the urogenital system like in the vagina can be subject to antibacterial treatment e.g. in the form of suppositories or lotions or washes.

Selective antibacterial treatment may also be applied in the bowels or in the gut wherein administration routes may include oral administration, however, anal administration may also be applied. In the latter case suppositories or capsules may be applied.

Other methods are also well known in the art.

The above treatments and therapeutic methods both in respect of neoplasms and against Gram-positive bacteria are contemplated as relating to the inventions.

Well known and recent delivery methods of antibiotics are described by Nainu F et al. (Nainu et al., 2021) and by Vassallo et al. (Vassallo et al., 2020).

As to non-medical antibacterial use of the complexes of the invention the following exemplary uses are listed herein.

The complexes of the invention may be used in veterinary medicine to treat cancers or infections caused by Gram-positive bacteria in various food and companion animal species.

In an embodiment the complexes can be used as antibacterial cleaning agent, for example for cleaning surfaces. In this application they can be added to cleaning compositions as a further active agent. Typically in such application other cleaning agents like surfactants are added to the composition. Preparation of such antibacterial cleaning compositions are well known in the art.

In another embodiment the complexes can be used as aseptic or disinfecting agents in such aseptic or disinfecting compositions. For example antibacterial aseptic compositions may be used for disinfecting laboratory or medical tools or apparatuses. Furthermore, the complexes of the invention can be used in sanitizers for artificial and/or biological surfaces e.g. in skin sanitizers including sanitizer of hand.

Another field of application is that of preservatives. In this embodiment the complexes of the invention may be used for preservation of biological material in the laboratory or in the medical field including organs or tissues or cultures.

Alternatively, preservative effect of the agents may be utilized in the food industry. Cleaning or sanitizing apparatus or tools is also a way of utilization here. Moreover, the antibacterial agents which are safe for human or animal health may be used as preservatives in food or animal feed.

The skilled person will understand that these are examples and will be able to identify other fields of use.

As a brief summary, apparently, the mode of action of the biologically active complexes of the invention to induce cytostasis differs for different classes of ruthenium complexes (Fernandes, 2019). Carbohydrate-ruthenium complexes were shown to induce apoptosis that the present inventors have not detected for our compounds in concentrations corresponding to the IC50 values (Hamala et al., 2020) and other complexes of the series have a similar effect.

Rather, the active compounds identified in the present invention led to oxidative stress marked by increased 4HNE expression, a marker for lipid peroxidation. The functional role of reactive oxygen species production was confirmed by using vitamin E. The source of reactive oxygen species was other than the mitochondria. Reactive oxygen species production was shown to be cytostatic in numerous carcinomas (Bakewell et al., 2020; Fernandes, 2019; Mihajlovic et al., 2020; Smolkova et al., 2020; Xu et al., 2018). The compounds of the invention appear to act through a membrane dependent mechanism as supported by the importance of the hydrophobicity of the sugar or sugar-analogue, the sensitivity to the apolar chain in case of the counter-effect of vitamin A.

While the oxidative stress appeared to be an essential part of the mode of action in this invention, the fact that only complexes with hydrophobic moieties and/or with high hydrophobicity were active plausibly suggest a membrane dependent mechanism which, however, is independent of mitochondria. This is in good agreement with the experiments that thiol-free antioxidant vitamin E abrogated the effect whereas Trolox, a derivative of vitamin E lacking the apolar phytyl chain did not.

The complexes may potentiate the effects of the elements of chemoradiotherapy that:
induce primary or secondary reactive oxygen or reactive nitrogen species production (e.g. doxorubicine (Pacher et al., 2003; Pacher et al., 2002), or any form or radiotherapy (Kim et al., 2019)).
inhibit the DNA damage response (e.g. PARP inhibitors (Curtin and Szabo, 2013; Curtin and Szabo, 2020)), can potentiate the DNA damage by any means or block pathways that interact with DNA damage response (e.g. PI3K inhibitors).
Any antimetabolite cytostatic drugs, or drugs that modulate directly or indirectly mitochondrial metabolism or modulate the intermediary metabolism.

### Synthesis of the Complexes of the Invention

For the formation of the transition metal complexes of the invention the sugar-based heterocyclic N,N-chelating ligands were prepared first.

The synthesis of 1-(β-D-glucopyranosyl)-4-hetaryl-1,2,3-triazoles was accomplished by the well-known copper(I)-catalyzed azide-alkyne cycloaddition (Meldal and Tornoe, 2008; Rostovtsev et al., 2002) (CuAAc). Thus, the easily available 2,3,4,6-tetra-O-acetyl-β-D-glucopyranosyl azide (Györgydeák and Thiem, 2006; Paulsen et al., 1974) (1) was treated with ethynyl heterocycles a and b in the presence of bis-triphenylphosphano-copper(I)-butyrate (Bokor et al., 2012; Gonda and Novak, 2010) to give the expected O-peracetylated 1-(β-D-glucopyranosyl)-4-(pyridin-2-yl)- and -4-(quinolin-2-yl)-1,2,3-triazoles (L-1a,b) in high yields (Scheme Table 1). Removal of the O-acetyl protecting groups of L-1a,b was effected by the Zemplén method resulting in the unprotected derivatives L-3a,b in good yields. O-Perbenzoylation of compound L-3a was then also carried out to give the 1-(2′,3′,4′,6′-tetra-O-benzoyl-β-D-glucopyranosyl)-4-(pyridin-2-yl)-1,2,3-triazole (L-2a) in excellent yield.

Scheme Table 1. Synthesis of 1-(β-D-glucopyranosyl)-4-hetaryl-1,2,3-triazoles

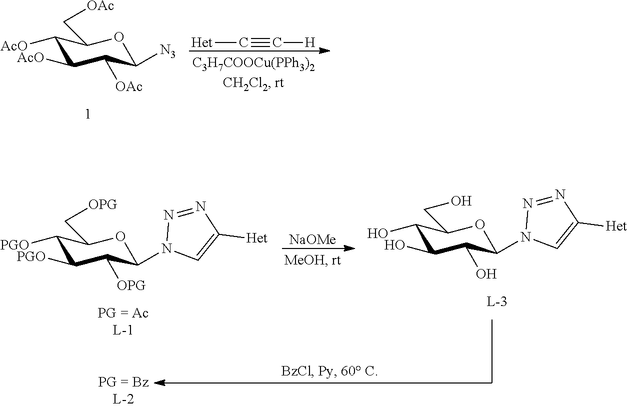

| | | Yields (%) | | |
|---|---|---|---|---|
| Het | | L-1 (PG = Ac) | L-2 (PG = Bz) | L-3 |
| 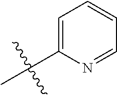 [SMILES: *c1ccccn1] | a | 95 | 87 | 76 |
| 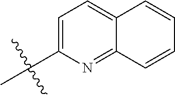 [SMILES: *c1ccc2ccccc2n1] | b | 85 | — | 85 |

The preparation of the sugar-based 5-(pyridin-2-yl)-1,3,4-oxadiazoles was carried out by the ring-transformation of the corresponding 5-substituted-tetrazoles following our previously reported procedure (Tóth et al., 2009). Thus, tetrazoles 2-5 were reacted with 2-picolinic acid in the presence of DCC under heating to furnish the desired 1,3,4-oxadiazoles L-4, L-6, L-7, L-9 in moderate yields (Table 2). The O-deprotected derivatives L-10-L-12 were then obtained upon treatment of L-4, L-6, L-7, respectively, with sodium methoxide in methanol. Under these conditions the open-chain sugar derivative L9 did not furnish the expected O-deacetylated derivative. This might be due to the ring-opening of the oxadiazole, as it was demonstrated for 2-(D-arabino-1,2,3,4-tetraacetoxybutyl)-5-methyl-1,3,4-oxadiazole (Viana et al., 2008). Acetylation of L-10 and benzoylation of L-12 by using standard methods afforded the expected O-peracetylated 2-glucosyl-1,3,4-oxadiazole L-5 and the 0-perbenzoylated 2-galactosyl-1,3,4-oxadiazole L-8, respectively, in high yields.

Scheme Table 2. Synthesis of the monosaccharide-based 5-(pyridin-2-yl)-1,3,4-oxadiazoles

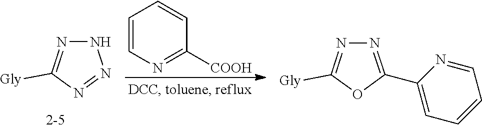

| | | | Yield (%) |
|---|---|---|---|
| 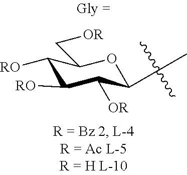 | 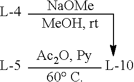 | L-4 L-5 L-6 | 35 90 58 |
| 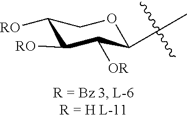 | 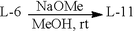 | L-7 L-8 | 50 81 |
| 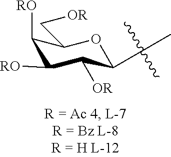 | 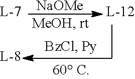 | L-9 L-10 L-11 L-12 | 13 87 35 78 |
| 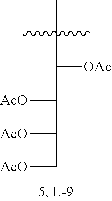 [SMILES: *C(OC(C)=O)C(OC(C)=O)C(COC(C)=O)OC(C)=O] | L-9 | | |

To get the desired cationic half-sandwich Ru(II) complexes, the above heterocyclic monosaccharide derivatives were reacted with the commercially available dichloro(η6-p-cymene)ruthenium(II) dimer ([(η6-p-cym)RuCl₂]₂, Ru-dimer).

The complexation reactions of the Ru-dimer with an equimolar amount or a slight excess of the 0-peracylated (L-1 and L-2) and the 0-unprotected (L-3) 1-(β-D-glucopyranosyl)-4-hetaryl-1,2,3-triazoles in the presence of the halide abstraction reagent TIPF₆ in a CH₂Cl₂-MeOH solvent mixture were smoothly accomplished at room temperature to give the PF₆ salts of the expected [(η⁶-p-cym)Ru^{II}(N—N)Cl]⁺ complexes Ru-1-Ru-3 in excellent yields (Scheme 1). The complexes containing the O-peracylated glucosyl-1,2,3-triazole ligands (Ru-1a,b and Ru-2a) were stable and inert enough to be purified by column chromatography on silica gel, while the isolation of the highly polar complexes having the 0-deprotected heterocyclic chelators (Ru-3a,b) could be effected by crystallisation. Due to the formation of a new stereogenic centre on the metal ion and the chiral nature of the glucose unit diastereomers of the complexes were formed in each case, whose separation could be achieved neither by column chromatography nor by crystallisation.

The skilled person will understand that the counter ion would not affect the activity of the compounds of the invention and any such ion of appropriate solubility may be used in the invention.

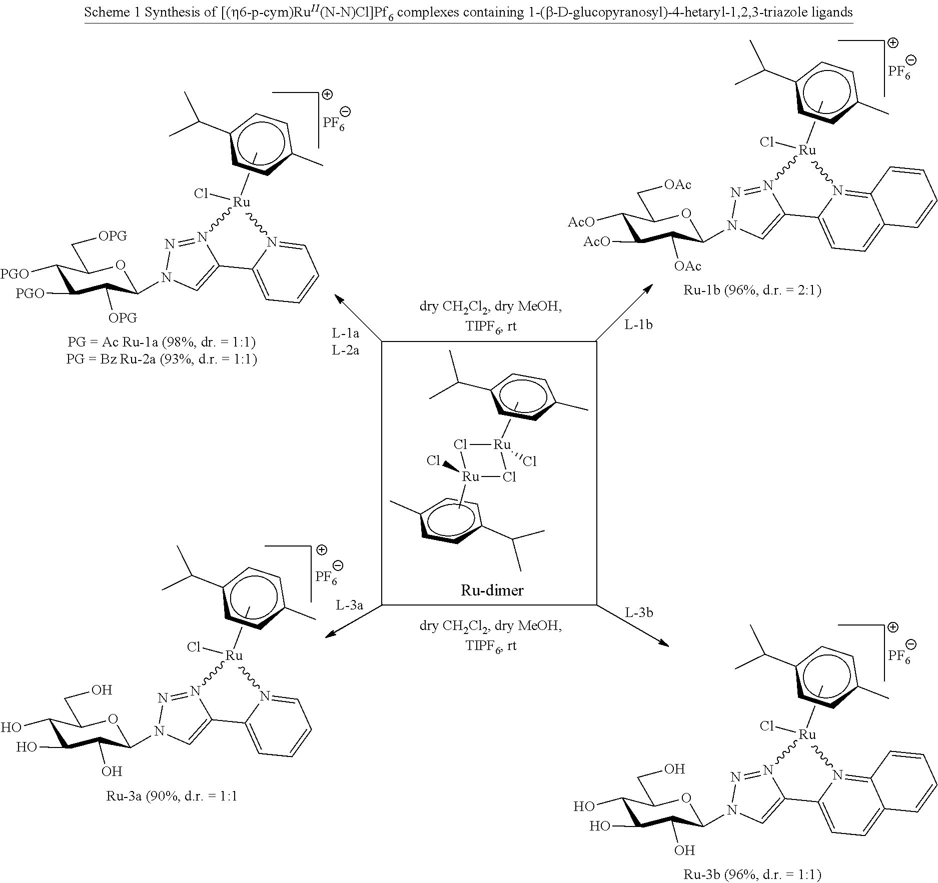

Complexation of the Ru-dimer with the monosaccharide-based 5-(pyridin-2-yl)-1,3,4-oxadiazoles (L-4-L-12) was also performed by applying the same procedure resulting in diastereomeric mixtures of the corresponding [(η⁶-p-cym)Ru^{II}(N—N)Cl]PF₆ half-sandwich type complex molecules (Table 3). Column chromatographic purification for compounds Ru-4-Ru-9 and recrystallization for Ru-10-Ru-12 furnished the test molecules in moderate to high yields.

It is to be noted that the compounds of the invention have proved to be stable which is a feature not evident in case of half-sandwich complexes of transition metals with heterocycle ligands e.g. with sugar or sugar-analogue moieties.

Scheme Table 3. Synthesis of [(η⁶-p-cym)Ru^{II}(N—N)Cl]PF₆ complexes containing the monosaccharide-based
5-(pyridin-2-yl)-1,3,4-oxadiazoles

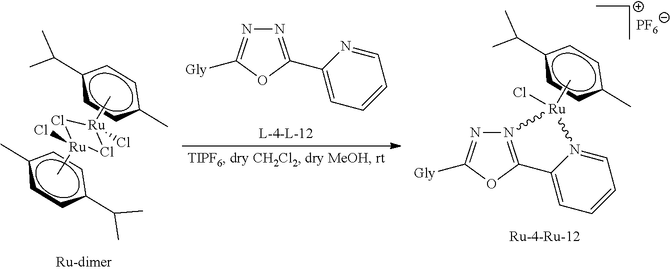

| Gly | Yield [%] (diastereomeric ratio) |
|---|---|
| 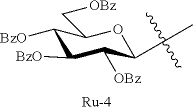 | 76 (2:1) |
| 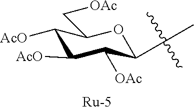 | 74 (4:3) |
| 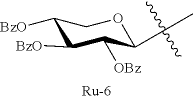 | 85 (3:2) |
| 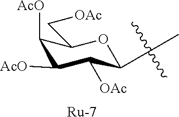 | 74 (2:1) |
| 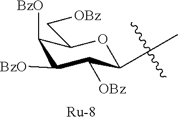 | 81 (5:4) |
| 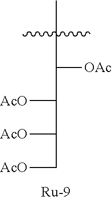 | 94 (1:1) |
| 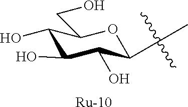 | 87 (1:1) |
| 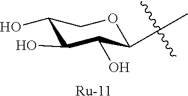 | 42 (1:1) |
| 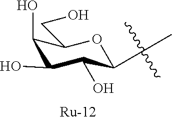 | 50 (1:1) |

The procedures for the formation of the [(η⁶-p-cym)M^{II}(N—N))Cl]PF₆ (M=Ru, Os) and [(η⁵-Cp*)M^{III}(N—N))Cl]PF₆ (M=Ir, Rh) type complexes containing O-peracylated glycosyl azole ligands were highly analogous to those for the preparation of the Ru complexes. Briefly, to a solution of the respective dimers ([(η⁶-p-cym)M^{II}Cl₂]₂(M=Ru, Os) or [(η⁵-Cp*)M^{III}Cl₂]₂(M=Ir, Rh)) in the corresponding O-peracylated glycosyl azole (2 eq.) and TIPF₆ (2 eq.) were added. Appropriate reaction time and solvent was applied to remove precipitate (here in the example TICI). While counter ions other than PF₆⁻ can also be used, clearly an inert ion is preferred and this one is particularly preferred. The reactions were carried out up to the disappearance of the half-sandwich dimer and after the complexation reaction the crude complex was purified, washed and dried to give the [(η⁶-p-cym)M^{II}(N—N))Cl]PF₆ (M=Ru, Os) or [(η⁵-Cp*)M^{III}(N—N))Cl]PF₆ (M=Ir, Rh) type complex, respectively. The crude product can be purified by column chromatography as well (9:1 or 95:5 CHCl₃-MeOH), but appears to be more time and solvent consuming and less pure.

The procedure for the formation of the [(η6-p-cym)MII(N—N))Cl]PF₆ (M=Ru, Os) and [(η5-Cp*)MIII(N—N))Cl] PF6 (M=Ir, Rh) type complexes containing unprotected glycosyl azole ligands were also analogous in case of all metal ions as follows.

To a solution of dimer ([(η6-p-cym)MIICl2]2 (M=Ru, Os) or [(η5-Cp*)MIIICl2]2 (M=Ir, Rh)) the corresponding unprotected glycosyl azole (2 eq.) and TIPF6 (2 eq.) were combined. Appropriate reaction time and solvent was applied to remove precipitate (here in the example TICI). After a sufficient level or total disappearance of the half-sandwich dimer compound and completion of the complexation reaction precipitate was removed and then the crude complex obtained after evaporation and purified to give the desired products [(η6-p-cym)MII(N—N))Cl]PF6 (M=Ru, Os) or [(η5-Cp*)MIII(N—N))Cl]PF6 (M=Ir, Rh) type complex.

As to the synthesis of the quinoline derivatives, specifically of the ligand 1-(2′,3′,4′,6′-tetra-O-benzoyl-D-D-glucopyranosyl)-4-(quinolin-2-yl)-1,2,3,-triazole (L-17) was carried out starting from the 0-unprotected derivative L-3b by using standard O-perbenzoylation conditions (Scheme Table 4). p-Cymene-containing Ru(II) and Os(II) and pentamethylcyclopentadienyl containing Ir(III) and Rh(III) cationic half-sandwich complexes with PF₆ counter ion were then prepared by the treatment of L-17 (L2-b,) with the appropriate dimeric platinum-group metal chloride precursors (Ru/Os-dimer and Ir/Rh-dimer, respectively) in the presence of TIPF₆ (Scheme Table 4).

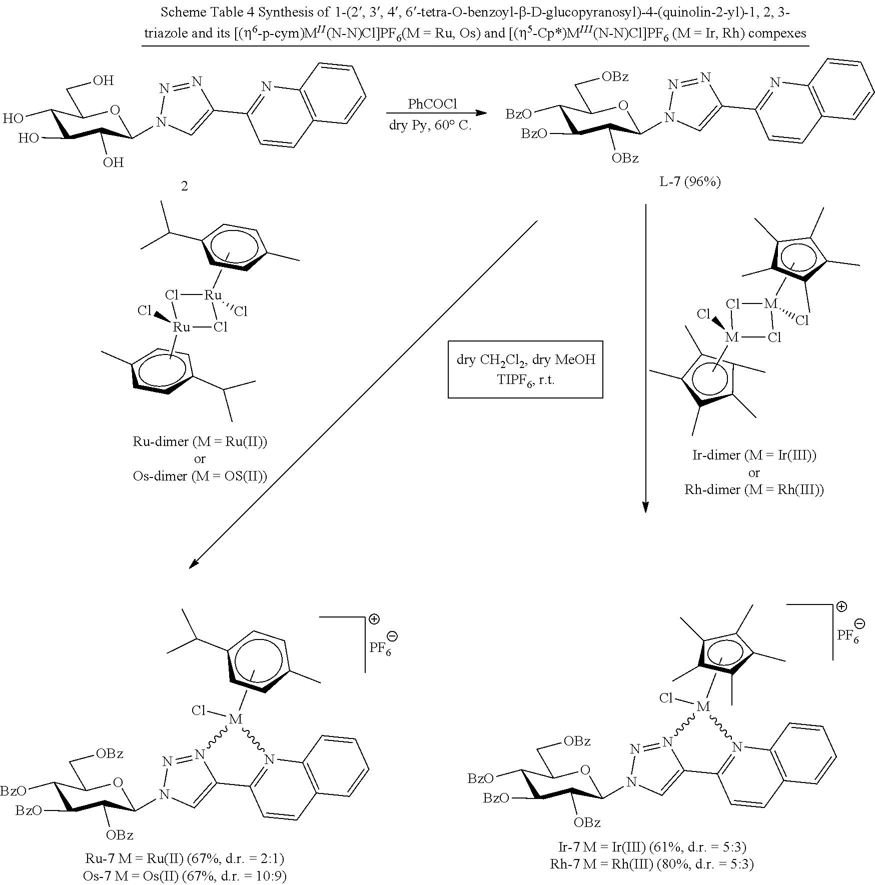

As to the synthesis of a complexes wherein the sugar moiety is substituted with C3-C7 alkyl chains, a series of O-peracylated 1-(β-D-glucopyranosyl)-4-(pyridin-2-yl)-1,2,3,-triazoles was prepared. Gentle heating of the corresponding unprotected glucosyl heterocycle 11, as described in the Examples, with aliphatic carboxylic acid chlorides in pyridine furnished the desired triazoles L-C3-L-C7 in good yields (Scheme Table 5). These compounds were then incorporated as N,N-bidentate ligands into [(η⁶-p-cym)Os^{II}(N—N)Cl]PF₆ type half-sandwich complexes by their reactions with dichloro(η⁶-p-cymene)osmium(II) dimer ([(η⁶-p-cym)OsCl₂]₂, Os-dimer) and TIPf₆. Similar to our previous studies on the synthesis of analogous half-sandwich complexes, the new test compounds L-C3-L-C7 were obtained in good to excellent yields as mixtures of two diastereoisomers (Table 1).

Scheme Table 5. Synthesis of O-peracylated 1-(β-D-glucopyranosyl)-4-(pyridin-2-yl)-1,2,3-triazoles
and their [(η6-p-cym)Os^{II}(N—N)Cl]PF6 compexes

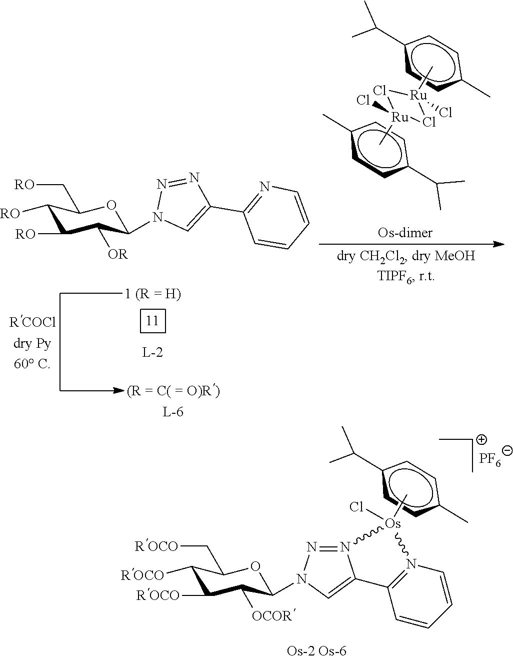

| | | Yield | | Yield | Diastereomeric |
|---|---|---|---|---|---|
| R′ | Ligand | (%) | Complex | (%) | ratio |
| —CH₂CH₂CH₃ | L-2 | 87 | Os-2 | 96 | 1:1 |
| —CH₂CH₂CH₂CH₃ | L-3 | 55 | Os-3 | 88 | 4:3 |
| —CH₂CH₂CH₂CH₂CH₃ | L-4 | 71 | Os-4 | 80 | 5:4 |
| —CH₂CH₂CH₂CH₂CH₂CH₃ | L-5 | 80 | Os-5 | 53 | 9:7 |
| —CH₂CH₂CH₂CH₂CH₂CH₂CH₃ | L-6 | 81 | Os-6 | 72 | 5:4 |

The skilled person will understand that analogous structures can be obtained by methods analogous to those above.

For example, other known bidentate heterocycle comprising ligands within the scope of the invention can be used whereas the sugar or sugar-analogue moiety with sufficiently bulky hydrophobic substituent can be used in the complexation reaction. Finding appropriate solvent, e.g. solvent of appropriate polarity, if needed solvent mixtures, as well as reaction temperatures and time are within the skills of a person skilled in the art of organic or metallo-organic chemistry.

Other reaction conditions like stirring as well as monitoring the procedure of the reaction are also a relatively easy task for the skilled person starting from the teaching provided herein and using her/his general knowledge. Specifically, a plethora of methods like TLC or spectroscopic methods, like MS, if needed combined with GC or other chromatography, and/or spectrophotometric method are at hand to monitor the progress of the reaction.

Isolation or purification methods include traditional methods in chemistry like evaporation, recrystallization, precipitation filtering, washing of crystals in appropriate non-solving solvents, etc. are very well known and applicable here.

If needed preparatory chromatographic methods can also be applied to purify the products of the invention.

As shown in the examples the products are relatively stable in solution which facilitates these procedures. It will be apparent for a person skilled in the art that analogues of monosacharides as defined in the appended claims also belong to the invention. In preferred embodiments relating to therapeutic as well as non-therapeutic uses of the complexes of the invention the skilled person is able to define functional activity of the compound based on the teaching provided herein. For example testing anti-neoplastic effect or anti-bacterial effect of any claimed compound is well at hand of the skilled person. Also defining hydrophobicity of the compounds is a task which can be easily accomplished by the skilled person based on the teaching provided herein. The methods disclosed herein can be used for such testing.

Also defining the contribution of a moiety to the hydrophobicity of the compounds of the invention is within the skills of a person skilled in the art. This can be done by synthesizing the molecules and measuring the hydrophobicity or by calculating properties, e.g. dipole moment or hydrophobic surface area of the moiety in question.

Other cap molecules, typically arenes can also be used as the appropriate dimer complexes can be prepared with the metal ions. It is well within the skills of the person skilled in the art to calculate appropriate oxidation state vs. charge requirements and find the appropriate compounds.

Also the selection of the halogen ion forming part of the complex and testing it by experiments are tasks at hand of a person skilled in the art.

### Pharmaceutical Preparations

The skilled person, in the knowledge of chemical properties of the complexes of the invention as well as the desired purpose of pharmaceutical application will be able to define and prepare the appropriate formulation. By way of illustration several examples of such formulation are given below.

### Solid Formulations

Tablets—a tablet is disc-shaped and prepared by compressing a granulated powder in a die of suitable machinery. They are mostly coated with inert substances like starch to help them disintegrate in the digestive tract of the patient. A binding agent, lubricating material, and flavors are added to the tablets to make them palatable.
Enteric Coated Tablets—are coated with a material that does not disintegrate in the acidic medium of the stomach but in the alkaline medium of the intestine. They can't be chewed but consumed only by swallowing.
Controlled Release Tablet—is designed to release the active ingredient of the drug in a specific amount over a specified period of time. Here, the amount of drug released is gradual over the day and does not depend upon the pH of the digestive tract of the patient. Thus, a uniform amount of drug is released at a uniform rate.
Sustained release preparations—release a fixed amount of drug over an extended period of time. Thus, they improve the treatment compliance by the patient.
Capsules—can be hard or soft. Hard capsules contain the drug in solid form, which gets dissolved easily in water. Soft capsules have the drug in liquid or semi-solid form, which is non-soluble in water and soluble in propylene or glycol.
Powders—can be divided (pre-portioned) or undivided (including that medical personnel or patient measures the dose).

As to the excipients the relatively highly apolar nature of the inventive complexes have to be considered and possibly emulsifiers or stabilizers added.

### Liquid and Semi-Solid Formulations

They are more readily absorbed than the solid formulations and can be administered by various routes like:

1. Oral preparations—Oral preparations are easier to swallow and administer medicines to children and old-age patients. Flavourings and sugar are added to some liquids to make them palatable. They are available as solutions, suspensions, or emulsions and must be shaken well before use.

2. Topical Preparations—The application of a drug to an area of the body for direct treatment is called topical application. It includes but not limited to:
Eye drops
Ear drops
Nasal drops
Nebulizers and inhalers
Creams and ointments for skin application
Gels and lotions
Pessaries for vaginal administration of the drug
Stents or other surfaces to which the agent is bound and released at the site of action.
Requirements for liquid applications like drops may differ from gels and creams etc. Suspension is a more appropriate form for the latter whereas in liquid preparations solution of the product may be necessary with solvents or solubilizers which are bio-compatible.
In cases wherein the drug is bound to a surface a solid form slowly releasing the drug is applied, typically from crystals or solidified suspensions.
Microcrystallized administration forms are also known for such agents.

3. Sublingual and Buccal Administration—It is useful for drugs which are active in very low concentration in the blood. Such drugs are administered as tablets under the tongue or between the cheek and the gum and allowed to dissolve. In this manner, the drug directly enters the bloodstream, bypassing the digestive tract and acts faster.

4. Rectal Administration
Suppositories: are used for drugs which are administered through the rectum. The drug is absorbed by the rectal mucosa and directly enters the bloodstream. The method is useful when a patient is unconscious, has nausea or difficulty in swallowing.
Enemas: are liquid preparations for rectal administration. They can be used for topical or systemic treatment and also for a bowel movement.

5. Parental Drug Administration—is drug administration outside the GI tract of the patient. Drugs can be inserted anywhere with the help of injections.
Intradermal administration: where the drug is inserted into the dermis. E.g. Local Anaesthesia
Subcutaneous injection: where the drug is inserted into the subcutaneous tissue or under the skin. It is mainly for the drugs that cannot be given through the mouth e.g. Insulin.
Intramuscular injection: where the drug is inserted into the skeletal system or the muscle. The system is highly vascular, and hence, drugs with low molecular weight can easily pass through by direct diffusion into the bloodstream.
Intravenous injection: is given directly in the vein and allows for a faster action of the drug.

6. Suppositories—Basic excipient may be a poly-alcohol like glycerine plus additives providing appropriate solution or suspension to provide the release at the site of administration with the desired speed.

7. Injections—injections are typically solvent form, however, in case of injections at the site of the neoplasm or bacterial infection may be a slow release solid or suspension form which may be used more often in this invention than intravenous injection.

Below the invention is described in more detail by way of examples which are non-limiting and the skilled person will be able to consider and use the whole teaching of the specification, including the document cited herein and information known in the given technical field as well as her/his general knowledge to reduce the invention into practice in the entire scope claimed.

### EXAMPLES

### Example 1—Chemical Properties of the Complexes of the Invention

The formation of the complexes and the existence of the diastereomeric pairs were confirmed by ¹H and ¹³C NMR spectroscopy in each case. As a representative, the superposition of the ¹H and ¹³C NMR spectra of Ru-7, the free ligand L-7 and the Ru-dimer, respectively, are presented in FIG. 1. Generally, the conversion of Ru-dimer into the corresponding [(η⁶-p-cym)Ru^{II}(N—N)Cl]PF₆ complexes (Ru-1-Ru-12) led to remarkable downfield shifts of the aromatic p-cymene signals both in the ¹H and ¹³C NMR spectra. No significant changes of the chemical shifts of the proton and carbon resonances of the sugar moiety were observed, except for those which were close to the coordination sphere. Thus, H-1′ protons of the sugar-based heterocyclic ligands usually displayed noticeable downfield shifts up to 0.05-0.27 ppm upon coordination. On the other hand, e.g. in case of the O-acyl protected glycosyl-1,3,4-oxadiazole derivatives L-4-L-8 the coordination resulted in either a downfield or upfield shift of the H-2′ resonances depending on the diastereomers formed. The formation of the 5-membered chelates with the participation of heterocyclic aglycone parts was also shown by the change of the chemical shift of several heteroaromatic signals. For example, in case of the 2-pyridyl substituted derivatives well traceable and consistent changes were observed in the appearance of the proton and carbon signals of the pyridine ring. For example, the H-6 and C-6 signals of the pyridine ring showed downfield shifts up to 0.48-0.83 ppm (¹H NMR) and 5.7-7.8 ppm (¹³C NMR), respectively, as a result of the coordination.

FIG. 1. Superposition of the ¹H NMR (A) and ¹³C NMR (B) spectra of Ru-7 (black), ligand L-7 (green) and Ru-dimer (blue) in CDCl₃

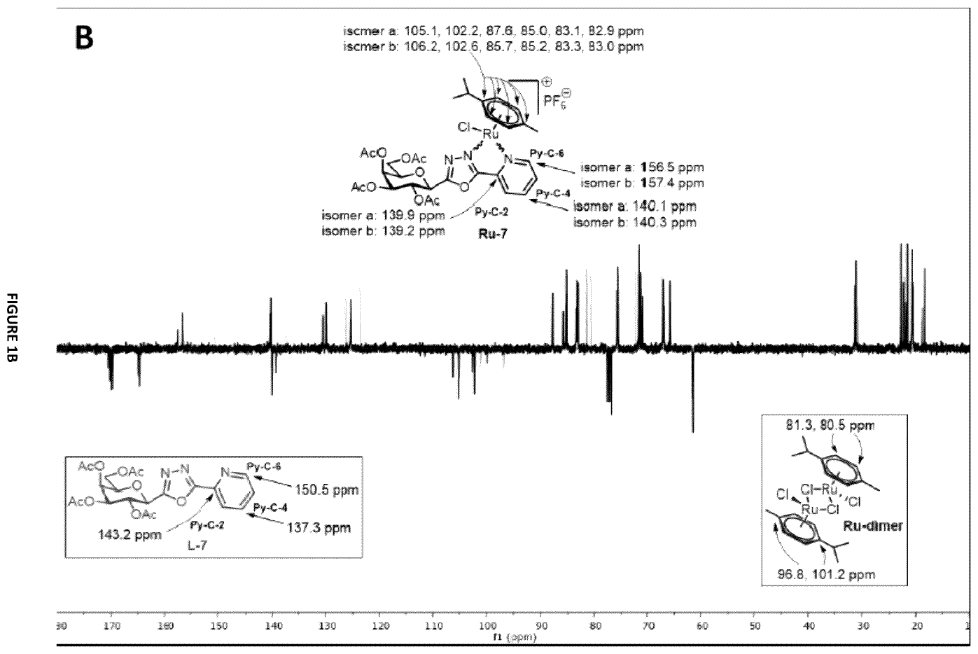

The aqueous stability of the complexes was also studied over time. As an example, time dependence of the NMR spectra of Ru-3a are shown in FIG. 2. The small shifts of signals attributable to the structural change of the complex can be observed but by adding KCl to the 2 days old equilibrated sample the original signals can be recovered. This unambiguously proves that only the exchange of the coordinating chloride ion by a water molecule occurred in a reversible manner without affecting the dissociation of the five-membered N,N chelate.

For comparative biological studies additional two Ru(II) complexes containing non-sugar based ligands (Scheme 2, Ru-13 and Ru-14) were also synthesized starting from Ru-dimer with 1-phenyl-4-(pyridine-2-yl)-1,2,3-triazole (Tawfiq et al., 2014) (L-13) and 2-phenyl-5-(pyridine-2-yl)-1,3,4-oxadiazole (Wei et al., 2010; Weiss et al., 2020) (L-14), respectively.

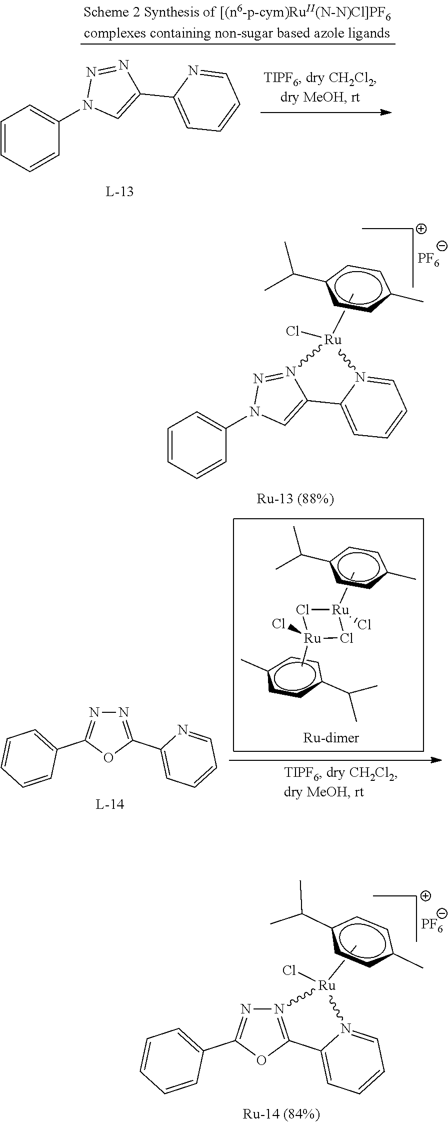

### Example 2—Ruthenium Compounds with Antineoplastic Properties

2.1 Screening of Ruthenium Compounds with Antineoplastic Properties

We screened 14 ligands and their ruthenium complexes bringing up the number of compounds to 28 in a concentration range of 100 μM-0.0017 μM. The compounds were tested in an assay aiming to assess short term toxicity (methylthiazolyldiphenyl-tetrazolium bromide (MTT) reduction assay, 4 hours) and a long term cytostasis or cytotoxicity (sulphorhodamine B (SRB) proliferation assay, 48 hours) on A2780 human ovarian carcinoma cells. Initially, four Ru complexes, Ru-2a, Ru-4, Ru-6, Ru-8 were found to have cytostatic properties both in long-term SRB and short-term MTT assays (FIG. 3A, B). The uncomplexed ligands L-2a, L-4, L-6, L-8 had no biological activity (FIG. 3A, B).

On FIG. 3 screening of ruthenium complexes for cytostatic properties is shown A2780 cells were treated with the compounds indicated for 4 hours then MTT assay was performed and another set of cells were treated with the compounds indicated for 48 hours then SRB assay was performed. Those molecules that were used in the subsequent studies are highlighted.

### 2.2 Testing Active Ru Complexes on Ovarian Cancer Cell Lines and on Primary Skin Fibroblasts for Cytostatic Effect and Toxicity

Next, we assessed the four active complexes, Ru-2a, Ru-4, Ru-6, Ru-8 and the corresponding ligands, L-2a, L-4, L-6, L-8 in detail. All compounds were tested on two ovarian cancer cell lines (A2780 and ID8) and on human primary skin fibroblasts (non-transformed, primary cells) in MTT and SRB assays. MTT assays were performed 4 hours post-treatment and indicated rapid toxicity of the compounds, while SRB assays were performed 2 days post-treatment and represent long term cytostasis or toxicity.

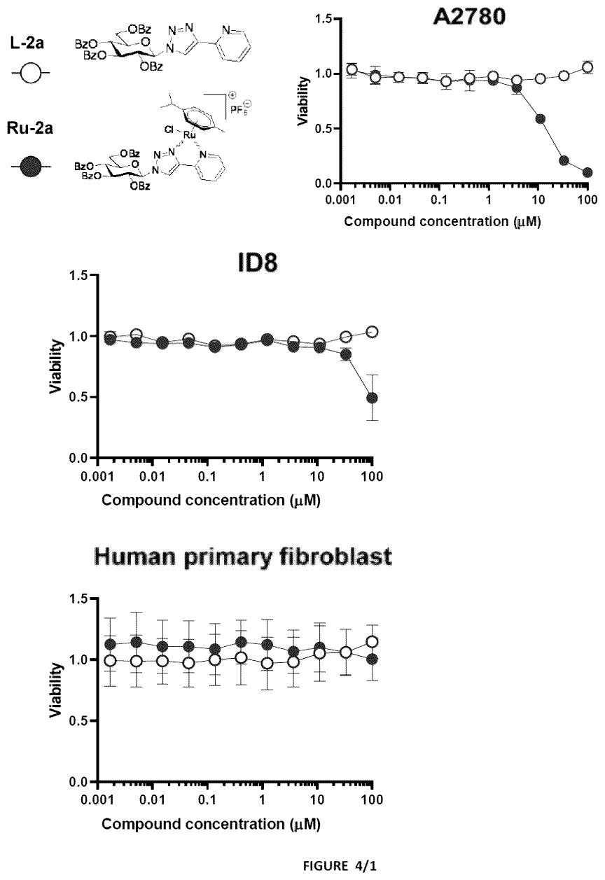

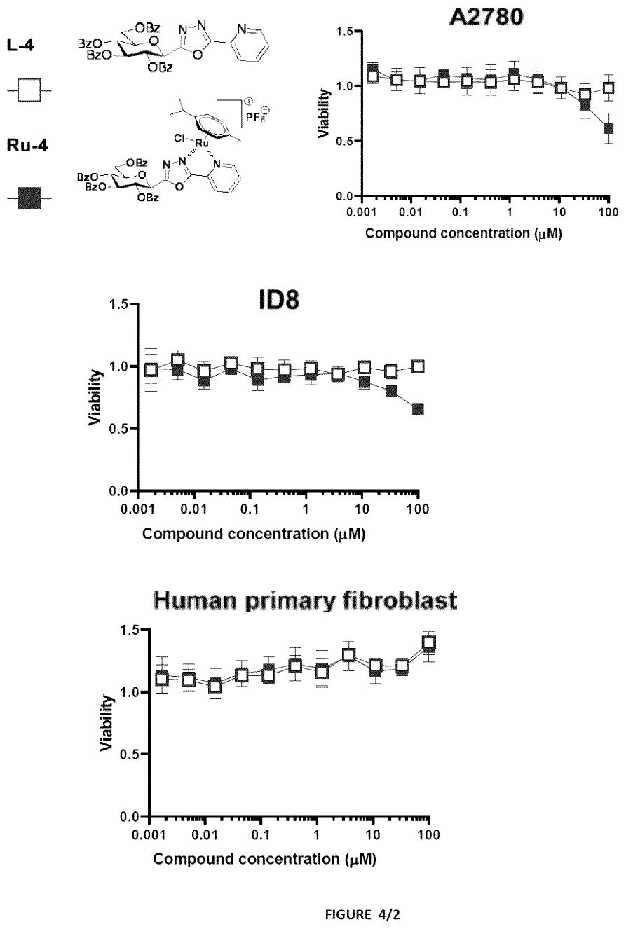

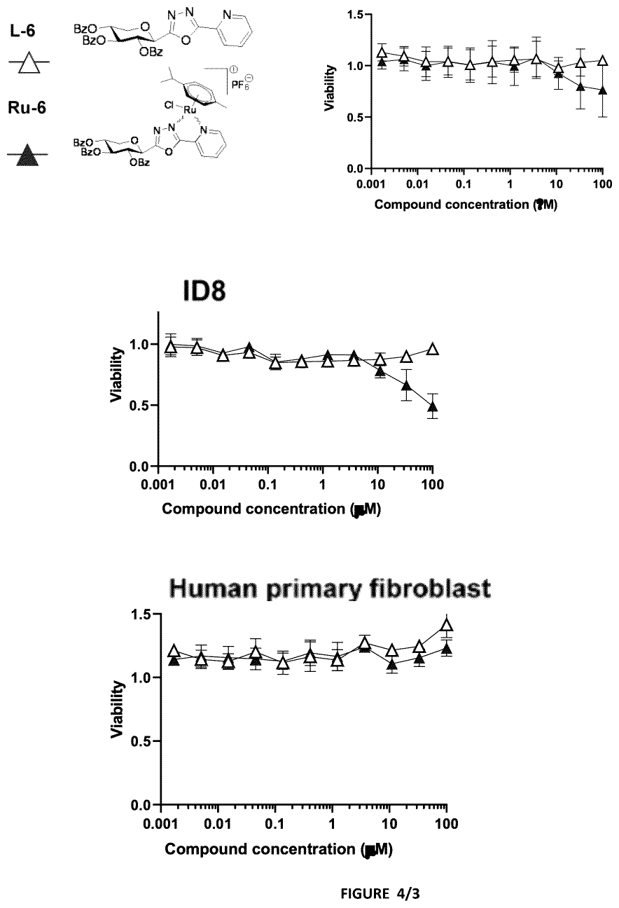

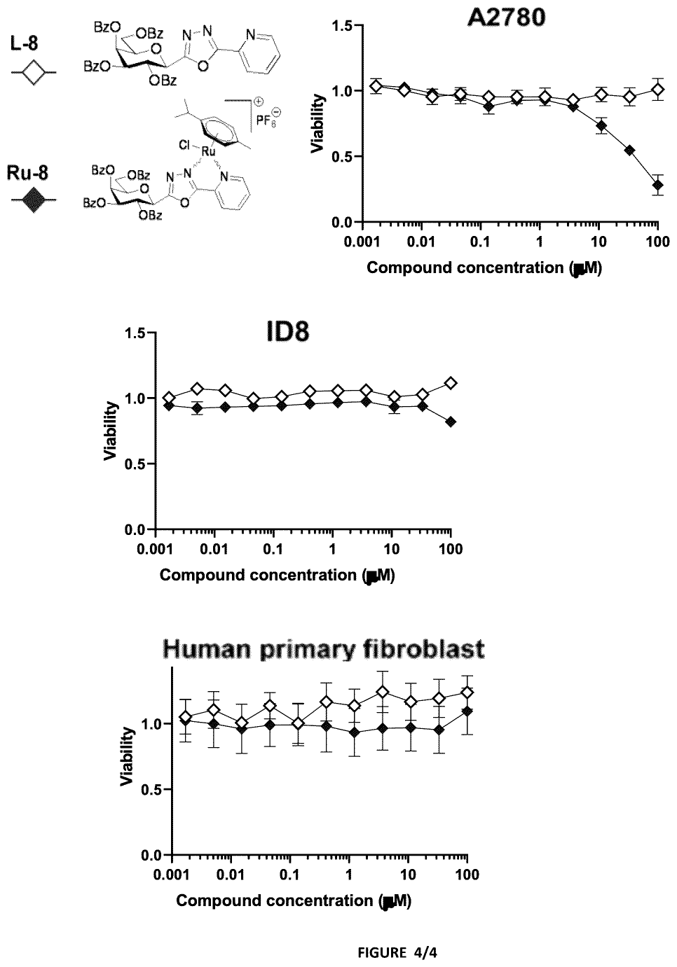

On FIG. 4 assessment of the bioactive ruthenium complexes for acute toxicity is shown in detail. Cells were treated with the compounds in the concentrations indicated for 4 hours, then MTT assay was performed. The following compounds were tested on A2780 and ID8 cells as well as on fibroblasts: Ru-4/L4, Ru-6/L-6, Ru-8/L-8, Ru-2a/L-2a.

None of the free ligands exerted either rapid toxicity or rapid cytostatic effect on any of the cell lines in short-term MTT assays (FIG. 4). However, the application of Ru-2a and Ru-8 above 10 μM concentration reduced the MTT signal in A2780 and ID8 cells. Ru-6 reduced the MTT signal in ID8 cells at 100 μM and a similar trend was observed on A2780 cells. Although it was not statistically significant, Ru-4 led to a similar reduction in MTT signal at 100 μM in A2780 and ID8 cells. Complexes Ru-2a, Ru-4, Ru-6 and Ru-8 had no effect on primary fibroblasts in MTT assays.

On FIG. 5 assessment of the bioactive ruthenium complexes for cytostatic activity is shown in detail. Cells were treated with the compounds in the concentrations indicated for 48 hours, then SRB assay was performed. The following compounds were tested on A2780 and ID8 cells as well as on fibroblasts: Ru-4/L4, Ru-6/L-6, Ru-8/L-8, Ru-2a/L-2a.

Ruthenium-complexes Ru-2a, Ru-4, Ru-6, Ru-8 were cytostatic, while none of the corresponding ligands (L-2a, L-4, L-6, L-8) had cytostatic properties in long-term SRB assays on A2780 and ID8 cells (FIG. 5). On primary human fibroblasts the ligands, L-2a, L-4, L-6, L-8 as well as two complexes, Ru-4 and Ru-8, had no effect. In contrast, Ru-2a and Ru-6 reduced the SRB signal on fibroblasts, however, at 100 μM only.

### 2.3 Annexin V-Propidium Iodide (PI) Double Staining Cell Death Measurements

Cytostasis on the long term can be due to enhanced cell death. To exclude that possibility, we performed Annexin V-propidium iodide (PI) double staining. Treating A2780 cells with Ru-2a, Ru-4, Ru-6 and Ru-8 did not increase largely the proportions of PI positive, Annexin V positive and double positive cells in contrast to hydrogen peroxide that was used as positive control either at 2 hours, 4 hours or 48 hours post treatment (FIG. 6). Four hours and 48 hours were chosen to be the same as the time points used in the previous assays. Two hours were assessed as in our previous studies 2 hours was found optimal for the induction of apoptosis (Bai et al., 2001; Virag et al., 1998b; Virag et al., 1998c).

FIG. 6 illustrates the fact that Ruthenium complexes do not induce cell death. A2780 cells were plated and were treated with the ruthenium complexes Ru-2a, Ru-4, Ru-6 and Ru-8 and hydrogen peroxide for 48 hours. Cells were then stained with Annexin V and propidium iodide (PI) and cells were subjected to flow cytometry. The percent of cells in the quadrants are plotted.

Thus, cytostatic effect of the Ru complexes is not caused by an enhanced cell death of A2780 cells.

### 2.4. Inhibitory Characteristics as Platinum Compounds

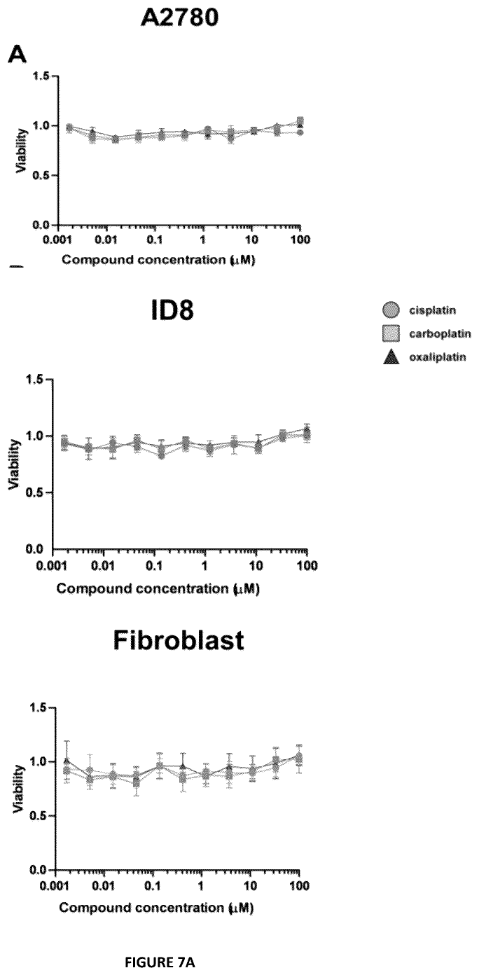

Since ruthenium complexes are regarded as alternatives to platinum-centered drugs (Kenny and Marmion, 2019), we used the currently therapeutically available platinum-based drugs, cisplatin (1), oxaliplatin (2) and carboplatin (3) as reference compounds and tested them on A2780 and ID8 cells as well as on primary human fibroblasts. Platinum drugs had no effect in MTT assays (FIG. 7A), while were cytostatic on A2780 and ID8 cells in SRB assays (FIG. 7B). Carboplatin and oxaliplatin had no effect on primary human fibroblasts in SRB assays, while cisplatin was cytostatic above 10 μM (FIG. 7).

FIG. 7 illustrates the determination of the cytotoxic and cytostatic activity of platinum compounds. A 1×10⁴ A2780 cells, 8×10³ ID8 cells and 6×10³ primary fibroblasts were plated to 96-well plates. Cells were treated with the compounds in the concentrations indicated for 4 hours then MTT assay was performed. B 3×10³ A2780 cells, 2×103 ID8 cells and 4×103 primary fibroblasts were plated to 96-well plates. Cells were treated with the compounds in the concentrations indicated for 48 hours then SRB assay was performed. Nonlinear regression was performed on the data. Values were normalized for vehicle treated cells. Absorbance for vehicle treated cells equal to 1.

Thus, it appears that typically the compounds of the invention may have even a somewhat better inhibitory potential and less toxicity than the examined platinum compounds of the art.

### 2.5 Inhibitory Properties and Cooperative Effect of Ru Complexes and Known Platinum Compounds

To compare the inhibitory properties of Ru-2a, Ru-4, Ru-6, Ru-8, we performed a nonlinear regression of the long-term cytostatic curves (SRB curves) (FIG. 5, FIG. 7B, Table 4). The IC50 values of Ru-2a, Ru-4, Ru-6 and Ru-8 fell between 0.9-9 μM and A2780 cells were more sensitive for ruthenium complexes as compared to ID8 cells (Table 4). Ru-2a had the lowest IC50 value in A2780 and ID8 cells (0.9 and 1.5 μM, respectively). The IC50 values of the ruthenium complexes (0.9-9 μM) were comparable to or lower than those of the platinum compounds (0.1-28 μM) (Table 4).

Besides the IC50 value, regression analysis yielded the Hill slope. Hill slope is a readout, characterized by the slope of the curve, that provides information on the binding characteristics of a compound (Gesztelyi et al., 2012). The Hill slope of platinum compounds on A2780 and ID8 cells was in the range of 0.6-1.6. In stark contrast to that, the Hill slope of the ruthenium complexes was in the range of 1.5-3.7 (Table 4). Higher Hill slope suggests higher cooperativity upon the binding of the ruthenium complexes to target biomolecules than in the case of platinum compounds.

TABLE 4
The lipophilicity parameters (logD) and kinetic properties of the biologically active ruthenium
complexes identified in the study and the reference platinum compounds (IC50 [μM])

| | | A2780 | | ID8 | | Fibroblast | | U251 | | MCF7 | | Capan2 | |
|---|---|---|---|---|---|---|---|---|---|---|---|---|---|
| | | | Hill | | Hill | | Hill | | Hill | | Hill | | Hill |
| Compound | logD | IC50 | slope | IC50 | slope | IC50 | slope | IC50 | slope | IC50 | slope | IC50 | slope |
| Ru-2a | 2.85 | 0.9 | 1.95 | 1.53 | 2.50 | N/A | 0.78 | 2.82 | 1.75 | 1.29 | 1.85 | 2.18 | 2.43 |
| Ru-4 | 2.41 | 6.19 | 2.62 | 5.66 | 3.04 | N/A | N/A | 4.17 | 2.46 | 3.74 | 2.01 | 3.27 | 1.81 |
| Ru-6 | 2.04 | 8.54 | 3.74 | 6.76 | 3.59 | 24.63 | 3.52 | 6.61 | 2.13 | 6.69 | 2.13 | 4.97 | 1.66 |
| Ru-8 | 2.44 | 4.27 | 3.50 | 7.94 | 3.31 | N/A | N/A | 4.49 | 2.50 | 3.75 | 2.06 | 3.82 | 1.66 |
| Ru-13 | 0.44 | 11.97 | 1.57 | | | | | | | | | | |
| Ru-14 | 1.63 | 513.5 | 0.77 | | | | | | | | | | |
| cisplatin | | 1.21 | 1.20 | 10.81 | 0.99 | 11.90 | 1.53 | | | | | | |
| carboplatin | | 27.98 | 1.02 | N/A | 0.62 | N/A | N/A | | | | | | |
| oxaliplatin | | 0.12 | 1.27 | 4.59 | 1.58 | 0.83 | 0.37 | | | | | | |

N/A—cannot be calculated/no effect

In comparison with these complexes Os and Ir analogues were also measured in cysplatin resistant A2780 cells as described herein. The results are shown on FIG. 26 as well as in Table 6 below.
The result of the SRB assay is also given in Table 5

TABLE 5

| | A2780 cisplatin resistant | | |
|---|---|---|---|
| | Max. | IC50 | HillSlope |
| Ru1 | 70.62 | 6.313 | 2.945 |
| Os1 | 63.68 | 6.754 | 3.346 |
| Ru2 | 80.79 | 7.245 | 2.489 |
| Os2 | 100 | 5.359 | 4.32 |
| Ru3 | 100 | 7.677 | 3.175 |
| Os3 | 79.73 | 7.306 | 4.426 |
| Ru4 | 77.51 | 1.643 | 3.324 |
| Os4 | 77.16 | 0.8742 | 1.439 |
| Ir4 | 77.46 | 1.808 | 1.779 |

As can be seen the IC50 values are roughly similar showing the activity of the compounds in cisplatin resistant cells.

### Example 3—the Biological Activity of Ruthenium Complexes is Dependent on Reactive Oxygen Species Production

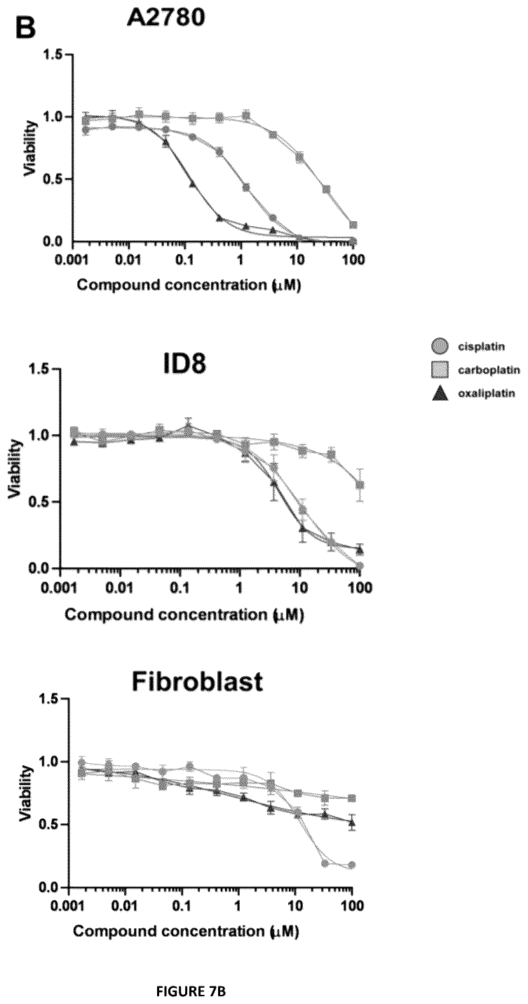

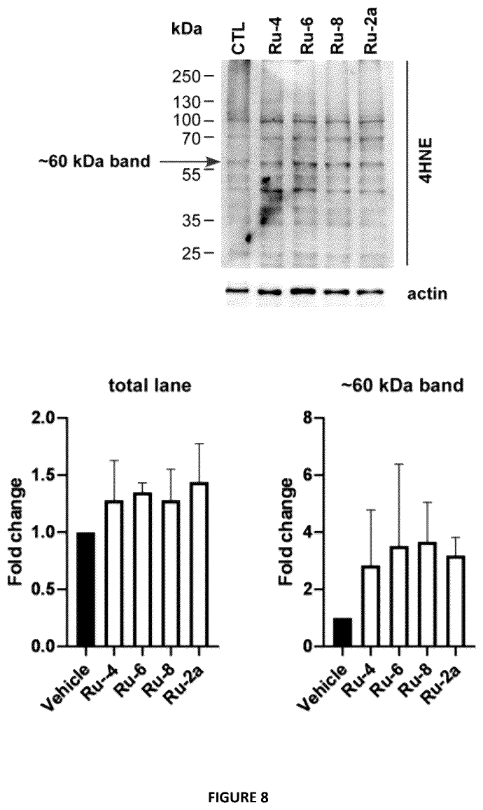

Currently available ruthenium complexes with antineoplastic activity have diverse modes of action involving (mitochondrial) reactive oxygen species production (Bakewell et al., 2020; Fernandes, 2019; Mihajlovic et al., 2020; Xu et al., 2018) and the induction of DNA damage (Parveen et al., 2019; Spillane et al., 2008). We detected an increase in the expression of an oxidative stress marker, 4-hydroxy-nonenal (4HNE) at the level of the whole lane, as well as, when a specific band was assessed, in A2780 cells treated with the active ruthenium complexes Ru-2a, Ru-4, Ru-6, Ru-8 corresponding to the IC50 values (FIG. 8).

FIG. 8 illustrates that ruthenium complexes induce reactive oxygen species production. 1×10⁶ A2780 cells were plated to 6-well plates. Cells were treated with Ru-2a at 1.3 μM, Ru-4 at 6.2 μM, Ru-6 at 8.5 μM and Ru-8 at 3.7 μM for 48 hours. Then cells were harvested, protein lysate was prepared. Subsequently, 20 μg protein was separated by SDS-PAGE on an 8% gel, followed by Western blotting and the blot was stained with 4HNE and actin antibodies.

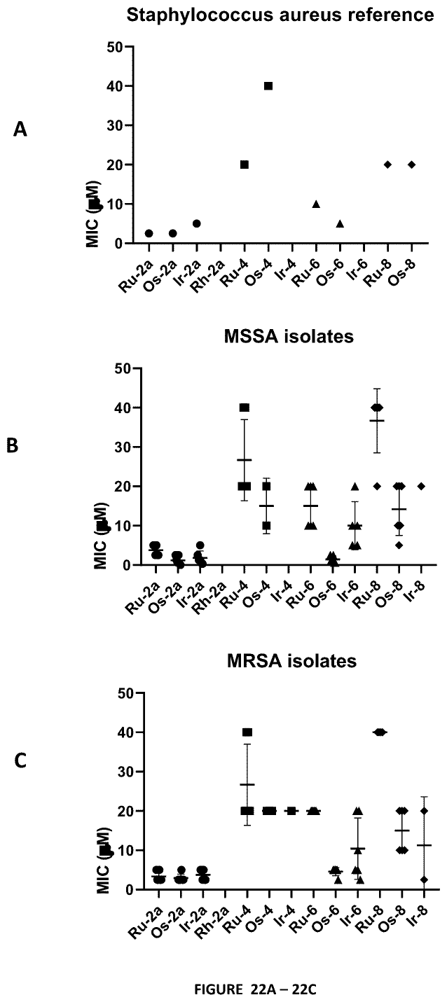

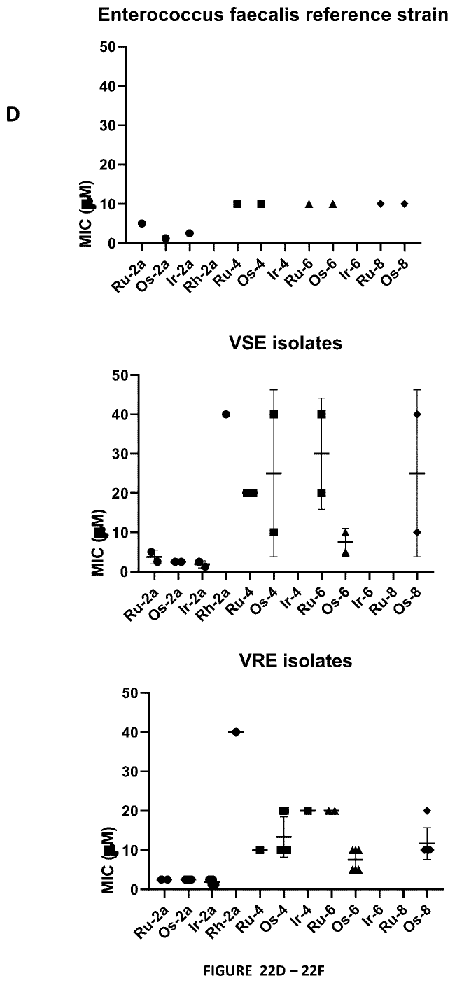

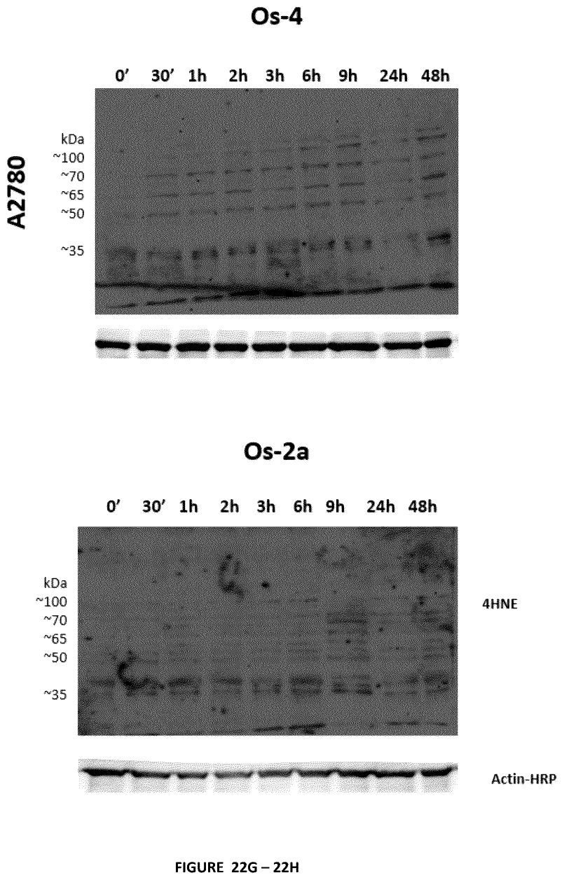

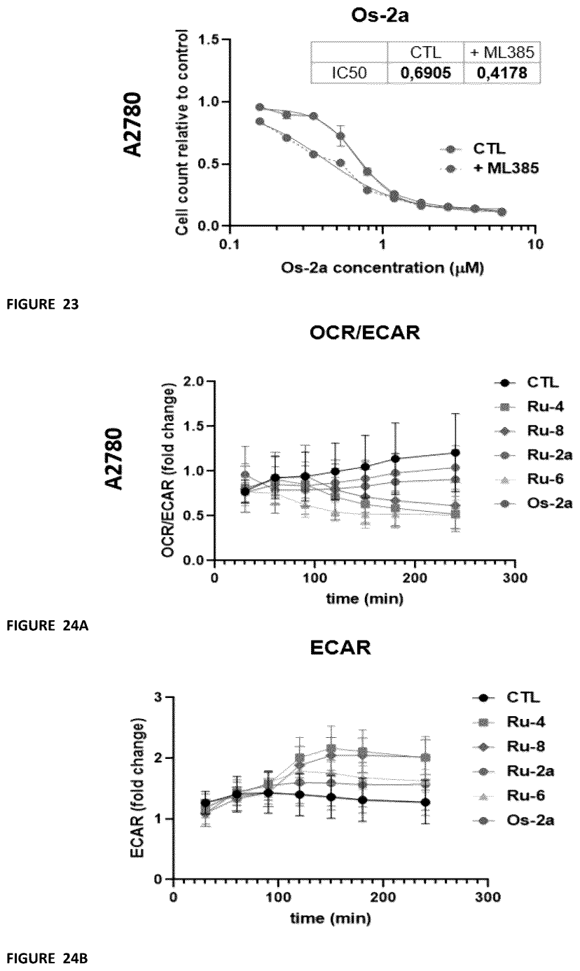

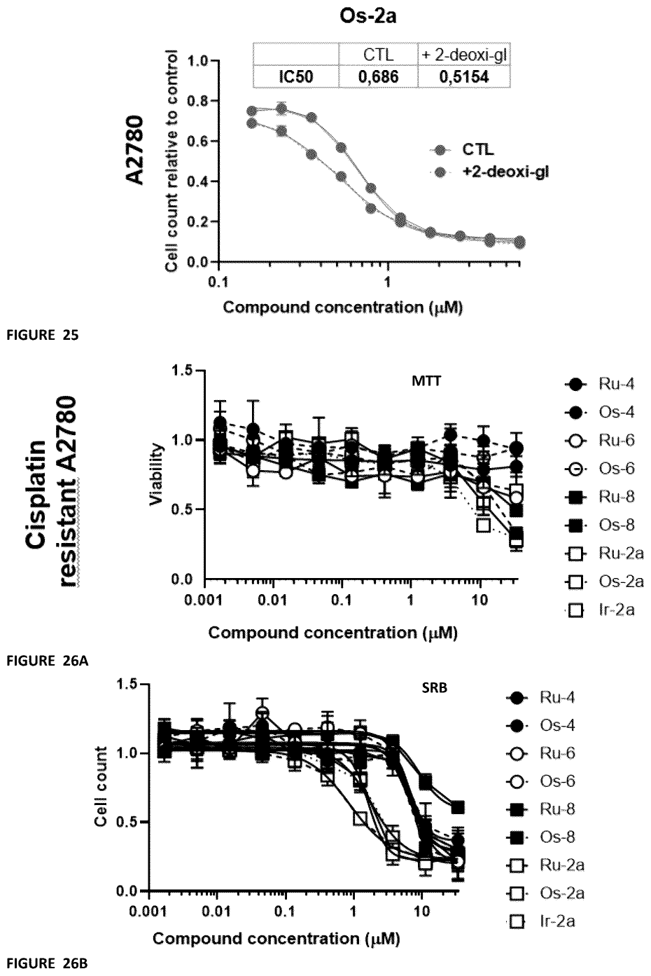

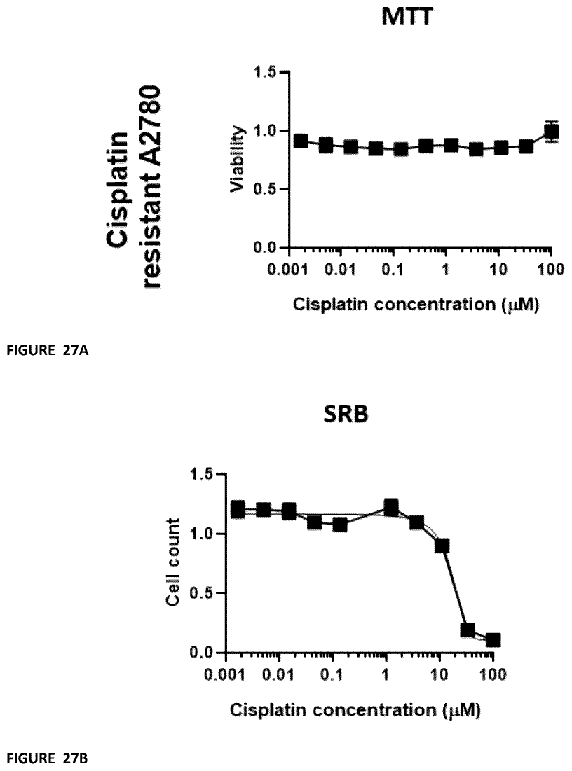

Osmium complexes also induced reactive oxygen species production (FIG. 22.). In this experiment 1×10⁶ A2780 cells were plated to 6-well plates. Cells were treated with Os-2a at 0.73 μM for the time indicated on FIG. 22. Then cells were harvested, protein lysate was prepared. Subsequently, 20 μg protein was separated by SDS-PAGE on an 8% gel, followed by Western blotting and the blot was stained with 4HNE and actin antibodies.

To further study whether cytostasis by the present compounds is ROS-related the ROS production mechanism, the interrelation between Nrf2, a key transcriptional regulator of the cellular antioxidant defense system (Smolková et al., 2020), and ruthenium/osmium complexes was studied.

A2780 cells were treated with Nrf2 inhibitor ML385 at 1.2 μM concentration in a combination with a concentration series of Os-2a for 48 hours, then SRB assay was performed. Data is represented as average ±SEM, from two biological replicates. Nonlinear regression was performed to obtain the IC50 values. Values were normalized for vehicle treated cells. It has been found that treatment of cells with an NRF2 inhibitor (ML385) potentiated the effects of the complexes that is in good correlation with the ROS production mechanism of cytostasis.

We assessed whether oxidative stress had a role in the cytostatic effects exerted by Ru-2a, Ru-4, Ru-6 and Ru-8. To that end, we tried to revert the cytostatic effects of ruthenium complexes by strong reductants as reduced glutathione (GSH) and N-acetyl-cysteine (NAC). Furthermore, we also tested MitoTEMPO, a mitochondrially targeted antioxidant that can efficiently detoxify mitochondria-derived reactive oxygen species (Bai, 2015; Hegedus et al., 2021; Sári et al., 2020). GSH and NAC co-treatment attenuated the cytostatic effects induced by Ru-2a, Ru-4, Ru-6 and Ru-8 (FIG. 9A) pointing out the causative role of reactive oxygen species production in cytostasis. Nevertheless, MitoTEMPO did not modulate the effects of Ru-2a, Ru-4, Ru-6 and Ru-8 (FIG. 9A) suggesting that mitochondria are not the source of the reactive oxygen species.

As thiols are soft Lewis bases and ruthenium is a soft Lewis acid, excess amounts of GSH or NAC can lead to the disassembly of ruthenium complexes. To provide evidence against this scenario, we applied another antioxidant, vitamin E that does not have thiol groups. The application of vitamin E, similar to GSH and NAC, attenuated the cytostatic effects of the bioactive ruthenium complexes (FIG. 9B) providing further evidence on the involvement of reactive oxygen species.

Interestingly, treatment of cells with Trolox, a derivative of vitamin E lacking the apolar phytyl chain, did not provide protection against ruthenium compounds (FIG. 9C). This suggests that the effect is membrane-related and may be linked with the fact that apolar protecting groups on the sugar moiety are necessary for the cytostatic effect.

FIG. 9 shows that reduced thiols and vitamin E can block the cytostatic effects of ruthenium complexes. A 3×10³ A2780 cells were plated to 96-well plates. Cells were treated with the compounds indicated in the concentrations indicated for 48 hours then SRB assay was performed.

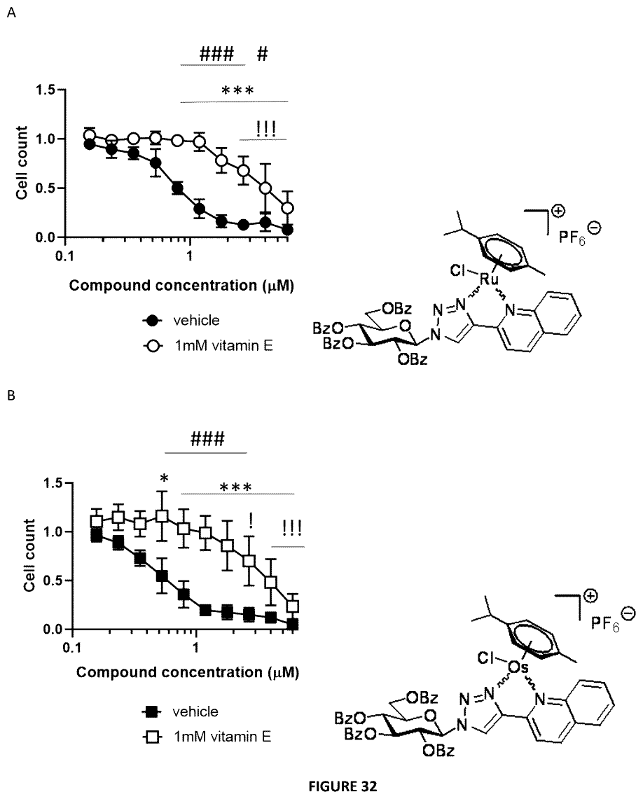

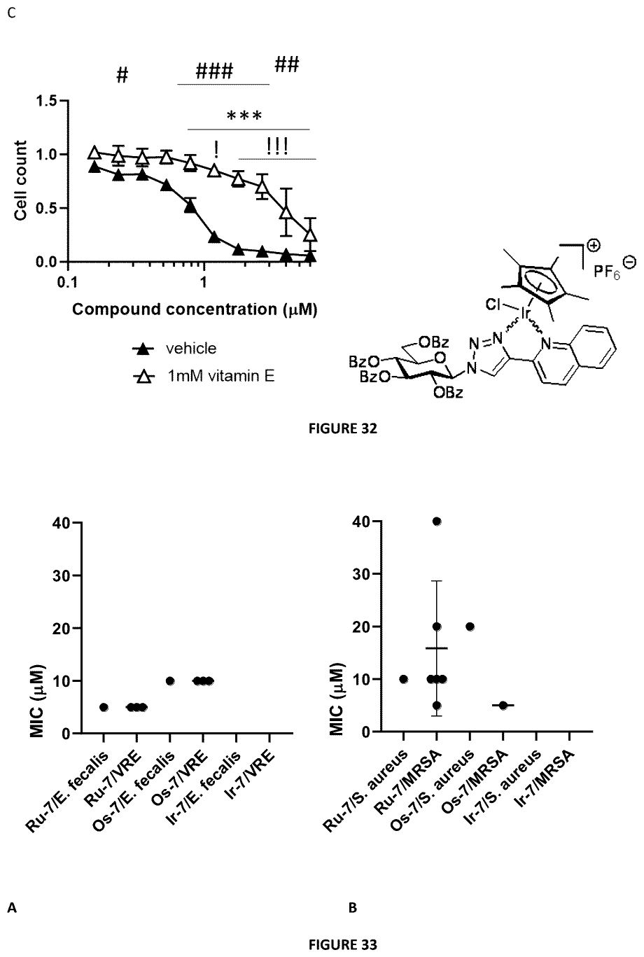

The effect of vitamin E, a lipid soluble antioxidant has also been studied with the quinoline containing compounds and has been found that it blocks the cytostatic activity of the bioactive complexes (FIG. 32). 1.5×103 A2780 cells were plated to 96 well plates. Cells were treated with the compounds in the concentrations indicated for 48 hours followed by an SRB assay. Data is represented as average ±SD, from four biological replicates; individual assays were performed in duplicates. Values were normalized for vehicle treated cells, absorbance for vehicle treated cells equals to 1. Normality was checked using the D'Agostino-Pearson normality test. The Ru-7 and the Os-7 dataset showed normal distribution, while the Ir-7 dataset was normalized using the Box-Cox normalization method. Statistical significance was assessed using two-way ANOVA test comparing all measurement points. * and *** represent statistical significance at p<0.05 and 0.001, respectively, between the lowest concentration and the higher concentrations among non-vitamin E-treated cells. ! and !!! represent statistical significance at p<0.05 and 0.001, respectively, between the lowest concentration and the higher concentrations among vitamin E-treated cells. #, ## and ### represent statistical significance at p<0.05, p<0.01 and 0.001, respectively, between the non-vitamin E-treated vs. vitamin E-treated cells.

Vitamin E treatment increased the IC₅₀ values of the complexes in all cases (FIG. 32). This suggests ROS production, similar to the pyridine containing compounds.

Reactive oxygen species production leads to DNA damage and poly(ADP-ribose) polymerase (PARP) activation (Bai, 2015; Curtin and Szabo, 2020). Certain ruthenium complexes were shown to potentiate the effects of PARP inhibitors (Bakewell et al., 2020; de Camargo et al., 2019; Xu et al., 2018; Yusoh et al., 2020). We assessed whether Ru-2a, Ru-4, Ru-6 and Ru-8 have similar properties by treating cells with 3 μM rucaparib, a clinically available potent PARP inhibitor.

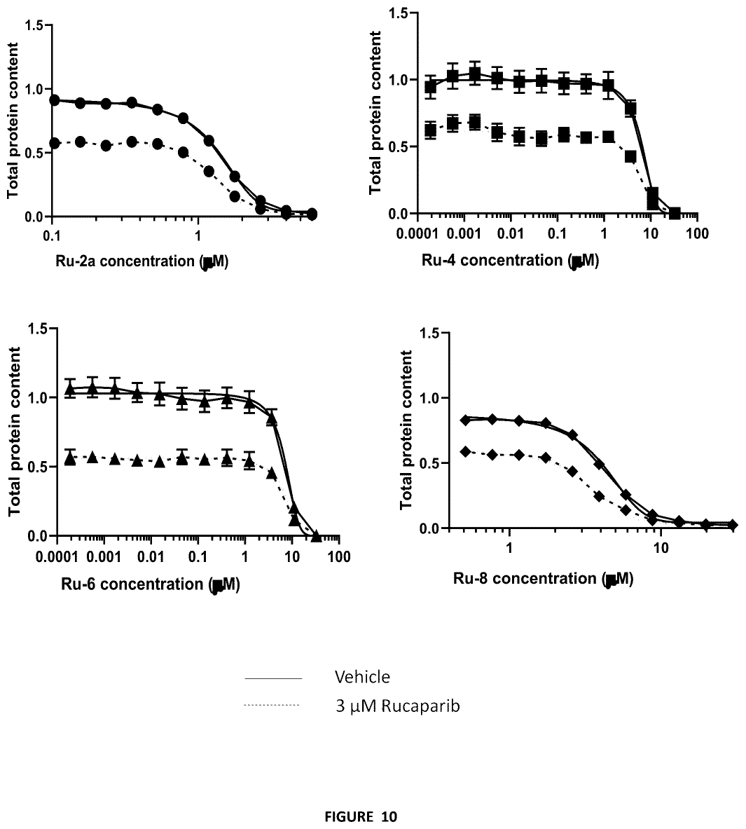

These results are illustrated on FIG. 10. 3×10³ A2780 cells were plated to 96-well plates. Cells were treated with Rucaparib at 3 μM concentration for 48 hours, then SRB assay was performed.

Rucaparib reduced cell proliferation in agreement with previous publications (e.g. (Zanjirband et al., 2017)) (FIG. 10). When we performed nonlinear regression analysis to assess the IC50 values we found that rucaparib potentiated the effects of all ruthenium complexes marked by a decrease in the IC50 values in combination treatment (FIG. 10). Nevertheless, it is of note that the potentiation was not as marked as the effects of antioxidants, hence, PARP-mediated effects probably have minor importance.

In an embodiment the compounds of the invention can be administered together with PARP inhibitors.

### Example 4—Bioactive Ruthenium Complexes can Cause Cytostasis in Breast Carcinoma, Pancreatic Adenocarcinoma and Glioblastoma Cell Lines

The question has been raised whether the sugar-based ruthenium complexes of the invention may be active in neoplastic diseases in general. This has been tested on other cell lines which are models for other neoplastic diseases. Breast cancer (modelled by MCF7 cells) has been selected as an option as prior art data (Hamala et al., 2020) suggested the potential effectiveness of other ruthenium complexes. Pancreatic adenocarcinoma (modelled by Capan2 cells) and glioblastoma (modelled by U251 cells), similar to ovarian cancer, are usually malign diseases where treatment options are limited (Kiss et al., 2020; Sipos et al., 2021; Tykocki and Eltayeb, 2018).

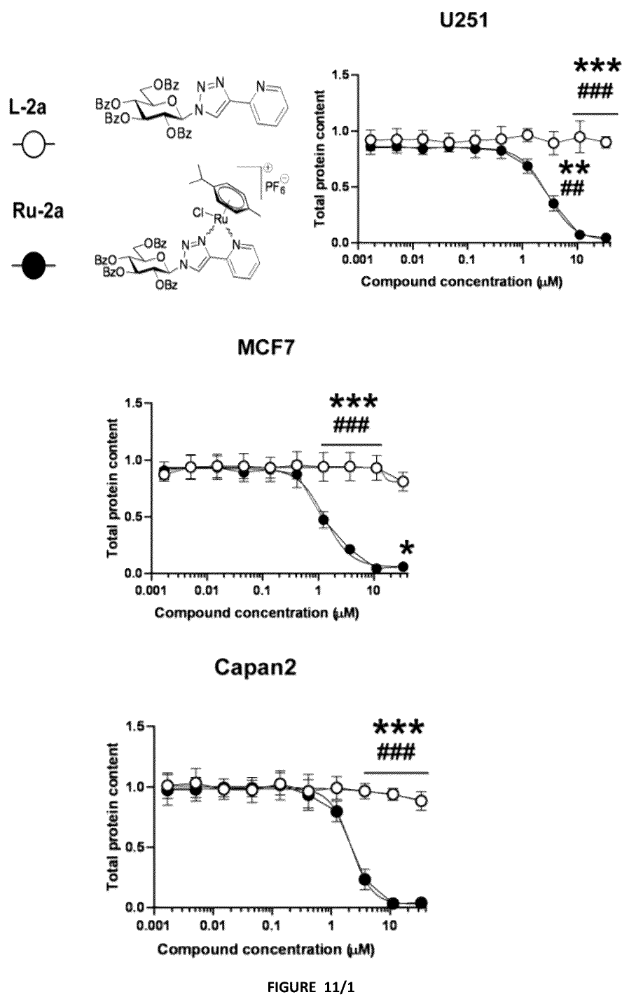

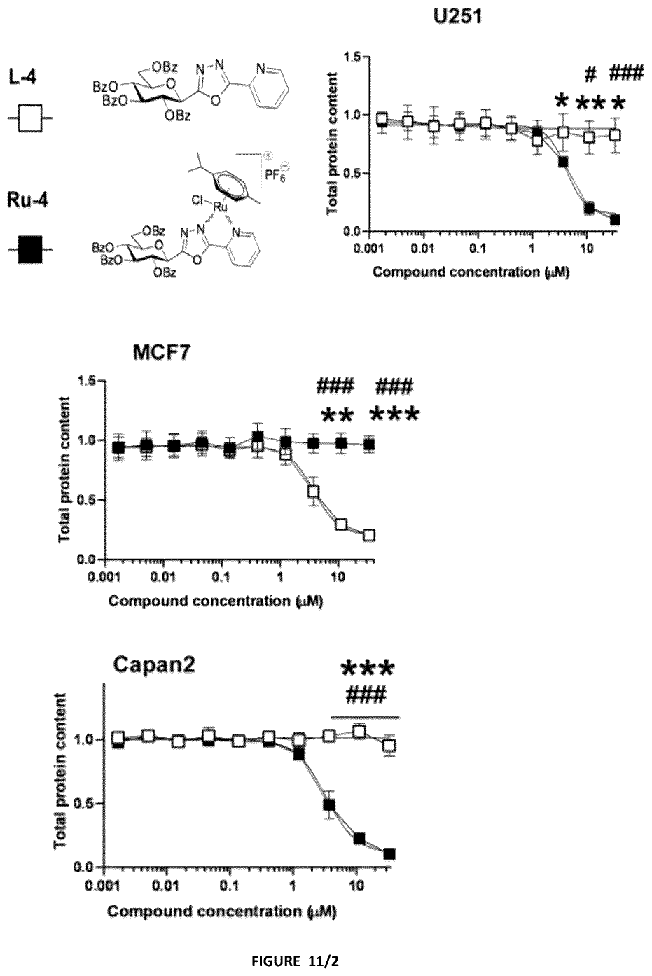

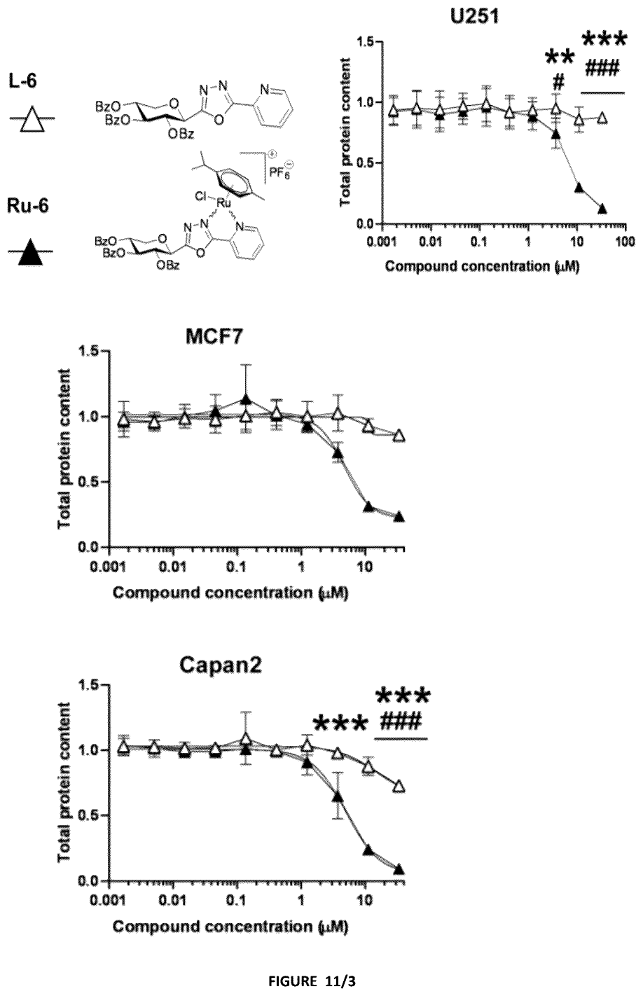

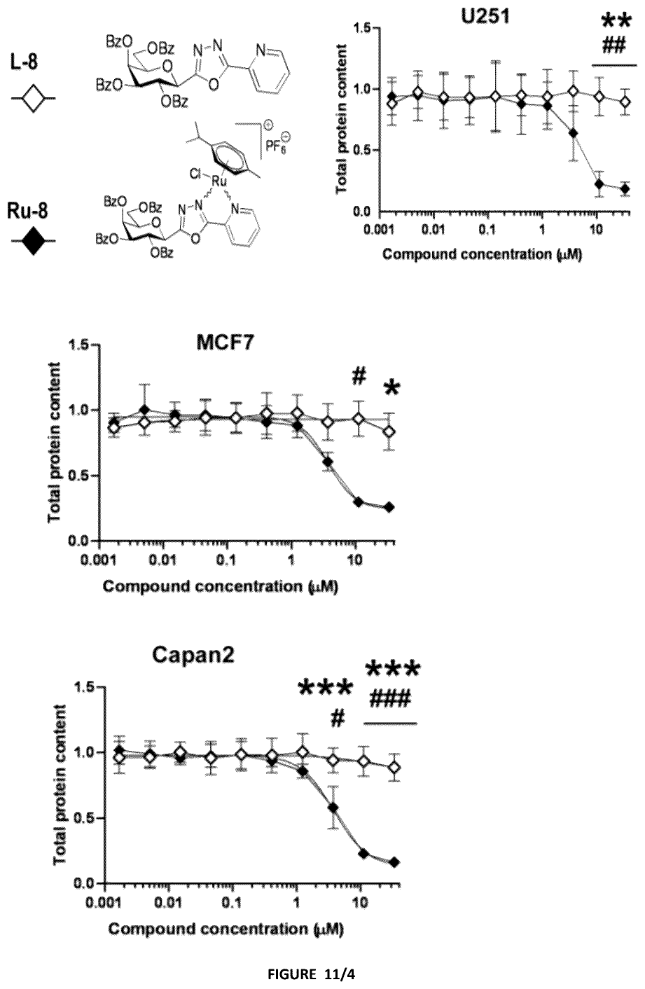

FIG. 11 describes assessment of the bioactive ruthenium complexes on models for glioblastoma, breast cancer and pancreatic adenocarcinoma for cytostatic activity). 2×10³ U251 cells (glioblastoma model), 3×10³ MCF7 cells (breast cancer) and 2×10³ Capan2 (pancreatic adenocarcinoma) were plated to 96-well plates. Cells were treated with the compounds in the concentrations indicated for 48 hours, then SRB assay was performed.

All active complexes (Ru-2a, Ru-4, Ru-6 and Ru-8) exerted cytostatic effects on all cell lines in proliferation assays (SRB) (see FIG. 11.) While the IC50 values of the ruthenium complexes on U251, MCF7 and Capan2 were higher than on A2780 or ID8 cells (Table 4), it has been found that the compounds of the invention are active in a broad range of neoplastic conditions.

### Example 5—the Carbohydrate Moiety is Important for the Bioactivity of the Ruthenium Complexes

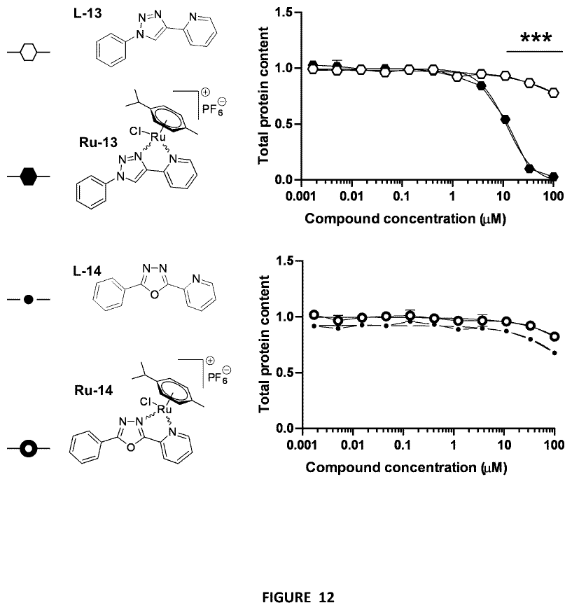

To assess the role of the monosaccharide moiety in the bioactive compounds we synthesized and assessed two molecules, where the monosaccharide unit was substituted with a phenyl group (Ru-13/L-13 and Ru-14/L-14). None of the free ligands, L-13 or L-14 had cytostatic activity on A2780 cells in SRB assays (FIG. 12).

FIG. 12 shows results on the assessment of non-sugar ruthenium complexes on A2780 cells for cytostatic activity. 3×10³ A2780 cells were plated to 96-well plates. Cells were treated with the compounds in the concentrations indicated for 48 hours, then SRB assay was performed.

The triazole-containing Ru-13 was cytostatic on A2780 cells with an IC50 value of ˜12 μM that is higher by one order of magnitude than the IC50 value of the sugar-containing molecule Ru-2a (FIG. 12, Table 4). Furthermore, the Hill coefficient of Ru-13 was 1.5 suggesting no or little cooperativity. The oxadiazole-containing Ru-14 had no cytostatic activity (FIG. 12).

### Example 6—Complexes with Quinoline Het2 Heterocycle

In a still further set of experiments, the quinoline complexes (Ru-2b, Os-2b, Ir-2b, Rh-2b) were tested on A2780 and ID8 ovarian cancer cells, and on non-transformed primary human fibroblasts, used as controls, in a concentration range up to 33.3 μM (FIG. 28). In concentrations exceeding 33.3 μM the complexes frequently precipitated.

In these experiments shown on FIG. 28 3×10³ A2780 cells, 2×10³ ID8 cells and 4×10³ primary fibroblasts were plated to 96 well plates. Cells were treated with the compounds in the concentrations indicated for either 4 hours for an MTT assay or for 48 hours for an SRB assay. Data is represented as average ±SD, from three biological replicates; individual assays were performed in duplicates. Values were normalized for vehicle treated cells, absorbance for vehicle treated cells equals to 1. Statistical significance was assessed using two-way ANOVA test comparing the free ligand and each complex as a complex-ligand pair. Before the test normality was assessed using the D'Agostino-Pearson test. The ID8 SRB Ir-2b+L-2b dataset had lognormal distribution. The A2780 MTT Ru-2b+L-2b, A2780 SRB Os-2b+L-2b, Fibroblast SRB Ru-2b+L-2b were transformed using the Box-Cox method to achieve normal distribution. All other datasets had normal distribution. Nonlinear regression was performed on the A2780 SRB and the ID8 SRB datasets. (FIG. 28)

All of the complexes and the free ligand exerted little rapid cytotoxicity in MTT assays, however, in SRB assays that detects cytostasis, the Ru-2b, Os-2b and Ir-2b complexes completely blocked cell proliferation with submicromolar or low micromolar IC₅₀ values both in A2780 and ID8 cells

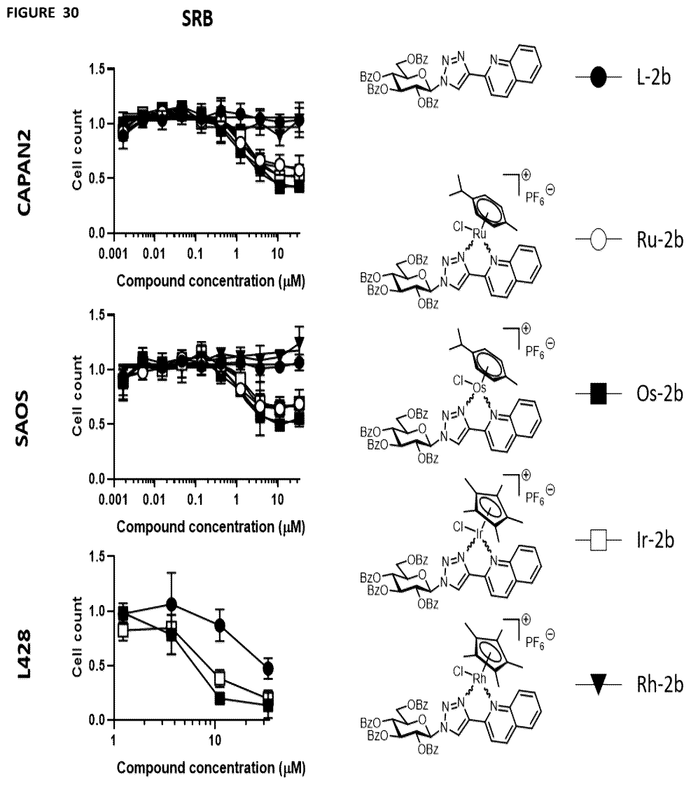

Quinoline-containing complexes Ru-2b, Os-2b, Ir-2b have been assessed for cytostatic activity on other cancer cell lines, too. 3×103 Capan2 cells, 2×103 Saos cells and 8×103 L428 were plated to 96 well plates. Cells were treated with the compounds in the concentrations for 48 hours. Then for Capan2 and Saos SRB assay was performed, L428 cells were counted using a Burker chamber. Data is represented as average ±SD, from three biological replicates; individual assays were performed in duplicates. Values were normalized for vehicle treated cells, absorbance for vehicle treated cells equals to 1. Normality was checked using the Shapiro-Wilk test. The Saos Os-7 dataset was normalized using the Box-Cox method, other datasets had normal distribution. Each complex was individually assessed for statistical significance using One-way ANOVA test followed by Dunnett's post-hoc test; all values were compared to the values of the lowest concentration. Nonlinear regression was performed on the Capan2 and Saos datasets (FIG. 30).

The data are presented in Table 6.

TABLE 6
Numeric values characterizing the complexes assessed in this study.

| | A2780 | | | | | | ID8 | | | |
|---|---|---|---|---|---|---|---|---|---|---|
| | MTT | | | SRB | | | MTT | | | SRB |
| | Max. | IC50 (μM) | Hill | Max. | IC50 (μM) | Hill | Max. | IC50 (μM) | Hill | Max. |
| L-2 | ND | ND | ND | 21.2 | ND | ND | | | | |
| Os-2 | 88 | ND | ND | 100 | 6.23 | 2.25 | | | | |
| L-3 | ND | ND | ND | 32.0 | ND | ND | | | | |
| Os-3 | 84.4 | ND | ND | 100 | 2.10 | 2.50 | | | | |
| L-4 | ND | ND | ND | 26.9 | ND | ND | | | | |
| Os-4 | 84.4 | ND | ND | 100 | 3.87 | 2.41 | | | | |
| L-5 | ND | ND | ND | 28.5 | ND | ND | | | | |
| Os-5 | 83.4 | ND | ND | 100 | 15.9 | 2.33 | | | | |

| | |
|---|---|
| L-6 | insoluble |

| | | | | | | | | | | |
|---|---|---|---|---|---|---|---|---|---|---|
| Os-6 | 76.9 | 16.5 | 1.89 | 88.4 | ND | ND | | | | |
| L-2b | ND | ND | ND | 16.9 | ND | ND | ND | ND | ND | ND |
| Ru-2b | 23.4 | ND | ND | 100 | 0.847 | 1.84 | 32.56 | ND | ND | 100 |
| Os-2b | 33.0 | ND | ND | 100 | 0.578 | 1.80 | 74.90 | ND | ND | 100 |
| Ir-2b | 23.2 | ND | ND | 100 | 0.891 | 1.84 | 19.23 | ND | ND | 100 |
| Rh-2b | 18.3 | ND | ND | 47.9 | ND | ND | ND | ND | ND | ND |
| Cisplatin | ND | ND | ND | 1000 | 1.21 | 1.20 | | | | |

| | ID8 | | cisplatin resistant A2780 | | | | | | |
|---|---|---|---|---|---|---|---|---|---|
| | SRB | | MTT | | | SRB | | | |
| | IC50 (μM) | Hill | Max. | IC50 (μM) | Hill | Max. | IC50 (μM) | Hill | LogD |
| L-2 | | | | | | | | | ND |
| Os-2 | | | | | | | | | 2.5 |
| L-3 | | | | | | | | | ND |
| Os-3 | | | | | | | | | 2.6 |
| L-4 | | | | | | | | | ND |
| Os-4 | | | | | | | | | 2.7 |
| L-5 | | | | | | | | | ND |
| Os-5 | | | | | | | | | 2.7 |
| L-6 | | | | | | | | | ND |
| Os-6 | | | | | | | | | 2.8 |
| L-2b | ND | ND | | | | | | | ND |
| Ru-2b | 1.14 | 3.78 | ND | ND | ND | 100 | 1.18 | 1.82 | 2.21 |
| Os-2b | 0.359 | 4.48 | ND | ND | ND | 100 | 0.426 | 1.17 | 2.33 |
| Ir-2b | 0.799 | 2.98 | ND | ND | ND | 100 | 1.535 | 2.40 | 2.17 |
| Rh-2b | ND | ND | | | | | | | 1.6 |
| Cisplatin | | | ND | ND | ND | 100 | 16.47 | ND | |

| | Capan2 | | | SAOS | | | L428 | | | Fibroblast | | | | | | |
|---|---|---|---|---|---|---|---|---|---|---|---|---|---|---|---|---|
| | SRB | | | SRB | | | SRB | | | MTT | | | SRB | | | |
| | Max. | IC50 (μM) | Hill | Max. | IC50 (μM) | Hill | Max. | IC50 (μM) | Hill | Max. | IC50 | Hill | Max. | IC50 (μM) | Hill | LogD |
| L-2 | | | | | | | | | | | | | | | | ND |
| Os-2 | | | | | | | | | | | | | | | | 2.5 |
| L-3 | | | | | | | | | | | | | | | | ND |
| Os-3 | | | | | | | | | | | | | | | | 2.6 |
| L-4 | | | | | | | | | | | | | | | | ND |
| Os-4 | | | | | | | | | | | | | | | | 2.7 |
| L-5 | | | | | | | | | | | | | | | | ND |
| Os-5 | | | | | | | | | | | | | | | | 2.7 |
| L-6 | | | | | | | | | | | | | | | | ND |
| Os-6 | | | | | | | | | | | | | | | | 2.8 |
| L-7 | | | | | | | | | | ND | ND | ND | ND | ND | ND | ND |
| Ru-7 | 39.80 | 1.39 | 2.08 | 33.64 | 1.18 | 5.16 | 52.73 | | | 23.93 | ND | ND | 50.04 | ND | ND | 2.21 |
| Os-7 | 59.11 | 1.35 | 1.26 | 48.04 | 1.29 | 1.22 | 100 | | | 30.97 | ND | ND | 48.94 | ND | ND | 2.33 |
| Ir-7 | 48.83 | 1.93 | 1.78 | 33 | 1.58 | 2.98 | 100 | | | 12.81 | ND | ND | 42.83 | ND | ND | 2.17 |
| Rh-7 | | | | | | | | | | 15.09 | ND | ND | ND | ND | ND | 1.6 |
| Cisplatin | | | | | | | | | | | | | | | | |

The IC50 value of cisplatin was obtained from measurements described above. Max—Maximal inhibition in %.

### Example 6.1—Complexes with Quinoline Het2 Heterocycle do not Act by Apoptosis

Also the toxic effects of the active Ru, Os and Ir complexes were verified by Annexin V-FITC propidium iodide (PI) double staining.

In the experiments shown on FIG. 29 2×10⁶ A2780 cells were plated in 12 well plates and were treated with the complexes (Ru-2b at 0,846 μM, Os-2b at 0,578 μM and Ir-2b at 0,891 μM) and 300 μM hydrogen peroxide for 2 hours. Cells were then stained with Annexin V and propidium iodide (PI) and cells were subjected to flow cytometry as described in Materials and Methods. The percent of cells in the quadrants are plotted. Data is represented as average ±SD, from three biological replicates, individual assays were performed in duplicates. Normal distribution was achieved by Box-Cox normalization of the data. Statistical significance was determined using a two-way ANOVA test, all measurement points were compared with each other. * indicate statistically significant differences between vehicle-treated (control) and treated cells (complexes or 300 μM H₂O₂) corresponding to the same quadrant (e.g. vehicle-treated double negative cells vs. H₂O₂-treated double negative cells) at p<0.05. (FIG. 29)

### Example 7—Quinoline-Containing Complexes are Active in Cisplatin-Resistant Cells

As noted above, one of the major drawbacks of platinum-based drugs is due to cisplatin resistance. We tested the three complexes with efficient cytostatic properties (Ru-2b, Os-2b and Ir-2b) on a cisplatin-resistant A2780 cell line.

In this experiment, 6×10³ cisplatin-resistant A2780 cells were plated to 96 well plates. Cells were treated with the compounds in the concentrations indicated for either 4 hours for an MTT assay or for 48 hours for an SRB assay. Data is represented as average ±SD, from three biological replicates; individual assays were performed in duplicates. Values were normalized for vehicle treated cells, absorbance for vehicle treated cells equals to 1. Each compound was assessed individually. Normality was assessed using the Shapiro-Wilk test. Normality was achieved using the Box-Cox transformation in the case of Os-2b and Ir-2b datasets, while Ru-2b dataset had normal distribution. Statistical significance was assessed using One-way ANOVA test comparing all points to the smallest concentration. Nonlinear regression was performed on the results of the SRB assay datasets.

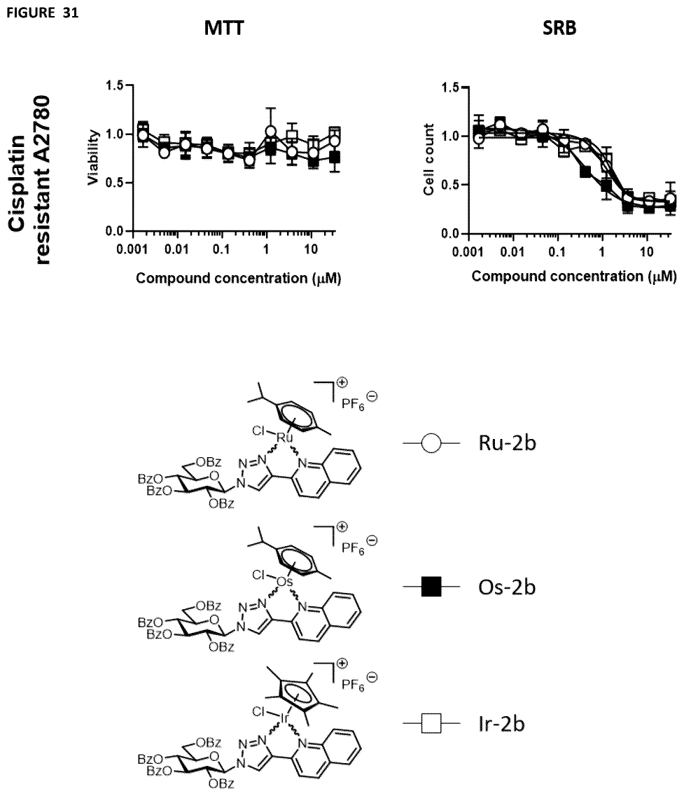

The complexes did not exert direct toxicity in MTT assays on the cisplatin-resistant cells (FIG. 31, Table 6).

Nevertheless, we have observed important differences in cell proliferation. The IC₅₀ value of cisplatin was 1.21 μM in our previous study on cisplatin-sensitive A2780 cells. The IC₅₀ value increased to 16.47 μM in the cisplatin resistant cell line (13.6 fold increase) in SRB assays. In contrast to that, the IC₅₀ value of Ru-2b (0.8466 μM vs. 1.183 μM, 1.40 fold change) and Ir-2b (0.891 μM vs. 1.535 μM, 1.72 fold change) increased, although not to the same extent as for cisplatin. Furthermore, the IC₅₀ value of Os-2b (0.5777 μM vs. 0.476 μM) was left technically unchanged when comparing the cisplatin sensitive and cisplatin resistant cell lines (FIG. 31, Table 6). Apparently, these complexes are not detoxified strongly in cisplatin resistant cells suggesting that these compounds can be used to overcome cisplatin resistance.

The case is similar with the complexes of the first stage of the study as shown by the IC50 values in Table 5 in comparison with the corresponding values with the non-cisplatin resistant A2780 strain.

### Example 8—Complexes with Alkanoyl Substituted Carbohydrate Moiety

In a further set of experiments a set of osmium complexes and their sugar-derived ligands, in which the benzoyl protective groups on the carbohydrate moiety were replaced by aliphatic acyl groups of different chain length (straight chain C₃-C₇—CO).

Cells were treated with the compounds in the concentrations indicated for either 4 hours for an MTT assay or for 48 hours for an SRB assay. Data is represented as average ±SD, from three biological replicates; individual assays were performed in duplicates. Correlation between the log D and the IC₅₀ values in alkyl-protected complexes is shown on FIG. 37. The black line represents the IC₅₀ values, while the red line represents the log D values.

Values were normalized for vehicle treated cells, absorbance for vehicle treated cells equals to 1. The MTT dataset for Os-C4+L-C4 and the SRB datasets for Os-2+L-2, Os-3+L-3 showed normal distribution, the MTT datasets for Os-C3+L-C3, Os-C5+L-C5, Os-C6+L-C6, Os-C7 and the SRB dataset for Os-C5+L-C5, Os-C6+L-C6, Os-C7 was normalized using the Box-Cox method. Except for the Os-C7 complex, Two-way ANOVA test was performed and all values were compared with each other (Tukey's post-hoc test). For Os-C7 One-way ANOVA was performed on Box-Cox normalized values followed by Dunnett's post-hoc comparing all values to the smallest treatment concentration. *, ** and *** indicate statistically significant differences between vehicle-treated (control) and the cells treated with a compound at p<0.05, p<0.01 and p<0.001, respectively. ## and ### indicate statistically significant differences between the free ligand and the corresponding complex at p<0.01 and p<0.001, respectively. Nonlinear regression was performed on the data (FIG. 34-36).

The compounds had high log D value indicating a strong apolar character up to the point that the free ligand L-C7 with the longest C₇H₁₅—CO alkanoyl protective groups proved to be insoluble, hence, it was not suitable for testing. All complexes Os-C3-Os-C7, but not the free ligands L-C3-L-C6, exerted rapid toxicity on A2780 cells in MTT assays (FIGS. 34 to 36) with cytotoxicity over 80%.

### Example 9—Antimicrobial Effect

Antimicrobial effect of the complexes has been examined in accordance with the microdilution method based on the proposals of the EUCAST (European Committee on Antimicrobial Susceptibility Testing).

While most complexes tested have turned out to show some activity, below the inevitably anti-bacterial complexes are showed by underlining and the most active ones in bold for each strain/isolates type. Complexes marked as (−) have not been tested in the experiment.

In case of pyridine-containing, benzoyl-substituted compounds ruthenium, osmium, iridium and rhodium complexes, having similar structure to the quinoline-based compounds identified in this study, have bacteriostatic activity on multiresistant Enterococcus and Staphylococcus aureus clinical isolates in low micromolar or submicromolar concentrations.

In a further stage of the experiments the bioactive Ru-2b, Os-2b and Ir-2b complexes for bacteriostatic activity have been tested.

Ru-2b, Os-2b and Ir-2b complexes exerted bacteriostatic activity on reference strains and clinical VRE and MRSA isolates. The MIC values of the complexes were determined on the reference strains of S. aureus (ATCC11007) and E. faecalis. (ATCC29112) and on clinical VRE and MRSA isolates were determined by microdilution assays (repeated at least twice in duplicates) as described in Materials and Methods. The numbers indicate how many isolates were susceptible to the compound out of those tested; i.e. 1/6 stands for 1 isolate was susceptible out of 6 tested (see FIG. 33).

The Ru-2b complex had a MIC value of 5 μM on the reference Enterococcus faecalis (ATCC29112) strain and a MIC of 10 μM on the Staphylococcus aureus (ATCC11007) reference strain. The Ru-7 complex was bacteriostatic on all of the multiresistant clinical VRE isolates with a MIC value of 5 μM, while inhibited the growth of all multiresistant clinical MRSA isolates with MIC values of 5 to 40 μM (FIG. 33, Table 3.

The Os-2b complex had a MIC value of 10 μM on the reference Enterococcus faecalis (ATCC29112) strain and a MIC value of 20 μM on the Staphylococcus aureus (ATCC11007) reference strain. The Os-7 complex was bacteriostatic on all of the multiresistant clinical VRE isolates with a MIC value of 10 μM, while inhibited the growth of 1 out of the 6 multiresistant clinical MRSA isolates with a MIC value of 5 μM (FIG. 33, Table 3.

The Ir-2b complex had no bacteriostatic activity on either reference strains or on the clinical isolates (FIG. 33).

TABLE 3
The clinical isolates used in the study and the MIC
values (in μM) of the complexes against the isolates.

| Species and strain identity | | Ru-7 | Os-7 | Ir-7 | Sample | Year |
|---|---|---|---|---|---|---|
| Reference | E. faecalis | 5 | 10 | 40< | | |
| VRE | 25 051 | 5 | 10 | 40< | Nephrostoma | 2018 |
| VRE | 27 085 | 5 | 10 | 40< | Wound | 2018 |
| VRE | 25 498 | 5 | 10 | 40< | Rectal swab for screening | 2018 |
| | | | | | for multiresistant | |
| | | | | | pathogens | |
| Reference | S. aureus | 10 | 20 | 40< | | |
| MRSA | 24 272 | 5 | 5 | 40< | Throat | 2018 |
| MRSA | 24 408 | 10 | 40< | 40< | Bronchial | 2018 |
| MRSA | 20 426 | 10 | 40< | 40< | Blood | 2020 |
| MRSA | 24 035 | 40 | 40< | 40< | Wound | 2018 |
| MRSA | 24 328 | 10 | 40< | 40< | Throat | 2018 |
| MRSA | 24 268 | 20 | 40< | 40< | Throat | 2018 |

VRE—vancomycin-resistant Enterococcus, MRSA—methicyllin-resistant Staphylococcus aureus.

Antimicrobial effect of complexes Os-C3 to Os-C6 is tested as described for Ru-2b, Os-2b and Ir-2b complexes.

### Example 10—Materials and Methods 1—Synthesis

### 10.1 General Methods

Optical rotations were measured on a Jasco P-2000 polarimeter (Jasco, Easton, MD, USA) at rt, and the data referred to the average of three parallel measurements. The ¹H and ¹³C NMR spectra of the newly synthesized compounds were recorded with Bruker DRX360 (360/90 MHz for ¹H/¹³C) or Bruker DRX400 (400/100 MHz for ¹H/¹³C) spectrometers. Chemical shifts are referenced to Me₄Si (¹H-NMR) or to the residual solvent signals (¹³C-NMR). Assignments of the proton- and carbon signals of the new compounds were based on COSY and HSQC correlations.

MS spectra were obtained by a Bruker maXis II (ESI-HRMS) spectrometer.

TLC analysis were carried out by using DC Kieselgel 60 F₂₅₄ plates (Sigma-Aldrich) and the spots were visualized under UV light and by gentle heating.

For column chromatographic purification Kieselgel 60 (Molar Chemicals, particle size 0.063-0.2 mm) silica gel was applied. Anhydrous pyridine was purchased from VWR Chemicals. Anhydrous solvents were obtained by using standard distillation procedures. Anhydrous CH₂Cl₂, CHCl₃ and toluene were produced by distillation from P₄O₁₀ and then stored over 4 Å molecular sieves (CH₂Cl₂, CHCl₃) or sodium wires (toluene). MeOH was dried by distillation over Mg turnings and iodine.

The 2-ethynylpyridine (TCI Chemicals) and the dichloro(η⁶-p-cymene)ruthenium(II) dimer (Ru-dimer, Strem Chemicals), dichloro(pentamethylcyclopentadienyl)iridium(III) dimer (Ir-dimer, Acros Organics), dichloro(pentamethylcyclopentadienyl)rhodium(III) dimer (Rh-dimer, Alfa Aesar) and TIPF₆ (Strem Chemicals) were purchased from the indicated suppliers.

The 2,3,4,6-tetra-O-acetyl-β-D-glucopyranosyl-azide (Györgydeák and Thiem, 2006; Paulsen et al., 1974) (1), the 5-(2′,3′,4′,6′-tetra-O-benzoyl-β-D-glucopyranosyl)-tetrazole (Hadady et al., 2004; Kun et al., 2011) (2), the 5-(2′,3′,4′-tri-O-benzoyl-D-D-xylopyranosyl)-tetrazole (Somsak et al., 2014) (3), the 5-(2′,3′,4′,6′-tetra-O-acetyl-D-D-galactopyranosyl)-tetrazole (Farkas et al., 1977) (4), the 2-(L-arabino-1′,2′,3′,4′-tetraacetoxybutyl)-tetrazole (Viana et al., 2008) (5), the 2-ethynylquinoline (Son et al., 2013), 1-phenyl-4-(pyridine-2-yl)-1,2,3-triazole (Wei et al., 2010) (Tawfiq et al., 2014) (L-13), 2-phenyl-5-(pyridine-2-yl)-1,3,4-oxadiazole (Wei et al., 2010; Weiss et al., 2020) (L-14) and the dichloro(η⁶-p-cymene)osmium(II) dimer (Os-dimer) (Godó et al., 2012) were synthesized according to literature procedures.

The 1-(β-D-glucopyranosyl)-4-(pyridin-2-yl)- and -(quinolin-2-yl)-1,2,3-triazoles (1 and 2, respectively) were synthesized according to our earlier described procedures [16].

### 10.2 General Procedure I for the Preparation of 1-(2′,3′,4′,6′-tetra-O-acetyl-β-D-glucopyranosyl)-4-hetaryl-1,2,3-triazoles

To a solution of 1-(2,3,4,6-tetra-O-acetyl-β-D-glucopyranosyl)-azide (Györgydeák and Thiem, 2006; Paulsen et al., 1974) (1) in CH₂Cl₂ (1 mL/50 mg azide) the appropriate 2-ethynylated heterocycle (1 eq.) and the C₃H₇COOCu(PPh₃)₂ catalyst (Bokor et al., 2012) (3 mol %) were added. The reaction mixture was stirred at rt for 1 day, and the completion of the reaction was judged by TLC (1:1 EtOAc-hexane). The solvent was then removed under diminished pressure and the residue was purified by column chromatography.

### 10.3 General Procedure II for the Synthesis of O-peracylated 2-glycosyl-5-(pyridin-2-yl)-1,3,4-oxadiazoles

The corresponding O-peracylated 5-glycosyl-tetrazole (2-5) was dissolved in dry toluene (1 mL/100 mg substrate), then 2-picolinic acid (2 eq.) and DCC (2 eq.) were added. The reaction mixture was stirred at boiling temperature until the TLC showed total consumption of the tetrazole. The insoluble materials were filtered off, washed with CH₂Cl₂ and the filtrate was evaporated under reduced pressure. The crude product was purified by column chromatography and crystallisation.

### 10.4 General Procedure III for the Zemplén Deacylation

An O-acyl protected glycosyl-azole was dissolved in a 1:1 mixture of dry MeOH and dry CHCl₃ (1 mL/25 mg substrate) and a few drops of ˜1 M solution of NaOMe in MeOH was added (pH=8-9). The reaction mixture was left at rt until the TLC indicated total conversion of the starting material. The neutralisation of the solution was carried out by the addition of a cation exchange resin (Amberlyst 15, H⁺ form). The resin was then filtered off and the solution was evaporated under reduced pressure. The crude product was purified by column chromatography or crystallisation.

### 10.5 General Procedure IV for the Formation of the [(η⁶-p-Cym)M^{II}(N—N))Cl]PF₆ (M=Ru, Os) and [(η⁵-Cp*)M^{III}(N—N))Cl]PF₆ (M=Ir, Rh) Type Complexes Containing O-Peracylated Glycosyl Azole Ligands

To a solution of dimer ([(η⁶-p-cym)M^{II}Cl₂]₂(M=Ru, Os) or [(η⁵-Cp*)M^{III}Cl₂]₂(M=Ir, Rh)) in CH₂Cl₂ (10 mg/1 mL) the corresponding O-peracylated glycosyl azole (2 eq.) and TIPF₆ (2 eq.) were added. Under stirring the same volume of methanol was added to this reaction mixture in order to accelerate the precipitation of the TICI. The reaction mixture was then stirred at rt for an hour, and the total disappearance of the half-sandwich dimer compound was judged by TLC (9:1 CHCl₃-MeOH). After completion of the complexation reaction the precipitated TICI was filtered off by membrane filter and the solution was evaporated in vacuo. The crude complex was purified by dissolving the compound in CHCl₃ (10 mg/mL) and the product was precipitated by the addition of Et₂O (CHCl₃:Et₂O=1:2 or 1:4 ratio), then the product was washed with CHCl₃-Et₂O (1:1 or 1:2) solvent mixture (30 mg/mL) and dried in vacuo affording [(η⁶-p-cym)M^{II}(N—N))Cl]PF₆ (M=Ru, Os) or [(η⁵-Cp*)M^{III}(N—N))Cl]PF₆ (M=Ir, Rh) type complex. The crude product can be purified by column chromatography as well (9:1 or 95:5 CHCl₃-MeOH), but appears to be more time and solvent consuming and less pure.

### 10.6 General Procedure for the Formation of the [(η⁶-p-cym)M^{II}(N—N))Cl]PF₆ (M=Ru, Os) and [(η⁵-Cp*)M^{III}(N—N))Cl]PF₆ (M=Ir, Rh) Type Complexes Containing Unprotected Glycosyl Azole Ligands

To a solution of dimer ([(η⁶-p-cym)M^{II}Cl₂]₂(M=Ru, Os) or [(η⁵-Cp*)M^{III}Cl₂]₂(M=Ir, Rh)) in CH₂Cl₂ (10 mg/1 mL) the corresponding unprotected glycosyl azole (2 eq.) and TIPF₆ (2 eq.) were suspended. Under stirring the same volume of methanol was added to this reaction mixture in order to dissolve the heterocyclic sugar derivative and accelerate the precipitation of the TICI. The reaction mixture was then stirred at rt for an hour, and the total disappearance of the half-sandwich dimer compound was judged by TLC (9:1 CHCl₃-MeOH). After completion of the complexation reaction the precipitated TICI was filtered off by membrane filter and the solution was evaporated in vacuo. The crude complex was purified by crystallisation from pure iPrOH or iPrOH: Et₂O=2:5 solvent mixture affording [(η⁶-p-cym)M^{II}(N—N))Cl]PF₆ (M=Ru, Os) or [(η⁵-Cp*)M^{III}(N—N))Cl]PF₆ (M=Ir, Rh) type complex.

### 10.7 General Procedure I for O-Peracylation of the 1-(β-D-glucopyranosyl)-4-hetaryl-1,2,3-triazoles

A solution of the appropriate 1-(β-D-glucopyranosyl)-4-hetaryl-1,2,3-triazole (1 or 2) in anhydrous pyridine (4 mL/50 mg substrate) was cooled down in an ice bath and the corresponding carboxylic acid chloride (4.8 equiv.) was added under stirring. The reaction mixture was then heated at 60° C. and monitored by TLC (1:1 CHCl₃-MeOH and 1:2 EtOAc-hexane). If the TLC showed incomplete conversion after one hour, an additional portion of acid chloride (4.8 equiv.) was added to the mixture. After completion of the reaction the pyridine was removed in vacuo. The residue was dissolved in CHCl₃ (30 mL) and extracted with sat. aq. solution of NaHCO₃ (2×30 mL) and with water (35 mL). The separated organic phase was dried over MgSO₄, filtered and evaporated. The residual crude product was purified by column chromatography.

### 10.8 General Procedure II for the Preparation of the [(η⁶-p-cym)M^{II}(N—N)Cl]PF₆ (M=Ru, Os) and [(η⁵-Cp*)M^{III}(N—N)Cl]PF₆ (M=Ir, Rh) Type Complexes

The corresponding O-peracylated 1-(β-D-gucopyranosyl)-4-hetaryl-1,2,3-triazole (L-2-L-7, 2.0 or 2.1 equiv.), the complex dimer (Ru—/Os-/Ir-/Rh-dimer, 1 equiv.) and TIPF₆ (2 equiv.) were dissolved in a 1:1 mixture of anhydrous CH₂Cl₂ and MeOH (1-1 mL/10 mg dimer). The reaction mixture was vigorously stirred until the TLC (95:5 CHCl₃-MeOH) showed complete disappearance of the starting dimer complex. After completion of the reaction, the precipitated TICI was filtered off and the solvents were removed. The residual crude product was purified by crystallization or by column chromatography.

### Example 11 Materials and Methods 2—NMR Data of the Complexes of the Invention

### 11.1 1-(2′,3′,4′,6′-Tetra-O-acetyl-β-D-glucopyranosyl)-4-(pyridin-2-yl)-1,2,3-triazole (L-1a) (Wilkinson et al., 2006)

Prepared from azide (Györgydeák and Thiem, 2006; Paulsen et al., 1974) 1 (100 mg, 0.27 mmol) and 2-ethynylpyridine (28 μL, 0.27 mmol) according to general procedure I. Purified by column chromatography (1:1→2:1 EtOAc-hexane) to give 121 mg white amorphous solid (95%). R_{f}=0.18 (1:1 EtOAc-hexane); [α]_{D}=−55 (c 0.21, CHCl₃). ¹H NMR (360 MHz, CDCl₃) δ (ppm): 8.61 (1H, d, J=4.8 Hz, Py-H-6), 8.42 (1H, s, Tria-H-5), 8.15 (1H, d, J=7.8 Hz, Py-H-3), 7.78 (1H, dt, J=7.8, 1.6 Hz, Py-H-4), 7.25 (1H, m, Py-H-5), 5.94 (1H, d, J=8.8 Hz, H-1′), 5.51 (1H, pt, J=9.5, 9.5 Hz, H-2′), 5.46 (1H, pt, J=9.5, 9.5 Hz, H-3′), 5.28 (1H, pt, J=9.5, 9.5 Hz, H-4′), 4.32 (1H, dd, J=12.6, 4.8 Hz, H-6′a), 4.17 (1H, dd, J=12.6, 1.7 Hz, H-6′b), 4.04 (1H, ddd, J=9.5, 4.8, 1.7 Hz, H-5′), 2.10, 2.08, 2.05, 1.90 (4×3H, 4 s, 4×CH₃); ¹³C NMR (90 MHz, CDCl₃) δ (ppm): 170.6, 170.1, 169.4, 168.9 (4×C═O), 149.8, 149.2 (Tria-C-4, Py-C-2), 149.7 (Py-C-6), 137.0 (Py-C-4), 123.3 (Py-C-5), 120.5 (Py-C-3), 120.7 (Tria-C-5), 86.0 (C-1′), 75.3 (C-5′), 72.8 (C-3′), 70.7 (C-2′), 67.8 (C-4′), 61.7 (C-6′), 20.8, 20.7 (2), 20.3 (4×CH₃). ESI-HRMS positive mode (m/z): calcd for C₂₁H₂₅N₄O₉⁺ [M+H]⁺ 477.1616; C₂₁H₂₄N₄NaO₉⁺ [M+Na]⁺ 499.1435. Found: [M+H]⁺ 477.1614; [M+Na]⁺ 499.1432.

### 11.2 1-(2′,3′,4′,6′-Tetra-O-acetyl-β-D-glucopyranosyl)-4-(quinolin-2-yl)-1,2,3-triazole (L-1b) (Kraft et al., 2015)

Prepared from azide (Györgydeák and Thiem, 2006; Paulsen et al., 1974) 1 (1.00 g, 2.68 mmol) and 2-ethynylquinoline (Son et al., 2013) (0.41 g, 2.68 mmol) according to general procedure I. Purified by column chromatography (1:1 EtOAc-hexane) to yield 1.20 g white amorphous solid (85%). R_{f}=0.23 (1:1 EtOAc-hexane); [α]_{D}=−90 (c 0.20, CHCl₃). ¹H NMR (360 MHz, CDCl₃) δ (ppm): 8.63 (1H, s, Tria-H-5), 8.31 (1H, d, J=8.6 Hz, Qu-H-3), 8.24 (1H, d, J=8.6 Hz, Qu-H-4), 8.08 (1H, d, J=8.5 Hz, Qu-H-5 or Qu-H-8), 7.82 (1H, d, J=8.0 Hz, Qu-H-5 or Qu-H-8), 7.72 (1H, pt, J=7.9, 7.6 Hz, Qu-H-6 or Qu-H-7), 7.53 (1H, pt, J=7.5, 7.4 Hz, Qu-H-6 or Qu-H-7), 5.99 (1H, d, J=9.2 Hz, H-1′), 5.59 (1H, pt, J=9.4, 9.2 Hz, H-2′), 5.48 (1H, pt, J=9.5, 9.4 Hz, H-3′), 5.31 (1H, pt, J=9.7, 9.5 Hz, H-4′), 4.35 (1H, dd, J=12.6, 4.8 Hz, H-6′a), 4.18 (1H, dd, J=12.6, 2.1 Hz, H-6′b), 4.07 (1H, ddd, J=9.7, 4.8, 2.1 Hz, H-5′), 2.11, 2.09, 2.05, 1.91 (4×3H, 4 s, 4×CH₃); ¹³C NMR (90 MHz, CDCl₃) δ (ppm): 170.6, 170.1, 169.4, 169.0 (4×C═O), 149.8, 149.4, 148.2 (Tria-C-4, Qu-C-2, Qu-C-8a), 137.0 (Qu-C-4), 129.9 (Qu-C-6 or Qu-C-7), 129.3 (Qu-C-5 or Qu-C-8), 128.0 (Qu-C-4a), 127.8 (Qu-C-5 or Qu-C-8), 126.6 (Qu-C-6 or Qu-C-7), 121.4 (Tria-C-5), 118.7 (Qu-C-3), 86.0 (C-1′), 75.3 (C-5′), 72.9 (C-3′), 70.7 (C-2′), 67.8 (C-4′), 61.7 (C-6′), 20.8, 20.6 (2), 20.3 (4×CH₃). ESI-HRMS positive mode (m/z): calcd for C₂₅H₂₇N₄O₉⁺ [M+H]⁺ 527.1773; C₂₅H₂₆N₄NaO₉⁺ [M+Na]⁺ 549.1592. Found: [M+H]⁺ 527.1773; [M+Na]⁺549.1593.

### 11.3 1-(2′,3′,4′,6′-Tetra-O-benzoyl-β-D-glucopyranosyl)-4-(pyridin-2-yl)-1,2,3-triazole (L-2a)

The 1-(β-D-glucopyranosyl)-4-(pyridin-2-yl)-1,2,3-triazole (L-3a, 20.0 mg, 0.065 mmol) was suspended in dry pyridine (0.5 mL) and benzoyl chloride (36 μL, 0.310 mmol) was added. The reaction mixture was stirred at 60° C. until the TLC (3:2 EtOAc-hexane) showed complete disappearance of the starting material (1 h). The solvent was removed under diminished pressure. The residue was dissolved in CH₂Cl₂ (20 mL) and extracted with sat. aq. NaHCO₃ (10 mL) and then with water (10 mL). The organic phase was dried over MgSO₄, filtered and evaporated. Purification by column chromatography (3:2 EtOAc-hexane) yielded 41 mg of white amorphous solid (87%). R_{f}=0.30 (3:2 EtOAc-hexane); [α]_{D}=−75 (c 0.20, CHCl₃). ¹H NMR (400 MHz, CDCl₃) δ (ppm): 8.61 (2H, 1 signal, Tria-H-5, Py-H-6), 8.17-7.21 (23H, m, Ar, Py-H-3-Py-H-5), 6.34 (1H, d, J=9.2 Hz, H-1′), 6.16 (1H, pt, J=9.6, 9.5 Hz, H-3′) 6.07 (1H, pt, J=9.5, 9.2 Hz, H-2′), 5.90 (1H, pt, J=9.6, 9.6 Hz, H-4′), 4.69-4.49 (3H, m, H-5′, H-6′a, H-6′b); ¹³C NMR (100 MHz, CDCl₃) δ (ppm): 166.2, 165.8, 165.2, 164.7 (4×C═O), 149.7, 149.0 (Tria-C-4, Py-C-2), 149.6 (Py-C-6), 137.1 (Py-C-4), 133.8, 133.7, 133.6, 133.5, 133.4, 130.2-128.2 (Ar), 123.3 (Py-C-5), 121.0 (Tria-C-5), 120.6 (Py-C-3), 86.4 (C-1′), 75.7 (C-5′), 73.2 (C-3′), 71.3 (C-2′), 69.0 (C-4′), 62.8 (C-6′). ESI-HRMS positive mode (m/z): calcd for C₄₁H₃₂N₄NaO₉⁺ [M+Na]⁺ 747.2061. Found: 747.2041.

### 11.4 1-(β-D-Glucopyranosyl)-4-(pyridin-2-yl)-1,2,3-triazole (L-3a) (Shen et al., 2010)

Prepared from compound L-1a (0.81 g, 1.70 mmol) according to general procedure III. Purified by column chromatography (7:2 CHCl₃-MeOH) to give 0.40 g white amorphous solid (76%). R_{f}=0.26 (7:2 CHCl₃-MeOH); [α]_{D}=−12 (c 0.20, MeOH). ¹H NMR (400 MHz, CD₃OD) δ (ppm): 8.63 (1H, s, Tria-H-5), 8.59 (1H, d, J=4.3 Hz, Py-H-6), 8.09 (1H, d, J=7.9 Hz, Py-H-3), 7.92 (1H, pt, J=7.9, 7.8 Hz, Py-H-4), 7.38 (1H, m, Py-H-5), 5.70 (1H, d, J=9.2 Hz, H-1′), 3.96 (1H, pt, J=9.1, 9.0 Hz, H-2′), 3.90 (1H, dd, J=12.2, 1.3 Hz, H-6′a), 3.74 (1H, dd, J=12.2, 5.3 Hz, H-6′b), 3.64-3.58 (2H, m, H-3′ or H-4′, H-5′), 3.54 (1H, pt, J=9.2, 9.1 Hz, H-3′ or H-4′); ¹³C N M R (90 MHz, CD₃OD) δ (ppm): 150.9, 148.6 (Tria-C-4, Py-C-2), 150.5 (Py-C-6), 138.9 (Py-C-4), 124.6 (Py-C-5), 123.4 (Tria-C-5), 121.7 (Py-C-3), 89.8 (C-1′), 81.2 (C-5′), 78.5 (C-3′ or C-4′), 74.1 (C-2′), 70.9 (C-3′ or C-4′), 62.4 (C-6′). ESI-HRMS positive mode (m/z): calcd for C₁₃H₁₆N₄NaO₅⁺ [M+Na]⁺ 331.1013; C₂₆H₃₂N₈NaO₁₀⁺ [2M+Na]⁺ 639.2134. Found: [M+Na]⁺ 331.1012; [2M+Na]⁺639.2135.

### 11.5 1-(β-D-Glucopyranosyl)-4-(quinolin-2-yl)-1,2,3-triazole (L-3b)

Prepared from compound L-1b (500 mg, 0.95 mmol) according to general procedure III. The crude product was recrystallised from MeOH to yield 290 mg of white amorphous solid (85%). R_{f}=0.44 (7:2 CHCl₃-MeOH); [α]_{D}=−4 (c 0.20, DMSO). ¹H NMR (400 MHz, DMSO-d₆+1-2 drops of D₂O) δ (ppm): 8.99 (1H, s, Tria-H-5), 8.51 (1H, d, J=8.6 Hz, Qu-H-4), 8.25 (1H, d, J=8.6 Hz, Qu-H-3), 8.06-8.01 (2H, m, Qu-H-5, Qu-H-8), 7.82 (1H, pt, J=7.8, 7.4 Hz, Qu-H-6 or Qu-H-7), 7.63 (1H, pt, J=7.6, 7.4 Hz, Qu-H-6 or Qu-H-7), 5.68 (1H, d, J=9.2 Hz, H-1′), 3.90 (1H, pt, J=9.2, 9.1 Hz, H-2′), 3.77-3.72 (1H, m, H-6′a), 3.56-3.49 (2H, m, H-5′, H-6′b), 3.46 (1H, pt, J=9.1, 9.0 Hz, H-3′), 3.34 (1H, pt, J=9.2, 9.0 Hz, H-4′); ¹³C NMR (90 MHz, DMSO-d₆) δ (ppm): 150.1, 147.5, 147.3 (Tria-C-4, Qu-C-2, Qu-C-8a), 137.3 (Qu-C-4), 130.1 (Qu-C-6 or Qu-C-7), 128.6, 128.1 (Qu-C-5, Qu-C-8), 127.3 (Qu-C-4a), 126.5 (Qu-C-6 or Qu-C-7) 123.3 (Tria-C-5), 118.3 (Qu-C-3), 87.8 (C-1′), 80.0 (C-5′), 76.8 (C-3′), 72.2 (C-2′), 69.5 (C-4′), 60.8 (C-6′). ESI-HRMS positive mode (m/z): calcd for C₁₇H₁₉N₄O₅⁺ [M+H]⁺ 359.1350; C₁₇H₁₈N₄NaO₅⁺ [M+Na]⁺ 381.1169; C₃₄H₃₆N₈NaO₁₀⁺ [2M+Na]⁺739.2447. Found: [M+H]⁺ 359.1349; [M+Na]⁺ 381.1168; [2M+Na]⁺ 739.2448.

### 11.6 2-(2′,3′,4′,6′-Tetra-O-benzoyl-β-D-glucopyranosyl)-5-(pyridin-2-yl)-1,3,4-oxadiazole (L-4)

Prepared from tetrazole (Hadady et al., 2004; Kun et al., 2011) 2 (5.00 g, 7.71 mmol) and 2-picolinic acid (1.90 g, 14.43 mmol) according to general procedure II. Reaction time: 5 hours. Purification by column chromatography (1:1 EtOAc-hexane) and crystallisation from EtOH gave 1.96 g white solid (35%). R_{f}=0.28 (1:1 EtOAc-hexane). [α]_{D}=−92 (c 0.20, CHCl₃). ¹H NMR (400 MHz, CDCl₃) δ (ppm): 8.81 (1H, d, J=4.8 Hz, Py-H-6), 8.21 (1H, d, J=7.8 Hz, Py-H-3), 8.03-7.81, 7.56-7.27 (22H, m, Ar, Py-H-4, Py-H-5), 6.10 (1H, pt, J=9.4, 9.4 Hz, H-3′), 6.05 (1H, pt, J=9.4, 9.4 Hz, H-2′), 5.86 (1H, pt, J=9.4, 9.4 Hz, H-4′), 5.28 (1H, d, J=9.4 Hz, H-1′), 4.67 (1H, dd, J=12.4, 2.3 Hz, H-6′a), 4.54 (1H, dd, J=12.4, 5.5 Hz, H-6′b), 4.37 (1H, ddd, J=9.4, 5.5, 2.3 Hz, H-5′); ¹³C NMR (90 MHz, CDCl₃) δ (ppm): 166.2, 165.8, 165.2, 165.1, 164.9, 162.2 (4×C═O, OD-C-2, OD-C-5), 150.5 (Py-C-6), 143.2 (Py-C-2), 137.3 (Py-C-4), 133.7, 133.6, 133.5, 133.2, 130.0-128.4 (Ar), 126.2 (Py-C-5), 123.5 (Py-C-3), 77.3 (C-5′), 73.8 (C-3′), 72.1 (C-1′), 70.6 (C-2′), 69.2 (C-4′), 63.3 (C-6′). ESI-HRMS positive mode (m/z): calcd for C₄₁H₃₁N₃NaO₁₀⁺ [M+Na]⁺748.1902. Found: 748.1907.

### 11.7. 2-(2′,3′,4′,6′-Tetra-O-acetyl-β-D-glucopyranosyl)-5-(pyridin-2-yl)-1,3,4-oxadiazole (L-5)

To a solution of the 2-(β-D-glucopyranosyl)-5-(pyridin-2-yl)-1,3,4-oxadiazole (L-10, 20 mg, 0.065 mmol) in dry pyridine (0.5 mL) acetic anhydride (0.06 mL, 0.635 mmol) was added and the mixture was stirred at 60° C. After one hour the TLC (1:1 EtOAc-hexane) showed total consumption of L-10. The solvent was removed under reduced pressure and the residue was purified by column chromatography (1:1 EtOAc-hexane). White amorphous solid, yield: 28 mg (90%). R_{f}=0.21 (1:1 EtOAc-hexane); [α]_{D}=−61 (c 0.19, CHCl₃). ¹H NMR (400 MHz, CDCl₃) δ (ppm): 8.82 (1H, d, J=4.4 Hz, Py-H-6), 8.26 (1H, d, J=7.9 Hz, Py-H-3), 7.91 (1H, dt, J=7.9, 1.1 Hz, Py-H-4), 7.49 (1H, m, Py-H-5), 5.56 (1H, pt, J=9.8, 9.7 Hz, H-2′), 5.40 (1H, pt, J=9.4, 9.3 Hz, H-3′), 5.24 (1H, pt, J=9.8, 9.7 Hz, H-4′), 4.92 (1H, d, J=10.1 Hz, H-1′), 4.30 (1H, dd, J=12.6, 5.1 Hz, H-6′a), 4.18 (1H, dd, J=12.6, 2.2 Hz, H-6′b), 3.91 (1H, ddd, J=9.7, 5.1, 2.2 Hz, H-5′), 2.09, 2.07, 2.04, 1.94 (4×3H, 4 s, 4×CH₃); ¹³C NMR (100 MHz, CDCl₃) δ (ppm): 170.7, 170.3, 169.4, 169.3 (4×C═O), 165.1, 162.1 (OD-C-2, OD-C-5), 150.6 (Py-C-6), 143.2 (Py-C-2), 137.4 (Py-C-4), 126.3 (Py-C-5), 123.6 (Py-C-3), 76.9 (C-5′), 73.5 (C-3′), 71.6 (C-1′), 69.7 (C-2′), 68.0 (C-4′), 62.0 (C-6′), 20.8, 20.7 (2), 20.5 (4×CH₃). ESI-HRMS positive mode (m/z): calcd for C₂₁H₂₃N₃NaO₁₀⁺ [M+Na]⁺ 500.1276. Found: 500.1275.

### 11.8 2-(2′,3′,4′-Tri-O-benzoyl-β-D-xylopyranosyl)-5-(pyridin-2-yl)-1,3,4-oxadiazole (L-6)

Prepared from tetrazole (Somsak et al., 2014) 3 (3.00 g, 5.83 mmol) and 2-picolinic acid (1.42 g, 11.53 mmol) according to general procedure II. Reaction time: 5 hours. Purification by column chromatography (2:1 EtOAc-hexane) and crystallisation from EtOH yielded 2.00 g white solid (58%). R_{f}=0.35 (4:1 EtOAc-hexane); [α]_{D}=−123 (c 0.20, CHCl₃). ¹H NMR (360 MHz, CDCl₃) δ (ppm): 8.80 (1H, d, J=4.8 Hz, Py-H-6), 8.21 (1H, d, 7.9 Hz, Py-H-3), 7.99-7.83, 7.57-7.30 (17H, m, Ar, Py-H-4, Py-H-5), 6.05 (1H, pt, J=9.2, 9.2 Hz, H-3′), 5.96 (1H, pt, J=9.2, 9.2 Hz, H-2′), 5.57 (1H, ddd, J=10.1, 9.2, 5.3 Hz, H-4′), 5.15 (1H, d, J=9.2 Hz, H-1′), 4.62 (1H, dd, J=11.4, 5.3 Hz, H-5′eq), 3.80 (1H, pt, J=11.4, 10.1 Hz, H-5′ax); ¹³C NMR (90 MHz, CDCl₃) δ (ppm): 165.8, 165.6, 165.1 (2), 162.5 (3×C═O, OD-C-2, OD-C-5), 150.6 (Py-C-6), 143.3 (Py-C-2), 137.3 (Py-C-4), 133.7, 133.6, 133.5, 130.0-128.5 (Ar), 126.2 (Py-C-5), 123.5 (Py-C-3), 72.9 (C-3′), 72.5 (C-1′), 70.4 (C-2′), 69.6 (C-4′), 67.5 (C-5′). ESI-HRMS positive mode (m/z): calcd for C₃₃H₂₅N₃NaO₈⁺ [M+Na]⁺ 614.1534. Found: 614.1535.

### 11.9 2-(2′,3′,4′,6′-Tetra-O-acetyl-β-D-galactopyranosyl)-5-(pyridin-2-yl)-1,3,4-oxadiazole (L-7)

Prepared from tetrazole (Farkas et al., 1977) 4 (0.50 g, 1.25 mmol) and 2-picolinic acid (0.31 g, 2.50 mmol) according to general procedure II. Reaction time: 2 hours. Purification by column chromatography (1:1 EtOAc-hexane) and crystallisation from EtOH yielded 0.30 g white solid (50%). R_{f}=0.17 (1:1 EtOAc-hexane); [α]_{D}=−41 (c 0.21, CHCl₃). ¹H NMR (360 MHz, CDCl₃) δ (ppm): 8.82 (1H, ddd, J=4.8, 1.8, 0.9 Hz, Py-H-6), 8.27 (1H, dd, J=7.9, 0.9 Hz, Py-H-3), 7.91 (1H, dt, J=7.9, 1.8 Hz, Py-H-4), 7.50 (1H, ddd, J=7.7, 4.8, 1.1 Hz, Py-H-5), 5.67 (1H, pt, J=10.1, 10.0 Hz, H-2′), 5.56 (1H, d, J=3.4 Hz, H-4′), 5.24 (1H, dd, J=10.1, 3.4 Hz, H-3′), 4.89 (1H, d, J=10.0 Hz, H-1′), 4.23-4.11 (3H, m, H-5′, H-6′a, H-6′b), 2.23, 2.06, 2.02, 1.96 (4×3H, 4 s, 4×CH₃); ¹³C NMR (90 MHz, CDCl₃) δ (ppm): 170.4, 170.3, 170.0, 169.4, 165.0, 162.3 (4×C═O, OD-C-2, OD-C-5), 150.5 (Py-C-6), 143.2 (Py-C-2), 137.3 (Py-C-4), 126.2 (Py-C-5), 123.6 (Py-C-3), 75.6 (C-5′), 72.2 (C-1′), 71.4 (C-3′), 67.3 (C-4′), 66.9 (C-2′), 61.6 (C-6′), 20.8, 20.7, 20.6, 20.5 (4×CH₃). ESI-HRMS positive mode (m/z): calcd for C₂₁H₂₄N₃O₁₀⁺ [M+H]⁺ 478.1456; C₂₁H₂₃N₃NaO₁₀⁺ [M+Na]⁺500.1276; C₄₂H₄₆N₆NaO₂₀⁺ [2M+Na]⁺ 977.2659. Found: [M+H]⁺ 478.1454; [M+Na]⁺ 500.1274; [2M+Na]⁺ 977.2653.

### 11.10 2-(2′,3′,4′,6′-Tetra-O-benzoyl-β-D-galactopyranosyl)-5-(pyridin-2-yl)-1,3,4-oxadiazole (L-8)

The 2-(β-D-galactopyranosyl)-5-(pyridin-2-yl)-1,3,4-oxadiazole (L-10, 20.0 mg, 0.065 mmol) was suspended in dry pyridine (0.5 mL) and benzoyl chloride (36 μL, 0.310 mmol) was added. The reaction mixture was stirred at 60° C. until the TLC (3:2 EtOAc-hexane) showed complete disappearance of the starting material (1 h). The solvent was removed under diminished pressure. The residue was dissolved in CH₂Cl₂ (20 mL) and extracted with sat. aq. NaHCO₃ (10 mL) and then with water (10 mL). The organic phase was dried over MgSO₄, filtered and evaporated. Purification by column chromatography (3:2 EtOAc-hexane) yielded 38 mg of white amorphous solid (81%). R_{f}=0.27 (3:2 EtOAc-hexane); [α]_{D}=+9 (c 0.20, CHCl₃). ¹H NMR (400 MHz, CDCl₃) δ (ppm): 8.81 (1H, d, J=4.8 Hz, Py-H-6), 8.21 (1H, d, J=7.9 Hz, Py-H-3), 8.17-7.23 (22H, m, Ar, Py-H-4, Py-H-5), 6.32 (1H, pt, J=10.1, 10.0 Hz, H-2′), 6.16 (1H, d, J=3.3 Hz, H-4′), 5.84 (1H, dd, J=10.1, 3.3 Hz, H-3′), 5.30 (1H, d, J=10.0 Hz, H-1′), 4.68 (1H, dd, J=11.1, 6.4 Hz, H-6′a), 4.58 (1H, pt, J=6.4, 5.9 Hz, H-5′), 4.49 (1H, dd, J=11.1, 5.9 Hz, H-6′b); ¹³C NMR (100 MHz, CDCl₃) δ (ppm): 166.2, 165.7, 165.6, 165.1, 165.0, 162.4 (4×C═O, OD-C-2, OD-C-5), 150.5 (Py-C-6), 143.3 (Py-C-2), 137.3 (Py-C-4), 133.8, 133.6, 133.5, 133.4, 130.2-130.0, 129.9-128.5 (Ar), 126.2 (Py-C-5), 123.6 (Py-C-3), 76.1 (C-5′), 72.5, 72.3 (C-1′, C-3′), 68.4 (C-4′), 67.9 (C-2′), 62.3 (C-6′). ESI-HRMS positive mode (m/z): calcd for C₄₁H₃₁N₃NaO₁₀⁺ [M+Na]⁺ 748.1902. Found: 748.1901.

### 11.11 2-(L-Arabino-1′,2′,3′,4′-tetraacetoxybutyl)-5-(pyridin-2-yl)-1,3,4-oxadiazole (L-9)

Prepared from tetrazole (Viana et al., 2008) 5 (3.50 g, 9.77 mmol) and 2-picolinic acid (2.41 g, 19.58 mmol) according to general procedure II. Reaction time: 5 hours. Purification by column chromatography (1:1→2:1→3:1 EtOAc-hexane) yielded 0.57 g of white amorphous solid (13%). R_{f}=0.27 (4:1 EtOAc-hexane); [α]_{D}=+6 (c 0.20, CHCl₃). ¹H NMR (400 MHz, CDCl₃) δ (ppm): 8.79 (1H, d, J=4.4 Hz, Py-H-6), 8.26 (1H, d, J=7.9 Hz, Py-H-3), 7.90 (1H, dt, J=7.8, 1.4 Hz, Py-H-4), 7.49 (1H, m, Py-H-5), 6.35 (1H, d, J=2.2 Hz, H-1′), 5.69 (1H, dd, J=9.4, 2.2 Hz, H-2′), 5.36 (1H, ddd, J=9.4, 3.9, 2.3 Hz, H-3′), 4.35 (1H, dd, J=12.6, 2.3 Hz, H-4′a), 4.23 (1H, dd, J=12.6, 3.9 Hz, H-4′b), 2.22, 2.11, 2.10, 2.06 (4×3H, 4 s, CH₃); ¹³C NMR (100 MHz, CDCl₃) δ (ppm): 170.6, 169.7, 169.6 (2) (4×C═O), 164.5, 162.6 (OD-C-2, OD-C-5), 150.4 (Py-C-6), 143.1 (Py-C-2), 137.4 (Py-C-4), 126.3 (Py-C-5), 123.5 (Py-C-3), 68.7 (C-2′), 67.8 (C-3′), 64.4 (C-1′), 61.5 (C-4′), 20.8, 20.7, 20.5 (2) (4×CH₃). ESI-HRMS positive mode (m/z): calcd for C₁₉H₂₂N₃O₉ [M+H]⁺ 436.1351; C₁₉H₂₁N₃NaO₉⁺ [M+Na]⁺ 458.1170. Found: [M+H]⁺ 436.1349; [M+Na]⁺ 458.1170.

### 11.12 2-(β-D-Glucopyranosyl)-5-(pyridin-2-yl)-1,3,4-oxadiazole (L-10)

Prepared from compound L-4 (0.60 g, 0.83 mmol) according to general procedure III. Purified by column chromatography (9:1 CHCl₃: MeOH) to give 0.22 g white amorphous solid (87%). R_{f}=0.29 (8:2 CHCl₃-MeOH); [α]_{D}=+17 (c 0.20, MeOH). ¹H NMR (360 MHz, CD₃OD) δ (ppm): 8.75 (1H, ddd, J=4.9, 1.7, 0.9 Hz, Py-H-6), 8.25 (1H, dd, J=7.9, 1.1 Hz, Py-H-3), 8.07 (1H, dt, J=7.9, 1.7 Hz, Py-H-4), 7.64 (1H, ddd, J=7.9, 4.9, 1.1 Hz, Py-H-5), 4.68 (1H, d, J=9.9 Hz, H-1′), 3.90 (1H, dd, J=12.2, 1.9 Hz, H-6′a), 3.84 (1H, dd, J=9.9, 8.7 Hz, H-2′), 3.70 (1H, J=12.2, 5.4 Hz, H-6′b), 3.54 (1H, pt, J=8.9, 8.6 Hz, H-3′), 3.54-3.50 (1H, m, H-5′), 3.46 (1H, pt, J=9.3, 8.8 Hz, H-4′); ¹³C NMR (90 MHz, CD₃OD) δ (ppm): 166.4, 165.7 (OD-C-2, OD-C-5), 151.4 (Py-C-6), 144.0 (Py-C.2), 139.3 (Py-C-4), 127.9 (Py-C-5), 124.5 (Py-C-3), 82.9 (C-5′), 79.1 (C-3′), 74.7 (C-1′), 73.4 (C-2′), 71.3 (C-4′), 62.8 (C-6′). ESI-HRMS positive mode (m/z): calcd for C₁₃H₁₅N₃NaO₆⁺ [M+Na]⁺ 332.0853. Found: 332.0844.

### 11.13 2-(β-D-Xylopyranosyl)-5-(pyridin-2-yl)-1,3,4-oxadiazole (L-11)

Prepared from compound L-6 (500 mg, 0.85 mmol) according to general procedure III. Purified by column chromatography (9:1 CHCl₃-MeOH) to give 82 mg white amorphous solid (35%). R_{f}=0.27 (9:1 CHCl₃-MeOH); [α]_{D}=−44 (c 0.20, MeOH). ¹H NMR (360 MHz, CD₃OD) δ (ppm): 8.74 (1H, ddd, J=4.9, 1.6, 0.9 Hz, Py-H-6), 8.24 (1H, ddd, J=7.9, 1.1, 0.9 Hz, Py-H-3), 8.06 (1H, dt, J=7.9, 1.6 Hz, Py-H-4), 7.63 (1H, ddd, J=7.7, 4.9, 1.1 Hz, Py-H-5), 4.59 (1H, d, J=9.8 Hz, H-1′), 4.03 (1H, dd, J=11.1, 5.4 Hz, H-5′eq), 3.84 (1H, pt, J=9.8, 9.1 Hz, H-2′), 3.66 (1H, td, J=10.1, 9.1, 5.4 Hz, H-4′), 3.47 (1H, pt, J=9.1, 9.1 Hz, H-3′), 3.41 (1H, pt, J=11.1, 10.1 Hz, H-5′ax); ¹³C NMR (90 MHz, CD₃OD) δ (ppm): 166.4, 165.7 (OD-C-2, OD-C-5), 151.4 (Py-C-6), 144.0 (Py-C-2), 139.3 (Py-C-4), 127.9 (Py-C-5), 124.5 (Py-C-3), 79.2 (C-3′), 75.4 (C-1′), 73.4 (C-2′), 71.7 (C-5′), 71.0 (C-4′). ESI-HRMS positive mode (m/z): calcd for C₁₂H₁₃N₃NaO₅⁺ [M+Na]⁺ 302.0747; C₂₄H₂₆N₆NaO₁₀⁺ [2M+Na]⁺ 581.1603. Found: [M+Na]⁺ 302.0747; [2M+Na]⁺ 581.1604.

### 11.14 2-(β-D-Galactopyranosyl)-5-(pyridin-2-yl)-1,3,4-oxadiazole (L-12)

Prepared from compound L-7 (350 mg, 0.73 mmol) according to general procedure III. Purified by column chromatography (7:2 CHCl₃-MeOH) to give 177 mg white amorphous solid (78%). R_{f}=0.33 (7:2 CHCl₃-MeOH); [α]_{D}=+25 (c 0.21, MeOH). ¹H NMR (400 MHz, CD₃OD) δ (ppm): 8.77 (1H, d, J=4.8 Hz, Py-H-6), 8.27 (1H, d, J=7.9 Hz, Py-H-3), 8.08 (1H, dt, J=7.9, 1.5 Hz, Py-H-4), 7.65 (1H, m, Py-H-5), 4.63 (1H, d, J=9.8 Hz, H-1′), 4.22 (1H, pt, J=9.8, 9.6 Hz, H-2′), 4.01 (1H, d, J=3.2 Hz, H-4′), 3.84-3.73 (3H, m, H-5′, H-6′a, H-6′b), 3.67 (1H, dd, J=9.6, 3.2 Hz, H-3′); ¹³C NMR (90 MHz, CD₃OD) δ (ppm): 166.5, 165.7 (OD-C-2, OD-C-5), 151.4 (Py-C-6), 144.1 (Py-C-2), 139.3 (Py-C-4), 127.9 (Py-C-5), 124.5 (Py-C-3), 81.7 (C-5′), 75.8 (C-3′), 75.1 (C-1′), 70.7 (C-4′), 70.2 (C-2′), 62.8 (C-6′). ESI-HRMS positive mode (m/z): calcd for C₁₃H₁₆N₃O₆⁺ [M+H]⁺ 310.1034; C₁₃H₁₅N₃NaO₆⁺ [M+Na]⁺ 332.0853; C₂₆H₃₀N₆NaO₁₂⁺ [2M+Na]⁺ 641.1814. Found: [M+H]⁺ 310.1035; [M+Na]⁺ 332.0852; [2M+Na]⁺ 641.1815.

### 11.15 Complex Ru-1a

Prepared from complex Ru-dimer (50 mg, 0.082 mmol), compound L-1a (86 mg, 0.181 mmol, 2.2 eq.) and TIPF₆ (57 mg, 0.163 mmol) according to general procedure IV. Purified by column chromatography (9:1 CHCl₃-MeOH) to give 143 mg (98%) yellow powder. R_{f}: 0.32 (9:1 CHCl₃-MeOH). Diastereomeric ratio: 1:1. ¹H NMR (400 MHz, CDCl₃) δ (ppm): 9.25, 9.22 (2×1H, 2 d, J=5.5 Hz in each, 2×Py-H-6), 8.92, 8.79 (2×1H, 2 s, 2×Tria-H-5), 7.96 (2H, t, J=7.6 Hz, 2×Py-H-4), 7.89 (2H, d, J=7.8 Hz, 2×Py-H-3), 7.57-7.52 (2H, m, 2×Py-H-5), 6.00, 5.99 (2×1H, 2 d, J=9.4 Hz in each, 2×H-1′), 5.95-5.83, 5.73-5.67 (10H, m, 2×4×p-cym-CH_{Ar}, 2×H-2′), 5.47, 5.46 (2×1H, 2 pt, J=9.4, 9.2 Hz in each, 2×H-3′), 5.35, 5.33 (2×1H, 2 pt, J=9.7, 9.7 Hz in each, 2×H-4′), 4.38, 4.32 (2×1H, 2 dd, J=12.8, 4.7 Hz in each, 2×H-6′a), 4.26-4.15 (4H, m, 2×H-5′, 2×H-6′b), 2.79, 2.74 (2×1H, 2 hept, J=6.9 Hz in each, 2×i-Pr—CH), 2.22, 2.20 (2×3H, 2 s, 2×C₆H₄—CH₃), 2.10, 2.09, 2.05, 1.95, 1.93 (24H, singlets, 2×4×COCH₃), 1.17-1.12 (12H, m, 2×2×i-Pr—CH₃); ¹³C NMR (90 MHz, CDCl₃) δ (ppm): 170.8, 170.7, 170.0, 169.9, 169.6, 169.5, 169.4, 169.2 (2×4×C═O), 155.5, 155.4 (2×Py-C-6), 147.6, 147.5, 147.1, 146.7 (2×Tria-C-4, 2×Py-C-2), 140.3, 140.2 (2×Py-C-4), 127.1, 126.9 (2×Py-C-5), 125.4, 125.3 (2×Tria-C-5), 122.9 (2) (2×Py-C-3), 106.3, 105.5, 103.1, 101.8 (2×2×p-cym-C_{qAr}), 86.8, 86.7 (2×C-1′), 86.4, 85.4, 85.3, 85.0, 84.8, 83.9, 83.9, 83.1 (2×4×p-cym-CH_{Ar}), 75.5, 75.3 (2×C-5′), 73.1, 73.0 (2×C-3′), 70.2, 69.8 (2×C-2′), 67.6, 67.5 (2×C-4′), 61.6, 61.5 (2×C-6′), 31.1, 31.0 (2×i- Pr—CH), 22.5, 22.2, 22.1, 21.7 (2×2×i-Pr—CH₃), 20.8-20.3 (2×4×COCH₃), 18.7 (2) (2×C₆H₄—CH₃). ESI-HRMS positive mode (m/z): calcd for C₃₁H₃₈ClN₄O₉Ru⁺ [M−PF₆]⁺ 747.1371. Found: 747.1370.

### 11.16 Complex Ru-1b

Prepared from complex Ru-dimer (50 mg, 0.082 mmol), compound L-1b (95 mg, 0.180 mmol, 2.2 eq.) and TIPF₆ (57 mg, 0.163 mmol) according to general procedure IV. Purified by column chromatography (9:1 CHCl₃-MeOH) to give 145 mg (96%) yellow powder. R_{f}: 0.55 (9:1 CHCl₃-MeOH). Diastereomeric ratio: 2:1. ¹H NMR (400 MHz, CDCl₃) δ (ppm): 9.24 (s, minor Tria-H-5), 9.03 (s, major Tria-H-5), 8.75 (d, J=8.8 Hz, major Qu-H-8), 8.68 (d, J=8.7 Hz, minor Qu-H-8), 8.39 (d, J=8.6 Hz, major Qu-H-4), 8.37 (d, J=8.9 Hz, minor Qu-H-4), 8.04-7.89, 7.75-7.70 (2 m, minor and major Qu-H-3, Qu-H-5-Qu-H-7), 6.11 (d, J=9.4 Hz, minor H-1′), 6.08 (d, J=9.2 Hz, major H-1′), 6.03 (pt, J=9.2, 9.0 Hz, major H-2′), 5.97 (pt, J=9.4, 9.0 Hz, minor H-2′), 5.96, 5.95 (2 d, J=6.0 Hz in each, major p-cym-CH_{Ar}) 5.88, 5.84 (2 d, J=6.0 Hz in each, minor p-cym-CH_{Ar}) 5.83-5.80 (m, minor and major p-cym-CH_{Ar}), 5.78 (d, J=6.0 Hz, minor p-cym-CH_{Ar}), 5.67 (d, J=5.9 Hz, major p-cym-CH_{Ar}), 5.52 (pt, J=9.2, 9.2 Hz, major H-3′), 5.49 (pt, J=9.5, 9.4 Hz, minor H-3′), 5.39 (pt, J=10.2, 9.7 Hz, minor H-4′), 5.34 (pt, J=10.0, 9.7 Hz, major H-4′), 4.44 (dd, J=12.7, 4.7 Hz, minor H-6′a), 4.33 (dd, J=12.7, 5.1 Hz, major H-6′a), 4.32-4.17 (m, minor and major H-5′, H-6′b), 2.57 (hept, J=6.9 Hz, major i-Pr—CH), 2.53 (hept, J=6.9 Hz, minor i-Pr—CH), 2.15 (s, minor C₆H₄—CH₃), 2.13, 2.09 (singlets, COCH₃), 2.07 (s, major C₆H₄—CH₃), 2.06, 2.02, 1.96 (singlets, COCH₃), 1.07, 1.04 (2 d, J=6.9 Hz in each, 2×major i-Pr—CH₃), 1.02, 1.00 (2 d, J=6.9 Hz in each, 2×minor i-Pr—CH₃); ¹³C NMR (90 MHz, CDCl₃) δ (ppm): 170.9, 170.0, 169.6, 169.4 (minor 4×C═O), 170.8, 170.1, 169.8, 169.7 (major 4×C═O), 149.6, 148.4, 147.5 (major Tria-C-4, Qu-C-2, Qu-C-8a), 149.2, 148.4, 148.9 (minor Tria-C-4, Qu-C-2, Qu-C-8a), 141.3 (minor Qu-C-4), 141.0 (major Qu-C-4), 133.1, 129.6, 129.2, 129.1, 129.1 (minor Qu-C-4a, Qu-C-5-Qu-C-8), 132.7, 129.5, 129.1, 129.1, 129.0 (major Qu-C-4a, Qu-C-5-Qu-C-8), 127.6 (major Tria-C-5), 127.5 (minor Tria-C-5), 119.1 (major Qu-C-3), 118.8 (minor Qu-C-3), 106.3, 102.1 (minor p-cym-C_{qAr}), 105.4, 102.7 (major p-cym-C_{qAr}), 88.1, 86.7, 86.5, 86.4, 85.7, 85.1, 84.6, 84.5, 84.3, 84.0 (minor and major p-cym-CH_{Ar}, minor and major C-1′), 75.6 (minor C-5′), 75.2 (major C-5′), 73.2 (minor C-3′), 73.0 (major C-3′), 70.2 (major C-2′), 69.8 (minor C-2′), 67.5 (major C-4′), 67.7 (minor C-4′), 61.6 (major C-6′), 61.5 (minor C-6′), 31.3 (minor i-Pr—CH), 31.1 (major i-Pr—CH), 22.7, 21.6 (major i-Pr—CH₃), 22.4, 21.8 (minor i-Pr—CH₃), 20.8, 20.7, 20.7, 20.6, 20.4 (2×4×COCH₃), 18.7 (minor C₆H₄—CH₃), 18.5 (major C₆H₄—CH₃). ESI-HRMS positive mode (m/z): calcd for C₃₅H₄₀ClN₄O₉Ru⁺ [M−PF₆]⁺ 797.1528. Found: 797.1531.

### 11.17 Complex Ru-2a

Prepared from complex Ru-dimer (10.0 mg, 0.0163 mmol), compound L-2a (24.9 mg, 0.0344 mmol, 2.1 eq.) and TIPF₆ (11.4 mg, 0.0326 mmol, 2 eq.) according to general procedure IV. Purified by column chromatography (95:5 CHCl₃-MeOH) to give 34.6 mg (93%) orange powder. R_{f}: 0.46 (95:5 CHCl₃-MeOH). Diastereomeric ratio: 1:1. ¹H NMR (400 MHz, CDCl₃) δ (ppm): 9.23, 9.20 (2×1H, 2 d, J=5.6 Hz in each, 2×Py-H-6), 9.02, 8.98 (2×1H, 2 s, 2×Tria-H-5), 8.11-7.75, 7.62-7.24 (46H, m, 2×20×Ar, 2×Py-H-3-Py-H-5), 6.60, 6.10 (2×1H, 2 pt, J=9.4, 9.3 Hz in each, 2×H-2′), 6.50, 6.43 (2×1H, 2 d, J=9.3 Hz in each, 2×H-1′), 6.22, 6.15 (2×1H, 2 pt, J=9.6, 9.4 Hz in each, 2×H-3′), 5.97, 5.94 (2×1H, 2 pt, J=9.7, 9.6 Hz in each, 2×H-4′), 5.81, 5.65 (2), 5.57 (2), 5.53, 5.47, 5.44 (2×4H, 2×4 d, J=6.1 Hz in each, 2×4×p-cym-CH_{Ar}), 4.78-4.52 (2×3H, m, 2×H-5′, 2×H-6′a, 2×H-6′b), 2.50, 2.36 (2×1H, 2 hept, J=6.9 Hz in each, 2×i-Pr—CH), 2.08, 2.00 (2×3H, 2 s, 2×C₆H₄—CH₃), 0.94, 0.91, 0.79, 0.74 (2×2×3H, 2×2 d, J=6.9 Hz in each 2×2×i-Pr—CH₃); ¹³C NMR (100 MHz, CDCl₃) δ (ppm): 166.3, 166.2, 165.6, 165.5, 165.2 (2), 164.9, 164.8 (2×4×C═O), 155.7, 155.5 (2×Py-C-6), 147.3, 147.2, 147.0, 146.6 (2×Tria-C-4, 2×Py-C-2), 140.1, 140.0 (2×Py-C-4), 134.4, 134.2, 133.9, 133.8, 133.7 (2), 133.6, 133.5, 133.3, 130.3-128.0 (Ar), 127.2, 126.9 (2×Py-C-5), 126.2, 123.8 (2×Tria-C-5), 122.7 (2×Py-C-3), 105.8, 105.3, 103.9, 102.6 (2×2×p-cym-C_{qAr}), 87.2, 86.7 (2×C-1′), 86.4, 85.8, 85.2, 85.0, 84.0, 83.9, 83.2, 82.5 (2×4×p-cym-CH_{Ar}), 76.1, 75.6 (2×C-5′), 73.6, 72.7 (2×C-3′), 71.7, 70.4 (2×C-2′), 68.7, 68.6 (2×C-4′), 62.8, 62.7 (2×C-6′), 31.0 (2) (2×i-Pr—CH), 22.5, 22.3, 21.7, 21.4 (2×2×i-Pr—CH₃), 18.8, 18.6 (2×C₆H₄—CH₃). ESI-HRMS positive mode (m/z): calcd for C₅₁H₄₆ClN₄O₉Ru⁺ [M−PF₆]⁺ 995.2002. Found: 995.1994.

### 11.18 Complex Ru-3a

Prepared from complex Ru-dimer (50.0 mg, 0.082 mmol), compound L-3a (50.4 mg, 0.163 mmol, 2 eq.) and TIPF₆ (57.0 mg, 0.163 mmol) according to general procedure V. Yield: 107 mg (90%). An analytically pure sample was obtained by recrystallisation from iPrOH to give 20 mg orange powder. Diastereomeric ratio: 1:1. ¹H NMR (400 MHz, CD₃OD) δ (ppm): 9.42 (2H, d, J=5.4 Hz, 2×Py-H-6), 9.22, 9.21 (2×1H, 2 s, 2×Tria-H-5), 8.19 (2H, pt, J=7.8, 7.7 Hz, 2×Py-H-4), 8.10 (2H, d, J=7.8 Hz, 2×Py-H-3), 7.69-7.66 (2H, m, 2×Py-H-5), 6.12-6.05 (4H, m, 2×p-cym-CH_{Ar}), 5.90-5.84 (4H, m, 2×p-cym-CH_{Ar}), 5.88 (2H, d, 2×H-1′), 3.95-3.89 (4H, m, 2×H-2′, 2×H-6′a), 3.77 (2H, dd, J=12.1, 5.3 Hz, 2×H-6′b), 3.69 (2H, ddd, J=9.5, 5.3, 1.9 Hz, 2×H-5′), 3.64 (2H, pt, J=9.5, 8.9 Hz, 2×H-3′), 3.56 (2H, pt, J=9.3, 9.1 Hz, 2×H-4′), 2.79-2.71 (2H, hept, J=6.7 Hz, 2×i-Pr—CH), 2.22 (6H, s, 2×C₆H₄—CH₃), 1.18-1.09 (12H, m, 2×2×i-Pr—CH₃); ¹³C NMR (100 MHz, CD₃OD) δ (ppm): 156.8 (2) (2×Py-C-6), 149.6 (2), 148.2, 148.1 (2×Py-C-2, 2×Tria-C-4), 141.5 (2) (2×Py-C-4), 127.6 (2) (2×Py-C-5), 125.5, 125.2 (2×Tria-C-5), 123.6 (2) (2×Py-C-3), 106.8, 106.6, 104.1, 103.8 (2×2×p-cym-C_{qAr}), 91.3, 91.2 (2×C-1′), 87.3, 87.2, 86.3, 86.1, 85.6, 85.5, 84.8, 84.6 (2×4×p-cym-CH_{Ar}), 81.7, 81.6 (2×C-5′), 78.2, 78.1 (2×C-3′), 74.5, 74.3 (2×C-2′), 70.8, 70.7 (2×C-4′), 62.3, 62.2 (2×C-6′), 32.3, 32.2 (2×i-Pr—CH), 22.6 (2), 22.0, 21.9 (2×2×i-Pr—CH₃), 18.8, 18.7 (2×C₆H₄—CH₃). ESI-HRMS positive mode (m/z): calcd for C₂₃H₃₀ClN₄O₅Ru⁺ [M−PF₆]⁺ 579.0946. Found: 579.0946.

### 11.19 Complex Ru-3b

Prepared from complex Ru-dimer (50.0 mg, 0.082 mmol), compound L-3b (58.8 mg, 0.164 mmol, 2 eq.) and TIPF₆ (57 mg, 0.163 mmol) according to general procedure V. Yield: 121 mg (96%). An analytically pure sample was obtained by recrystallisation from iPrOH to give 34 mg orange powder. Diastereomeric ratio: 1:1. ¹H NMR (400 MHz, CD₃OD) δ (ppm): 9.42, 9.41 (2×1H, 2 s, 2×Tria-H-5), 8.80 (2H, d, J=8.8 Hz, 2×Qu-H-8), 8.70, 8.68 (2×1H, 2 d, J=8.5 Hz in each, 2×Qu-H-4), 8.15-8.09, 7.88-7.84 (2×4H, m, Qu-H-3, Qu-H-5-Qu-H-7), 6.13-5.96 (2×4H, m, 2×p-cym-CH_{Ar}), 5.95, 5.93 (2×1H, 2 d, J=9.1 Hz in each, 2×H-1′), 4.00, 3.99 (2×1H, 2 pt, J=9.2, 9.1 Hz in each, 2×H-2′), 3.97, 3.95 (2×1H, 2 dd, J=12.1, 2.1 Hz in each, 2×H-6′a), 3.80 (2H, dd, J=12.1, 5.3 Hz, 2×H-6′b), 3.74, 3.71 (2×1H, ddd, J=9.3, 5.3, 2.1 Hz in each, 2×H-5′), 3.68, 3.67 (2×1H, 2 pt, J=9.0, 8.9 Hz in each, 2×H-3′), 3.60, 3.59 (2×1H, 2 pt, J=9.3, 9.2 Hz in each, 2×H-4′), 2.48, 2.46 (2×1H, 2 hept, J=6.9 Hz in each, 2×i-Pr—CH), 2.20 (6H, s, 2×C₆H₄—CH₃), 1.00, 0.98, 0.92 (2) (2×2×3H, 4 d, J=6.9 Hz in each, 2×2×i-Pr—CH₃); ¹³C NMR (90 MHz, CD₃OD) δ (ppm): 151.0 (2), 149.7 (2), 149.1 (2), (2×Tria-C-4, 2×Qu-C-2, 2×Qu-C-8a), 142.6 (2×Qu-C-4), 134.0, 130.7, 130.6 (3), 130.5, 130.1 (2×Qu-C-4a, 2×Qu-C-5-Qu-C-8), 127.1, 126.9 (2×Tria-C-5), 119.5, 119.4 (2×Qu-C-3), 106.5, 106.3, 104.9, 104.7 (2×2×p-cym-C_{qAr}), 91.4, 91.2 (2×C-1′), 88.6, 88.5, 86.5, 86.3, 86.2, 85.8, 84.6, 84.3 (2×4×p-cym-CH_{Ar}), 81.8, 81.7 (2×C-5′), 78.2, 78.1 (2×C-3′), 74.4, 74.3 (2×C-2′), 70.7 (2) (2×C-4′), 62.3, 62.20 (2×C-6′), 32.3, 32.2 (2×i-Pr—CH), 22.7, 22.6, 21.6, 21.5 (2×2×i-Pr—CH₃), 18.8, 18.7 (2×C₆H₄—CH₃). ESI-ESI-HRMS positive mode (m/z): calcd for C₂₇H₃₂ClN₄O₅Ru⁺ [M−PF₆]⁺: 629.1103. Found: 629.1103.

### 11.20 Complex Ru-4

Prepared from complex Ru-dimer (10.0 mg, 0.016 mmol), compound L-4 (24.9 mg, 0.034 mmol, 2.1 eq.) and TIPF₆ (11.4 mg, 0.033 mmol) according to general procedure IV. After filtration and removal of the solvent the residue was dissolved in CHCl₃ (3 mL) and Et₂O (6 mL) was added. The precipitated product was filtered off to give 28.4 mg (76%) yellow powder. The product can also be obtained by column chromatographic purification (9:1 CHCl₃-MeOH), albeit in lower yield (52%). R_{f}: 0.38 (9:1 CHCl₃-MeOH). Diastereomeric ratio: 2:1. ¹H NMR (400 MHz, CDCl₃) δ (ppm): 9.50 (s, minor Py-H-6), 9.26 (s, major Py-H-6), 8.18-7.70, 7.60-7.28 (m, minor and major Ar, Py-H-3-Py-H-5), 6.19 (pt, J=9.8, 9.7 Hz, minor H-3′), 6.16 (pt, J=9.8, 9.5 Hz, major H-2′), 6.08 (pt, J=9.5, 9.5 Hz, major H-3′), 5.88 (pt, J=9.7, 9.5 Hz, major H-4′), 5.86 (pt, J=9.7, 9.6 Hz, minor H-4′), 5.90-5.84 (m, 2×minor p-cym-CH_{Ar}), 5.76-5.72 (m, major p-cym-CH_{Ar}, minor H-2′), 5.68-5.65 (m, 2×minor p-cym-CH_{Ar}), 5.53-5.51 (m, major p-cym-CH_{Ar}), 5.46 (d, J=9.8 Hz, major H-1′), 5.35 (d, J=9.8 Hz, minor H-1′), 5.31-5.29 (m, 2×major p-cym-CH_{Ar}), 4.74-4.68 (m, minor and major H-6′a), 4.57-4.52 (m, minor and major H-6′b), 4.50-4.42 (m, minor and major H-5′), 2.78 (hept, J=6.8 Hz, major i-Pr—CH), 2.72 (hept, J=6.8 Hz, minor i-Pr—CH), 2.02 (s, minor C₆H₄—CH₃), 1.95 (s, major C₆H₄—CH₃), 1.16, 1.15, 1.12, 1.08 (4 d, J=6.8 Hz in each, minor and major 2×i-Pr—CH₃); ¹³C NMR (90 MHz, CDCl₃) δ (ppm): 166.2, 165.8, 165.7, 165.7, 165.3, 165.2, 165.2, 164.9, 164.8, 164.7 (minor and major C═O, OD-C-2, OD-C-5), 158.3 (minor Py-C-6), 156.6 (major Py-C-6), 140.3 (minor Py-C-4), 140.2 (major Py-C-2), 140.1 (major Py-C-4), 139.1 (minor Py-C-2), 134.6, 134.0, 133.9, 133.8, 133.7, 133.5, 133.4, 131.1, 130.6, 130.2-128.5, 127.8 (minor and major Ar, Py-C-5), 125.3 (minor Py-C-3), 125.2 (major Py-C-3), 107.0, 102.3 (minor p-cym-C_{qAr}), 104.8, 102.1 (major p-cym-C_{qAr}), 88.7, 84.6, 83.2 (2) (major p-cym-CH_{Ar}), 85.5, 85.2, 83.8, 83.6 (minor p-cym-CH_{Ar}), 77.8 (minor C-5′), 77.4 (major C-5′), 73.8, 71.5, 69.8 (major C-1′-C-3′), 72.8, 71.9, 71.4 (minor C-1′-C-3′), 68.8 (major C-4′), 68.7 (minor C-4′), 63.0 (minor C-6′), 62.6 (major C-6′), 31.3 (minor i-Pr—CH), 31.1 (major i-Pr—CH), 23.1, 21.3 (major i-Pr—CH₃), 22.2, 22.1 (minor i-Pr—CH₃), 18.8 (minor C₆H₄—CH₃), 18.1 (major C₆H₄—CH₃). ESI-HRMS positive mode (m/z): calcd for C₅₁H₄₅ClN₃O₁₀Ru⁺ [M−PF₆]⁺ 996.1843; C₅₃H₅₂N₃O₁₂Ru⁺ [M−PF₆−Cl+OMe+MeOH]⁺ 1024.2604. Found: [M−PF₆]⁺ 996.1861; [M−PF₆−Cl+OMe+MeOH]⁺ 1024.2598.

### 11.21 Complex Ru-5

Prepared from complex Ru-dimer (10.0 mg, 0.0163 mmol), compound L-5 (17.9 mg, 0.0375 mmol, 2.3 eq.) and TIPF₆ (11.4 mg, 0.0326 mmol) according to general procedure IV. Purified by column chromatography (9:1 CHCl₃-MeOH) to give 21.7 mg (74%) yellow powder. R_{f}: 0.54 (9:1 CHCl₃-MeOH). Diastereomeric ratio: 4:3. ¹H NMR (400 MHz, CDCl₃) δ (ppm): 9.45 (d, J=3.9 Hz, minor Py-H-6), 9.30 (d, J=4.4 Hz, major Py-H-6), 8.23-8.08 (m, minor and major Py-H-3, Py-H-4), 7.88 (m, minor Py-H-5), 7.80 (m, major Py-H-5), 6.01-5.96, 5.82-5.69 (m, minor and major p-cym-CH_{Ar}), 5.75 (pt, J=9.7, 9.6 Hz, major H-2′), 5.44, (pt, J=9.3, 9.2 Hz, minor H-2′), 5.40 (pt, J=9.4, 9.0 Hz, minor and major H-3′), 5.24 (pt, J=9.8, 9.2 Hz, minor H-4′), 5.23 (pt, J=9.8, 9.8 Hz, major H-4′), 5.03 (d, J=10.0 Hz, major H-1′), 5.00 (d, J=8.7 Hz, minor H-1′), 4.30 (dd, J=12.8, 4.8 Hz, minor and major H-6′a), 4.17 (dd, J=12.8, 2.4 Hz, minor and major H-6′b), 3.99 (ddd, J=10.2, 4.8, 2.4 Hz, minor and major H-5′), 3.03 (hept, J=6.8 Hz, major i-Pr—CH), 2.91 (hept, J=6.8 Hz, minor i-Pr—CH), 2.23, 2.17, 2.10-2.02 (singlets, minor and major C₆H₄—CH₃, COCH₃), 1.36 (d, J=6.8 Hz, major 2×i-Pr—CH₃), 1.30, 1.25 (2 d, J=6.8 Hz in each, minor 2×i-Pr—CH₃); ¹³C NMR (90 MHz, CDCl₃) δ (ppm): 170.7, 170.6, 170.1, 170.0, 169.8, 169.6, 169.4 (2×4×C═O), 164.9, 164.6 (major OD-C-2, OD-C-5), 164.9, 164.7 (minor OD-C-2, OD-C-5), 157.6 (minor Py-C-6), 156.4 (major Py-C-6), 140.5 (minor Py-C-4), 140.3 (major Py-C-4), 140.3, 139.5 (minor and major Py-C-2), 130.8 (minor Py-C-5), 130.0 (major Py-C-5), 125.5 (minor Py-C-3), 125.4 (major Py-C-3), 106.4 (minor p-cym-C_{qAr}), 105.0 (major p-cym-C_{qAr}), 102.7 (minor p-cym-C_{qAr}), 102.4 (major p-cym-C_{qAr}), 88.2, 86.0, 85.6, 83.5, 83.2, 83.1 (minor and major p-cym-CH_{Ar}), 77.2 (minor C-5′), 77.0 (major C-5′), 73.7, 70.9 (major C-1′ and C-3′), 73.0, 71.2, 70.0 (minor C-1′-C-3′), 68.8 (major C-2′), 67.7 (minor C-4′), 67.6 (major C-4′), 61.9 (minor C-6′), 61.7 (major C-6′), 31.3 (minor i-Pr—CH), 31.2 (major i-Pr—CH), 22.9, 21.7 (major i-Pr—CH₃), 22.4, 22.0 (minor i-Pr—CH₃), 20.8, 20.7, 20.6, 20.6, 20.5 (2×4×COCH₃), 18.8 (minor C₆H₄—CH₃), 18.3 (major C₆H₄—CH₃). ESI-HRMS positive mode (m/z): calcd for C₃₁H₃₇ClN₃O₁₀Ru⁺ [M−PF₆]⁺ 748.1211. Found: 748.1212.

### 11.22 Complex Ru-6

Prepared from complex Ru-dimer (50 mg, 0.082 mmol), compound L-6 (97 mg, 0.164 mmol, 2 eq.) and TIPF₆ (57 mg, 0.163 mmol) according to general procedure IV. Purified by column chromatography (9:1 CHCl₃-MeOH) to give 140 mg (85%) yellow powder. R_{f}: 0.67 (9:1 CHCl₃-MeOH). Diastereomeric ratio: 3:2. ¹H NMR (400 MHz, CDCl₃) δ (ppm): 9.46 (d, J=4.1 Hz, minor Py-H-6), 9.26 (d, J=4.2 Hz, major Py-H-6), 8.18-7.73, 7.60-7.32 (m, minor and major Ar, Py-H-3-Py-H-5), 6.11 (pt, J=9.1, 9.1, major H-2′), 6.06-6.02 (m, major and minor H-3′), 5.86 (d, J=5.4 Hz, minor p-cym-CH_{Ar}), 5.80-5.76 (m, minor and major p-cym-CH_{Ar}), 5.74 (pt, J=9.3, 9.1 Hz, minor H-2′), 5.65, 5.63 (2 d, J=6.1 Hz in each, minor p-cym-CH_{Ar}), 5.61-5.53 (m, major and minor H-4′), 5.53 (d, J=5.4 Hz, major p-cym-CH_{Ar}), 5.37-5.35 (m, major p-cym-CH_{Ar}), 5.28 (d, J=9.1 Hz, major H-1′), 5.24 (d, J=9.1 Hz, minor H-1′), 4.64 (dd, J=11.8, 5.1 Hz, minor H-5′eq), 4.60 (dd, J=11.8, 5.4 Hz, major H-5′eq), 3.87 (pt, J=10.9, 10.1, minor and major H-5′ax), 2.80 (hept, J=6.9 Hz, major i-Pr—CH), 2.75 (hept, J=6.9 Hz, minor i-Pr—CH), 2.04 (s, minor C₆H₄—CH₃), 1.99 (s, major C₆H₄—CH₃), 1.19-1.11 (m, minor and major 2×i-Pr—CH₃); ¹³C NMR (90 MHz, CDCl₃) δ (ppm): 165.7, 165.6 (2), 165.5, 165.4, 165.3, 165.2, 164.9, 164.7, 164.6 (minor and major C═O, OD-C-2, OD-C-5), 157.9 (minor Py-C-6), 157.0 (major Py-C-6), 140.4 (minor Py-C-4), 140.3 (major Py-C-4), 140.0 (major Py-C-2), 139.3 (minor Py-C-2), 134.4, 133.9, 133.8 (2), 133.7, 133.5, 130.8-128.0 (minor and major Ar, Py-C-5), 125.2 (minor Py-C-3), 125.1 (major Py-C-3), 106.3, 104.9, 102.4, 102.1 (minor and major p-cym-C_{qAr}), 88.4, 86.0, 85.1, 84.3, 83.5, 83.4, 83.3, 83.1 (minor and major p-cym-CH_{Ar}), 73.1, 72.1, 71.9, 71.8, 70.5, 69.7, 69.5, 69.0 (minor and major C-1′-C-4′), 67.8, 67.4 (minor and major C-5′), 31.2, 31.0 (minor and major i-Pr—CH), 23.1, 22.4, 21.8, 21.1 (minor and major i-Pr—CH₃), 18.6, 18.1 (minor and major C₆H₄—CH₃). ESI-HRMS positive mode (m/z): calcd for C₄₃H₃₉ClN₃O₈Ru⁺ [M−PF₆]⁺ 862.1473; C₄₅H₄₆N₃O₁₀Ru⁺ [M−PF₆−Cl+OMe+MeOH]⁺ 890.2234. Found: [M−PF₆]⁺ 862.1470; [M−PF₆−Cl+OMe+MeOH]⁺ 890.2231.

### 11.23 Complex Ru-7

Prepared from complex Ru-dimer (50 mg, 0.082 mmol), compound L-7 (78 mg, 0.163 mmol, 2 eq.) and TIPF₆ (57 mg, 0.163 mmol) according to general procedure IV. Purified by column chromatography (9:1 CHCl₃-MeOH) to give 108 mg (74%) yellow powder. R_{f}: 0.54 (9:1 CHCl₃-MeOH). Diastereomeric ratio: 2:1. ¹H NMR (400 MHz, CDCl₃) δ (ppm): 9.46 (d, J=4.4 Hz, minor Py-H-6), 9.34 (d, J=4.4 Hz, major Py-H-6), 8.24-8.10 (m, minor and major Py-H-4, Py-H-3), 7.87 (m, minor Py-H-5), 7.80 (m, major Py-H-5), 6.01-5.95 (m, minor and major p-cym-CH_{Ar}), 5.85 (pt, J=10.2, 10.0 Hz major H-2′), 5.82-5.71 (m, minor and major p-cym-CH_{Ar}), 5.57-5.55 (m, minor and major H-4′), 5.47 (pt, J=10.0, 10.0 Hz minor H-2′), 5.29 (dd, J=10.0, 3.2 Hz, minor H-3′), 5.25 (dd, J=10.0, 3.2 Hz, major H-3′), 5.01 (d, J=10.2 Hz, major H-1′), 4.98 (d, J=10.0 Hz, minor H-1′), 4.23-4.10 (m, minor and major H-5′, H-6′a, H-6′b), 3.01 (hept, J=6.8 Hz, major i-Pr—CH), 2.89 (hept, J=6.8 Hz, minor i-Pr—CH), 2.24-2.23, 2.17, 2.07-2.04 (singlets, minor and major C₆H₄—CH₃, COCH₃), 1.34 (d, J=6.8 Hz, 2×major i-Pr—CH₃), 1.28, 1.24 (2 d, J=6.8 Hz in each, 2×minor i-Pr—CH₃); ¹³C NMR (90 MHz, CDCl₃) δ (ppm): 170.3, 170.2, 170.1, 170.0, 169.8, 169.7 (2×4×C═O), 164.8, 164.7, 164.5 (2) (minor and major OD-C-2, OD-C-5), 157.4 (minor Py-C-6), 156.5 (major Py-C-6), 140.3 (minor Py-C-4), 140.1 (major Py-C-4), 139.9 (major Py-C-2), 139.2 (minor Py-C-2), 130.5 (minor Py-C-5), 129.9 (major Py-C-5), 125.3 (minor Py-C-3), 125.2 (major Py-C-3), 106.2, 102.6 (minor p-cym-C_{qAr}), 105.1, 102.2 (major p-cym-C_{qAr}), 87.6, 85.0, 83.1, 82.9 (major p-cym-CH_{Ar}), 85.7, 85.2, 83.3, 83.0 (minor p-cym-CH_{Ar}), 75.6 (minor C-5′), 75.4 (major C-5′), 71.4, 71.0, 67.0 (major C-1′, C-3′, C-4′), 71.3, 70.7, 67.1, 66.9 (minor C-1′-C-4′), 65.8 (major C-2′), 61.4 (minor C-6′), 61.3 (major C-6′), 31.1 (minor i-Pr—CH), 31.0 (major i-Pr—CH), 22.6, 21.4 (major i-Pr—CH₃), 22.2, 21.8 (minor i-Pr—CH₃), 20.6, 20.6, 20.5, 20.5, 20.4, 20.4 (2×4×COCH₃), 18.5 (minor C₆H₄—CH₃), 18.1 (major C₆H₄—CH₃). ESI-HRMS positive mode (m/z): calcd for C₃₃H₄₄N₃O₁₂Ru⁺ [M−PF₆−Cl+OMe+MeOH]⁺ 776.1973. Found: 776.1973.

### 11.24 Complex Ru-8

Prepared from complex Ru-dimer (10.0 mg, 0.0163 mmol), compound L-8 (24.9 mg, 0.0343 mmol, 2.1 eq.) and TIPF₆ (11.4 mg, 0.0326 mmol) according to general procedure IV. Purified by column chromatography (95:5 CHCl₃-MeOH) to give 30.3 mg (81%) yellow powder. R_{f}: 0.49 (95:5 CHCl₃-MeOH). Diastereomeric ratio: 5:4. ¹H NMR (400 MHz, CDCl₃) δ (ppm): 9.49 (d, J=4.7 Hz, minor Py-H-6), 9.26 (d, J=5.1 Hz, major Py-H-6), 8.21-7.25 (m, minor and major Ar, Py-H-3-Py-H-5), 6.43 (pt, J=10.1, 10.0 Hz, major H-2′), 6.20 (d, J=3.3 Hz, major H-4′), 6.16 (d, J=2.6 Hz, minor H-4′), 6.02-5.94 (m, minor H-2′ and H-3′), 5.84-5.75 (m, minor and major p-cym-CH_{Ar}, major H-3′), 5.67, 5.64 (2 d, J=6.1 Hz in each, minor p-cym-CH_{Ar}) 5.52 (d, J=6.1 Hz, major p-cym-CH_{Ar}), 5.47 (d, J=10.1 Hz, major H-1′), 5.37 (d, J=8.7 Hz, minor H-1′), 5.32, 5.27 (2 d, J=6.1 Hz in each, major p-cym-CH_{Ar}), 4.72-4.62 (m, minor and major H-5′, H-6′a), 4.53 (dd, J=10.3, 4.1 Hz, minor H-6′b), 4.44 (dd, J=10.3, 4.0 Hz, major H-6′b), 2.80 (hept, J=6.9 Hz, major i-Pr—CH), 2.73 (hept, J=6.9 Hz, minor i-Pr—CH), 2.03 (s, minor C₆H₄—CH₃), 1.94 (s, major C₆H₄—CH₃), 1.17, 1.15 (2 d, J=6.9 Hz in each, 2×major i-Pr—CH₃), 1.14, 1.09 (2 d, J=6.9 Hz in each, 2×minor i-Pr—CH₃); ¹³C NMR (100 MHz, CDCl₃) δ (ppm): 166.2, 166.1, 165.9, 165.7, 165.5 (2), 165.4, 165.3, 164.9, 164.8 (2), 164.7 (2×4×C═O, 2×OD-C-2, 2×OD-C-5), 157.9 (minor Py-C-6), 156.3 (major Py-C-6), 140.4 (major Py-C-2), 140.3 (minor Py-C-4), 140.2 (major Py-C-4), 139.3 (minor Py-C-2), 134.5, 134.2, 133.9, 133.8 (2), 133.6, 133.5 (2), 130.9-128.1 (minor and major Ar, Py-C-5), 125.3 (minor Py-C-3), 125.2 (major Py-C-3), 106.8, 104.7, 102.3, 102.0 (minor and major p-cym-C_{qAr}), 88.8, 85.4 (2), 84.5, 83.6, 83.5, 83.0 (2) (minor and major p-cym-CH_{Ar}), 76.7 (minor C-5′), 76.3 (major C-5′), 72.5 (major C-3′), 72.2 (minor C-1′), 71.7 (major C-1′), 71.3, 68.7 (minor C-2′, C-3′), 68.3 (major C-4′), 68.2 (minor C-4′), 66.8 (major C-2′), 62.4 (minor C-6′), 62.1 (major C-6′), 31.3 (minor i-Pr—CH), 31.1 (major i-Pr—CH), 23.2, 21.3 (major i-Pr—CH₃), 22.2, 22.0 (minor i-Pr—CH₃), 18.7 (minor C₆H₄—CH₃), 18.0 (major C₆H₄—CH₃). ESI-HRMS positive mode (m/z): calcd for C₅₁H₄₅ClN₃O₁₀Ru⁺ [M−PF₆]⁺ 996.1843. Found: 996.1842.

### 11.25 Complex Ru-9

Prepared from complex Ru-dimer (50 mg, 0.082 mmol), compound L-9 (78 mg, 0.179 mmol, 2.2 eq.) and TIPF₆ (57 mg, 0.163 mmol) according to general procedure IV. Purified by column chromatography (9:1 CHCl₃-MeOH) to give 130 mg (94%) yellow powder. R_{f}: 0.54 (9:1 CHCl₃-MeOH). Diastereomeric ratio: 1:1. ¹H NMR (400 MHz, CDCl₃) δ (ppm): 9.33 (2H, d, J=4.2 Hz, 2×Py-H-6), 8.15 (2H, t, J=7.4 Hz, 2×Py-H-4), 8.07 (2H, d, J=7.4 Hz, 2×Py-H-3), 7.79 (2H, m, 2×Py-H-5), 6.52, 6.46 (2×1H, 2 d, J=2.3 Hz in each, 2×H-1′), 6.01-5.95 (4H, m, 2×p-cym-CH_{Ar}), 5.84-5.71 (6H, m, 2×p-cym-CH_{Ar}, 2×H-2′), 5.34, 5.30 (2×1H, 2 ddd, J=8.9, 4.1, 2.9 Hz in each, 2×H-3′), 4.36, 4.35 (2×1H, 2 dd, J=12.5, 2.9 Hz in each, 2×H-4′a), 4.21, 4.20 (2×1H, 2 dd, J=12.5, 4.1 in each, 2×H-4′b), 2.98, 2.97 (2×1H, 2 hept, J=6.9 Hz in each, 2×i-Pr—CH), 2.29, 2.24, 2.21, 2.18, 2.13, 2.12, 2.11, 2.10, 2.08, 2.08 (30H, singlets, 2×i-Pr—CH₃, 2×4×COCH₃), 1.33-1.29 (12H, m, 2×2×i-Pr—CH₃); ¹³C NMR (90 MHz, CDCl₃) δ (ppm): 170.7, 170.6, 169.9, 169.8, 169.6, 169.5, 169.4, 169.1 (2×4×C═O), 165.2, 165.1, 164.5 (2) (2×OD-C-2, 2×OD-C-5), 156.9, 156.8 (2×Py-C-6), 140.4 (2) (2×Py-C-4), 139.8, 139.7 (2×Py-C-2), 130.3 (2) (2×Py-C-5), 125.4, 125.3 (2×Py-C-3), 105.9, 105.7, 102.4, 102.3 (2×2×p-cym-C_{qAr}), 87.2, 86.6, 85.4, 85.1, 83.5 (2), 83.4, 83.3 (2×4×p-cym-CH_{Ar}), 68.9, 68.7 (2×C-2′), 68.0, 67.8 (2×C-3′), 64.3, 63.7 (2×C-1′), 61.5 (2) (2×C-4′), 31.3 (2) (2×i-Pr—CH), 22.8, 22.6, 21.9, 21.8 (2×2×i-Pr—CH₃), 21.0, 20.9 (2), 20.8, 20.7, 20.6, 20.5 (2) (2×4×COCH₃), 18.5, 18.4 (2×C₆H₄—CH₃). ESI-HRMS positive mode (m/z): calcd for C₂₉H₃₅ClN₃O₉Ru⁺ [M−PF₆]⁺ 706.1105; C₃₁H₄₂N₃O₁₁Ru⁺ [M−PF₆−Cl+OMe+MeOH]⁺ 734.1866. Found: [M−PF₆]⁺ 706.1106; [M−PF₆−Cl+OMe+MeOH]⁺ 734.1868.

### 11.26 Complex Ru-10

Prepared from complex Ru-dimer (39 mg, 0.064 mmol), compound L-10 (39 mg, 0.126 mmol, 2 eq.) and TIPF₆ (44 mg, 0.126 mmol) according to general procedure V. The crude product was triturated with Et₂O to give 80 mg (87%) orange powder. Diastereomeric ratio: 1:1. ¹H NMR (400 MHz, CD₃OD) δ (ppm): 9.54 (2H, d, J=5.5 Hz, 2×Py-H-6), 8.40-8.36 (4H, m, 2×Py-H-3, 2×Py-H-4), 7.98-7.93 (2H, m, 2×Py-H-5), 6.19-6.13 (2×2H, m, 2×p-cym-CH_{Ar}), 5.94-5.92 (2×2H, m, 2×p-cym-CH_{Ar}), 4.85, 4.83 (2×1H, 2 d, J=9.8 Hz in each, 2×H-1′), 3.92, 3.91 (2×1H, 2 dd, J=12.1, 6.1 Hz in each, 2×H-6′a), 3.83, 3.81 (2×1H, 2 pt, J=9.5, 9.3 Hz in each, 2×H-2′), 3.71, 3.70 (2×1H, 2 dd, J=12.1, <1 Hz in each, 2×H-6′b), 3.59-3.54 (4H, m, 2×H-3′, 2×H-5′), 3.45, 3.45 (2×1H, 2 pt, J=9.8, 9.5 Hz in each, 2×H-4′), 2.91, 2.90 (2×1H, 2 hept, J=6.9 Hz in each, 2×i-Pr—CH), 2.23 (6H, s, 2×C₆H₄—CH₃), 1.28-1.23 (12H, m, 2×2×i-Pr—CH₃); ¹³C NMR (90 MHz, CD₃OD) δ (ppm): 168.5 (2), 166.0 (2) (2×OD-C-2, 2×OD-C-5), 158.3, 158.3 (2×Py-C-6), 142.0 (2) (2×Py-C-4), 141.6, 141.5 (2×Py-C-2), 131.1 (2) (2×Py-C-5), 126.6, 126.5 (2×Py-C-3), 107.3, 107.2, 103.5, 103.4 (2×2×p-cym-C_{qAr}), 87.4, 87.2, 85.9, 85.8, 85.1, 85.0, 84.9, 84.8 (2×4×p-cym-CH_{Ar}), 83.2, 83.1 (2×C-5′), 78.8, 78.7 (2×C-3′), 74.6 (2) (2×C-1′), 73.3, 73.2 (2×C-2′), 71.1 (2) (2×C-4′), 62.6 (2) (2×C-6′), 32.4 (2) (2×i-Pr—CH), 22.8, 22.7, 22.1, 22.0 (2×2×i-Pr—CH₃), 18.8 (2) (2×C₆H₄—CH₃). ESI-HRMS positive mode (m/z): calcd for C₂₃H₂₉ClN₃O₆Ru⁺ [M−PF₆]⁺ 580.0786; C₂₅H₃₆N₃O₈Ru⁺ [M−PF₆−Cl+OMe+MeOH]⁺ 608.1548. Found: [M−—PF₆]⁺ 580.0787; [M−PF₆−Cl+OMe+MeOH]⁺ 608.1545.

### 11.27 Complex Ru-11

Prepared from complex Ru-dimer (10.0 mg, 0.016 mmol), compound L-11 (9.1 mg, 0.033 mmol, 2 eq.) and TIPF₆ (11.4 mg, 0.033 mmol) according to general procedure V. Purified by recrystallisation from an iPrOH-Et₂O solvent mixture (2 mL and 5 mL, respectively) to give 9.5 mg (42%) yellow powder. Diastereomeric ratio: 1:1. ¹H NMR (400 MHz, CD₃OD) δ (ppm): 9.53 (2H, d, J=5.4 Hz, 2×Py-H-6), 8.38-8.34 (4H, m, 2×Py-H-3, 2×Py-H-4), 7.95-7.92 (2H, m, 2×Py-H-5), 6.18-6.12 (2×2H, m, 2×p-cym-CH_{Ar}), 5.94-5.90 (2×2H, m, 2×p-cym-CH_{Ar}), 4.77, 4.76 (2×1H, 2 d, J=9.6 Hz in each, 2×H-1′), 4.07, 4.06 (2×1H, 2 dd, J=11.1, 5.3 Hz in each, 2×H-5′eq), 3.81, 3.80 (2×1H, 2 pt, J=9.6, 9.0 Hz in each, 2×H-2′), 3.68, 3.65 (2×1H, 2 ddd, J=10.5, 8.9, 5.3 Hz in each, 2×H-4′), 3.51 (2H, pt, J=9.0, 8.9 Hz, 2×H-3′), 3.47, 3.46 (2×1H, 2 pt, J=11.1, 10.5 Hz in each, 2×H-5′ax), 2.91 (2H, 2 hept, J=6.8 Hz, 2×i-Pr—CH), 2.22 (6H, s, 2×C₆H₄—CH₃), 1.28-1.23 (12H, m, 2×2×i-Pr—CH₃); ¹³C NMR (100 MHz, CD₃OD) δ (ppm): 168.8, 168.6, 166.1, 166.0 (2×OD-C-2, 2×OD-C-5), 158.2 (2) (2×Py-C-6), 142.0 (2) (2×Py-C-4), 141.6, 141.5 (2×Py-C-2), 131.1 (2) (2×Py-C-5), 126.6, 126.5 (2×Py-C-3), 107.4 (2), 103.5, 103.4 (2×2×p-cym-C_{qAr}), 87.4, 87.3, 86.0, 85.9, 85.0 (2), 84.9, 84.8 (2×4×p-cym-CH_{Ar}), 79.0, 78.9 (2×C-3′), 75.4, 75.3 (2×C-1′), 73.5, 73.3, (2×C-2′), 71.8, 71.7 (2×C-5′), 70.8 (2) (2×C-4′), 32.4 (2) (2×i-Pr—CH), 22.7 (2), 22.0 (2) (2×2×i-Pr—CH₃), 18.7 (2) (2×C₆H₄—CH₃). ESI-HRMS positive mode (m/z): calcd for C₂₂H₂₇ClN₃O₅Ru⁺ [M−PF₆]⁺ 550.0680; C₂₄H₃₄N₃O₇Ru⁺ [M−PF₆−Cl+OMe+MeOH]⁺ 578.1442. Found: [M−PF₆]⁺ 550.0672; [M−PF₆−Cl+OMe+MeOH]⁺ 578.1432.

### 11.28 Complex Ru-12

Prepared from complex Ru-dimer (10.0 mg, 0.016 mmol), compound L-12 (10.1 mg, 0.033 mmol, 2 eq.) and TIPF₆ (11.4 mg, 0.033 mmol) according to general procedure V. Purified by recrystallisation from an iPrOH-Et₂O solvent mixture (2 mL and 5 mL, respectively) to give 11.9 mg (50%) yellow powder. Diastereomeric ratio: 1:1. ¹H NMR (360 MHz, CD₃OD) δ (ppm): 9.55 (2H, d, J=5.6 Hz, 2×Py-H-6), 8.40-8.35 (4H, m, 2×Py-H-3, 2×Py-H-4), 7.96-7.93 (2H, m, 2×Py-H-5), 6.19-6.13 (2×2H, m, 2×p-cym-CH_{Ar}), 5.94-5.92 (2×2H, m, 2×p-cym-CH_{Ar}), 4.78, 4.76 (2×1H, 2 d, J=9.8 Hz in each, 2×H-1′), 4.18, 4.15 (2×1H, 2 pt, J=9.8, 9.4 in each, 2×H-2′), 4.01 (2H, d, J=3.2 Hz, 2×H-4′), 3.84-3.73 (6H, m, 2×H-5′, 2×H-6′a, 2×H-6′b), 3.69, 3.68 (2×1H, 2 dd, J=9.4, 3.2 in each, 2×H-3′), 2.91 (2H, 2 hept, J=6.9 Hz, 2×i-Pr—CH), 2.23 (6H, s, 2×C₆H₄—CH₃), 1.27-1.23 (12H, m, 2×2×i-Pr—CH₃); ¹³C NMR (90 MHz, CD₃OD) δ (ppm): 168.7, 168.6, 166.0, 165.9 (2×OD-C-2, 2×OD-C-5), 158.1 (2) (2×Py-C-6), 141.9 (2) (2×Py-C-4), 141.6, 141.5 (2×Py-C-2), 131.1 (2) (2×Py-C-5), 126.5, 126.4 (2×Py-C-3), 107.4, 107.3, 103.5, 103.4 (2×2×p-cym-C_{qAr}), 87.2, 87.1, 86.0, 85.8, 85.0 (2), 84.8, 84.7 (2×4×p-cym-CH_{Ar}), 82.0, 81.9 (2×C-5′), 75.6, 75.5, 75.1, 75.0 (2×C-1′, 2×C-3′), 70.6, 70.5, 70.2, 70.0 (2×C-2′, 2×C-4′), 62.7 (2) (2×C-6′), 32.4 (2) (2×i-Pr—CH), 22.7, 22.6, 22.1, 22.0 (2×2×i-Pr—CH₃), 18.7 (2) (2×C₆H₄—CH₃). ESI-HRMS positive mode (m/z): calcd for C₂₃H₂₉ClN₃O₆Ru⁺ [M−PF₆]⁺ 580.0786; C₂₅H₃₆N₃O₈Ru⁺ [M−PF₆−Cl+OMe+MeOH]⁺ 608.1548. Found: [M−PF₆]⁺ 580.0792; [M−PF₆−Cl+OMe+MeOH]⁺ 608.1553.

### 11.29 Complex Ru-13

Prepared from complex Ru-dimer (20.0 mg, 0.033 mmol), 1-phenyl-4-(pyridine-2-yl)-1,2,3-triazole (Tawfiq et al., 2014) (L-13, 14.5 mg, 0.065 mmol, 2 eq.) and TIPF₆ (22.8 mg, 0.065 mmol) according to general procedure V. Purified by recrystallisation from a CHCl₃-Et₂O solvent mixture (3 mL and 6 mL, respectively) to give 36.8 mg (88%) yellow powder. Racemic mixture. ¹H NMR (400 MHz, acetone-d₆) δ (ppm): 9.62 (1H, s, Tria-H-5), 9.57 (1H, ddd, J=5.6, 1.4, 0.9 Hz, Py-H-6), 8.29 (1H, dt, J=7.6, 1.4 Hz, Py-H-4), 8.25 (1H, ddd, J=7.9, 1.9, 0.9 Hz, Py-H-3), 8.12-8.09 (2H, m, Ph), 7.79-7.68 (4H, m, Ph, Py-H-5), 6.27, 6.24, 6.04, 5.99 (4×1H, 4 d, J=6.2 Hz in each, 4×p-cym-CH_{Ar}), 2.91 (1H, hept, J=6.9 Hz, i-Pr—CH), 2.29 (3H, s, C₆H₄—CH₃), 1.23, 1.19 (2×3H, 2 d, J=6.9 Hz in each, 2×i-Pr—CH₃); ¹³C NMR (90 MHz, acatone-d₆) δ (ppm): 156.6 (Py-C-6), 149.2, 148.2 (Tria-C-4, Py-C-2), 141.1 (Py-C-4), 131.6, 131.2, 127.3, 124.0, 123.3, 121.9 (Ph, Py-C-3, Py-C-5, Tria-C-5), 106.2, 103.4 (2×p-cym-C_{qAr}), 87.4, 85.8, 85.1, 84.5 (4×p-cym-CH_{Ar}), 31.9 (i-Pr—CH), 22.6, 21.9 (2×i-Pr—CH₃), 18.7 (C₆H₄—CH₃). ESI-HRMS positive mode (m/z): calcd for C₂₃H₂₄ClN₄Ru⁺ [M−PF₆]⁺: 493.0730. Found: 493.0709.

### 11.30 Complex Ru-14

Prepared from complex Ru-dimer (20.0 mg, 0.033 mmol), 2-phenyl-5-(pyridine-2-yl)-1,3,4-oxadiazole (Wei et al., 2010; Weiss et al., 2020) (L-14, 14.6 mg, 0.065 mmol, 2 eq.) and TIPF₆ (22.8 mg, 0.065 mmol) according to general procedure V. Purified by recrystallisation from a CHCl₃-Et₂O solvent mixture (3 mL and 6 mL, respectively) to give 34.9 mg (84%) yellow powder. Racemic mixture. ¹H NMR (360 MHz, acatone-d₆) δ (ppm): 9.69 (1H, ddd, J=5.6, 1.3, 0.8 Hz, Py-H-6), 8.54 (1H, ddd, J=7.8, 1.6, 0.8 Hz, Py-H-3), 8.48 (1H, dt, J=7.8, 1.3 Hz, Py-H-4), 8.33-8.29 (2H, m, Ph), 8.01 (1H, ddd, J=7.8, 5.6, 1.6 Hz, Py-H-5), 7.85-7.72 (3H, m, Ph), 6.30-6.27, 6.07-6.04 (2×2H, 2 m, 4×p-cym-CH_{Ar}), 3.03 (1H, hept, J=6.9 Hz, i-Pr—CH), 2.31 (3H, s, C₆H₄—CH₃), 1.32, 1.30 (2×3H, 2 d, J=6.9 Hz in each, 2×i-Pr—CH₃); ¹³C NMR (90 MHz, acatone-d₆) δ (ppm): 168.2, 164.7 (OD-C-2, OD-C-5), 157.8 (Py-C-6), 141.6 (Py-C-4), 141.5 (Py-C-2), 134.9, 130.7, 130.3, 128.5, 125.9, 122.7 (Ph, Py-C-3, Py-C-5), 106.7, 102.9 (2×p-cym-C_{qAr}), 86.9, 85.6, 84.7, 84.5 (4×p-cym-CH_{Ar}), 32.0 (i-Pr—CH), 22.7, 22.0 (2×i-Pr—CH₃), 18.8 (C₆H₄—CH₃). ESI-HRMS positive mode (m/z): calcd for C₂₃H₂₃ClN₃ORu⁺ [M−PF₆]⁺: 494.0556. Found: 494.0553.

### 11.31 Complex Os-2a

Prepared from complex Os-dimer (20.0 mg, 0.0253 mmol), compound L-2a (36.7 mg, 0.0506 mmol, 2.0 eq.) and TIPF₆ (17.6 mg, 0.0504 mmol) according to general procedure IV. After filtration and removal of the solvent the residue was dissolved in CHCl₃ (5 mL) and Et₂O (10 mL) was added. The precipitated product was filtered off, washed with a solvent mixture of CHCl₃-Et₂O=1:1 (2 mL) to give 52.9 mg (85%) yellow powder. R_{f}: 0.57 (9:1 CHCl₃-MeOH). Diastereomeric ratio: 1:1. ¹H NMR (400 MHz, CDCl₃) δ (ppm): 9.19 (2H, d, J=5.3 Hz, 2×Py-H-6), 9.07, 9.05 (2×1H, 2 s, 2×Tria-H-5), 8.05-7.75, 7.53-7.23 (46H, m, 2×20×Ar, 2×Py-H-3-Py-H-5), 6.58, 6.24-5.60 (14H, m, 2×H-2′, 2×H-3′, 2×H-4′, 2×4×p-cym-CH_{Ar}), 6.49, 6.43 (2×1H, 2 d, J=9.2 and 9.0 Hz, respectively, 2×H-1′), 4.79-4.53 (6H, m, 2×H-5′, 2×H-6′a, 2×H-6′b), 2.35, 2.16 (2×1H, 2 hept, J=6.9 Hz in each, 2×i-Pr—CH), 2.14, 2.07 (2×3H, 2 s, 2×C₆H₄—CH₃), 0.89, 0.86, 0.75, 0.63 (2×2×3H, 2×2 d, J=6.9 Hz in each, 2×2×i-Pr—CH₃); ¹³C NMR (100 MHz, CDCl₃) δ (ppm): 166.3, 166.2, 165.6, 165.5, 165.2 (2), 164.9, 164.8 (2×4×C═O), 155.8, 155.7 (2×Py-C-6), 148.4, 148.1, 148.0, 147.9 (2×Tria-C-4, 2×Py-C-2), 140.3, 140.2 (2×Py-C-4), 134.3, 134.2, 133.9, 133.8, 133.7, 133.6, 133.5, 133.3, 130.2-128.4, 128.0, 127.9, 127.7, 127.6 (Ar, 2×Py-C-5, 2×Tria-C-5), 122.5, 122.4 (2×Py-C-3), 97.0, 96.4, 96.1, 95.2 (2×2×p-cym-C_{qAr}), 87.2, 86.7 (2×C-1′), 78.2, 77.7, 76.9, 76.8, 76.1, 75.7, 74.9, 74.5, 73.7, 73.6, 72.8, 72.7, 71.7, 70.4, 68.7, 68.6 (2×4×p-cym-CH_{Ar}, 2×C-1′-C-5′), 62.8, 62.7 (2×C-6′), 31.1 (2) (2×i-Pr—CH), 22.8, 22.6, 21.9, 21.5 (2×2×i-Pr—CH₃), 18.6, 18.5 (2×C₆H₄—CH₃). ESI-HRMS positive mode (m/z): calcd for C₅₁H₄₆ClN₄O₉Os⁺ [M−PF₆]⁺ 1085.2566. Found: 1085.2555.

### 11.32 Complex Os-4

Prepared from complex Os-dimer (10.0 mg, 0.0126 mmol), compound L-4 (18.4 mg, 0.0254 mmol, 2.0 eq.) and TIPF₆ (8.8 mg, 0.0252 mmol) according to general procedure IV. After filtration and removal of the solvent the residue was dissolved in CHCl₃ (3 mL) and Et₂O (12 mL) was added. The precipitated product was filtered off, washed with a solvent mixture of CHCl₃-Et₂O=1:2 (1 mL) to give 22.1 mg (71%) orange powder. R_{f}: 0.44 (95:5 CHCl₃-MeOH). Diastereomeric ratio: 7:6. ¹H NMR (400 MHz, CDCl₃) δ (ppm): 9.45 (d, J=5.6 Hz, major Py-H-6), 9.18 (d, J=5.6 Hz, minor Py-H-6), 8.18-7.28 (m, minor and major Ar, Py-H-3-Py-H-5), 6.21-6.05, 5.90-5.84, 5.77-5.72 (m, J=9.8, 9.7 Hz, minor and major H-2′, H-3′, H-4′, 4×major p-cym-CH_{Ar}, 2×minor p-cym-CH_{Ar}), 5.57, 5.51 (2 d, J=5.7 Hz in each, 2×minor p-cym-CH_{Ar}), 5.46 (d, J=9.8 Hz, minor H-1′), 5.35 (d, J=9.9 Hz, major H-1′), 4.73, 4.72 (2 dd, J=12.6, 2.6 Hz in each, minor and major H-6′a), 4.56, 4.55 (2 dd, J=12.6, 5.6 Hz in each, minor and major H-6′b), 4.48, 4.45 (2 ddd, J=9.9, 5.6, 2.6 Hz in each, minor and major H-5′), 2.63 (hept, J=6.9 Hz, minor i-Pr—CH), 2.57 (hept, J=6.9 Hz, major i-Pr—CH), 2.09 (s, major C₆H₄—CH₃), 2.01 (s, minor C₆H₄—CH₃), 1.14, 1.10, 1.05, 1.04 (4 d, J=6.9 Hz in each, minor and major 2×i-Pr—CH₃); ¹³C NMR (100 MHz, CDCl₃) δ (ppm): 169.1, 168.8, 166.3, 165.8, 165.7, 165.3, 165.2 (2), 164.9, 164.8 (minor and major C═O, OD-C-2, OD-C-5), 158.3 (minor Py-C-6), 156.5 (major Py-C-6), 140.3 (major Py-C-4), 140.1 (minor Py-C-2), 140.1 (minor Py-C-4), 139.0 (major Py-C-2), 134.6, 134.0 (2), 133.8, 133.7, 133.5 (2), 133.4, 131.8-127.7 (minor and major Ar, Py-C-5), 125.3 (2) (minor and major Py-C-3), 98.2, 94.9 (major p-cym-C_{qAr}), 95.5, 93.7 (minor p-cym-C_{qAr}), 81.0, 77.8, 77.7, 77.5, 77.4, 76.7, 75.4, 74.9, 74.8, 74.4, 73.8, 72.8, 71.8, 71.4 (2), 69.7, 68.8, 68.7 (minor and major p-cym-CH_{Ar}, C-1′-C-5′), 63.0 (major C-6′), 62.6 (minor C-6′), 31.4 (major i-Pr—CH), 31.1 (minor i-Pr—CH), 23.5, 21.3 (minor i-Pr—CH₃), 22.5, 22.2 (major i-Pr—CH₃), 18.7 (major C₆H₄—CH₃), 18.0 (minor C₆H₄—CH₃). ESI-HRMS positive mode (m/z): calcd for C₅₁H₄₅ClN₃O₁₀Os⁺ [M−PF₆]⁺ 1086.2406. Found: [M−PF₆]⁺ 1086.2391.

### 11.33 Complex Os-6

Prepared from complex Os-dimer (10.0 mg, 0.0126 mmol), compound L-6 (15.0 mg, 0.0254 mmol, 2.0 eq.) and TIPF₆ (8.8 mg, 0.0252 mmol) according to general procedure IV. After filtration and removal of the solvent the residue was dissolved in CHCl₃ (3 mL) and Et₂O (12 mL) was added. The precipitated product was filtered off, washed with a solvent mixture of CHCl₃-Et₂O=1:2 (2 mL) to give 23.3 mg (84%) orange powder. R_{f}: 0.34 (95:5 CHCl₃-MeOH). Diastereomeric ratio: 7:5. ¹H NMR (400 MHz, CDCl₃) δ (ppm): 9.40 (d, J=5.6 Hz, major Py-H-6), 9.22 (d, J=5.6 Hz, minor Py-H-6), 8.17-7.32 (m, minor and major Ar, Py-H-3-Py-H-5), 6.11 (pt, J=9.0, 9.0 Hz, major H-2′ or H-3′), 6.08-6.03 (m, major and minor H-2′ or H-3′ and p-cym-CH_{Ar}), 5.91 (d, J=5.8 Hz, minor p-cym-CH_{Ar}), 5.86, 5.84 (2 d, J=6.2 Hz in each, 2×major p-cym-CH_{Ar}), 5.76 (pt, J=9.1, 9.0 Hz, minor H-2′ or H-3′), 5.75 (m, major p-cym-CH_{Ar}), 5.64 (d, J=5.8 Hz, minor p-cym-CH_{Ar}), 5.62-5.51 (m, major and minor H-4′, minor p-cym-CH_{Ar}), 5.30 (d, J=9.7 Hz, minor H-1′), 5.25 (d, J=9.0 Hz, major H-1′), 4.66 (dd, J=11.7, 5.2 Hz, major H-5′eq), 4.60 (dd, J=11.4, 5.5 Hz, minor H-5′eq), 3.88 (pt, J=11.4, 9.7 Hz, major H-5′ax), 3.87 (pt, J=11.4, 10.4 Hz, minor H-5′ax), 2.63 (hept, J=6.9 Hz, minor i-Pr—CH), 2.60 (hept, J=6.9 Hz, major i-Pr—CH), 2.11 (s, major C₆H₄—CH₃), 2.04 (s, minor C₆H₄—CH₃), 1.14, 1.12, 1.06, 1.05 (4 d, J=6.9 Hz in each, minor and major 2×i-Pr—CH₃); ¹³C NMR (100 MHz, CDCl₃) δ (ppm): 167.0, 168.8, 165.8, 165.7 (2), 165.6, 165.5, 165.4, 165.3, 165.1 (minor and major C═O, OD-C-2, OD-C-5), 158.0 (major Py-C-6), 156.7 (minor Py-C-6), 140.3 (major Py-C-4), 140.2 (minor Py-C-4), 140.0 (minor Py-C-2), 139.2 (major Py-C-2), 134.5, 134.0, 133.9 (2), 133.8, 133.6, 131.5-128.0 (minor and major Ar, Py-C—S), 125.3 (2) (minor and major Py-C-3), 97.7, 94.9 (major p-cym-C_{qAr}), 95.7, 93.9 (minor p-cym-C_{qAr}), 80.7, 78.3, 77.3, 76.7, 75.2, 74.9, 74.8, 74.4, 73.0, 72.1, 71.9, 71.8, 70.7, 69.7, 69.6, 69.0 (minor and major C-1′-C-4′), 67.9 (minor C-5′), 67.5 (major C-5′), 31.3 (major i-Pr—CH), 31.1 (minor i-Pr—CH), 23.4, 21.4 (minor i-Pr—CH₃), 22.7, 22.1 (major i-Pr—CH₃), 18.6 (major C₆H₄—CH₃), 18.0 (minor C₆H₄—CH₃). ESI-HRMS positive mode (m/z): calcd for C₄₃H₃₉ClN₃O₈Os⁺ [M−PF₆]⁺ 952.2037. Found: [M−PF₆]⁺ 952.2025.

### 11.34 Complex Os-8

Prepared from complex Os-dimer (20.0 mg, 0.0253 mmol), compound L-8 (36.7 mg, 0.0506 mmol, 2.0 eq.) and TIPF₆ (17.6 mg, 0.0504 mmol) according to general procedure IV. After filtration and removal of the solvent the residue was dissolved in CHCl₃ (5 mL) and Et₂O (10 mL) was added. The precipitated product was filtered off, washed with a solvent mixture of CHCl₃-Et₂O=1:2 (2 mL) to give 48.4 mg (78%) orange powder. R_{f}: 0.69 (9:1 CHCl₃-MeOH). Diastereomeric ratio: 3:2. ¹H NMR (400 MHz, CDCl₃) δ (ppm): 9.43 (d, J=5.6 Hz, major Py-H-6), 9.22 (d, J=5.6 Hz, minor Py-H-6), 8.21-7.24 (m, minor and major Ar, Py-H-3-Py-H-5), 6.42 (pt, J=10.1, 10.0 Hz, minor H-2′), 6.20 (d, J=3.4 Hz, minor H-4′), 6.17 (d, J=2.6 Hz, major H-4′), 6.06, 6.04 (2 d, J=5.8 Hz in each, 2×major p-cym-CH_{Ar}), 6.03-5.95 (m, major H-2′ and H-3′), 5.88-5.84 (m, minor and 2×major p-cym-CH_{Ar}), 5.80 (dd, J=10.1, 3.4 Hz, minor H-3′), 5.76, 5.56, 5.53 (3 d, J=5.8 Hz in each, 3×minor p-cym-CH_{Ar}), 5.48 (d, J=10.2 Hz, minor H-1′), 5.39 (d, J=9.7 Hz, major H-1′), 4.73-4.63 (m, minor and major H-5′, H-6′a), 4.54 (dd, J=10.3, 4.1 Hz, major H-6′b), 4.48 (m, minor H-6′b), 2.63 (hept, J=6.9 Hz, minor i-Pr—CH), 2.57 (hept, J=6.9 Hz, major i-Pr—CH), 2.10 (s, major C₆H₄—CH₃), 2.00 (s, minor C₆H₄—CH₃), 1.13, 1.06 (2 d, J=6.9 Hz in each, 2×minor i-Pr—CH₃), 1.10, 1.04 (2 d, J=6.9 Hz in each, 2×major i-Pr—CH₃); ¹³C N M R (100 MHz, CDCl₃) δ (ppm): 169.0, 168.8, 166.2, 166.1, 165.9, 165.7, 165.6, 165.5, 165.4, 165.3, 165.0, 164.9 (minor and major C═O, OD-C-2, OD-C-5), 158.1 (major Py-C-6), 156.6 (minor Py-C-6), 140.4 (major Py-C-4), 140.2 (minor Py-C-4), 140.1 (minor Py-C-2), 139.1 (major Py-C-2), 134.5, 134.2, 133.9, 133.8 (2), 133.6, 133.5 (2), 131.6-128.0 (minor and major Ar, Py-C-5), 125.4 (major Py-C-3), 125.3 (minor Py-C-3), 97.9, 94.9 (major p-cym-C_{qAr}), 95.6, 93.7 (minor p-cym-C_{qAr}), 80.9, 78.0, 77.3, 76.7, 76.6, 76.3, 75.3, 74.9 (2), 74.5, 72.4, 72.1, 71.7, 71.3, 68.7, 68.4, 68.3, 66.8 (minor and major p-cym-CH_{Ar}, C-1′-C-5′), 62.4 (major C-6′), 62.2 (minor C-6′), 31.3 (major i-Pr—CH), 31.0 (minor i-Pr—CH), 23.5, 21.3 (minor i-Pr—CH₃), 22.6, 22.1 (major i-Pr—CH₃), 18.6 (major C₆H₄—CH₃), 17.9 (minor C₆H₄—CH₃). ESI-HRMS positive mode (m/z): calcd for C₅₁H₄₅ClN₃O₁₀Os⁺ [M−PF₆]⁺ 1086.2406. Found: [M−PF₆]⁺ 1086.2390.

### 11.35 Complex Ir-2a

Prepared from complex Ir-dimer (20.0 mg, 0.0251 mmol), compound L-2a (36.4 mg, 0.0502 mmol, 2.0 eq.) and TIPF₆ (17.5 mg, 0.0501 mmol) according to general procedure IV. After filtration and removal of the solvent the residue was dissolved in CHCl₃ (5 mL) and Et₂O (10 mL) was added. The precipitated product was filtered off, washed with a solvent mixture of CHCl₃-Et₂O=1:1 mixture (2 mL) to give 51.9 mg (84%) yellow powder. R_{f}: 0.58 (9:1 CHCl₃-MeOH). Diastereomeric ratio: 2:1. ¹H NMR (400 MHz, CDCl₃) δ (ppm): 9.20 (s, minor Tria-H-5), 9.13 (s, major Tria-H-5), 8.68 (1 signal, minor and major Py-H-6), 8.05-7.82, 7.58-7.23 (m, minor and major Ar, Py-H-3-Py-H-5), 6.53 (d, J=9.3 Hz, major H-1′), 6.48 (d, J=8.5 Hz, minor H-1′), 6.46 (pt, J=9.4, 9.2 Hz, major H-2′ or H-3′ or H-4′), 6.22 (pt, J=9.2, 9.2 Hz, minor H-2′ or H-3′ or H-4′), 6.19 (pt, J=9.2, 8.7 Hz, minor H-2′ or H-3′ or H-4′), 6.13 (pt, J=9.5, 9.5 Hz, major H-2′ or H-3′ or H-4′), 5.98 (pt, J=9.5, 9.5 Hz, minor H-2′ or H-3′ or H-4′), 5.92 (pt, J=9.9, 9.7 Hz, major H-2′ or H-3′ or H-4′), 4.78-4.54 (minor and major H-5′, H-6′a, H-6′b), 1.62 (s, major Cp*—CH₃), 1.57 (s, minor Cp*—CH₃); ¹³C NMR (100 MHz, CDCl₃) δ (ppm): 166.3, 165.6, 165.2, 164.6 (minor C═O), 166.2, 165.5, 165.3, 164.7 (major C═O), 151.5 (2) (minor and major Py-C-6), 148.5, 147.8 (minor Tria-C-4, Py-C-2), 148.1, 147.9 (major Tria-C-4, Py-C-2), 140.6 (minor Py-C-4), 140.5 (major Py-C-4), 134.1, 134.0, 133.8, 133.7, 133.6, 133.5, 133.4, 133.3, 130.2-127.8 (minor and major Ar, Py-C-5, Tria-C-5), 123.0 (minor Py-C-3), 122.9 (major Py-C-3), 89.6 (major Cp*), 89.5 (minor Cp*), 87.3 (minor C-1′), 87.0 (major C-1′), 76.1, 72.9, 71.4, 68.7 (minor C-2′-C-5′), 75.8, 73.6, 70.3, 68.6 (major C-2′-C-5′), 62.8 (minor C-6′), 62.7 (major C-6′), 8.7 (minor and major Cp*—CH₃). ESI-HRMS positive mode (m/z): calcd for C₅₁H₄₇ClN₄O₉Ir⁺ [M−PF₆]⁺ 1087.2655. Found: [M−PF₆]⁺ 1087.2647.

### 11.36 Complex Ir-4

Prepared from complex Ir-dimer (20.0 mg, 0.0251 mmol), compound L-4 (36.4 mg, 0.0502 mmol, 2.0 eq.) and TIPF₆ (17.5 mg, 0.0501 mmol) according to general procedure IV. Purified by column chromatography (95:5 CHCl₃-MeOH) to give 49.3 mg (80%) yellow powder. R_{f}: 0.32 (95:5 CHCl₃-MeOH). Diastereomeric ratio: 5:2. ¹H NMR (400 MHz, CDCl₃) δ (ppm): 8.95 (d, J=5.5 Hz, minor Py-H-6), 8.78 (d, J=5.5 Hz, major Py-H-6), 8.22-7.78, 7.57-7.26 (m, minor and major Ar, Py-H-3-Py-H-5), 6.32 (pt, J=9.7, 9.6 Hz, major H-2′ or H-3′ or H-4′), 6.19 (pt, J=9.7, 9.5 Hz, minor H-2′ or H-3′ or H-4′), 6.04 (pt, J=9.5, 9.5 Hz, major H-2′ or H-3′ or H-4′), 5.87 (pt, J=9.8, 9.7 Hz, minor H-2′ or H-3′ or H-4′), 5.86 (pt, J=9.8, 9.7 Hz, major H-2′ or H-3′ or H-4′), 5.78 (pt, J=9.7, 9.7 Hz, minor H-2′ or H-3′ or H-4′), 5.51 (d, J=10.2 Hz, major H-1′), 5.42 (d, J=9.9 Hz, minor H-1′), 4.72 (dd, J=12.8, 2.4 Hz, minor H-6′a), 4.67 (dd, J=12.6, 2.4 Hz, major H-6′a), 4.57 (dd, J=12.8, 5.6 Hz, minor H-6′b), 4.53 (dd, J=12.6, 4.3 Hz, major H-6′b), 4.47 (ddd, J=9.8, 4.3, 2.4 Hz, major H-5′), 4.46 (m, minor H-5′), 1.61 (s, minor Cp*—CH₃), 1.50 (s, major Cp*—CH₃); ¹³C NMR (90 MHz, CDCl₃) δ (ppm): 169.4, 166.2, 165.8, 165.3, 165.2, 165.0 (major C═O, OD-C-2, OD-C-5), 169.2, 166.2, 165.8, 165.7, 165.6, 165.4 (minor C═O, OD-C-2, OD-C-5), 153.8 (minor Py-C-6), 152.5 (major Py-C-6), 140.8 (minor Py-C-4), 140.4 (major Py-C-4), 140.0 (major Py-C-2), 138.9 (minor Py-C-2), 134.4, 133.9, 133.8, 133.7, 133.6, 133.5, 133.4, 133.3, 132.0, 130.9, 130.5, 130.1-127.8 (minor and major Ar, Py-C-5), 125.8 (major Py-C-3), 125.7 (minor Py-C-3), 90.3 (minor Cp*), 90.0 (major Cp*), 77.7, 72.9, 71.8, 71.3, 68.8 (minor C-1′-C-5′), 77.3, 74.2, 71.2, 69.1, 68.7 (major C-1′-C-5′), 63.0 (minor C-6′), 62.6 (major C-6′), 8.8 (minor Cp*—CH₃), 8.5 (major Cp*—CH₃). ESI-HRMS positive mode (m/z): calcd for C₅₃H₅₃N₃O₁₂Ir⁺ [M−PF₆−Cl+OMe+MeOH]⁺ 1116.3253. Found: [M−PF₆−Cl+OMe+MeOH]⁺ 1116.3248.

### 11.37 Complex Ir-6

Prepared from complex Ir-dimer (20.0 mg, 0.0251 mmol), compound L-6 (29.7 mg, 0.0502 mmol, 2.0 eq.) and TIPF₆ (17.5 mg, 0.0501 mmol) according to general procedure IV. After filtration and removal of the solvent the residue was dissolved in CHCl₃ (5 mL) and Et₂O (10 mL) was added. The precipitated product was filtered off, washed with a solvent mixture of CHCl₃-Et₂O=1:2 (1 mL) to give 34.6 mg (72%) yellow powder. R_{f}: 0.44 (95:5 CHCl₃-MeOH). Diastereomeric ratio: 3:1. ¹H NMR (400 MHz, CDCl₃) δ (ppm): 8.93 (d, J=5.5 Hz, minor Py-H-6), 8.80 (d, J=5.5 Hz, major Py-H-6), 8.23-7.80, 7.57-7.30 (m, minor and major Ar, Py-H-3-Py-H-5), 6.21 (pt, J=9.7, 9.5 Hz, major H-2′ or H-3′), 6.11 (pt, J=9.2, 9.2 Hz, minor H-2′ or H-3′), 6.00 (pt, J=9.6, 9.5 Hz, major H-2′ or H-3′), 5.78 (pt, J=9.2, 9.2 Hz, minor H-2′ or H-3′), 5.59 (ddd, J=10.1, 9.6, 5.4 Hz, minor H-4′), 5.51 (ddd, J=10.4, 9.9, 5.4 Hz, major H-4′), 5.33 (d, J=10.0 Hz, major H-1′), 5.29 (d, J=9.3 Hz, minor H-1′), 4.65 (dd, J=11.6, 5.4 Hz, minor H-5′eq), 4.57 (dd, J=11.3, 5.4 Hz, major H-5′eq), 3.87 (pt, J=11.3, 10.4 Hz, major H-5′ax), 3.86 (pt, J=11.6, 10.1 Hz, minor H-5′ax), 1.63 (s, minor Cp*—CH₃), 1.52 (s, major Cp*—CH₃); ¹³C NMR (90 MHz, CDCl₃) δ (ppm): 169.4, 169.1, 165.8 (2), 165.7, 165.6 (3), 165.5, 165.1 (minor and major C═O, OD-C-2, OD-C-5), 153.5 (minor Py-C-6), 152.4 (major Py-C-6), 140.8 (minor Py-C-4), 140.5 (major Py-C-4), 140.1 (major Py-C-2), 139.2 (minor Py-C-2), 134.3, 133.8, 133.7, 133.5, 131.7, 130.8-128.1 (minor and major Ar, Py-C-5), 125.9 (major Py-C-3), 125.6 (minor Py-C-3), 90.3 (minor Cp*), 90.0 (major Cp*), 73.5, 71.7, 69.5, 69.0 (major C-1′-C-4′), 72.3, 72.2, 70.8, 69.2 (minor C-1′-C-4′), 67.8 (major C-5′), 67.7 (minor C-5′), 8.8 (minor Cp*—CH₃), 8.6 (major Cp*—CH₃). ESI-HRMS positive mode (m/z): calcd for C₄₅H₄₇ClN₃O₁₀Ir⁺ [M−PF₆]⁺ 954.2128. Found: [M−PF₆]⁺ 954.2121.

### 11.38 Complex Ir-8

Prepared from complex Ir-dimer (20.0 mg, 0.0251 mmol), compound L-8 (36.7 mg, 0.0506 mmol, 2.0 eq.) and TIPF₆ (17.5 mg, 0.0501 mmol) according to general procedure IV. After filtration and removal of the solvent the residue was dissolved in CHCl₃ (5 mL) and Et₂O (10 mL) was added. The precipitated product was filtered off, washed with a solvent mixture of CHCl₃-Et₂O=1:1 (2 mL) to give 47.5 mg (77%) yellow powder. R_{f}: 0.60 (9:1 CHCl₃-MeOH). Diastereomeric ratio: 2:1. ¹H NMR (400 MHz, CDCl₃) δ (ppm): 8.94 (d, J=5.5 Hz, minor Py-H-6), 8.79 (d, J=5.5 Hz, major Py-H-6), 8.22-7.79, 7.69-7.24 (m, minor and major Ar, Py-H-3-Py-H-5), 6.54 (pt, J=10.2, 10.1 Hz, major H-2′), 6.19 (dd, J=3.3, 1.0 Hz, major H-4′), 6.17 (dd, J=3.3, 1.0 Hz, minor H-4′), 6.05 (pt, J=10.1, 9.8 Hz, minor H-2′), 5.94 (dd, J=10.1, 3.3 Hz, minor H-3′), 5.75 (dd, J=10.1, 3.3 Hz, major H-3′), 5.50 (d, J=10.2 Hz, major H-1′), 5.44 (d, J=9.8 Hz, minor H-1′), 4.74-4.42 (m, minor and major H-5′, H-6′a, H-6′b), 1.62 (s, minor Cp*—CH₃), 1.51 (s, major Cp*—CH₃); ¹³C NMR (100 MHz, CDCl₃) δ (ppm): 169.4, 166.1, 165.7, 165.5, 165.3, 165.1 (major C═O, OD-C-2, OD-C-5), 169.2, 166.2, 165.7, 165.6, 165.5, 165.4 (minor C═O, OD-C-2, OD-C-5), 153.5 (minor Py-C-6), 152.3 (major Py-C-6), 140.8 (minor Py-C-4), 140.5 (major Py-C-4), 140.2 (major Py-C-2), 139.2 (minor Py-C-2), 134.3, 134.2, 133.8 (2), 133.7, 133.6, 133.5, 133.4, 131.7, 130.7-128.2 (minor and major Ar, Py-C-5), 125.9 (major Py-C-3), 125.7 (minor Py-C-3), 90.3 (minor Cp*), 90.0 (major Cp*), 76.6, 72.1, 71.5, 68.5, 68.2 (minor C-1′-C-5′), 76.2, 72.8, 71.4, 68.3, 66.1 (major C-1′-C-5′), 62.4 (minor C-6′), 62.2 (major C-6′), 8.8 (minor Cp*—CH₃), 8.5 (major Cp*—CH₃). ESI-HRMS positive mode (m/z): calcd for C₅₁H₄₆ClN₃O₁₀Ir⁺ [M−PF₆]⁺ 1088.2491. Found: [M−PF₆]⁺ 1088.2492.

### 11.39 Complex Rh-2a

Prepared from complex Rh-dimer (20.0 mg, 0.0324 mmol), compound L-2a (46.9 mg, 0.0647 mmol, 2.0 eq.) and TIPF₆ (22.6 mg, 0.0647 mmol) according to general procedure IV. After filtration and removal of the solvent the residue was dissolved in CHCl₃ (6 mL) and Et₂O (12 mL) was added. The precipitated product was filtered off, washed with a solvent mixture of CHCl₃-Et₂O=1:1 (2 mL) to give 67.5 mg (91%) orange powder. R_{f}: 0.33 (9:1 CHCl₃-MeOH). Diastereomeric ratio: 2:1. ¹H NMR (400 MHz, CDCl₃) δ (ppm): 9.06 (s, minor Tria-H-5), 9.03 (s, major Tria-H-5), 8.67 (1 signal, minor and major Py-H-6), 8.04-7.82, 7.60-7.22 (m, minor and major Ar, Py-H-3-Py-H-5), 6.53-6.46, 6.25-6.11, 5.99-5.90 (minor and major H-1′-H-4′), 4.77-4.52 (minor and major H-5′, H-6′a, H-6′b), 1.62 (s, major Cp*—CH₃), 1.55 (s, minor Cp*—CH₃); ¹³C NMR (100 MHz, CDCl₃) δ (ppm): 166.3, 165.6, 165.2, 164.6 (minor C═O), 166.2, 165.5, 165.2, 164.7 (major C═O), 151.5 (2) (minor and major Py-C-6), 146.9, 146.8, 146.2 (2) (minor and major Tria-C-4, Py-C-2), 140.4 (minor Py-C-4), 140.2 (major Py-C-4), 134.3, 134.1, 133.9, 133.8, 133.7, 133.5, 133.4, 133.3, 130.2-128.5, 127.9, 127.8, 127.5, 127.4 (minor and major Ar, Py-C-5, Tria-C-5), 122.9 (minor Py-C-3), 122.8 (major Py-C-3), 97.5 (minor Cp*), 97.4 (major Cp*), 87.3 (minor C-1′), 86.8 (major C-1′), 76.1, 72.8, 71.6, 68.7 (minor C-2′-C-5′), 75.7, 73.7, 70.3, 68.6 (major C-2′-C-5′), 62.9 (minor C-6′), 62.7 (major C-6′), 9.0 (minor and major Cp*—CH₃). ESI-HRMS positive mode (m/z): calcd for C₅₁H₄₇ClN₄O₉Rh⁺ [M−PF₆]⁺ 997.2081. Found: [M−PF₆]⁺ 997.2068. 11.40 Complex Rh-4 Prepared from complex Rh-dimer (10.0 mg, 0.0162 mmol), compound L-4 (23.5 mg, 0.0324 mmol, 2.0 eq.) and TIPF₆ (11.3 mg, 0.0323 mmol) according to general procedure IV. After filtration and removal of the solvent the residue was dissolved in CHCl₃ (3 mL) and Et₂O (12 mL) was added. The precipitated product was filtered off, washed with a solvent mixture of CHCl₃-Et₂O=1:2 (4 mL) to give 32.6 mg (88%) orange powder. R_{f}: 0.62 (95:5 CHCl₃-MeOH). Diastereomeric ratio: 3:1. ¹H NMR (400 MHz, CDCl₃) δ (ppm): 8.96 (d, J=5.4 Hz, minor Py-H-6), 8.76 (d, J=5.4 Hz, major Py-H-6), 8.22-7.79, 7.59-7.28 (m, minor and major Ar, Py-H-3-Py-H-5), 6.33 (pt, J=10.0, 9.7 Hz, major H-2′ or H-3′ or H-4′), 6.18 (pt, J=9.6, 9.6 Hz, minor H-2′ or H-3′ or H-4′), 6.04 (pt, J=9.5, 9.4 Hz, major H-2′ or H-3′ or H-4′), 5.85 (2 pt, J=9.7, 9.7 Hz in each, minor and major H-2′ or H-3′ or H-4′), 5.74 (pt, J=9.9, 9.7 Hz, minor H-2′ or H-3′ or H-4′), 5.48 (d, J=10.1 Hz, major H-1′), 5.37 (d, J=10.0 Hz, minor H-1′), 4.73-4.66 (m, minor and major H-6′a), 4.57-4.51 (m, minor and major H-6′b), 4.48-4.40 (m, minor and major H-5′), 1.61 (s, minor Cp*—CH₃), 1.52 (s, major Cp*—CH₃); ¹³C NMR (100 MHz, CDCl₃) δ (ppm): 166.3, 166.2, 165.8, 165.7, 165.3, 165.2, 165.1, 165.0, 164.9 (minor and major C═O, OD-C-2, OD-C-5), 153.6 (minor Py-C-6), 152.1 (major Py-C-6), 140.6 (minor Py-C-4), 140.3 (major Py-C-4), 140.1 (major Py-C-2), 139.0 (minor Py-C-2), 134.6, 133.9, 133.8 (2), 133.7, 133.5, 133.4, 133.3, 131.4-127.8 (minor and major Ar, Py-C-5), 125.5 (major Py-C-3), 125.2 (minor Py-C-3), 98.1, 98.0 (minor Cp*), 97.8, 97.7 (major Cp*), 77.7, 72.8, 71.9, 71.3, 68.8 (minor C-1′-C-5′), 77.2, 74.2, 71.2, 69.1, 68.7 (major C-1′-C-5′), 63.0 (minor C-6′), 62.5 (major C-6′), 9.1 (minor Cp*—CH₃), 8.8 (major Cp*—CH₃). ESI-HRMS positive mode (m/z): calcd for C₅₁H₄₆ClN₃O₁₀Rh⁺ [M−PF₆]⁺ 998.1921. Found: [M−PF₆]⁺ 998.1905.

### 11.41 Complex Rh-6

Prepared from complex Rh-dimer (10.0 mg, 0.0162 mmol), compound L-6 (19.1 mg, 0.0323 mmol, 2.0 eq.) and TIPF₆ (11.3 mg, 0.0323 mmol) according to general procedure IV. After filtration and removal of the solvent the residue was dissolved in CHCl₃ (3 mL) and Et₂O (12 mL) was added. The precipitated product was filtered off, washed with a solvent mixture of CHCl₃-Et₂O=1:1 (2 mL) to give 28.3 mg (87%) orange powder. R_{f}: 0.34 (95:5 CHCl₃-MeOH). Diastereomeric ratio: 5:2. ¹H NMR (400 MHz, CDCl₃) δ (ppm): 8.95 (d, J=5.3 Hz, minor Py-H-6), 8.80 (d, J=5.3 Hz, major Py-H-6), 8.21-7.82, 7.59-7.31 (m, minor and major Ar, Py-H-3-Py-H-5), 6.22 (pt, J=9.6, 9.7 Hz, major H-2′ or H-3′), 6.12 (pt, J=9.3, 9.3 Hz, minor H-2′ or H-3′), 6.00 (pt, J=9.4, 9.3 Hz, major H-2′ or H-3′), 5.75 (pt, J=9.4, 9.3 Hz, minor H-2′ or H-3′), 5.59 (ddd, J=10.7, 9.7, 5.4 Hz, minor H-4′), 5.51 (ddd, J=10.7, 9.7, 5.5 Hz, major H-4′), 5.31 (d, J=10.0 Hz, major H-1′), 5.25 (d, J=9.4 Hz, minor H-1′), 4.64 (dd, J=11.6, 5.4 Hz, minor H-5′eq), 4.56 (dd, J=11.3, 5.5 Hz, major H-5′eq), 3.87 (pt, J=11.3, 10.7 Hz, major H-5′ax), 3.84 (pt, J=11.6, 10.7 Hz, minor H-5′ax), 1.63 (s, minor Cp*—CH₃), 1.53 (s, major Cp*—CH₃); ¹³C NMR (100 MHz, CDCl₃) δ (ppm): 165.8, 165.7, 165.6 (2), 165.5 (2), 165.2, 165.1, 164.9 (2) (minor and major C═O, OD-C-2, OD-C-5), 153.5 (minor Py-C-6), 152.2 (major Py-C-6), 140.6 (minor Py-C-4), 140.4 (major Py-C-4), 140.1 (major Py-C-2), 139.2 (minor Py-C-2), 134.5, 133.9, 133.8, 133.7, 133.5, 131.2-128.0 (minor and major Ar, Py-C-5), 125.5 (major Py-C-3), 125.1 (minor Py-C-3), 98.1, 98.0 (minor Cp*), 97.8, 97.7 (major Cp*), 73.5, 71.7, 69.6, 69.0 (major C-1′-C-4′), 72.4, 72.2, 70.8, 69.2 (minor C-1′-C-4′), 67.7 (2) (minor and major C-5′), 9.1 (minor Cp*—CH₃), 8.8 (major Cp*—CH₃). ESI-HRMS positive mode (m/z): calcd for C₄₃H₄₀ClN₃O₈Rh⁺ [M−PF₆]⁺ 864.1553. Found: [M−PF₆]⁺ 864.1552.

### 11.42 Complex Rh-8

Prepared from complex Rh-dimer (20.0 mg, 0.0324 mmol), compound L-8 (47.0 mg, 0.0648 mmol, 2.0 eq.) and TIPF₆ (22.6 mg, 0.0647 mmol) according to general procedure IV. After filtration and removal of the solvent the residue was dissolved in CHCl₃ (6 mL) and Et₂O (12 mL) was added. The precipitated product was filtered off, washed with a solvent mixture of CHCl₃-Et₂O=1:2 (2 mL) to give 63.0 mg (85%) orange powder. R_{f}: 0.29 (95:5 CHCl₃-MeOH). Diastereomeric ratio: 9:2. ¹H NMR (400 MHz, CDCl₃) δ (ppm): 8.95 (d, J=5.4 Hz, minor Py-H-6), 8.81 (d, J=5.3 Hz, major Py-H-6), 8.22-7.24 (m, minor and major Ar, Py-H-3-Py-H-5), 6.55 (pt, J=10.2, 10.1 Hz, major H-2′), 6.19 (d, J=3.4 Hz, major H-4′), 6.16 (d, J=3.3 Hz, minor H-4′), 6.02 (pt, J=10.1, 9.7 Hz, minor H-2′), 5.94 (dd, J=10.1, 3.3 Hz, minor H-3′), 5.76 (dd, J=10.1, 3.4 Hz, major H-3′), 5.47 (d, J=10.2 Hz, major H-1′), 5.40 (d, J=9.7 Hz, minor H-1′), 4.71-4.40 (m, minor and major H-5′, H-6′a, H-6′b), 1.61 (s, minor Cp*—CH₃), 1.52 (s, major Cp*—CH₃); ¹³C NMR (100 MHz, CDCl₃) δ (ppm): 166.2, 165.8, 165.6, 165.4 (2), 164.9 (minor C═O, OD-C-2, OD-C-5), 166.1, 165.7, 165.5, 165.3, 165.2, 165.0 (major C═O, OD-C-2, OD-C-5), 153.5 (minor Py-C-6), 152.2 (major Py-C-6), 140.6 (minor Py-C-4), 140.4 (major Py-C-4), 140.1 (major Py-C-2), 139.2 (minor Py-C-2), 134.5, 134.2, 133.8 (2), 133.6, 133.5 (2), 131.2-128.1 (minor and major Ar, Py-C-5), 125.5 (major Py-C-3), 125.2 (minor Py-C-3), 98.1, 98.0 (minor Cp*), 97.8, 97.7 (major Cp*), 76.6, 72.2, 71.5, 68.5, 68.3 (minor C-1′-C-5′), 76.1, 72.8, 71.4, 68.3, 66.2 (major C-1′-C-5′), 62.4 (minor C-6′), 62.1 (major C-6′), 9.0 (minor Cp*—CH₃), 8.8 (major Cp*—CH₃). ESI-HRMS positive mode (m/z): calcd for C₅₁H₄₆ClN₃O₁₀Rh⁺ [M−PF₆]⁺ 998.1921. Found: [M−PF₆]⁺ 998.1920.

### 11.43 1-(2′,3′,4′,6′-Tetra-O-butanoyl-β-D-glucopyranosyl)-4-(pyridin-2-yl)-1,2,3-triazole (L-C3)

Prepared from triazole 11[[?]](0.050 g, 0.16 mmol) and butyryl chloride (2×81 μL, 2×0.78 mmol) according to general procedure I. Reaction time: 3 h. Purified by column chromatography (1:3 EtOAc-hexane) to give 82 mg (87%) white amorphous solid. R_{f}=0.47 (1:2 EtOAc-hexane). ¹H NMR (360 MHz, CDCl₃) δ (ppm): 8.61 (1H, d, J=4.7 Hz, Py-H-6), 8.43 (1H, s, Tria-H-5), 8.13 (1H, d, J=7.9 Hz, Py-H-3), 7.77 (1H, t, J=7.6 Hz, Py-H-4), 7.25 (1H, dd, J=7.6, 4.7 Hz, Py-H-5), 5.97 (1H, d, J=8.7 Hz, H-1′), 5.54, 5.50, 5.32 (3×1H, 3 pt, J=9.6, 9.5 Hz in each, H-2′, H-3′, H-4′), 4.30 (1H, dd, J=12.6, 4.8 Hz, H-6′a), 4.18 (1H, dd, J=12.6, <1 Hz, H-6′b), 4.05 (1H, ddd, J=9.6, 4.8, <1 Hz, H-5′), 2.36-2.23 (6H, m, 3×CH₂), 2.11 (2H, t, J=7.4 Hz, CH₂), 1.70-1.53 (6H, m, 3×CH₂), 1.41 (2H, sext, J=7.4 Hz, CH₂), 0.96-0.88 (9H, m, 3×CH₃), 0.72 (3H, t, J=7.4 Hz, CH₃); ¹³C N M R (90 MHz, CDCl₃) δ (ppm): 173.0, 172.3, 171.8, 171.3 (4×C═O), 149.5, 148.8 (Tria-C-4, Py-C-2), 149.5 (Py-C-6), 136.8, 123.0, 120.2 (Py-C-3, Py-C-4, Py-C-5), 120.5 (Tria-C-5), 85.9 (C-1′), 75.2, 72.2, 70.1, 67.3 (C-2′-C-5′), 61.2 (C-6′), 35.7 (2), 35.6, 35.3 (4×CH₂), 18.1, 18.0 (2), 17.9 (4×CH₂), 13.5 (2), 13.4, 13.2 (4×CH₃). ESI-HRMS positive mode (m/z): cald for: C₂₉H₄₀N₄O₉Na⁺ [M+Na]⁺ 611.2686. Found: 611.2687.

### 11.44 1-(2′,3′,4′,6′-Tetra-O-pentanoyl-β-D-glucopyranosyl)-4-(pyridin-2-yl)-1,2,3-triazole (L-C4)

Prepared from triazole 11[[?]](0.050 g, 0.16 mmol) and valeryl chloride (2×95 μL, 2×0.78 mmol) according to general procedure I. Reaction time: 1.5 h. Purified by column chromatography (1:3 EtOAc-hexane) to give 57 mg (55%) pale yellow amorphous solid. R_{f}=0.58 (3:7 EtOAc-hexane). ¹H NMR (360 MHz, CDCl₃) δ (ppm): 8.61 (1H, d, J=4.5 Hz, Py-H-6), 8.41 (1H, s, Tria-H-5), 8.14 (1H, d, J=7.9 Hz, Py-H-3), 7.78 (1H, dt, J=7.9, 1.5 Hz, Py-H-4), 7.25 (1H, ddd, J=7.9, 4.5, 1.5 Hz, Py-H-5), 5.95 (1H, d, J=8.7 Hz, H-1′), 5.53, 5.49, 5.31 (3×1H, 3 pt, J=9.5, 9.4 Hz in each, H-2′, H-3′, H-4′), 4.28 (1H, dd, J=12.6, 4.8 Hz, H-6′a), 4.18 (1H, dd, J=12.6, 1.2 Hz, H-6′b), 4.03 (1H, ddd, J=9.6, 4.8, 1.2 Hz, H-5′), 2.38-2.24 (6H, m, 3×CH₂), 2.13 (2H, t, J=7.4 Hz, CH₂), 1.64-1.24 (14H, m, 7×CH₂), 1.36 (2H, sext, J=7.3 Hz, CH₂), 0.94-0.87 (9H, m, 3×CH₃), 0.75 (3H, t, J=7.3 Hz, CH₃); ¹³C NMR (90 MHz, CDCl₃) δ (ppm): 173.3, 172.6, 172.0, 171.6 (4×C═O), 149.5, 148.9 (Tria-C-4, Py-C-2), 149.4 (Py-C-6), 136.8, 123.1, 120.2 (Py-C-3, Py-C-4, Py-C-5), 120.5 (Tria-C-5), 85.9 (C-1′), 75.2, 72.2, 70.2, 67.3 (C-2′-C-5′), 61.3 (C-6′), 33.6, 33.5 (2), 33.3 (4×CH₂), 26.7 (3), 26.5 (4×CH₂), 22.1 (3), 21.8 (4×CH₂), 13.6, 13.5 (2), 13.4 (4×CH₃). ESI-HRMS positive mode (m/z): cald for: C₃₃H₄₈N₄O₉Na⁺ [M+Na]⁺ 667.3313. Found: 667.3311.

### 11.45 1-(2′,3′,4′,6′-Tetra-O-hexanoyl-β-D-glucopyranosyl)-4-(pyridin-2-yl)-1,2,3-triazole (L-C5)

Prepared from triazole 11[[?]](0.050 g, 0.16 mmol) and hexanoyl chloride (2×110 μL, 2×0.78 mmol) according to general procedure I. Reaction time: 3 h. Purified by column chromatography (1:4 EtOAc-hexane) to give 80 mg (71%) pale yellow amorphous solid. R_{f}=0.69 (3:7 EtOAc-hexane). ¹H NMR (360 MHz, CDCl₃) δ (ppm): 8.61 (1H, d, J=4.9 Hz, Py-H-6), 8.40 (1H, s, Tria-H-5), 8.14 (1H, d, J=7.9 Hz, Py-H-3), 7.78 (1H, dt, J=7.7, 1.7 Hz, Py-H-4), 7.25 (1H, ddd, J=7.7, 4.9, 1<Hz, Py-H-5), 5.94 (1H, d, J=8.9 Hz, H-1′), 5.52, 5.47, 5.29 (3×1H, 3 pt, J=9.6, 9.5 Hz in each, H-2′, H-3′, H-4′), 4.27 (1H, dd, J=12.6, 4.7 Hz, H-6′a), 4.18 (1H, dd, J=12.6, 1.9 Hz, H-6′b), 4.03 (1H, ddd, J=9.6, 4.7, <1 Hz, H-5′), 2.36-2.23 (6H, m, 3×CH₂), 2.12 (2H, t, J=7.5 Hz, CH₂), 1.65-1.20, (20H, m, 10×CH₂), 1.19-1.11 (2H, m, CH₂), 1.07-1.01 (2H, m, CH₂), 0.92-0.86 (9H, m, 3×CH₃), 0.75 (3H, t, J=7.3 Hz, CH₃); ¹³C NMR (90 MHz, CDCl3) δ (ppm): 173.3, 172.6, 172.0, 171.6 (4×C═O), 149.6, 148.9 (Tria-C-4, Py-C-2), 149.5 (Py-C-6), 136.8, 123.1, 120.3 (Py-C-3, Py-C-4, Py-C-5), 120.5 (Tria-C-5), 85.9 (C-1′), 75.3, 72.3, 70.2, 67.4 (C-2′-C-5′), 61.3 (C-6′), 33.9, 33.8 (2), 33.5 (4×CH₂), 31.2 (2), 31.1, 30.8 (4×CH₂), 24.3 (3), 24.2 (4×CH₂), 22.2 (2), 22.1, 22.0 (4×CH₂), 13.9, 13.8, 13.7, 13.6 (4×CH₃). ESI-HRMS positive mode (m/z): cald for: C₃₇H₅₆N₄O₉Na⁺ [M+Na]⁺ 723.3940. Found: 723.3937.

### 11.46 1-(2′,3′,4′,6′-Tetra-O-heptanoyl-β-D-glucopyranosyl)-4-(pyridin-2-yl)-1,2,3-triazole (L-C6)

Prepared from triazole 11[[?]](0.050 g, 0.16 mmol) and heptanoyl chloride (2×110 L, 2×0.71 mmol) according to general procedure I. Reaction time: 2 h. Purified by column chromatography (2:7 EtOAc-hexane) to give 97 mg (80%) yellow amorphous solid. R_{f}=0.51 (3:7 EtOAc-hexane). ¹H NMR (360 MHz, CDCl₃) δ (ppm): 8.61 (1H, d, J=4.2 Hz, Py-H-6), 8.42 (1H, s, Tria-H-5), 8.14 (1H, d, J=7.8 Hz, Py-H-3), 7.77 (1H, t, J=7.8 Hz, Py-H-4), 7.26-7.23 (1H, m, Py-H-5), 5.97 (1H, d, J=8.6 Hz, H-1′), 5.54, 5.49, 5.31 (3×1H, 3 pt, J=9.5, 9.4 Hz in each, H-2′, H-3′, H-4′), 4.29 (1H, dd, J=12.6, 4.7 Hz, H-6′a), 4.17 (1H, dd, J=12.6, <1 Hz, H-6′b), 4.07-4.04 (1H, m, H-5′), 2.37-2.24 (6H, m, 3×CH₂), 2.12 (2H, t, J=7.4 Hz, CH₂), 1.61-0.87 (41H, m, 16×CH₂, 3×CH₃), 0.78 (3H, t, J=7.3 Hz, CH₃); ¹³C NMR (90 MHz, CDCl₃) δ (ppm): 173.2, 172.5, 171.9, 171.5 (4×C═O), 149.5, 148.8 (Tria-C-4, Py-C-2), 149.4 (Py-C-6), 136.7, 123.0, 120.2 (Py-C-3, Py-C-4, Py-C-5), 120.4 (Tria-C-5), 85.8 (C-1′), 75.2, 72.2, 70.1, 67.3 (C-2′-C-5′), 61.2 (C-6′), 33.9, 33.8 (2), 33.5 (4×CH₂), 31.3 (3), 31.1 (4×CH₂), 28.6 (3), 28.3 (4×CH₂), 24.6, 24.5 (2), 24.4 (4×CH₂), 22.3, 22.3 (2), 22.2 (4×CH₂), 13.9 (3), 13.8 (4×CH₃). ESI-HRMS positive mode (m/z): cald for: C₄₁H₆₄N₄O₉Na⁺ [M+Na]⁺ 779.4566. Found: 779.4560.

### 11.47 1-(2′,3′,4′,6′-Tetra-O-octanoyl-β-D-glucopyranosyl)-4-(pyridin-2-yl)-1,2,3-triazole (L-C7)

Prepared from triazole 11[[?]](0.051 g, 0.17 mmol) and octanoyl chloride (2×135 μL, 2×0.78 mmol) according to general procedure I. Reaction time: 2.5 h. Purified by column chromatography (1:9 EtOAc-hexane) to give 105 mg (81%) pale yellow amorphous solid. R_{f}=0.45 (3:7 EtOAc-hexane). ¹H NMR (360 MHz, CDCl₃) δ (ppm): 8.62 (1H, dd, J=4.9, 1.6 Hz, Py-H-6), 8.46 (1H, s, Tria-H-5), 8.16 (1H, d, J=7.9 Hz, Py-H-3), 7.78 (1H, dt, J=7.9, 1.6 Hz, Py-H-4), 7.26 (1H, ddd, J=7.5, 4.9, <1 Hz, Py-H-5), 5.95 (1H, d, J=8.9 Hz, H-1′), 5.54, 5.48, 5.30 (3×1H, 3 pt, J=9.9, 9.5 Hz in each, H-2′, H-3′, H-4′), 4.28 (1H, dd, J=12.6, 4.9 Hz, H-6′a), 4.18 (1H, dd, J=12.6, 1.8 Hz, H-6′b), 4.04 (1H, ddd, J=9.9, 4.9, <1 Hz, H-5′), 2.40-2.23 (6H, m, 3×CH₂), 2.12 (2H, t, J=7.4 Hz, CH₂), 1.71-0.78 (60H, m, 24×CH₂, 4×CH₃); ¹³C NMR (90 MHz, CDCl₃) δ (ppm): 173.3, 172.6, 171.9, 171.5 (4×C═O), 149.4, 148.5 (Tria-C-4, Py-C-2), 149.2 (Py-C-6), 137.0, 123.1, 120.4 (Py-C-3, Py-C-4, Py-C-5), 120.7 (Tria-C-5), 85.9 (C-1′), 75.3, 72.3, 70.1, 67.3 (C-2′-C-5′), 61.3 (C-6′), 34.0, 33.9, 33.8, 33.5 (4×CH₂), 31.6, 31.5 (2), 31.4 (4×CH₂), 29.0 (3), 28.8 (3), 28.7, 28.6 (8×CH₂), 24.7, 24.6, 24.6, 24.5 (4×CH₂), 22.5 (3), 22.4 (4×CH₂), 14.0 (3), 13.9, (4×CH₃). ESI-HRMS positive mode (m/z): cald for: C₄₅H₇₂N₄O₉Na⁺ [M+Na]⁺ 835.5192. Found: 835.5192.

### 11.48 1-(2′,3′,4′,6′-Tetra-O-benzoyl-β-D-glucopyranosyl)-4-(quinolin-2-yl)-1,2,3-triazole (L-2b)

Prepared from triazole 12 [[?42)]](100 mg, 0.279 mmol) and benzoyl chloride (157 μL, 1.35 mmol) according to general procedure I. Reaction time: 2 h. Purified by column chromatography (2:3 EtOAc-hexane) to give 207 mg (96%) white amorphous solid. R_{f}=0.40 (2:3 EtOAc-hexane). ¹H NMR (400 MHz, CDCl₃) δ (ppm): 8.80 (H, s, Tria-H-5), 8.27-7.26 (26H, m, Ar, Qu), 6.37 (1H, d, J=8.8 Hz, H-1′), 6.17 (1H, pt, J=9.5, 9.2 Hz, H-2′ or H-3′ or H-4′), 6.11 (1H, pt, J=9.4, 9.0 Hz, H-2′ or H-3′ or H-4′), 5.92 (1H, pt, J=9.5, 9.4 Hz, H-2′ or H-3′ or H-4′), 4.72-4.50 (3H, m, H-5′, H-6′a, H-6′b); ¹³C NMR (100 MHz, CDCl₃) δ (ppm): 166.2, 165.8, 165.3, 164.8 (4×C═O), 149.9, 149.4, 148.2 (Tria-C-4, Qu-C-2, Qu-C-8a), 137.1, 133.8, 133.7, 133.6, 133.4, 130.3-128.5, 128.1, 128.0, 127.8, 126.7, 121.7, 118.9 (Tria-C-5, Qu-C-3-Qu-C-8, Qu-C-4a, Ar), 86.5 (C-1′), 75.8, 73.2, 71.4, 69.0 (C-2′-C-5′), 62.9 (C-6′). ESI-HRMS positive mode (m/z): calcd for: C₄₅H₃₅N₄O₉⁺ [M+H]⁺ 775.2399; C₄₅H₃₄N₄O₉Na⁺ [M+Na]⁺ 797.2218. Found: [M+H]⁺ 775.2395; [M+Na]⁺ 797.2211.

### 11.49 Complex Os-C3

Prepared from ligand L-C3 (30.4 mg, 0.052 mmol), Os-dimer (21.0 mg, 0.026 mmol) and TIPF₆ (17.7 mg, 0.051 mmol) according to general procedure II. Reaction time: 1 h. Purified by column chromatography (95:5 CHCl₃-MeOH) to yield 56 mg (96%) yellow syrup. Diastereomeric ratio: 1:1. R_{f}=0.34 (95:5 CHCl₃-MeOH). ¹H NMR (400 MHz, CDCl₃) δ (ppm): 9.25, 9.23 (2×1H, 2 d, J=6.5 Hz in each, 2×Py-H-6), 9.02, 8.89 (2×1H, 2 s, 2×Tria-H-5), 8.04-8.01 (2H, m, 2×Py-H-3), 7.99-7.94 (2H, m, 2×Py-H-4), 7.55, 7.52 (2×1H, 2 dd, J=7.4, 6.5 Hz, 2×Py-H-5), 6.20-5.86 (12H, m, 2×H-1′, 2×(H-2′ or H-3′ or H-4′), 2×4×p-cym-CH), 5.50, 5.48, 5.39, 5.37 (2×2H, 2×2 pt, J=9.4, 9.2 Hz in each, H-2′ and/or H-3′ and/or H-4′), 4.38-4.14 (6H, m, 2×H-5′, 2×H-6′a, 2×H-6′b), 2.63, 2.59 (2×1H, 2 hept, J=6.9 Hz in each, 2×i-Pr—CH), 2.37-0.76 (74H, m, 16×CH₂, 14×CH₃); ¹³C NMR (90 MHz, CDCl₃) δ (ppm): 173.3, 173.2, 172.4, 172.3, 172.1, 172.0, 171.9, 171.6 (2×4×C═O), 155.7, 155.6 (2×Py-C-6), 148.4, 148.3 (2), 147.9 (2×Tria-C-4, 2×Py-C-2), 140.5, 140.4 (2×Py-C-4), 129.8 (2) (2×Tria-C-5), 127.7, 127.6 (2×Py-C-5), 122.6 (2) (2×Py-C-3), 96.9, 96.5, 95.7, 94.4 (2×2×p-cym-C_{qAr}), 87.0, 86.6 (2×C-1′), 78.4, 77.2, 77.0, 76.8, 75.7, 75.6, 75.5, 74.8, 74.4, 73.6, 72.9, 72.6, 70.1, 69.3, 67.3, 67.1 (2×(C-2′-C-5′), 2×4×p-cym-CH_{Ar}), 61.3, 61.2 (2×C-6′), 35.9 (4), 35.8 (2), 35.7, 35.4 (2×4×CH₂), 31.2, 31.1 (2×i-Pr—CH), 22.9, 22.5, 22.3, 21.9 (2×2×i-Pr—CH₃), 18.6, 18.5 (2×C₆H₄—CH₃), 18.4, 18.3 (4), 18.2, 18.1 (2) (2×4×CH₂), 13.7 (3), 13.6 (3), 13.6, 13.5 (2×4×CH₃). ESI-HRMS positive mode (m/z): cald for: C39H54ClN4O9Os⁺ [M−PF₆]⁺ 949.3180. Found: 949.3180.

### 11.50 Complex Os-C4

Prepared from ligand L-C4 (33.1 mg, 0.052 mmol), Os-dimer (20.0 mg, 0.025 mmol) and TIPF₆ (17.6 mg, 0.050 mmol) according to general procedure II. Reaction time: 1 h. Purified by column chromatography (95:5 CHCl₃-MeOH) to yield 52 mg (88%) yellow syrup. Diastereomeric ratio: 4:3. R_{f}=0.38 (95:5 CHCl₃-MeOH). ¹H NMR (400 MHz, CDCl₃) δ (ppm): 9.25, 9.23 (2×1H, 2 d, J=6.6 Hz in each, 2×Py-H-6), 9.03, 8.90 (2×1H, 2 s, 2×Tria-H-5), 8.04-8.01 (2H, m, 2×Py-H-3), 7.99-7.94 (2H, m, 2×Py-H-4), 7.55-7.50 (2H, m, 2×Py-H-5), 6.20-5.86 (12H, m, 2×H-1′, 2×(H-2′ or H-3′ or H-4′), 2×4×p-cym-CH), 5.51, 5.47, 5.40, 5.35 (2×2H, 2×2 pt, J=9.4, 9.2 Hz in each, H-2′ and/or H-3′ and/or H-4′), 4.38-4.14 (6H, m, 2×H-5′, 2×H-6′a, 2×H-6′b), 2.67-2.55 (2H, m, 2×i-Pr—CH), 2.40- 0.77 (90H, m, 24×CH₂, 14×CH₃); ¹³C NMR (90 MHz, CDCl₃) δ (ppm): 173.4, 173.3, 172.5, 172.4, 172.2, 172.1, 172.0, 171.7 (2×4×C═O), 155.7, 155.5 (2×Py-C-6), 148.3, 148.2 (2), 147.8 (2×Tria-C-4, 2×Py-C-2), 140.4, 140.3 (2×Py-C-4), 129.8, 129.6 (2×Tria-C-5), 127.6, 127.5 (2×Py-C-5), 122.6 (2) (2×Py-C-3), 96.8, 96.4, 95.8, 94.3 (2×2×p-cym-C_{qAr}), 86.9, 86.5 (2×C-1′), 78.4, 76.9 (3), 75.7 (2), 75.6, 74.7, 74.3, 73.4, 72.8, 72.5, 70.0, 69.3, 67.2, 67.0 (2×(C-2′-C-5′), 2×4×p-cym-CH_{Ar}), 61.3, 61.1 (2×C-6′), 33.7 (3), 33.6 (2), 33.5, 33.3 (2) (2×4×CH₂), 31.1, 31.0 (2×i-Pr—CH), 26.8 (2), 26.8 (2), 26.7 (2), 26.6, 26.6 (2×4×CH₂), 22.9, 22.4, 22.3, 21.8 (2×2×i-Pr—CH₃), 22.2 (4), 22.1 (2), 22.0 (2) (2×4×CH₂), 18.6 (2) (2×C₆H₄—CH₃), 13.8 (3), 13.7 (3), 13.6 (2), (2×4×CH₃). ESI-HRMS positive mode (m/z): cald for: C₄₃H₆₂ClN₄O₉Os⁺ [M−PF₆]⁺ 1005.3807. Found: 1005.3806.

### 11.51 Complex Os-C5

Prepared from ligand L-C5 (18.6 mg, 0.027 mmol), Os-dimer (10.0 mg, 0.013 mmol) and TIPF₆ (8.8 mg, 0.025 mmol) according to general procedure II. Reaction time: 1 h. Purified by column chromatography (95:5 CHCl₃-MeOH) to yield 25 mg (80%) yellow syrup. Diastereomeric ratio: 5:4. R_{f}=0.40 (95:5 CHCl₃-MeOH). ¹H NMR (400 MHz, CDCl₃) δ (ppm): 9.22, 9.20 (2×1H, 2 d, J=6.4 Hz in each, 2×Py-H-6), 8.99, 8.86, (2×1H, 2 s, 2×Tria-H-5), 8.03-8.00 (2H, m, 2×Py-H-3), 7.97-7.93 (2H, m, 2×Py-H-4), 7.53-7.48 (2×1H, m, 2×Py-H-5), 6.18-5.86 (12H, m, 2×H-1′, 2×H-2′ or H-3′ or H-4′, 2×4×p-cym-CH), 5.49, 5.47 (2×1H, 2 pt, J=9.1, 9.1 Hz in each, H-2′ or H-3′ or H-4′), 5.37, 5.35 (2×1H, 2 pt, J=10.1, 10.1 Hz in each, H-2′ or H-3′ or H-4′), 4.39-4.13 (6H, m, 2×H-5′, 2×H-6′a, 2×H-6′b), 2.69-2.56 (2H, m, 2×i-Pr—CH), 2.38-0.78 (106H, m, 32×CH₂, 14×CH₃); ¹³C NMR (90 MHz, CDCl₃) δ (ppm): 173.5, 173.4, 172.6, 172.4, 172.3, 172.2, 172.1, 171.8 (2×4×C═O), 155.6, 155.4 (2×Py-C-6), 148.4 (3), 147.8 (2×Tria-C-4, 2×Py-C-2), 140.5, 140.4 (2×Py-C-4), 127.7, 127.5 (2×Py-C-5), 126.1, 125.8 (2×Tria-C-5), 122.8, 122.7 (2×Py-C-3), 97.0, 96.5, 95.7, 94.3 (2×2×p-cym-C_{qAr}), 87.1, 86.7 (2×C-1′), 78.5, 77.2, 77.0, 76.8, 75.9, 75.8, 75.7, 74.9, 74.5, 73.6, 72.9, 72.6, 70.1, 69.4, 67.3, 67.2 (2×(C-2′-C-5′), 2×4×p-cym-CH_{Ar}), 61.4, 61.2 (2×C-6′), 34.1 (4), 34.9 (2), 34.8, 33.6 (2×4×CH₂), 31.4 (4), 31.3 (3), 31.2 (2×4×CH₂), 31.2 (2) (2×i-Pr—CH), 24.5 (3), 24.4 (2). 24.3 (3) (2×4×CH₂), 22.9, 22.5, 22.4, 21.9 (2×2×i-Pr—CH₃), 22.4 (4), 22.3 (2), 22.2 (2) (2×4×CH₂), 18.7, 18.6 (2×C₆H₄—CH₃), 14.0 (2), 13.9 (4), 13.8 (2) (2×4×CH₃). ESI-HRMS positive mode (m/z): cald for: C₄₇H₇₀ClN₄O₉Os⁺ [M−PF₆]⁺ 1061.4433. Found: 1061.4432.

### 11.52 Complex Os-C6

Prepared from ligand L-C6 (38.4 mg, 0.051 mmol), Os-dimer (20.2 mg, 0.025 mmol) and TIPF₆ (17 mg, 0.049 mmol) according to general procedure II. Reaction time: 1 h. Purified by column chromatography (95:5 CHCl₃-MeOH) to yield 34 mg (53%) yellow syrup. Diastereomeric ratio: 9:7. R_{f}=0.48 (95:5 CHCl₃-MeOH). ¹H NMR (400 MHz, CDCl₃) δ (ppm): 9.23, 9.21 (2×1H, 2 d, J=6.6 Hz in each, 2×Py-H-6), 9.02, 8.88 (2×1H, 2 s, 2×Tria-H-5), 8.03, 8.01 (2×1H, 2 d, J=7.3 Hz, 2×Py-H-3), 7.95, 7.95 (2×1H, 2 t, J=7.7 Hz, 2×Py-H-4), 7.53-7.49 (2H, m, 2×Py-H-5), 6.18-5.85 (12H, m, 2×H-1′, 2×(H-2′ or H-3′ or H-4′), 2×4×p-cym-CH), 5.50, 5.45 (2×1H, 2 pt, J=9.1, 9.1 Hz in each, H-2′ or H-3′ or H-4′), 5.39, 5.34 (2×1H, 2 pt, J=10.1, 10.1 Hz in each, H-2′ or H-3′ or H-4′), 4.39-4.13 (6H, m, 2×H-5′, 2×H-6′a, 2×H-6′b), 2.69-2.56 (2×1H, m, 2×i-Pr—CH), 2.38-0.78 (122H, m, 40×CH₂, 14×CH₃); ¹³C NMR (90 MHz, CDCl₃) δ (ppm): 173.4, 173.3, 172.5, 172.4, 172.2, 172.1, 172.1, 171.7 (2×4×C═O), 155.5, 155.3 (2×Py-C-6), 148.3, 148.2, 147.7 (2) (2×Tria-C-4, 2×Py-C-2), 140.4, 140.3 (2×Py-C-4), 129.8, 129.7 (2×Tria-C-5), 127.5, 127.4 (2×Py-C-5), 122.7, 122.6 (2×Py-C-3), 96.9, 96.4, 95.6, 94.2 (2×2×p-cym-C_{qAr}), 87.0, 86.6 (2×C-1′), 78.4, 76.9, 75.8, 75.7, 75.7, 75.6, 74.8, 74.4, 73.5, 72.8, 72.6 (2), 70.0, 69.3, 67.2, 67.0 (2×(C-2′-C-5′), 2×4×p-cym-CH_{Ar}), 61.3, 61.1 (2×C-6′), 34.0 (3), 33.9 (2), 33.8, 33.6 (2) (2×4×CH₂), 31.5 (2), 31.4 (4), 31.4, 31.3 (2×4×CH₂), 31.1, 31.0 (2×i-Pr—CH), 28.8 (2), 28.8 (4), 28.7, 28.6 (2×4×CH₂), 24.8 (2), 24.7, 24.7 (2), 24.6, 24.6, 24.5 (2×4×CH₂), 22.9, 22.3 (2), 21.9 (2×2×i-Pr—CH₃), 22.5 (4), 22.4 (4), (2×4×CH₂), 18.6 (2) (2×C₆H₄—CH₃), 14.1 (3), 14.0 (4), 14.0 (2×4×CH₃). ESI-HRMS positive mode (m/z): cald for: C₅₁H₇₈ClN₄O₉Os⁺ [M−PF₆]⁺ 1117.5060. Found: 1117.5059.

### 11.53 Complex Os-C7

Prepared from ligand L-C7 (42.2 mg, 0.052 mmol), Os-dimer (20.0 mg, 0.025 mmol) and TIPF₆ (17.0 mg, 0.049 mmol) according to general procedure II. Reaction time: 1 h. Purified by column chromatography (95:5 CHCl₃-MeOH) to yield 48 mg (72%) yellow syrup. Diastereomeric ratio: 5:4. R_{f}=0.38 (95:5 CHCl₃-MeOH). ¹H NMR (400 MHz, CDCl₃) δ (ppm): 9.24, 9.23 (2×1H, 2 d, J=6.2 Hz in each, 2×Py-H-6), 9.03, 8.90 (2×1H, 2 s, 2×Tria-H-5), 8.04-8.01 (2H, m, 2×Py-H-3), 7.95, 7.95 (2×1H, 2 pt, J=7.4 Hz in each, 2×Py-H-4), 7.54-7.49 (2H, m, 2×Py-H-5), 6.19-5.86 (12H, m, 2×H-1′, 2×(H-2′ or H-3′ or H-4′), 2×4×p-cym-CH), 5.50, 5.46 (2×1H, 2 pt, J=9.4, 9.5 Hz in each, H-2′ or H-3′ or H-4′), 5.39, 5.34 (2×1H, 2 pt, J=9.9, 10.0 Hz in each, H-2′ or H-3′ or H-4′), 4.40-4.15 (6H, m, 2×H-5′, 2×H-6′a, 2×H-6′b), 2.62, 2.60 (2×1H, 2 hept, J=6.9 Hz in each, 2×i-Pr—CH), 2.38-0.81 (138H, m, 48×CH₂, 14×CH³); ¹³C NMR (90 MHz, CDCl₃) δ (ppm): 173.4, 173.3, 172.5, 172.4, 172.2, 172.1, 172.0, 171.8 (2×4×C═O), 155.7, 155.6 (2×Py-C-6), 148.3, 148.2 (2), 147.8 (2×Tria-C-4, 2×Py-C-2), 140.4, 140.3 (2×Py-C-4), 129.8, 129.7 (2×Tria-C-5), 127.6, 127.5 (2×Py-C-5), 122.7, 122.6 (2×Py-C-3), 96.9, 96.4, 95.8, 94.3 (2×2×p-cym-C_{qAr}), 87.0, 86.6 (2×C-1′), 78.4, 77.0, 76.9, 76.7, 75.7 (2), 75.6, 74.8, 74.4, 73.4, 72.8, 72.6, 70.0, 69.3, 67.2, 67.0 (2×(C-2′-C-5′), 2×4×p-cym-CH_{Ar}), 61.3, 61.1 (2×C-6′), 34.0 (2), 34.0 (2), 33.9 (2), 33.8, 33.6 (2×4×CH₂), 31.7 (2), 31.7 (4), 31.6, 31.6 (2×4×CH₂), 31.1, 31.0 (2×i-Pr—CH), 29.1, 29.1 (2), 29.0, 29.0, 28.9, 28.9, 28.8 (2×4×CH₂), 24.8 (2), 24.8 (2), 24.7, 24.7, 24.6, 24.6 (2×4×CH₂), 22.9, 22.4 (2), 22.3, 21.9 (2×2×i-Pr—CH₃), 22.6 (5), 22.6, 22.5 (2×4×CH₂), 18.6 (2) (2×C₆H₄—CH₃), 14.1 (8) (2×4×CH₃). ESI-HRMS positive mode (m/z): cald for: C₅₅H₈₆ClN₄O₉Os⁺ [M−PF₆]⁺ 1173.5686. Found: 1173.5687.

### 11.53 Complex Ru-2b

Prepared from ligand L-7 (26.6 mg, 0.0343 mmol, 2.1 eq.), Ru-dimer (10 mg, 0.0163 mmol) and TIPF₆ (11.4 mg, 0.0326 mmol) according to general procedure II. After filtration and removal of the solvent, the residue was dissolved in CHCl₃ (3 mL) and Et₂O (6 mL) was added. The precipitated product was filtered off, then washed with a solvent mixture of CHCl₃-Et₂O=1:1 (1 mL) to give 26.2 mg (67%) brown powder. Diastereomeric ratio: 2:1. R_{f}=0.42 (95:5 CHCl₃-MeOH). ¹H NMR (400 MHz, CDCl₃) δ (ppm): 9.32 (s, minor Tria-H-5), 9.28 (s, major Tria-H-5), 8.63 (d, J=8.8 Hz, major Qu-H-8), 8.60 (d, J=8.9 Hz, minor Qu-H-8), 8.27 (d, J=8.5 Hz, major Qu-H-4), 8.26 (d, J=8.4 Hz, minor Qu-H-4), 8.07-7.28 (m, minor and major Qu-H-3, Qu-H-5-Qu-H-7, Ar), 6.66 (pt, J=9.3, 9.3 Hz, major H-2′ or H-3′ or H-4′), 6.57 (d, J=9.3 Hz, major H-1′), 6.53 (d, J=8.8 Hz, minor H-1′), 6.28 (pt, J=9.5, 9.5 Hz, minor H-2′ or H-3′ or H-4′), 6.17 (pt, J=9.5, 9.4 Hz, major H-2′ or H-3′ or H-4′), 6.16 (pt, J=9.5, 9.2 Hz, minor H-2′ or H-3′ or H-4′), 6.01 (pt, J=10.0, 9.6 Hz, minor H-2′ or H-3′ or H-4′), 5.95 (pt, J=10.0, 9.6 Hz, major H-2′ or H-3′ or H-4′), 5.72- 5.45 (m, minor and major 4×p-cym-CH_{Ar}), 4.83-4.55 (m, minor and major H-5′, H-6′a, H-6′b), 2.35 (hept, J=6.9 Hz, minor i-Pr—CH), 2.12 (hept, J=6.9 Hz, major i-Pr—CH), 2.10 (s, major C₆H₄—CH₃), 2.02 (s, minor C₆H₄—CH₃), 0.85, 0.81 (2 d, J=6.9 Hz in each, 2×minor i-Pr—CH₃), 0.69, 0.60 (2 d, J=6.9 Hz in each, 2×major i-Pr—CH₃); ¹³C NMR (100 MHz, CDCl₃) δ (ppm): 166.3, 165.6, 165.2, 165.0 (minor 4×C═O), 166.2, 165.5, 165.3, 165.2 (major 4×C═O), 149.2, 148.4, 147.5 (major Tria-C-4, Qu-C-2, Qu-C-8a), 149.1, 148.3, 147.8 (minor Tria-C-4, Qu-C-2, Qu-C-8a), 141.1, 134.3, 133.8 133.5, 133.3, 133.0, 130.6-128.0, 118.8, 118.7 (minor and major Tria-C-5, Qu-C-5-Qu-C-8, Qu-C-4a, Ar), 105.6, 103.0 (minor p-cym-C_{qAr}), 105.6, 103.6 (major p-cym-C_{qAr}), 87.3, 87.2, 87.0, 86.7, 85.1, 85.0, 84.5, 83.4, 82.8 (minor and major p-cym-CH_{Ar}, C-1′), 76.2, 72.7, 72.2, 68.8 (minor C-2′-C-5′), 75.8, 73.6, 70.5, 68.6 (major C-2′-C-5′), 62.9 (minor C-6′), 62.7 (major C-6′), 31.1 (minor i-Pr—CH), 31.0 (major i-Pr—CH), 22.5, 21.4 (minor i-Pr—CH₃), 22.3, 21.4 (major i-Pr—CH₃), 18.8 (major C₆H₄—CH₃), 18.7 (minor C₆H₄—CH₃). ESI-HRMS positive mode (m/z): calcd for: C₅₅H₄₈ClN₄O₉Ru⁺ [M−PF₆]⁺ 1045.2165. Found: 1045.2163.

### 11.54 Complex Os-2b

Prepared from ligand L-2b (20.6 mg, 0.0266 mmol, 2.1 eq.), Os-dimer (10.0 mg, 0.0126 mmol) and TIPF₆ (8.8 mg, 0.0252 mmol) according to general method II. After filtration and removal of the solvent, the residue was dissolved in CHCl₃ (3 mL) and Et₂O (6 mL) was added. The precipitated product was filtered off, then washed with a solvent mixture of CHCl₃-Et₂O=1:1 (2 mL) to give 21.7 mg (67%) brown powder. Diastereomeric ratio: 10:9. R_{f}=0.38 (95:5 CHCl₃-MeOH). ¹H NMR (400 MHz, CDCl₃) δ (ppm): 9.36, 9.26 (2×1H, 2 s, 2×Tria-H-5), 8.52, 8.50 (2×1H, 2 d, J=8.9 Hz in both, 2×Qu-H-8), 8.24, 8.22 (2×1H, 2 d, J=8.9 Hz in both, 2×Qu-H-4), 8.08-7.28 (48H, m, 2×20×Ar, 2×Py-H-3-Py-H-5), 6.55, 6.51 (2×1H, 2 d, J=9.3 Hz in each, 2×H-1′), 6.65, 6.26, 6.18, 6.17, 6.03-5.93 (2×3H, 4 pt, m, J=9.4 Hz in each, 2×H-2′, H-3′, H-4′), 6.03-5.93, 5.82, 5.79, 5.74, 5.71, 5.69, 5.66 (2×4H, m, 6 d, J=5.7 Hz in each, 2×4×p-cym-CH_{Ar}), 4.84-4.55 (2×3H, m, 2×H-5′, 2×H-6′a, 2×H-6′b), 2.22, 2.13 (2×3H, 2 s, 2×C₆H₄—CH₃), 2.20, 1.91 (2×1H, 2 hept, J=6.9 Hz in each, 2×i-Pr—CH), 0.80, 0.73, 0.54, 0.51 (2×2×3H, 2×2 d, J=6.9 Hz in each 2×2×i-Pr—CH₃); ¹³C NMR (100 MHz, CDCl₃) δ (ppm): 166.4, 166.2, 165.6, 165.4, 165.3, 165.2, 165.1, 164.9 (2×4×C═O), 149.8, 148.9, 148.6, 148.1 (2×Tria-C-4, Qu-C-2, Qu-C-8a), 141.5, 141.3 (2×Qu-C-2), 134.3, 134.2, 133.8, 133.6, 133.5, 133.4, 133.3, 133.2, 130.4-127.9 (2×Qu-C-4a, Qu-C-5-Qu-C-8, Tria-C-5, Ar), 118.5, 118.4, (2×Qu-C-3), 97.2, 96.7, 96.6, 96.1 (2×2×p-cym-C_{qAr}), 87.3, 86.8 (2×C-1′), 78.9, 78.7, 76.7, 76.2, 75.8, 75.4, 75.2, 74.1, 73.5, 73.2, 72.7, 72.0, 70.4, 68.8, 68.6 (2×4×p-cym-CH_{Ar}, 2×C-2-C-5′), 62.8, 62.7 (2×C-6′), 31.2, 31.1 (2×i-Pr—CH), 22.9, 22.6, 21.7, 21.6 (2×2×i-Pr—CH₃), 18.7, 18.6 (2×C₆H₄—CH₃). ESI-HRMS positive mode (m/z): calcd for: C₅₅H₄₈ClN₄O₉Os⁺ [M−PF₆]⁺ 1135.2725. Found: 1135.2724.

### 11.55 Complex Ir-2b

Prepared from ligand L-2b (20.4 mg, 0.0264 mmol, 2.1 eq.), Ir-dimer (10.0 mg, 0.0126 mmol) and TIPF₆ (8.7 mg, 0.0249 mmol) according to general method II. After filtration and removal of the solvent, the residue was dissolved in CHCl₃ (3 mL) and Et₂O (6 mL) was added. The precipitated product was filtered off, then washed with a solvent mixture of CHCl₃-Et₂O=1:1 (1 mL) to give 19.7 mg (61%) yellow powder. Diastereomeric ratio: 5:3. R_{f}=0.41 (95:5 CHCl₃-MeOH). ¹H NMR (400 MHz, CDCl₃) δ (ppm): 9.41 (s, minor Tria-H-5), 9.34 (s, major Tria-H-5), 8.38-7.24 (m, minor and major Qu-H-3-Qu-H-8, Ar), 6.61 (d, J=9.3 Hz, major H-1′), 6.58 (d, J=9.9 Hz, minor H-1′), 6.46 (pt, J=9.4, 9.4 Hz, major H-2′ or H-3′ or H-4′), 6.29-6.23 (m, minor H-2′ and/or H-3′ and/or H-4′), 6.15 (pt, J=9.4, 9.4 Hz, major H-2′ or H-3′ or H-4′), 6.02 (pt, J=9.8, 9.7 Hz, minor H-2′ or H-3′ or H-4′), 5.93 (pt, J=9.8, 9.7 Hz, minor H-2′ or H-3′ or H-4′), 4.81-4.54 (m, minor and major H-5′, H-6′a, H-6′b), 1.52 (s, major Cp*—CH₃), 1.48 (s, minor Cp*—CH₃); ¹³C NMR (100 MHz, CDCl₃) δ (ppm): 166.3, 165.5, 165.3, 165.0 (minor and major 4×C═O), 149.7, 148.9, 145.7, (minor Tria-C-4, Qu-C-2, Qu-C-8a), 149.3, 149.1, 145.6 (major Tria-C-4, Qu-C-2, Qu-C-8a), 141.8, 141.6, 134.2, 134.1, 134.0, 133.8, 133.5, 133.4, 133.3, 132.7, 132.6, 130.3-127.9, 127.3, 118.8 (minor and major Tria-C-5, Qu-C-3-Qu-C-8, Qu-C-4a, Ar), 90.0 (major Cp*), 89.9 (minor Cp*), 87.4 (minor C-1′), 87.2 (major C-1′), 76.1, 75.9, 73.7, 72.9, 71.7, 70.5, 68.7, 68.5 (minor and major C-2′-C-5′), 62.8 (major C-6′), 62.7 (minor C-6′), 9.1 (minor and major Cp*—CH₃). ESI-HRMS positive mode (m/z): calcd for: C₅₅H₄₉ClN₄O₉Ir⁺ [M−PF₆]⁺ 1137.2813. Found: [M−PF₆]⁺ 1137.2810.

### 11.56 Complex Rh-2b

Prepared from ligand L-2b(26.3 mg, 0.0340 mmol, 2.1 eq.), Rh-dimer (10.0 mg, 0.0162 mmol) and TIPF₆ (11.3 mg, 0.0323 mmol) according to general method II. After filtration and removal of the solvent, the residue was dissolved in CHCl₃ (3 mL) and Et₂O (6 mL) was added. The precipitated product was filtered off, then washed with a solvent mixture of CHCl₃-Et₂O=1:1 (2 mL) to give 26.2 mg (80%) orange powder. Diastereomeric ratio: 5:3. R_{f}=0.38 (95:5 CHCl₃-MeOH). ¹H NMR (400 MHz, CDCl₃) δ (ppm): 9.33 (s, minor Tria-H-5), 9.28 (s, major Tria-H-5), 8.40-7.22 (m, minor and major Qu-H-3-Qu-H-8, Ar), 6.61 (d, J=9.3 Hz, major H-1′), 6.57 (d, J=8.6 Hz, minor H-1′), 6.52 (pt, J=9.3, 9.2 Hz, major H-2′ or H-3′ or H-4′), 6.28 (pt, J=9.6, 9.4 Hz, minor H-2′ or H-3′ or H-4′), 6.23 (pt, J=9.5, 9.0 Hz, minor H-2′ or H-3′ or H-4′), 6.17 (pt, J=9.5, 9.4 Hz, major H-2′ or H-3′ or H-4′), 6.02 (pt, J=9.5, 9.5 Hz, minor H-2′ or H-3′ or H-4′), 5.94 (pt, J=9.8, 9.7 Hz, major H-2′ or H-3′ or H-4′), 4.80-4.54 (m, minor and major H-5′, H-6′a, H-6′b), 1.51 (s, major Cp*—CH₃), 1.44 (s, minor Cp*—CH₃); ¹³C NMR (100 MHz, CDCl₃) δ (ppm): 166.2 (2), 165.7, 165.5 156.3, 165.2, 165.0, 164.7 (minor and major 4×C═O), 148.1, 148.0, 147.9, 147.5, 145.9 (2) (minor and major Tria-C-4, Qu-C-2, Qu-C-8a), 141.3, 141.2, 134.1, 133.8, 133.7, 133.6, 133.5, 133.4, 133.3, 133.2, 130.3-127.8, 127.3, 119.0 (minor and major Tria-C-5, Qu-C-3-Qu-C-8, Qu-C-4a, Ar), 97.9, 97.8 (minor and major Cp*), 87.5 (minor C-1′), 87.0 (major C-1′), 76.2, 75.8, 73.7, 72.8, 71.8, 70.6, 68.7, 68.6 (minor and major C-2′-C-5′), 62.8 (minor C-6′), 62.7 (major C-6′), 9.3 (major Cp*—CH₃), 9.2 (minor Cp*—CH₃). ESI-HRMS positive mode (m/z): calcd for: C₅₅H₄₉ClN₄O₉Rh⁺ [M−PF₆]⁺ 1047.2243. Found: [M−PF₆]⁺ 1047.2240.

### Example 12—Materials and Methods 3—Assays and Analysis

### 12.1 Determination of the Distribution Coefficients (log D)

Prior the experiments n-octanol was saturated with aqueous PBS solution (pH=7.40) and vice versa. The corresponding complex (approximately 0.2-0.3 mg) was dissolved in a mixture of 2.50 mL pre-saturated n-octanol and 2.50 mL pre-saturated PBS buffer, and the mixture was vigorously stirred for 3 days. Based on the NMR stability measurements, this time was necessary to reach equilibrium between the various ionic complex species. Due to the lipophilic/hydrophilic character of the complexes those with benzoyl protection or the non-sugar derivative containing ones could mostly be found in the n-octanol, while the acetyl- and non-protected complexes in the aqueous PBS phase. The appropriate separated solution was centrifuged (ScanSpeed 406G instrument, 4000 RPM for 5 minutes) and the absorption of the “stock solution” was measured (VWR UV-1600PC Spectrophotometer, 270-420 nm). Then 2.00 mL stock solution was stirred vigorously with 16.00 mL pre-saturated, clean n-octanol or PBS solution. After one day the phases were separated, centrifuged and absorption of the solution was measured again. From the absorption difference of the stock solutions the distribution coefficient (D_{PH}, here measured at pH 7.) was calculated according to the previously described formulae (Kozsup et al., 2020).

After phase separation, log D is calculated from the absorbance values of the UV-Vis spectrum and the volume of the phases as required.

### 12.3 Cell Culture Materials and the Source of the Platinum Compounds

Carboplatin, oxaliplatin, cisplatin, rucaparib, Trolox and MitoTEMPO were from Sigma-Aldrich (St. Louis, MO, USA). All other materials for cellular experiments were from Sigma-Aldrich, unless otherwise stated.

### 12.4 Cell Culture

Cells were cultured under standard cell culture conditions, 37° C., 5% CO₂, humidified atmosphere.

A2780 cells were cultured in RMPI 1640 medium supplemented with 10% fetal calf serum, 2 mM glutamine, 1% penicillin-streptomycin (Sigma-Aldrich).

ID8 cells were cultured in high glucose DMEM (4.5 g/L glucose) medium supplemented with 4% fetal calf serum, 2 mM glutamine, 1% penicillin-streptomycin (Sigma-Aldrich), 1% ITS supplement (Sigma-Aldrich I3146).

U251 cells were maintained in MEM (Sigma-Aldrich), 10% fetal bovine serum (Sigma-Aldrich), 1% Penicillin/Streptomycin (Invitrogene), 2 mM Glutamine.

MCF7 cells were maintained in MEM (Sigma-Aldrich), 10% fetal bovine serum (Sigma-Aldrich), 1% Penicillin/Streptomycin (Invitrogene), 2 mM Glutamine.

Capan2 cells were maintained in MEM (Sigma-Aldrich), 10% fetal bovine serum (Sigma-Aldrich), 1% Penicillin/Streptomycin (Invitrogene), 2 mM Glutamine.

Human primary dermal fibroblasts were cultured in low glucose DMEM (1 g/L glucose) medium supplemented with 20% fetal calf serum, 2 mM glutamine, 1% penicillin-streptomycin (Sigma-Aldrich).

L428 cells were maintained in RPMI 1640 medium supplemented with 10% fetal calf serum, 2 mM glutamine, 1% penicillin-streptomycin.

Saos cells were maintained in DMEM (4.5 g/L glucose) medium supplemented with 10% fetal calf serum, 2 mM glutamine, 1% penicillin-streptomycin.

Cisplatin resistant A2780 cells were grown in RMPI 1640 medium supplemented with 10% fetal calf serum, 2 mM glutamine, 1% penicillin-streptomycin. Cisplatin resistant cells underwent selection (1 μM cisplatin) once a week for 3 days before plating for any assay.

### 12.5 Bacterial Culture and Broth Microdilution Experiments

Microdilution experiments were performed according to the standards of EUCAST.

(EUCAST (EUROPEAN COMMITTEE ON ANTIMICROBIAL SUSCEPTIBILITY TESTING, European Society of Clinical Microbiology and Infectious Diseases; see EUCAST reading guide for broth microdilution, Version 3.0 January 2021, also available at https://eucast.org/ast_of_bacteria/mic_determination/) recommends testing according to the International Standard ISO 20776-1. See: “MIC determination of non-fastidious and fastidious organisms.”)

The bacterial isolates to be tested were grown on Mueller-Hinton agar plates. Inoculum density of bacteria was set at 5.0×10⁵ CFU/mL in microtiter plates in a final volume of 200 μL Mueller-Hinton broth. Tested concentration range was 0.08-40 μM (10 concentrations, two-fold serial dilutions), drug-free growth control and inoculum-free negative control were included. The inoculated plates were incubated for 24 hours at 37° C. then were assessed visually. Minimum inhibitory concentration (MIC) was defined as the lowest concentration with 50%<inhibitory effect compared to the growth control. All experiments were performed at least twice in duplicates.

Results are recorded as the lowest concentration of antimicrobial agent that inhibits visible growth of a microorganism, the Minimum Inhibitory Concentration (MIC), expressed in mg/L or μg/mL.

### The Following Bacteria Strains Examined:

The examined reference strains are the following:
Staphylococcus aureus ref. strain (ATCC11002)
Staphylococcus aureus ref. strain (ATCC43300)
Staphylococcus aureus ref. strain (ATCC11007)
Enterococcus faecalis ref. strain (ATCC29112)
Candida albicans (SC5314) (ATCC Cat. No. MYA-2876™)
Candida auris (ATCC21092)
ATCC strains were or can be purchased from ATCC (Manassas, VA, USA).

### The Examined Clinical Isolates are Listed Below.

| | Species | | Year | Sample |
|---|---|---|---|---|
| 20627 | S. aureus | MSSA | 2018 | ear |
| 20559 | S. aureus | MSSA | 2018 | wound |
| 20650 | S. aureus | MSSA | 2018 | nasal |
| 20904 | S. aureus | MSSA | 2018 | abscessus |
| 20276 | S. aureus | MSSA | 2018 | wound |
| 20478 | S. aureus | MSSA | 2018 | bronchial |
| 20426 | S. aureus | MRSA | 2020 | blood |
| 24408 | S. aureus | MRSA | 2018 | bronchial |
| 24268 | S. aureus | MRSA | 2018 | throat |
| 24328 | S. aureus | MRSA | 2018 | throat |
| 24272 | S. aureus | MRSA | 2018 | throat |
| 24035 | S. aureus | MRSA | 2018 | wound |
| 25051 | E. faecium | VRE | 2018 | nephrostoma |
| 27085 | E. faecium | VRE | 2018 | wound |
| 25498 | E. faecium | VRE | 2018 | screening for multiresistant |
| | | | | pathogens |
| 25342 | E. faecium | VRE | 2021 | urine |
| 28209 | E. faecium | VRE | 2021 | urine |
| 28046 | E. faecium | VSE | 2021 | abdominal |
| 28386 | E. faecium | VSE | 2021 | urine |
| 28085 | E. faecium | VRE | 2021 | urine |

### Tested Materials:

The concentrations were identical in case of each microorganism tested:
40 μM; 20 μM; 10 μM; 5 μM; 2.5 μM; 1.25 μM; 0.625 μM; 0.3125 μM; 0.1563 μM; 0.07813 μM
Growth control (positive control): without test compounds (complex or ligand)
Negative control: neither test compound nor bacterium or fungus suspension is added.

### Method:

In case of bacteria Mueller-Hinton broth has been applied as medium. From 0.5 McFarland (corresponding to 1.5×10⁸ CFU/ml) bacterium suspension 100× dilution was prepared in Mueller-Hinton broth, and into each well 5.0×10⁵ CFU/mL was added by measuring 100 μL diluted bacterium inoculum into 200 μl end volume as defined by the EUCAST guidelines.

From each test compounds 160 μM concentrations were prepared in 2 mL end volume in Mueller-Hinton broth, from which 100 μL was also measured into the first wells of each row of wells. Then up to the tenth well of each row 2× dilution was carried out with Mueller-Hinton broth to obtain the above described concentrations.

Thereafter the diluted bacterium suspensions were measured into the wells so as to obtain the advised CFU except in case of the negative control. The ready-made plates were incubated for 24 h at 37° C. and then read. Upon comparison the Growth control was used as a base. Each well wherein an at least 50% growth inhibition was found has been selected and marked.

Examination was made at least in two parallels in each case.

### 12.6 Methylthiazolyldiphenyl-Tetrazolium Bromide (MTT) Reduction Assay

MTT reduction assay assesses the activity of mitochondrial complex I and was used to assess rapid toxicity. MTT reduction assay performed similarly to (Bakondi et al., 2003) as follows. Cells were seeded in 96-well plates. The next day cells were treated with the compounds for 4 hours in the concentrations indicated in a cell incubator. At the end of treatment MTT was added in 0.5 mg/ml final concentration and cells were incubated at 37° C. in a cell incubator. Then culture media was removed and the reduced MTT dye was resolved in dimethyl-sulfoxide (DMSO) and plates were measured in a plate photometer (Thermo Scientific Multiscan GO spectrophotometer, Waltham, MA, USA) at 540 nm. On each plate wells were designed to contain untreated/vehicle-treated cells. In calculations the readings for these wells was considered as 1 and all readings were expressed relative to these values.

### 12.7 Sulphorhodamine B (SRB) Proliferation Assay

SRB accumulation assay assesses protein content in a sample and was used to assess cell proliferation. SRB accumulation assay was performed similarly to (Fodor et al., 2016) as follows. Cells were seeded in 96-well plates. The next day cells were treated with the compounds for 48 hours in the concentrations indicated in a cell incubator. At the end of treatment cells were fixed with 10% trichloroacetic acid (TCA). Fixed cells were stained with SRB (0.4 m/V % dissolved in 1% acetic acid) for 60 minutes. Fixed cells were washed in 1% acetic acid 3 times; acetic acid was removed and cells were left to dry. Protein-bound SRB was released by adding 100 μl 10 mM Tris base. Plates were measured in a plate photometer (Thermo Scientific Multiscan GO spectrophotometer, Waltham, MA, USA) at 540 nm. On each plate wells were designed to contain untreated cells. In calculations the readings for these wells was considered as 1 and all readings were expressed relative to these values.

### 12.8 AnnexinV-FITC, PI Double Staining

AnnexinV-PI double staining was applied to assess apoptotic and necrotic cell death similar to (Bai et al., 2001; Virag et al., 1998a; Virag et al., 1998c). A2780 cells were treated with the indicated compounds at the concentration corresponding to their IC50 value for 2, 4 and 48 hours. The 4 and 48 hours time point corresponds to the time points for the MTT and SRB assays, while the early 2 hours time point reflects an optimum time point for the detection of apoptotic or necrotic cell death (Bai et al., 2001; Virag et al., 1998a; Virag et al., 1998c). Quadrants were set based on the FITC and PI values observed for the vehicle-treated cells.

### 12.9 Western Blotting

The preparation of protein extracts, the separation of protein extracts in SDS-polyacrylamide gel electrophoresis and the subsequent Western blotting was performed as described in (Marton et al., 2018) using the antibodies in Table 6. Enhanced chemiluminescence was developed using ChemiDoc Imager, (Bio-Rad, Hercules, California, USA). Densitometry was performed using Image Lab Touch Software, Bio-Rad, Hercules, California, USA)

TABLE 6
The list of antibodies used in the examples of the invention

| | | Catalog | |
|---|---|---|---|
| Antibody | Company | number | Dilution |
| 4-Hydroxynonenal (4HNE) | Abcam | ab46545 | 1:1000 |
| Anti-mouse IgG, HRP-linked | Sigma-Aldrich | A9044 | 1:2000 |
| Anti-rabbit IgG, HRP-linked | Cell Signaling | 7074 | 1:2000 |
| | Technology | | |
| Anti-β-actin-Peroxidase | Sigma-Aldrich | A3854 | 1:20000 |

### 12.10 Statistical Analysis

Statistical analysis was performed using 8.0.1 version of Graphpad Prism. Values were tested for normal distribution using the D'Agostino and Pearson normality test. When necessary, values were log normalized or were normalized using the Box-Cox normalization method (Box and Cox, 1964), as indicated in the figure captions. The following statistical test, post hoc test and the level of significance is indicated in the figure captions. Nonlinear regression was performed using the built-in “[Inhibitor] vs. response—Variable slope (four parameters), least square fit” utility of Graphpad that yielded IC50 and Hill slope values.

### 12.11 Seahorse Oximetry

General consideration for Seahorse oximetry can be found at (Miko et al., 2017) and at https://www.agilent.com/en/product/cell-analysis/real-time-cell-metabolic-analysis/xf-analyzers.

A2780 cells were plated into specialized Seahorse cell plates and were subjected to oximetry. First, baseline oxygen consumption was recorded. Then cells received the complexes in concentrations corresponding to their respective IC50 values and OCR and ECAR values were collected every 30 minutes. At the end of the experiment cells received antimycin.

The first step in calculation was to subtract antimycin-resistant respiration rates from the baseline or complex-induced respiration. The first two baseline respiration (OCR) and extracellular acidification rates (ECAR) rates were omitted. All respiration and ECAR value was expressed as a fold change compared to controls.

### 12.12 Assessment of Cell Proliferation on L428 Cells

Cells were seeded in 96 well plates the day before the assay. Cells were treated with the compounds for 96 hours. Cells were counted using a Burker chamber. On each plate wells were designed to contain untreated cells. In calculations the readings for these wells was considered as 1 and all readings were expressed relative to these values.

### REFERENCES

Bai, P. (2015). Biology of Poly(ADP-Ribose) Polymerases: The Factotums of Cell Maintenance. Molecular Cell 58, 947-958.
Bai, P., Bakondi, E., Szabo, E., Gergely, P., Szabo, C., and Virag, L. (2001). Partial protection by poly(ADP-ribose) polymerase inhibitors from nitroxyl-induced cytotoxity in thymocytes. Free Radical Biology and Medicine 31, 1616-1623.
Bai, P., Nagy, L., Fodor, T., Liaudet, L., and Pacher, P. (2015). Poly(ADP-ribose) polymerases as modulators of mitochondrial activity. Trends in Endocrinology and Metabolism 26, 75-83.
Bakewell, S., Conde, I., Fallah, Y., McCoy, M., Jin, L., and Shajahan-Haq, A. N. (2020). Inhibition of DNA Repair Pathways and Induction of ROS Are Potential Mechanisms of Action of the Small Molecule Inhibitor BOLD-100 in Breast Cancer. Cancers (Basel) 12, 2647.
Bakondi, E., Gonczi, M., Szabo, E., Bai, P., Pacher, P., Gergely, P., Kovacs, L., Hunyadi, J., Szabo, C., Csernoch, L., et al. (2003). Role of intracellular calcium mobilization and cell-density-dependent signaling in oxidative-stress-induced cytotoxicity in HaCaT keratinocytes. J Invest Dermatol 121, 88-95.
Berger, I., Hanif, M., Nazarov, A. A., Hartinger, C. G., John, R. O., Kuznetsov, M. L., Groessl, M., Schmitt, F., Zava, O., Biba, F., et al. (2008). In Vitro Anticancer Activity and Biologically Relevant Metabolization of Organometallic Ruthenium Complexes with Carbohydrate-Based Ligands. Chemistry—A European Journal 14, 9046-9057.
Bokor, E., Docsa, T., Gergely, P., and Somsák, L. (2010). Synthesis of 1-(D-glucopyranosyl)-1,2,3-triazoles and their evaluation as glycogen phosphorylase inhibitors. Bioorg Med Chem 18, 1171-1180.
Bokor, É., Koppány, C., Gonda, Z., Novák, Z., and Somsák, L. (2012). Evaluation of bis-triphenylphosphano-copper(I)-butyrate (C₃H₇COOCu(PPh₃)₂) as catalyst for the synthesis of 1-(D-glycopyranosyl)-4-substituted-1,2,3-triazoles. Carbohydr Res 351, 42-48.
Bononi, G., Iacopini, D., Cicio, G., Di Pietro, S., Granchi, C., Di Bussolo, V., and Minutolo, F. (2020). Glycoconjugated Metal Complexes as Cancer Diagnostic and Therapeutic Agents. Chemmedchem 16, 30-64.
Box, G. E. P., and Cox, D. R. (1964). An analysis of transformations. Journal of the Royal Statistical Society, B 26, 211-234.
Böge, M., Fowelin, C., Bednarski, P., and Heck, J. (2015). Diaminohexopyranosides as Ligands in Half-Sandwich Ruthenium(II), Rhodium(III), and Iridium(III) Complexes. Organometallics 34, 1507-1521.
Burris, H. A., Bakewell, S., Bendell, J. C., Infante, J., Jones, S. F., Spigel, D. R., Weiss, G. J., Ramanathan, R. K., Ogden, A., and Von Hoff, D. (2016). Safety and activity of IT-139, a ruthenium-based compound, in patients with advanced solid tumours: a first-in-human, open-label, dose-escalation phase I study with expansion cohort. ESMO Open 1, e000154.
Chrysina, E. D., Bokor, É., Alexacou, K.-M., Charavgi, M.-D., Oikonomakos, G. N., Zographos, S. E., Leonidas, D. D., Oikonomakos, N. G., and Somsak, L. (2009). Amide-1,2,3-triazole bioisosterism: the glycogen phosphorylase case. Tetrahedron: Asymmetry 20, 733-740.
Conklin, K. A. (2004). Chemotherapy-associated oxidative stress: impact on chemotherapeutic effectiveness. Integrative cancer therapies 3, 294-300.
Curtin, N., and Szabo, C. (2013). Therapeutic Applications of PARP Inhibitors: Anticancer Therapy and Beyond. Molecular Aspects of Medicine 6, 1043-1258.
Curtin, N. J., and Szabo, C. (2020). Poly(ADP-ribose) polymerase inhibition: past, present and future. Nature Reviews Drug Discovery 19, 711-736.
de Camargo, M. S., De Grandis, R. A., da Silva, M. M., da Silva, P. B., Santoni, M. M., Eismann, C. E., Menegario, A. A., Cominetti, M. R., Zanelli, C. F., Pavan, F. R., et al. (2019). Determination of in vitro absorption in Caco-2 monolayers of anticancer Ru(II)-based complexes acting as dual human topoisomerase and PARP inhibitors. Biometals. 32, 89-100.
Farkas, I., Szabó, I. F., and Bognár, R. (1977). Conversion of acetylated glycosyl cyanides into C-glycosyl derivatives of benzothiazole and tetrazole. Carbohydr Res 56, 404-406.
Fernandes, A. C. (2019). Synthesis, Biological Activity and Medicinal Applications of Ruthenium Complexes Containing Carbohydrate Ligands. Curr Med Chem 26, 6412-6437.
Florindo, P., Marques, I. J., Nunes, C. D., and Fernandes, A. C. (2014). Synthesis, characterization and cytotoxicity of cyclopentadienyl ruthenium(II) complexes containing carbohydrate-derived ligands. Journal of Organometallic Chemistry 760, 240-247.
Florindo, P. R., Pereira, D. M., Borralho, P. M., Costa, P. J., Piedade, M. F. M., Rodrigues, C. M. P., and Fernandes, A. C. (2016). New (eta(5)-C5H₅)Ru(N—N)(PPh3) PF6 compounds: colon anticancer activity and GLUT-mediated cellular uptake of carbohydrate-appended complexes. Dalton Transactions 45, 11926-11930.
Florindo, P. R., Pereira, D. M., Borralho, P. M., Rodrigues, C. M. P., Piedade, M. F. M., and Fernandes, A. C. (2015).
Cyclopentadienyl-Ruthenium(II) and Iron(II) Organometallic Compounds with Carbohydrate Derivative Ligands as Good Colorectal Anticancer Agents. Journal of Medicinal Chemistry 58, 4339-4347.
Fodor, T., Szanto, M., Abdul-Rahman, O., Nagy, L., Der, A., Kiss, B., and Bai, P. (2016). Combined Treatment of MCF-7 Cells with AICAR and Methotrexate, Arrests Cell Cycle and Reverses Warburg Metabolism through AMP-Activated Protein Kinase (AMPK) and FOXO1. PLoS One 11, e0150232.
Gano, L., Pinheiro, T., Matos, A. P., Tortosa, F., Jorge, T. F., Gongalves, M. S., Martins, M., Morais, T. S., Valente, A., Tomaz, A. I., et al. (2019). Antitumour and Toxicity Evaluation of a Ru(II)-Cyclopentadienyl Complex in a Prostate Cancer Model by Imaging Tools. Anti-Cancer Agents in Medicinal Chemistry 19, 1262-1275.
Gesztelyi, R., Zsuga, J., Kemeny-Beke, A., Varga, B., Juhasz, B., and Tosaki, A. (2012). The Hill equation and the origin of quantitative pharmacology. Arch Hist Exact Sci 66, 427-438.
Godó, A. J., Bényei, A. C., Duff, B., Egan, D. A., and Buglyó, P. (2012). Synthesis and X-ray diffraction structures of novel half-sandwich Os(ii)- and Ru(ii)-hydroxamate complexes. RSC Advances 2, 1486-1495.
Gonda, Z., and Novak, Z. (2010). Highly active copper-catalysts for azide-alkyne cycloaddition. Dalton transactions (Cambridge, England: 2003) 39, 726-729.
Györgydeák, Z., and Thiem, J. (2006). Synthesis and transformation of glycosyl azides. Adv Carbohydr Chem Biochem 60, 103-182.
Habtemariam, A., Melchart, M., Fernandez, R., Parsons, S., Oswald, I. D., Parkin, A., Fabbiani, F. P., Davidson, J. E., Dawson, A., Aird, R. E., et al. (2006). Structure-activity relationships for cytotoxic ruthenium(II) arene complexes containing N,N-, N,O-, and O,O-chelating ligands. J Med Chem 49, 6858-6868.
Hadady, Z., Tóth, M., and Somsák L. (2004). C-(b-D-glucopyranosyl) heterocycles as potential glycogen phosphorylase inhibitors. Arkivoc (vii), 140-149.
Hamala, V., Martišová, A., Červenková Š\acute{t}astná, L., Karban, J., Dančo, A., Šimarek, A., Lamač, M., Horácěk, M., Kolářová, T., Hrstka, R., et al. (2020). Ruthenium tetrazene complexes bearing glucose moieties on their periphery: Synthesis, characterization, and in vitro cytotoxicity. Applied Organometallic Chemistry 34, e5896.
Hanahan, D., and Weinberg, R. A. (2000). The hallmarks of cancer. Cell. 100, 57-70.
Hanahan, D., and Weinberg, R. A. (2011). Hallmarks of cancer: the next generation. Cell 144, 646-674.
Hanif, M., Meier, S., Nazarov, A., Risse, J., Legin, A., Casini, A., Jakupec, M., Keppler, B., and Hartinger, C. (2013).
Influence of the π-coordinated arene on the anticancer activity of ruthenium(II) carbohydrate organometallic complexes. Frontiers in Chemistry 1, 27.
Hartinger, C. G., Nazarov, A. A., Ashraf, S. M., Dyson, P. J., and Keppler, B. K. (2008). Carbohydrate-Metal Complexes and their Potential as Anticancer Agents. Current Medicinal Chemistry 15, 2574-2591.
Hegedűs, C., Juhász, T., Fidrus, E., Janka, E. A., Juhász, G., Boros, G., Paragh, G., Uray, K., Emri, G., Remenyik, É., et al. (2021). Cyclobutane pyrimidine dimers from UVB exposure induce a hypermetabolic state in keratinocytes via mitochondrial oxidative stress. Redox Biol 38, 101808.
Hill, S., Sataranatarajan, K., and Van Remmen, H. (2018). Role of Signaling Molecules in Mitochondrial Stress Response. Front Genet 9, 225.
Holditch, S. J., Brown, C. N., Lombardi, A. M., Nguyen, K. N., and Edelstein, C. L. (2019). Recent Advances in Models, Mechanisms, Biomarkers, and Interventions in Cisplatin-Induced Acute Kidney Injury. Int J Mol Sci 20, 3011.
Kenny, R. G., and Marmion, C. J. (2019). Toward Multi-Targeted Platinum and Ruthenium Drugs-A New Paradigm in Cancer Drug Treatment Regimens?Chemical reviews 119, 1058-1137.
Kim, W., Lee, S., Seo, D., Kim, D., Kim, K., Kim, E., Kang, J., Seong, K. M., Youn, H., and Youn, B. (2019). Cellular Stress Responses in Radiotherapy. Cells 8.
Kiss, B., Mikó, E., Sebő, É., Toth, J., Ujlaki, G., Szabó, J., Uray, K., Bai, P., and Árkosy, P. (2020). Oncobiosis and Microbial Metabolite Signaling in Pancreatic Adenocarcinoma. Cancers (Basel) 12, E1068.
Kozsup, M., Domotor, O., Nagy, S., Farkas, E., Enyedy, E. A., and Buglyo, P. (2020). Synthesis, characterization and albumin binding capabilities of quinizarin containing ternary cobalt(III) complexes. Journal of inorganic biochemistry 204.
Kraft, J., Schmollinger, D., Maudrich, J., and Ziegler, T. (2015). Synthesis of Sugar-Derived Triazole- and Pyridine-Based Metal Complex Ligands. Synthesis-Stuttgart 47, 199-208.
Kumar, V., Abbas, A., and Aster, J. (2017). Basic pathology (Elsevier).
Kun, S., Nagy, G. Z., Tóth, M., Czecze, L., Nguyen van Nhien, A., Docsa, T., Gergely, P., Charavgi, M.-D., Skourti, P. V.,
Chrysina, E. D., et al. (2011). Synthesis of variously coupled conjugates of D-glucose, 1,3,4-oxadiazole, and 1,2,3-triazole for inhibition of glycogen phosphorylase. Carbohydr Res 346, 1427-1438.
Liu, J., Lai, H., Xiong, Z., Chen, B., and Chen, T. (2019). Functionalization and cancer-targeting design of ruthenium complexes for precise cancer therapy. Chemical communications (Cambridge, England) 55, 9904-9914.
Manohar, S., and Leung, N. (2018). Cisplatin nephrotoxicity: a review of the literature. Journal of nephrology 31, 15-25.
Marton, J., Fodor, T., Nagy, L., Vida, A., Kis, G., Brunyanszki, A., Antal, M., Luscher, B., and Bai, P. (2018). PARP10 (ARTD10) modulates mitochondrial function. PLoS One 13, e0187789.
McMullen, M., Madariaga, A., and Lheureux, S. (2020). New approaches for targeting platinum-resistant ovarian cancer. Semin Cancer Biol, doi: 10.1016/j.semcancer.2020.1008.1013.
Meier-Menches, S. M., Gerner, C., Berger, W., Hartinger, C. G., and Keppler, B. K. (2018). Structure-activity relationships for ruthenium and osmium anticancer agents—towards clinical development. Chemical Society Reviews 47, 909-928.
Melchart, M., and Sadler, P. J. (2006). Ruthenium Arene Anticancer Complexes. In Bioorganometallics. G. Jaouen, ed. (Wiley-VCH Verlag GmbH & Co. KGaA), pp. 39-64.
Meldal, M., and Tornoe, C. W. (2008). Cu-catalyzed azide-alkyne cycloaddition. Chemical reviews 108, 2952-3015.
Mello-Andrade, F., Cardoso, C. G., Silva, C. R. E., Chen-Chen, L., Melo-Reis, P. R., Lima, A. P., Oliveira, R., Ferraz, I. B. M., Grisolia, C. K., Almeida, M. A. P., et al. (2018). Acute toxic effects of ruthenium (II)/amino acid/diphosphine complexes on Swiss mice and zebrafish embryos. Biomedicine & pharmacotherapy=Biomedecine & pharmacotherapie 107, 1082-1092.
Mihajlovic, K., Milosavljevic, I., Jeremic, J., Savic, M., Sretenovic, J., Srejovic, I. M., Zivkovic, V. I., Jovicic, N., Paunovic, M., Bolevich, S., et al. (2020). Redox and apoptotic potential of novel ruthenium complexes in the rat blood and heart. Canadian journal of physiology and pharmacology, doi: 10.1139/cjpp-2020-0349.
Miko, E., Kovacs, T., Fodor, T., and Bai, P. (2017). Methods to Assess the Role of Poly(ADP-Ribose) Polymerases in Regulating Mitochondrial Oxidation. Methods Mol Biol 1608, 185-200.
Modis, K., Gero, D., Erdelyi, K., Szoleczky, P., Dewitt, D., and Szabo, C. (2012). Cellular bioenergetics is regulated by PARP1 under resting conditions and during oxidative stress. Biochem Pharmacol 83, 633-643.
Mohamed, A. I.e.a. (2017). Synthesis and antibacterial activity of 1-N-(b-D-glucopyranosyl)-4-((1-substituted-1H-1,2, 3-triazol-4-yl)ethoxymethyl)-1,2,3-triazoles. Arabian Journal of Chemistry 10, S3508-S3514.
Mukherjea, D., Dhukhwa, A., Sapra, A., Bhandari, P., Woolford, K., Franke, J., Ramkumar, V., and Rybak, L. (2020). Strategies to reduce the risk of platinum containing antineoplastic drug-induced ototoxicity. Expert opinion on drug metabolism & toxicology 16, 965-982.
Nainu, F., Permana, A. D., Djide, N. J. N., Anjani, Q. K., Utami, R. N., Rumata, N. R., Zhang, J., Emran, T. B., and Simal-Gandara, J. (2021). Pharmaceutical Approaches on Antimicrobial Resistance: Prospects and Challenges. Antibiotics (Basel, Switzerland) 10, 981.
Ndagi, U., Mhlongo, N., and Soliman, M. E. (2017). Metal complexes in cancer therapy—an update from drug design perspective. Drug design, development and therapy 11, 599-616.
Pacher, P., Liaudet, L., Bai, P., Mabley, J. G., Kaminski, P. M., Virag, L., Deb, A., Szabo, E., Ungvari, Z., Wolin, M. S., et al. (2003). Potent metalloporphyrin peroxynitrite decomposition catalyst protects against the development of doxorubicin-induced cardiac dysfunction. Circulation. 107, 896-904.
Pacher, P., Liaudet, L., Bai, P., Virag, L., Mabley, J. G., Hasko, G., and Szabo, C. (2002). Activation of poly(ADP-ribose) polymerase contributes to development of doxorubicin-induced heart failure. Journal of Pharmacology and Experimental Therapeutics 300, 862-867.
Parveen, S., Hanif, M., Leung, E., Tong, K. K. H., Yang, A., Astin, J., De Zoysa, G. H., Steel, T. R., Goodman, D., Movassaghi, S., et al. (2019). Anticancer organorhodium and -iridium complexes with low toxicity in vivo but high potency in vitro: DNA damage, reactive oxygen species formation, and haemolytic activity. Chem Commun (Camb) 55, 12016-12019.
Paulsen, H., Györgydeák, Z., and Friedmann, M. (1974). Exo-Anomerer Effekt und Circulardichroismus von Glycopyranosylaziden. Chem Ber 107, 1568-1578.
Rostovtsev, V. V., Green, L. G., Fokin, V. V., and Sharpless, K. B. (2002). A Stepwise Huisgen Cycloaddition Process: Copper(I)-Catalyzed Regioselective “Ligation” of Azides and Terminal Alkynes. Angew Chem 41, 2596-2599. Šamšulová, V., Poláková, M., Horák, R., Šedivá, M., Kvapil, L., and Hradil, P. (2019). Synthetic approach to novel glycosyltriazole-3-hydroxyquinolone conjugates and their antimicrobial properties. Journal of Molecular Structure 1177, 16-25.
Sári, Z., Mikó, E., Kovács, T., Boratkó, A., Ujlaki, G., Jankó, L., Kiss, B., Uray, K., and Bai, P. (2020). Indoxylsulfate, a Metabolite of the Microbiome, Has Cytostatic Effects in Breast Cancer via Activation of AHR and PXR Receptors and Induction of Oxidative Stress Cancers (Basel) 12, 2915.
Shelburne, S., and Musher, D. M. (2011). Management of Gram-Positive Bacterial Disease: Staphylococcus aureus, Streptococcal, Pneumococcal and Enterococcal Infections. Principles and Practice of Cancer Infectious Diseases, 409-421.
Shen, C., Zheng, H., Zhang, P. F., and Chen, X. Z. (2010). Synthesis of Some Novel Glucosyl Triazoles from 2,3,4,6-Tetra-O-pivaloyl-D-glucopyranosyl Azide. J Carbohydr Chem 29, 155-163.
Sipos, A., Ujlaki, G., Mikó, E., Maka, E., Szabó, J., Uray, K., Krasznai, Z., and Bai, P. (2021). The role of the microbiome in ovarian cancer: mechanistic insights into oncobiosis and to bacterial metabolite signaling. Molecular medicine (Cambridge, Mass.) 27, 33.
Smolková, K., Mikó, E., Kovács, T., Leguina-Ruzzi, A., Sipos, A., and Bai, P. (2020). NRF2 in regulating cancer metabolism. Antioxidants and Redox Signaling 33, 966-997.
Somsák, L., Bokor, É., Czibere, B., Czifrák, K., Koppány, C., Kulcsár, L., Kun, S., Szilágyi, E., Tóth, M., Docsa, T., et al. (2014). Synthesis of C-Xylopyranosyl- and Xylopyranosylidene-Spiro-Heterocycles for Inhibition of Glycogen Phosphorylase. Carbohydr Res 399, 38-48.
Somsák et al: Synthesis of New C- and N-Glycopyranosyl Azoles and Their HalĹSandwich Ru(II) Complexes, EUROCARB XX, PZI4 (2019)
Son, M. H., Kim, J. Y., Lim, E. J., Baek, D. J., Choi, K., Lee, J. K., Pae, A. N., Min, S. J., and Cho, Y. S. (2013). Synthesis and biological evaluation of 2-(arylethynyl)quinoline derivatives as mGluR5 antagonists for the treatment of neuropathic pain. Bioorg Med Chem Lett 23, 1472-1476.
Spillane, C. B., Dabo, M. N., Fletcher, N. C., Morgan, J. L., Keene, F. R., Haq, I., and Buurma, N. J. (2008). The dichotomy in the DNA-binding behaviour of ruthenium(II) complexes bearing benzoxazole and benzothiazole groups. Journal of inorganic biochemistry 102, 673-683.
Tawfiq, K. M., Miller, G. J., Al-Jeboori, M. J., Fennell, P. S., Coles, S. J., Tizzard, G. J., Wilson, C., and Potgieter, H. (2014). Comparison of the structural motifs and packing arrangements of six novel derivatives and one polymorph of 2-(1-phenyl-1H-1,2,3-triazol-4-yl)pyridine. Acta Crystallographica Section B-Structural Science Crystal Engineering and Materials 70, 379-389.
Tóth, M., Kun, S., Bokor, É., Benltifa, M., Tallec, G., Vidal, S., Docsa, T., Gergely, P., Somsák, L., and Praly, J.-P. (2009). Synthesis and structure-activity relationships of C-glycosylated oxadiazoles as inhibitors of glycogen phosphorylase. Bioorg Med Chem 17, 4773-4785.
Tykocki, T., and Eltayeb, M. (2018). Ten-year survival in glioblastoma. A systematic review. J Clin Neurosci. 54:7-13., 10.1016/j.jocn.2018.1005.1002. Epub 2018 May 1023.
Vassallo, A., Silletti, M. F., Faraone, I., and Milella, L. (2020). Nanoparticulate Antibiotic Systems as Antibacterial Agents and Antibiotic Delivery Platforms to Fight Infections. Journal of Nanomaterials 2020, 6905631.
Viana, R. M. R., Prado, M. A. F., and Alves, R. J. (2008). Synthesis and modifications of heterocyclic derivatives of D-arabinose: potential inhibitors of glucose-6-phosphate isomerase and glucosamine-6-phosphate synthase. Quim Nova 31, 1710-1713.
Virag, L., Marmer, D. J., and Szabo, C. (1998a). Crucial role of apopain in the peroxynitrite-induced apoptotic DNA fragmentation. Free Radic. Biol. Med. 25, 1075-1082.
Virag, L., Salzman, A. L., and Szabo, C. (1998b). Poly(ADP-ribose) synthetase activation mediates mitochondrial injury during oxidant-induced cell death. J. Immunol. 161, 3753-3759.
Virag, L., Scott, G. S., Cuzzocrea, S., Marmer, D., Salzman, A. L., and Szabo, C. (1998c). Peroxynitrite-induced thymocyte apoptosis: the role of caspases and poly (ADP-ribose) synthetase (PARS) activation. Immunology. 94, 345-355.
Wei, F. X., Fang, L., and Huang, Y. (2010). Synthesis, characterization, crystal structures, and photophysical properties of a series of room-temperature phosphorescent copper(I) complexes with oxadiazole-derived diimine ligand. Inorganica Chimica Acta 363, 2600-2605.
Weiss, V. C., Farias, G., Amorim, A. L., Xavier, F. R., Camargo, T. P., Bregalda, M. B., Haukka, M., Nordlander, E., de Souza, B., and Peralta, R. A. (2020). Luminescent PhotoCORMs: Enabling/Disabling CO Delivery upon Blue Light Irradiation. Inorganic Chemistry 59, 13078-13090.
Wilkinson, B. L., Bornaghi, L. F., Poulsen, S. A., and Houston, T. A. (2006). Synthetic utility of glycosyl triazoles in carbohydrate chemistry. Tetrahedron 62, 8115-8125.
Xiao, N., Cheng, A., Zhu, Q. G., Cheng, Q., Wu, R. B., Yu, B. R., and Wang, Z. (2017). Synthesis of Homoleptic and Heteroleptic Ruthenium Complexes Appended with Glucosyl Ligand by the Click-to-Chelate Approach. Russian Journal of General Chemistry 87, 3036-3042.
Xu, Z., Kong, D., He, X., Guo, L., Ge, X., Liu, X., Zhang, H., Li, J., Yang, Y., and Liu, Z. (2018). Mitochondria-targeted half-sandwich rutheniumll diimine complexes: anticancer and antimetastasis via ROS-mediated signalling. Inorg Chem Front 5, 2100-2105.
Yu, C., Wang, Z., Sun, Z., Zhang, L., Zhang, W., Xu, Y., and Zhang, J. J. (2020). Platinum-Based Combination Therapy: Molecular Rationale, Current Clinical Uses, and Future Perspectives. J Med Chem 63, 13397-13412.
Yusoh, N. A., Ahmad, H., and Gill, M. R. (2020). Combining PARP Inhibition with Platinum, Ruthenium or Gold Complexes for Cancer Therapy. Chem Med Chem 15, 2121-2135.
Zanjirband, M., Curtin, N., Edmondson, R. J., and Lunec, J. (2017). Combination treatment with rucaparib (Rubraca) and MDM2 inhibitors, Nutlin-3 and RG7388, has synergistic and dose reduction potential in ovarian cancer. In Oncotarget, pp. 69779-69796.
Zeng, L., Gupta, P., Chen, Y., Wang, E., Ji, L., Chao, H., and Chen, Z.-S. (2017). The development of anticancer ruthenium(II) complexes: from single molecule compounds to nanomaterials. Chemical Society Reviews 46, 5771-5804.

1. A half-sandwich type compound of general formula (I) 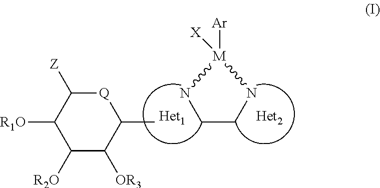
wherein in said formula
M is a transition metal ion selected from the group consisting of Ru, Os, Rh, Ir, Pd and Pt, preferably selected from Ru, Os, Rh, Ir,
X is a halogen selected from the group consisting of Cl, Br and I, preferably Cl,
Ar is a capping molecule having a C₅ or C₆ aromatic ring substituted with one or more, preferably one to five or one to six straight or branched chain C₁-C₆ alkyl, preferably C₁-C₃ alkyl, respectively,
Q is O, S or CH₂, preferably O or CH₂, more preferably O,
Het₁ is a 5 or 6, preferably 5 membered aromatic N-heterocycle,
Het₂ is a 5 to 12 membered heterocycle comprising a 5 or 6 membered, preferably 6 membered aromatic N-heterocycle,
wherein the nitrogens of Het₁ and Het₂ as shown in the formula are forming (N,N) chelate with the transition metal ion,
R₁, R₂, and R₃ are selected from H, C₁-C₂₀ alkyl, C₂-C₂₀ alkenyl, C₂-C₂₀ alkynyl, C₂-C₂₀ alkyl-carbonyl, C₃-C₂₀ alkenyl-carbonyl, C₃-C₂₀ alkynyl-carbonyl, C₆-C₁₀ aryl, C₂-C₂₀ aralkyl, C₈-C₂₀ aralkyl-carbonyl, C₇-C₁₁ aryl-carbonyl (aroyl), wherein at least one, preferably at least two of R₁, R₂, and R₃ is different from H,
wherein the alkyl, alkenyl and aralkyl groups may be of branched or straight chain, unless specifically defined differently and
wherein any aryl group may be unsubstituted or substituted with one or more C₁-C₁₀ alkyl, C₂-C₁₀ alkenyl, C₂-C₁₀ alkynyl, wherein the number of carbon atoms in R₁, R₂ or R₃ is not more than 20,
Z is selected from H, C₁-C₁₀ alkyl, C₂-C₁₀ alkenyl, C₂-C₁₀ alkynyl, —CH₂OR₄, wherein R₄ is selected from any group as defined for R₁, R₂, and R₃,
or any salt or solvate thereof.

2. The compound of claim 1 wherein in said formula R₁, R₂, and R₃ are each a C₇-C₁₁ aryl-carbonyl (aroyl).

3. The compound of claim 1 wherein in said formula,
M is a metal ion selected from the group consisting of Ru, Os, Rh, Ir, preferably Ru, Os, Ir, in particular Ru, Os,
Ar is a capping molecule having a C₆ or C₅ aromatic ring substituted with one or more, preferably one to six or one to five C₁-C₃ alkyl, respectively,
preferably Ar is a C₆ aromatic ring substituted with one or more, preferably one to six, preferably 1 to 3, preferably 2, C₁-C₃ alkyl, and Me is Ru or Os,
X is a halogen selected from the group consisting of Cl and Br,
Q is O, S or CH₂, preferably O,
Het₁ is a 5 membered aromatic N-heterocycle preferably comprising at least one further N,
optionally comprising at least one, preferably one further heteroatom selected from O, S, N,
Het₂ is a 6 membered aromatic N-heterocycle
R₁, R₂, and R₃ are selected from H, C₁-C₁₀ alkyl, C₂-C₁₀ alkenyl, C₂-C₁₀ alkynyl, C₂-C₁₀ alkyl-carbonyl, C₃-C₁₀ alkenyl-carbonyl, C₃-C₁₀ alkynyl-carbonyl, C₆-C₁₀ aryl, C₇-C₁₅ aralkyl, C₈-C₁₅ aralkyl-carbonyl, C₇-C₁₁ aryl-carbonyl, in particular C₇-C₁₁ aryl-carbonyl, wherein at least one, preferably at least two of R₁, R₂, and R₃ is different from H,
wherein the alkyl, alkenyl and aralkyl groups may be of branched or straight chain and
wherein any aryl group may be unsubstituted or substituted with one or more C₁-C₆ alkyl, C₂-C₆ alkenyl, C₂-C₆ alkynyl, wherein the number of carbon atoms in R₁, R₂ or R₃ is not more than 15,
wherein at least one or preferably at least two of R₁, R₂, and R₃ is selected from C₄-C₁₀ alkyl, C₄-C₁₀ alkenyl, C₄-C₁₀ alkynyl, C₅-C₁₀ alkyl-carbonyl, C₅-C₁₀ alkenyl-carbonyl, C₅-C₁₀ alkynyl-carbonyl, C₆-C₁₀ aryl, C₇-C₁₅ aralkyl, C₈-C₁₅ aralkyl-carbonyl, C₇-C₁₁ aryl-carbonyl,
Z is selected from H, C₁-C₁₀ alkyl, C₂-C₁₀ alkenyl, C₂-C₁₀ alkynyl, —CH₂OR₄, or OR₄, wherein R₄ is selected from H, C₁-C₁₀ alkyl, C₂-C₁₀ alkenyl, C₂-C₁₀ alkynyl, C₂-C₁₀ alkyl-carbonyl, C₃-C₁₀ alkenyl-carbonyl, C₃-C₁₀ alkynyl-carbonyl, C₆-C₁₀ aryl, C₇-C₁₅ aralkyl, C₈-C₁₅ aralkyl-carbonyl, C₇-C₁₁ aryl-carbonyl, preferably R₄ is different from H,
wherein the alkyl, alkenyl and aralkyl groups in Z may be of branched or straight chain and
wherein any aryl group may be unsubstituted or substituted with one or more C₁-C₆ alkyl, C₂-C₆ alkenyl, C₂-C₆ alkynyl, wherein the number of carbon atoms in R₄ is not more than 15,

4. The compound of claim 1 having general formula (II) 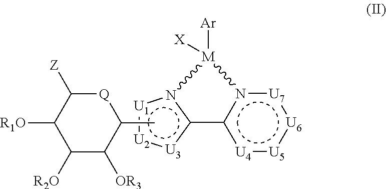
wherein
M is a metal ion selected from the group consisting of Ru, Os, Rh and Ir, Ru, Os, and Ir, preferably Ru and Os,
Ar is a capping molecule having a C₆ or C₅ aromatic ring substituted with one or more, preferably one to six or one to five C₁-C₃ alkyl,
preferably
Ar is a capping molecule having a C₆ aromatic ring substituted with one or more, preferably one to six or one to five C₁-C₃ alkyl, provided that Me is Ru(II) or Os(II)
Ar is a capping molecule having a C₅ aromatic ring substituted with one or more, preferably one to five C₁-C₃ alkyl, provided that Me is Rh(III) or Ir(III)
X is a halogen selected from the group consisting of Cl and Br,
Q, is as defined in claim 1, preferably Q is O or CH₂, more preferably O,
U₁, U₂, and U₃ are selected from N, NH, O, S, C and CH, wherein they, together with the rest of the Het₁ ring moiety form an aromatic five-membered N-heterocycle,
U₄, U₅, U₆ and U₇ are selected from N, C and CH, or any of the pairs selected from the group consisting of U₄ and
U₅; U₅ and U₆ as well as U₆ and U₇ is part of a fused benzene ring, in particular a quinoline,
R₁, R₂ and R₃ are as defined in claim 1, preferably at least two of R₁, R₂, and R₃ is selected from
C₄-C₁₀ alkyl, C₄-C₁₀ alkenyl, C₄-C₁₀ alkynyl, C₅-C₁₀ alkyl-carbonyl, C₅-C₁₀ alkenyl-carbonyl, C₅-C₁₀ alkynyl-carbonyl, C₆-C₁₀ aryl, C₇-C₁₅ aralkyl, C₈-C₁₅ aralkyl-carbonyl, C₇-C₁₁ aryl-carbonyl, and the aryl groups are substituted or unsubstituted such that wherein in said formula R₁, R₂, and R₃ are each a C₇-C₁₁ aryl-carbonyl (aroyl),
more preferably each of R₁, R₂ and R₃ is, independently, selected from
C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl (including alkylaryl), C₈-C₁₁ aralkyl-carbonyl, C₇-C₁₁ aryl-carbonyl wherein any aryl group may be unsubstituted or substituted with one or more C₁-C₄ alkyl, C₂-C₄ alkenyl, C₂-C₄ alkynyl, wherein the number of carbon atoms in R₁, R₂ or R₃ is not more than 12,
in particular a C₇-C₁₁ aryl-carbonyl,
Z is as defined in claim 1, preferably Z is selected from H, C₁-C₁₀ alkyl, C₁-C₁₀ alkenyl, C₂-C₁₀ alkynyl, —CH₂OR₄, or OR₄ wherein R₄ is a group as defined herein for any of R₁, R₂ and R₃,
wherein more preferably Z is selected from H, C₁-C₈ alkyl, C₂-C₈ alkenyl, C₂-C₈ alkynyl, —CH₂OR₄, wherein R₄ is selected from C₁-C₈ alkyl, C₂-C₈ alkenyl, C₂-C₈ alkynyl, C₂-C₈ alkyl-carbonyl, C₃-C₈ alkenyl-carbonyl, C₃-C₈ alkynyl-carbonyl, C₆-C₁₀ aryl, C₇-C₁₅ aralkyl, C₈-C₁₅ aralkyl-carbonyl, C₇-C₁₁ aryl-carbonyl,
in particular a C₇-C₁₁ aryl-carbonyl,
more preferably R₄ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl (including alkylaryl), C₈-C₁₁ aralkyl-carbonyl, C₇-C₁₁ aryl-carbonyl, in particular a C₇-C₁₁ aryl-carbonyl, wherein any aryl group may be unsubstituted or substituted with one or more C₁-C₄ alkyl, C₂-C₄ alkenyl, C₂-C₄ alkynyl, wherein the number of carbon atoms in R₄ is not more than 12.

5. The compound of claim 3, said compound having general formula (II.1) 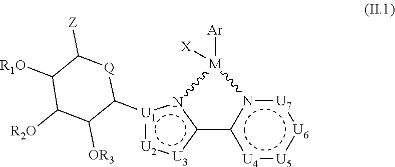
wherein M, Ar, X, Q, U₁, U₂, U₃, U₄, U₅, U₆ and U₇, R₁, R₂ and R₃ and Z are as defined in claim 3,
preferably
M is a metal ion selected from the group consisting of Ru, Os, Ir and Rh, in particular Ru, Os, and Ir in particular wherein a neoplastic disease is treated, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
U₁ is C or N and U₂ and U₃ are selected from N, NH, O, C and CH, wherein they, together with the rest of the Het₁ ring moiety form an aromatic five-membered N-heterocycle, more preferably U₁ is C, U₂ is CH or N and U₃ is O or NH, respectively,
U₄, U₅, U₆ and U₇ are selected from N, NH, O, C and CH, wherein U₄, U₅, U₆ and U₇ together with the rest of the Het₂ ring moiety form an aromatic six-membered N-heterocycle, more preferably each of U₄, U₅, U₆ and U₇ are CH,
R₁, R₂ and R₃ are, selected from H, C₁-C₂₀ alkyl, C₂-C₂₀ alkenyl, C₂-C₂₀ alkynyl, C₂-C₂₀ alkyl-carbonyl, C₃-C₂₀ alkenyl-carbonyl, C₃-C₂₀ alkynyl-carbonyl, C₆-C₁₀ aryl, C₇-C₂₀ aralkyl, C₈-C₂₀ aralkyl-carbonyl, C₇-C₁₁ aryl-carbonyl (aroyl), wherein at least one, preferably at least two of R₁, R₂, and R₃ is different from H,
wherein the alkyl, alkenyl and aralkyl groups may be of branched or straight chain, unless specifically defined differently and
wherein any aryl group may be unsubstituted or substituted with one or more C₁-C₁₀ alkyl, C₂-C₁₀ alkenyl, C₂-C₁₀ alkynyl, wherein the number of carbon atoms in R₁, R₂ or R₃ is not more than 20 wherein at least two of R₁, R₂, and R₃ is selected from C₄-C₈ alkyl, C₄-C₈ alkenyl, C₄-C₈ alkynyl, C₄-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, wherein any aryl group may be unsubstituted or substituted with one or more C₁-C₄ alkyl, C₂-C₄ alkenyl, C₂-C₄ alkynyl, wherein the number of carbon atoms in R₁, R₂ or R₃ is not more than 12.
Z is selected from H, C₁-C₈ alkyl, C₂-C₈ alkenyl, C₂-C₈ alkynyl, —CH₂OR₄, or OR₄, wherein R₄ is selected from C₁-C₈ alkyl, C₂-C₈ alkenyl, C₂-C₈ alkynyl, C₂-C₈ alkyl-carbonyl, C₃-C₈ alkenyl-carbonyl, C₃-C₈ alkynyl-carbonyl, C₆-C₁₀ aryl, C₇-C₁₅ aralkyl, C₈-C₁₅ aralkyl-carbonyl, C₇-C₁₁ aryl-carbonyl,
preferably R₄ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl
wherein any aryl group may be unsubstituted or substituted with one or more C₁-C₄ alkyl, C₂-C₄ alkenyl, C₂-C₄ alkynyl, wherein the number of carbon atoms in R₄ is not more than 12.

6. The compound of claim 3, said compound having general formula (II.2) 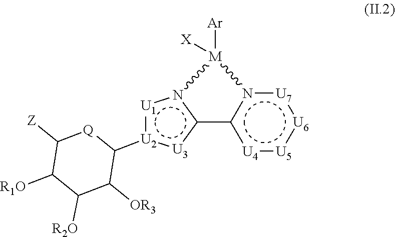
wherein M, Ar, X, Q, U₁, U₂, U₃, U₄, U₅, U₆ and U₇, R₁, R₂ and R₃ and Z are as defined in claim 3,
preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
U₂ is C or N and U₁, and U₃ are selected from N, NH, O, C and CH, wherein they, together with the rest of the Het₁ ring moiety form an aromatic five-membered N-heterocycle, more preferably U₁ is N and U₃ is O or CH,
U₄, U₅, U₆ and U₇ are selected from N, NH, O, C and CH, wherein U₄, U₅, U₆ and U₇ together with the rest of the Het₂ ring moiety form an aromatic six-membered N-heterocycle, more preferably each of U₄, U₅, U₆ and U₇ are CH,
R₁, R₂ and R₃ are as defined in claim 1, wherein at least two of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl, wherein preferably each aryl group is unsubstituted,
Z is selected from H, C₁-C₈ alkyl, C₂-C₈ alkenyl, C₂-C₈ alkynyl, —CH₂OR₄, or OR₄, wherein R₄ is selected from C₁-C₈ alkyl, C₂-C₈ alkenyl, C₂-C₈ alkynyl, C₂-C₈ alkyl-carbonyl, C₃-C₈ alkenyl-carbonyl, C₃-C₈ alkynyl-carbonyl, C₆-C₁₀ aryl, C₇-C₁₅ aralkyl, C₈-C₁₅ aralkyl-carbonyl, C₇-C₁₁ aryl-carbonyl, wherein preferably each aryl group is unsubstituted,
preferably R₄ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl.

7. The compound of claim 3, said compound having formula (III.1) 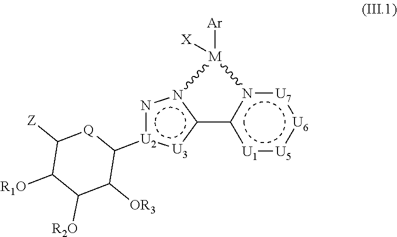
wherein M, Ar, X, Q, U₂, U₃, R₁, R₂ and R₃ and Z are as defined in claim 3,
preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
U₂ is C or N and U₃ is O or NH, provided that U₂ is C, and U₃ is CH or N provided that U₂ is N,
R₁, R₂ and R₃ are as defined in claim 3, wherein preferably each of R₁, R₂, and R₃ is selected from C₄-C₈ alkyl, C₄-C₈ alkenyl, C₄-C₈ alkynyl, C₅-C₈ alkyl-carbonyl, C₅-C₈ alkenyl-carbonyl, C₅-C₈ alkynyl-carbonyl, C₆ aryl, C₇-C₁₀ aralkyl, C₈-C₁₁ aralkyl-carbonyl, C₇-C₁₁ aryl-carbonyl,
more preferably each of R₁, R₂ and R₃ are selected from C₄-C₈ alkyl, C₅-C₈ alkyl-carbonyl, C₆ aryl, C₇-C₁₀ aralkyl, C₈-C₁₁ aralkyl-carbonyl, C₇-C₁₁ aryl-carbonyl,
Z is selected from H, C₁-C₈ alkyl, C₂-C₈ alkenyl, C₂-C₈ alkynyl, or —CH₂OR₄, wherein R₄ is selected from any group as defined for R₁, R₂, and R₃
wherein preferably each aryl group is unsubstituted.

8. The compound of claim 6, said compound having formula (IV) 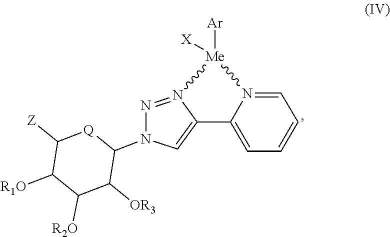
wherein M, Ar, X, Q, R₁, R₂ and R₃ and Z are as defined in claim 6,
preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
R₁, R₂ and R₃ are as defined in claim 6, wherein preferably each of R₁, R₂, and R₃ is selected from C₄-C₈ alkyl, C₅-C₈ alkyl-carbonyl, C₅-C₈ alkenyl-carbonyl, C₅-C₈ alkynyl-carbonyl, C₈-C₁₁ aralkyl-carbonyl, C₇-C₁₁ aryl-carbonyl,
more preferably each of R₁, R₂ and R₃ are selected from C₄-C₈ alkyl, C₅-C₈ alkyl-carbonyl, C₈-C₁₁ aralkyl-carbonyl, C₇-C₁₁ aryl-carbonyl,
Z is selected from H, C₁-C₈ alkyl, C₂-C₈ alkenyl, C₂-C₈ alkynyl, —CH₂OR₄, or OR₄, wherein R₄ is selected from any group as defined for R₁, R₂, and R₃

9. The compound of claim 7, said compound having a formula selected from formula (IV.1) and (IV.2) 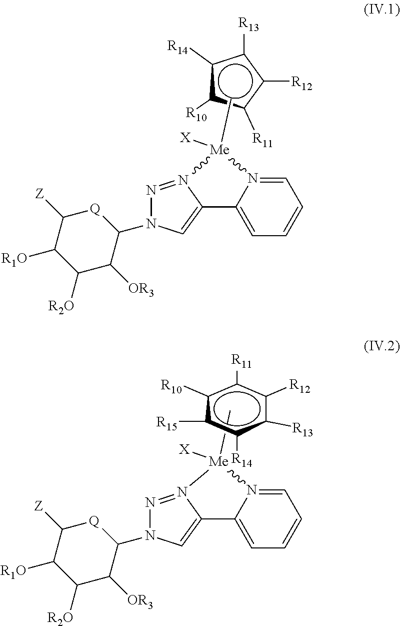
wherein in formula (IV.1)
X, Q, R₁, R₂ and R₃ and Z are as defined in claim 7,
Me is Ir or Rh, and
R₁₀, R₁₁, R₁₂, R₁₃, and R₁₄ are selected from, independently, H and C₁-C₃ alkyl groups, preferably C₁-C₂ alkyl groups, preferably each of R₁₀, R₁₁, R₁₂, R₁₃, and R₁₄ being identical, preferably methyl,
preferably having the formula of (IV. 1.1) 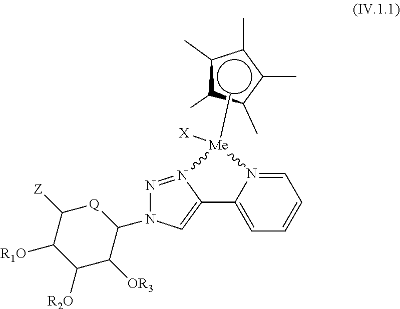
wherein in formula (IV.1.1)
X, Q, R₁, R₂ and R₃ and Z are as defined in claim 7,
preferably
X is Cl,
Q is O,
each of R₁, R₂, and R₃ is selected from C₄-C₈ alkyl, C₅-C₈ alkyl-carbonyl, C₈-C₁₁ aralkyl-carbonyl, C₇-C₁₁ preferably C7 aryl-carbonyl,
Z is selected from H, C₁-C₆ alkyl, C₂-C₆ alkenyl, C₂-C₆ alkynyl, —CH₂OR₄, or OR₄, wherein R₄ is selected from any group as defined for R₁, R₂, and R₃.
M is Ir or Rh,
OR
wherein in formula (IV.2)
X, Q, R₁, R₂ and R₃ and Z are as defined in claim 7,
M is Ru or Os, and
R₁₀, R₁₁, R₁₂, R₁₃, R₁₄ and R₁₅ are selected from, independently, H and C₁-C₃ alkyl groups, preferably at least two of R₁₀, R₁₁, R₁₂, R₁₃, R₁₄ and R₁₅, preferably two, being selected from C₁-C₃ alkyl groups, preferably from methyl and isopropyl, highly preferably in a para position,
preferably having the formula of (IV.2.1) 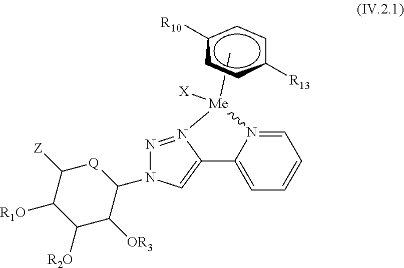
wherein in formula (IV.2.1)
X, Q, R₁, R₂ and R₃ and Z are as defined in claim 7,
preferably
X is Cl,
Q is O,
each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
Z is selected from H, C₁-C₆ alkyl, C₂-C₆ alkenyl, C₂-C₆ alkynyl, —CH₂OR₄, or OR₄, wherein R₄ is selected from any group as defined for R₁, R₂, and R₃
M is Ru or Os, and
R₁₀, and R₁₃ are selected from C₁-C₃ alkyl, preferably methyl and isopropyl wherein each aryl group is unsubstituted.

10. The compound of claim 6, said compound having formula (V) 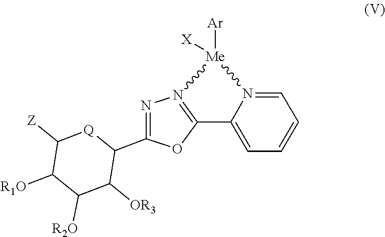
wherein Me, Ar, X, Q, R₁, R₂ and R₃ and Z are as defined in claim 6, preferably
M is a metal ion selected from the group consisting of Ru, Os, Ir and Rh, in particular Ru, Os, and Ir, preferably Ru and Os,
Ar is a capping molecule having a C₆ or C₅ aromatic ring substituted with two to six or five, respectively, preferably two or five, respectively, C₁-C₃ alkyl groups,
X is a halogen selected from the group consisting of Cl and Br, preferably Cl
Q, is O or CH₂, preferably O,
R₁, R₂ and R₃ are as defined in claim 6, wherein preferably each of R₁, R₂, and R₃ is selected from C₄-C₈ alkyl, alkyl-carbonyl, C₅-C₈ alkenyl-carbonyl, C₅-C₈ alkynyl-carbonyl, C₈-C₁₁ aralkyl-carbonyl, C₇-C₁₁ aryl-carbonyl,
more preferably each of R₁, R₂ and R₃ are selected from C₄-C₈ alkyl, C₅-C₈ alkyl-carbonyl, C₈-C₁₁ aralkyl-carbonyl, C₇-C₁₁ aryl-carbonyl,
Z is selected from H, C₁-C₈ alkyl, C₂-C₈ alkenyl, C₂-C₈ alkynyl, —CH₂OR₄, or OR₄, wherein R₄ is selected from any group as defined for R₁, R₂, and R₃.

11. The compound of claim 9, said compound having a formula selected from formula (V.1) and (V.2) 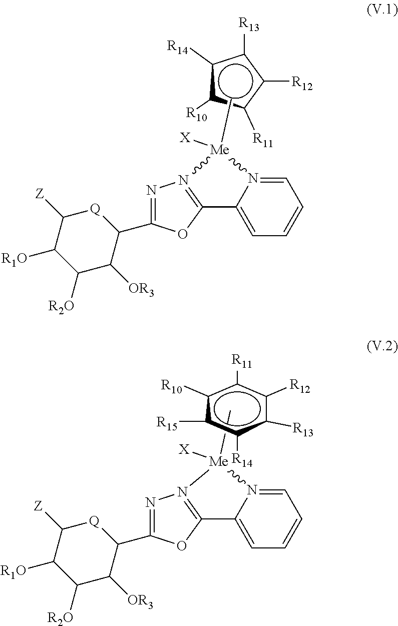
wherein in formula (V.1)
X, Q, R₁, R₂ and R₃ and Z are as defined in claim 9,
Me is Ir or Rh, and
R₁₀, R₁₁, R₁₂, R₁₃, and R₁₄ are selected from, independently, H and C₁-C₃ alkyl groups, preferably C₁-C₂ alkyl groups, preferably each of R₁₀, R₁₁, R₁₂, R₁₃, and R₁₄ being identical, preferably methyl,
preferably having the formula of (V.1.1) 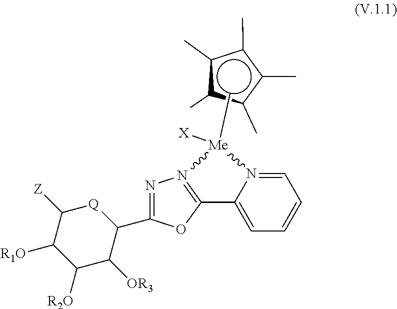
wherein in formula (V.1.1)
X, Q, R₁, R₂ and R₃ and Z are as defined in claim 9,
preferably
X is Cl,
Q is O,
each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
Z is selected from H, C₁-C₆ alkyl, C₂-C₆ alkenyl, C₂-C₆ alkynyl, —CH₂OR₄, or OR₄, wherein R₄ is as defined in this claims for any of R₁, R₂, and R₃
M is Ir or Rh,
OR
wherein in formula (V.2)
X, Q, R₁, R₂ and R₃ and Z are as defined in claim 9,
M is Ru or Os, and
R₁₀, R₁₁, R₁₂, R₁₃, R₁₄ and R₁₅ are selected from, independently, H and C₁-C₃ alkyl groups, preferably at least two of R₁₀, R₁₁, R₁₂, R₁₃, R₁₄ and R₁₅, preferably two, being selected from C₁-C₃ alkyl groups, preferably from methyl and isopropyl, highly preferably in a para position,
preferably having the formula of (V.2.1) 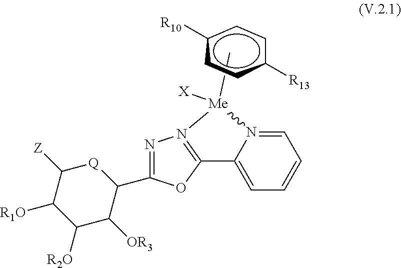
wherein in formula (V.2.1)
X, Q, R₁, R₂ and R₃ and Z are
such that
X is Cl,
Q is O,
each of R₁, R₂, and R₃ is selected from C₄-C₈ alkyl, C₅-C₈ alkyl-carbonyl, C₈-C₁₁ aralkyl-carbonyl, C₇-C₁₁ preferably C₇ aryl-carbonyl,
Z is selected from H, C₁-C₆ alkyl, C₂-C₆ alkenyl, C₂-C₆ alkynyl, —CH₂OR₄, or OR₄, wherein R₄ is selected from any group as defined for R₁, R₂, and R₃.
M is Ru or Os, and
R₁₀, and R₁₃ are selected from C₁-C₃ alkyl, preferably methyl and isopropyl,
wherein each aryl group is unsubstituted.

12. The compound of claim 1, wherein in the formulae the 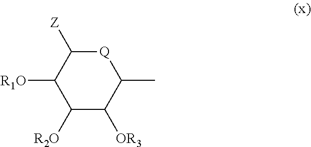
moiety is a glycopyranosyl ring O-substituted with R₁, R₂, R₃ and R₄ as defined in claim 1, respectively.

13. The compound of claim 1, said compound having a distribution coefficient (log D) of at least 1.4, preferably 1.8, if measured between n-octanol saturated with aqueous PBS solution (pH=7.40), and aqueous PBS solution.

14. The compound of claim 1 wherein said compound is selected from the group consisting of a complex of a transition metal ion selected from the group consisting of Ru, Os, Rh, Ir, Pd and Pt, preferably selected from Ru, Os, Rh, Ir with any of the following ligands:
1-(2′,3′,4′,6′-Tetra-O-benzoyl-β-D-glucopyranosyl)-4-(pyridin-2-yl)-1,2,3-triazole,
2-(2′,3′,4′,6′-Tetra-O-benzoyl-β-D-glucopyranosyl)-5-(pyridin-2-yl)-1,3,4-oxadiazole,
2-(2′,3′,4′-Tri-O-benzoyl-β-D-xylopyranosyl)-5-(pyridin-2-yl)-1,3,4-oxadiazole,
2-(2′,3′,4′,6′-Tetra-O-benzoyl-β-D-galactopyranosyl)-5-(pyridin-2-yl)-1,3,4-oxadiazole,
with a halogen selected from the group consisting of Cl, Br and I, preferably Cl and Br, and
with a substituted or unsubstituted penta- or hexahapto coordinating cap molecule having at least one 5 or 6 membered aromatic ring.

15. A medicament in human or veterinary medicine comprising a compound of claim 1.

16. An anti-neoplastic agent comprising a compound of claim 1 for delivery a subject having a neoplasm or a patient endangered by development of a neoplasm or recurrence of a neoplasm wherein preferably the subject is a mammalian subject.

17. The anti-neoplastic agent of claim 16, wherein the neoplasm is selected from a carcinoma, a sarcoma a haematological neoplasm, preferably the neoplasm is selected from an ovarian cancer, a breast cancer, a skin cancer, a pancraetic cancer, a glioblastoma and adenocarcinoma.

18. A method of treating a patient that has a pathogenic bacterial infection comprising administering to said patient an effective amount of the medicament of claim 15.

19. The method of claim 18, wherein the pathogenic bacteria are Gram-positive bacteria.

20. The method of claim 19, wherein the Gram-positive bacteria are antibiotic resistant, preferably methicillin-resistant or vancomycin resistant, more preferably a methicillin-resistant Staphylococcus or a vancomycin-resistant Enterococcus.

21. (canceled)

22. (canceled)

23. A pharmaceutical preparation comprising a compound according to claim 1 comprising a pharmaceutically acceptable carrier.

24. An antibacterial composition comprising a compound according to claim 1 and a carrier.
